SEQUENCE LISTING (21 sequences)

SEQ ID NO: 1 - DNA, 15 bp, synthetic construct
  misc_feature 1..15: note = Synthetic: 15 mer oligo
  modified_base 1: mod_base = OTHER; note = 5 -monophosphate
  modified_base 14: mod_base = OTHER; note = dTAm = 2'-deoxy-5-(N-(6-aminohexyl)prop-2-enamide) thymidine
  modified_base 15: mod_base = OTHER; note = ddC = 2',3'-dideoxy Cytidine
  source 1..15: mol_type = other DNA
aaaaaaaaaaaaatc

SEQ ID NO: 2 - DNA, 39 bp, synthetic construct
  misc_feature 1..39: note = Synthetic: 39 mer oligo
  source 1..39: mol_type = other DNA
aaaaaaaaaaaaaaaaaaaaaaaaaaaaaaaaaaaaaaa

SEQ ID NO: 3-19 - skipped (17 entries)

SEQ ID NO: 20 - RNA, 1921 nt, synthetic construct
  misc_feature 1..1921: note = Synthetic: FLuc mRNA
  modified_base 7: mod_base = OTHER; note = 5-methoxy uridine
  modified_base 25: mod_base = OTHER; note = 5-methoxy uridine
  modified_base 35: mod_base = OTHER; note = 5-methoxy uridine
  modified_base 37: mod_base = OTHER; note = 5-methoxy uridine
  modified_base 49: mod_base = OTHER; note = 5-methoxy uridine
  modified_base 67: mod_base = OTHER; note = 5-methoxy uridine
  modified_base 87..88: mod_base = OTHER; note = 5-methoxy uridine
  modified_base 90: mod_base = OTHER; note = 5-methoxy uridine
  modified_base 97: mod_base = OTHER; note = 5-methoxy uridine
  modified_base 124: mod_base = OTHER; note = 5-methoxy uridine
  modified_base 136: mod_base = OTHER; note = 5-methoxy uridine
  modified_base 144: mod_base = OTHER; note = 5-methoxy uridine
  modified_base 151: mod_base = OTHER; note = 5-methoxy uridine
  modified_base 154: mod_base = OTHER; note = 5-methoxy uridine
  modified_base 166: mod_base = OTHER; note = 5-methoxy uridine
  modified_base 171..172: mod_base = OTHER; note = 5-methoxy uridine
  modified_base 187: mod_base = OTHER; note = 5-methoxy uridine
  modified_base 193: mod_base = OTHER; note = 5-methoxy uridine
  modified_base 199: mod_base = OTHER; note = 5-methoxy uridine
  modified_base 204: mod_base = OTHER; note = 5-methoxy uridine
  modified_base 213: mod_base = OTHER; note = 5-methoxy uridine
  modified_base 216..217: mod_base = OTHER; note = 5-methoxy uridine
  modified_base 223: mod_base = OTHER; note = 5-methoxy uridine
  modified_base 229: mod_base = OTHER; note = 5-methoxy uridine
  modified_base 235: mod_base = OTHER; note = 5-methoxy uridine
  modified_base 247: mod_base = OTHER; note = 5-methoxy uridine
  modified_base 255: mod_base = OTHER; note = 5-methoxy uridine
  modified_base 262: mod_base = OTHER; note = 5-methoxy uridine
  modified_base 280: mod_base = OTHER; note = 5-methoxy uridine
  modified_base 283: mod_base = OTHER; note = 5-methoxy uridine
  modified_base 286: mod_base = OTHER; note = 5-methoxy uridine
  modified_base 288: mod_base = OTHER; note = 5-methoxy uridine
  modified_base 304: mod_base = OTHER; note = 5-methoxy uridine
  modified_base 309..310: mod_base = OTHER; note = 5-methoxy uridine
  modified_base 312..313: mod_base = OTHER; note = 5-methoxy uridine
  modified_base 316: mod_base = OTHER; note = 5-methoxy uridine
  modified_base 322: mod_base = OTHER; note = 5-methoxy uridine
  modified_base 325: mod_base = OTHER; note = 5-methoxy uridine
  modified_base 334: mod_base = OTHER; note = 5-methoxy uridine
  modified_base 336..337: mod_base = OTHER; note = 5-methoxy uridine
  modified_base 340: mod_base = OTHER; note = 5-methoxy uridine
  modified_base 346: mod_base = OTHER; note = 5-methoxy uridine
  modified_base 352: mod_base = OTHER; note = 5-methoxy uridine
  modified_base 370: mod_base = OTHER; note = 5-methoxy uridine
  modified_base 372: mod_base = OTHER; note = 5-methoxy uridine
  modified_base 388: mod_base = OTHER; note = 5-methoxy uridine
  modified_base 391: mod_base = OTHER; note = 5-methoxy uridine
  modified_base 400: mod_base = OTHER; note = 5-methoxy uridine
  modified_base 406: mod_base = OTHER; note = 5-methoxy uridine
  modified_base 421: mod_base = OTHER; note = 5-methoxy uridine
  modified_base 424: mod_base = OTHER; note = 5-methoxy uridine
  modified_base 426..427: mod_base = OTHER; note = 5-methoxy uridine
  modified_base 430: mod_base = OTHER; note = 5-methoxy uridine
  modified_base 445: mod_base = OTHER; note = 5-methoxy uridine
  modified_base 454: mod_base = OTHER; note = 5-methoxy uridine
  modified_base 457: mod_base = OTHER; note = 5-methoxy uridine
  modified_base 463: mod_base = OTHER; note = 5-methoxy uridine
  modified_base 475: mod_base = OTHER; note = 5-methoxy uridine
  modified_base 481: mod_base = OTHER; note = 5-methoxy uridine
  modified_base 484: mod_base = OTHER; note = 5-methoxy uridine
  modified_base 493: mod_base = OTHER; note = 5-methoxy uridine
  modified_base 496: mod_base = OTHER; note = 5-methoxy uridine
  modified_base 499: mod_base = OTHER; note = 5-methoxy uridine
  modified_base 502: mod_base = OTHER; note = 5-methoxy uridine
  modified_base 519: mod_base = OTHER; note = 5-methoxy uridine
  modified_base 528..529: mod_base = OTHER; note = 5-methoxy uridine
  modified_base 538: mod_base = OTHER; note = 5-methoxy uridine
  modified_base 540: mod_base = OTHER; note = 5-methoxy uridine
  modified_base 546..547: mod_base = OTHER; note = 5-methoxy uridine
  modified_base 550: mod_base = OTHER; note = 5-methoxy uridine
  modified_base 562: mod_base = OTHER; note = 5-methoxy uridine
  modified_base 573..574: mod_base = OTHER; note = 5-methoxy uridine
  modified_base 582: mod_base = OTHER; note = 5-methoxy uridine
  modified_base 588..589: mod_base = OTHER; note = 5-methoxy uridine
  modified_base 592: mod_base = OTHER; note = 5-methoxy uridine
  modified_base 603..604: mod_base = OTHER; note = 5-methoxy uridine
  modified_base 622: mod_base = OTHER; note = 5-methoxy uridine
  modified_base 628: mod_base = OTHER; note = 5-methoxy uridine
  modified_base 631: mod_base = OTHER; note = 5-methoxy uridine
  modified_base 634: mod_base = OTHER; note = 5-methoxy uridine
  modified_base 658: mod_base = OTHER; note = 5-methoxy uridine
  modified_base 670: mod_base = OTHER; note = 5-methoxy uridine
  modified_base 676: mod_base = OTHER; note = 5-methoxy uridine
  modified_base 693: mod_base = OTHER; note = 5-methoxy uridine
  modified_base 697: mod_base = OTHER; note = 5-methoxy uridine
  modified_base 702..703: mod_base = OTHER; note = 5-methoxy uridine
  modified_base 724: mod_base = OTHER; note = 5-methoxy uridine
  modified_base 726..727: mod_base = OTHER; note = 5-methoxy uridine
  modified_base 739: mod_base = OTHER; note = 5-methoxy uridine
  modified_base 742: mod_base = OTHER; note = 5-methoxy uridine
  modified_base 757: mod_base = OTHER; note = 5-methoxy uridine
  modified_base 760: mod_base = OTHER; note = 5-methoxy uridine
  modified_base 766: mod_base = OTHER; note = 5-methoxy uridine
  modified_base 769: mod_base = OTHER; note = 5-methoxy uridine
  modified_base 774..775: mod_base = OTHER; note = 5-methoxy uridine
  modified_base 786..787: mod_base = OTHER; note = 5-methoxy uridine
  modified_base 793: mod_base = OTHER; note = 5-methoxy uridine
  modified_base 795..796: mod_base = OTHER; note = 5-methoxy uridine
  modified_base 805: mod_base = OTHER; note = 5-methoxy uridine
  modified_base 810: mod_base = OTHER; note = 5-methoxy uridine
  modified_base 814: mod_base = OTHER; note = 5-methoxy uridine
  modified_base 817: mod_base = OTHER; note = 5-methoxy uridine
  modified_base 819: mod_base = OTHER; note = 5-methoxy uridine
  modified_base 825..826: mod_base = OTHER; note = 5-methoxy uridine
  modified_base 832: mod_base = OTHER; note = 5-methoxy uridine
  modified_base 835: mod_base = OTHER; note = 5-methoxy uridine
  modified_base 838: mod_base = OTHER; note = 5-methoxy uridine
  modified_base 841: mod_base = OTHER; note = 5-methoxy uridine
  modified_base 843: mod_base = OTHER; note = 5-methoxy uridine
  modified_base 849..850: mod_base = OTHER; note = 5-methoxy uridine
  modified_base 862: mod_base = OTHER; note = 5-methoxy uridine
  modified_base 864..865: mod_base = OTHER; note = 5-methoxy uridine
  modified_base 868: mod_base = OTHER; note = 5-methoxy uridine
  modified_base 877: mod_base = OTHER; note = 5-methoxy uridine
  modified_base 885: mod_base = OTHER; note = 5-methoxy uridine
  modified_base 892: mod_base = OTHER; note = 5-methoxy uridine
  modified_base 904: mod_base = OTHER; note = 5-methoxy uridine
  modified_base 907: mod_base = OTHER; note = 5-methoxy uridine
  modified_base 910: mod_base = OTHER; note = 5-methoxy uridine
  modified_base 919: mod_base = OTHER; note = 5-methoxy uridine
  modified_base 921..922: mod_base = OTHER; note = 5-methoxy uridine
  modified_base 927..928: mod_base = OTHER; note = 5-methoxy uridine
  modified_base 930..931: mod_base = OTHER; note = 5-methoxy uridine
  modified_base 946: mod_base = OTHER; note = 5-methoxy uridine
  modified_base 949: mod_base = OTHER; note = 5-methoxy uridine
  modified_base 957: mod_base = OTHER; note = 5-methoxy uridine
  modified_base 964: mod_base = OTHER; note = 5-methoxy uridine
  modified_base 973: mod_base = OTHER; note = 5-methoxy uridine
  modified_base 982: mod_base = OTHER; note = 5-methoxy uridine
  modified_base 1003: mod_base = OTHER; note = 5-methoxy uridine
  modified_base 1015: mod_base = OTHER; note = 5-methoxy uridine
  modified_base 1027: mod_base = OTHER; note = 5-methoxy uridine
  modified_base 1038..1039: mod_base = OTHER; note = 5-methoxy uridine
  modified_base 1045: mod_base = OTHER; note = 5-methoxy uridine
  modified_base 1054: mod_base = OTHER; note = 5-methoxy uridine
  modified_base 1065: mod_base = OTHER; note = 5-methoxy uridine
  modified_base 1072: mod_base = OTHER; note = 5-methoxy uridine
  modified_base 1093: mod_base = OTHER; note = 5-methoxy uridine
  modified_base 1096: mod_base = OTHER; note = 5-methoxy uridine
  modified_base 1099: mod_base = OTHER; note = 5-methoxy uridine
  modified_base 1132: mod_base = OTHER; note = 5-methoxy uridine
  modified_base 1141: mod_base = OTHER; note = 5-methoxy uridine
  modified_base 1144: mod_base = OTHER; note = 5-methoxy uridine
  modified_base 1149..1150: mod_base = OTHER; note = 5-methoxy uridine
  modified_base 1152..1153: mod_base = OTHER; note = 5-methoxy uridine
  modified_base 1165: mod_base = OTHER; note = 5-methoxy uridine
  modified_base 1168: mod_base = OTHER; note = 5-methoxy uridine
  modified_base 1174: mod_base = OTHER; note = 5-methoxy uridine
  modified_base 1192: mod_base = OTHER; note = 5-methoxy uridine
  modified_base 1198: mod_base = OTHER; note = 5-methoxy uridine
  modified_base 1216: mod_base = OTHER; note = 5-methoxy uridine
  modified_base 1218: mod_base = OTHER; note = 5-methoxy uridine
  modified_base 1222: mod_base = OTHER; note = 5-methoxy uridine
  modified_base 1234: mod_base = OTHER; note = 5-methoxy uridine
  modified_base 1237: mod_base = OTHER; note = 5-methoxy uridine
  modified_base 1240: mod_base = OTHER; note = 5-methoxy uridine
  modified_base 1248: mod_base = OTHER; note = 5-methoxy uridine
  modified_base 1252: mod_base = OTHER; note = 5-methoxy uridine
  modified_base 1279: mod_base = OTHER; note = 5-methoxy uridine
  modified_base 1282: mod_base = OTHER; note = 5-methoxy uridine
  modified_base 1296: mod_base = OTHER; note = 5-methoxy uridine
  modified_base 1300: mod_base = OTHER; note = 5-methoxy uridine
  modified_base 1315: mod_base = OTHER; note = 5-methoxy uridine
  modified_base 1320: mod_base = OTHER; note = 5-methoxy uridine
  modified_base 1323: mod_base = OTHER; note = 5-methoxy uridine
  modified_base 1341..1342: mod_base = OTHER; note = 5-methoxy uridine
  modified_base 1344..1345: mod_base = OTHER; note = 5-methoxy uridine
  modified_base 1348: mod_base = OTHER; note = 5-methoxy uridine
  modified_base 1351: mod_base = OTHER; note = 5-methoxy uridine
  modified_base 1360: mod_base = OTHER; note = 5-methoxy uridine
  modified_base 1369: mod_base = OTHER; note = 5-methoxy uridine
  modified_base 1372: mod_base = OTHER; note = 5-methoxy uridine
  modified_base 1377: mod_base = OTHER; note = 5-methoxy uridine
  modified_base 1386: mod_base = OTHER; note = 5-methoxy uridine
  modified_base 1393: mod_base = OTHER; note = 5-methoxy uridine
  modified_base 1408: mod_base = OTHER; note = 5-methoxy uridine
  modified_base 1417: mod_base = OTHER; note = 5-methoxy uridine
  modified_base 1420: mod_base = OTHER; note = 5-methoxy uridine
  modified_base 1423: mod_base = OTHER; note = 5-methoxy uridine
  modified_base 1438: mod_base = OTHER; note = 5-methoxy uridine
  modified_base 1440..1441: mod_base = OTHER; note = 5-methoxy uridine
  modified_base 1453: mod_base = OTHER; note = 5-methoxy uridine
  modified_base 1462: mod_base = OTHER; note = 5-methoxy uridine
  modified_base 1486: mod_base = OTHER; note = 5-methoxy uridine
  modified_base 1498: mod_base = OTHER; note = 5-methoxy uridine
  modified_base 1501: mod_base = OTHER; note = 5-methoxy uridine
  modified_base 1504: mod_base = OTHER; note = 5-methoxy uridine
  modified_base 1507: mod_base = OTHER; note = 5-methoxy uridine
  modified_base 1525: mod_base = OTHER; note = 5-methoxy uridine
  modified_base 1540: mod_base = OTHER; note = 5-methoxy uridine
  modified_base 1543: mod_base = OTHER; note = 5-methoxy uridine
  modified_base 1548: mod_base = OTHER; note = 5-methoxy uridine
  modified_base 1552: mod_base = OTHER; note = 5-methoxy uridine
  modified_base 1564: mod_base = OTHER; note = 5-methoxy uridine
  modified_base 1582: mod_base = OTHER; note = 5-methoxy uridine
  modified_base 1594: mod_base = OTHER; note = 5-methoxy uridine
  modified_base 1597: mod_base = OTHER; note = 5-methoxy uridine
  modified_base 1599..1600: mod_base = OTHER; note = 5-methoxy uridine
  modified_base 1603: mod_base = OTHER; note = 5-methoxy uridine
  modified_base 1612: mod_base = OTHER; note = 5-methoxy uridine
  modified_base 1624: mod_base = OTHER; note = 5-methoxy uridine
  modified_base 1636: mod_base = OTHER; note = 5-methoxy uridine
  modified_base 1651: mod_base = OTHER; note = 5-methoxy uridine
  modified_base 1660: mod_base = OTHER; note = 5-methoxy uridine
  modified_base 1663: mod_base = OTHER; note = 5-methoxy uridine
  modified_base 1666: mod_base = OTHER; note = 5-methoxy uridine
  modified_base 1690: mod_base = OTHER; note = 5-methoxy uridine
  modified_base 1696: mod_base = OTHER; note = 5-methoxy uridine
  modified_base 1698: mod_base = OTHER; note = 5-methoxy uridine
  modified_base 1701..1702: mod_base = OTHER; note = 5-methoxy uridine
  modified_base 1705..1706: mod_base = OTHER; note = 5-methoxy uridine
  modified_base 1711: mod_base = OTHER; note = 5-methoxy uridine
  modified_base 1715..1716: mod_base = OTHER; note = 5-methoxy uridine
  modified_base 1718: mod_base = OTHER; note = 5-methoxy uridine
  modified_base 1726..1727: mod_base = OTHER; note = 5-methoxy uridine
  modified_base 1731..1732: mod_base = OTHER; note = 5-methoxy uridine
  modified_base 1734: mod_base = OTHER; note = 5-methoxy uridine
  modified_base 1740: mod_base = OTHER; note = 5-methoxy uridine
  modified_base 1745..1746: mod_base = OTHER; note = 5-methoxy uridine
  modified_base 1748..1749: mod_base = OTHER; note = 5-methoxy uridine
  modified_base 1751: mod_base = OTHER; note = 5-methoxy uridine
  modified_base 1753: mod_base = OTHER; note = 5-methoxy uridine
  modified_base 1757..1758: mod_base = OTHER; note = 5-methoxy uridine
  modified_base 1764: mod_base = OTHER; note = 5-methoxy uridine
  modified_base 1766: mod_base = OTHER; note = 5-methoxy uridine
  modified_base 1770: mod_base = OTHER; note = 5-methoxy uridine
  modified_base 1772..1773: mod_base = OTHER; note = 5-methoxy uridine
  modified_base 1776: mod_base = OTHER; note = 5-methoxy uridine
  modified_base 1778..1780: mod_base = OTHER; note = 5-methoxy uridine
  modified_base 1784: mod_base = OTHER; note = 5-methoxy uridine
  modified_base 1791: mod_base = OTHER; note = 5-methoxy uridine
  modified_base 1795: mod_base = OTHER; note = 5-methoxy uridine
  source 1..1921: mol_type = other RNA
aggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgattaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 21 - RNA, 996 nt, synthetic construct
  misc_feature 1..996: note = Synthetic: eGFP mRNA
  modified_base 1: mod_base = OTHER; note = N6-methyl-adenine (m6A)
  modified_base 7: mod_base = OTHER; note = N1-methyl pseudouridine
  modified_base 25: mod_base = OTHER; note = N1-methyl pseudouridine
  modified_base 35: mod_base = OTHER; note = N1-methyl pseudouridine
  modified_base 37: mod_base = OTHER; note = N1-methyl pseudouridine
  modified_base 49: mod_base = OTHER; note = N1-methyl pseudouridine
  modified_base 52: mod_base = OTHER; note = N1-methyl pseudouridine
  modified_base 70: mod_base = OTHER; note = N1-methyl pseudouridine
  modified_base 72..73: mod_base = OTHER; note = N1-methyl pseudouridine
  modified_base 82: mod_base = OTHER; note = N1-methyl pseudouridine
  modified_base 85: mod_base = OTHER; note = N1-methyl pseudouridine
  modified_base 91: mod_base = OTHER; note = N1-methyl pseudouridine
  modified_base 94: mod_base = OTHER; note = N1-methyl pseudouridine
  modified_base 97: mod_base = OTHER; note = N1-methyl pseudouridine
  modified_base 103: mod_base = OTHER; note = N1-methyl pseudouridine
  modified_base 115: mod_base = OTHER; note = N1-methyl pseudouridine
  modified_base 129..130: mod_base = OTHER; note = N1-methyl pseudouridine
  modified_base 136: mod_base = OTHER; note = N1-methyl pseudouridine
  modified_base 138: mod_base = OTHER; note = N1-methyl pseudouridine
  modified_base 158: mod_base = OTHER; note = N1-methyl pseudouridine
  modified_base 165: mod_base = OTHER; note = N1-methyl pseudouridine
  modified_base 175: mod_base = OTHER; note = N1-methyl pseudouridine
  modified_base 181: mod_base = OTHER; note = N1-methyl pseudouridine
  modified_base 186..187: mod_base = OTHER; note = N1-methyl pseudouridine
  modified_base 190: mod_base = OTHER; note = N1-methyl pseudouridine
  modified_base 192: mod_base = OTHER; note = N1-methyl pseudouridine
  modified_base 208: mod_base = OTHER; note = N1-methyl pseudouridine
  modified_base 214: mod_base = OTHER; note = N1-methyl pseudouridine
  modified_base 219: mod_base = OTHER; note = N1-methyl pseudouridine
  modified_base 229: mod_base = OTHER; note = N1-methyl pseudouridine
  modified_base 232: mod_base = OTHER; note = N1-methyl pseudouridine
  modified_base 241: mod_base = OTHER; note = N1-methyl pseudouridine
  modified_base 246: mod_base = OTHER; note = N1-methyl pseudouridine
  modified_base 253: mod_base = OTHER; note = N1-methyl pseudouridine
  modified_base 258: mod_base = OTHER; note = N1-methyl pseudouridine
  modified_base 261..262: mod_base = OTHER; note = N1-methyl pseudouridine
  modified_base 270: mod_base = OTHER; note = N1-methyl pseudouridine
  modified_base 283: mod_base = OTHER; note = N1-methyl pseudouridine
  modified_base 297..298: mod_base = OTHER; note = N1-methyl pseudouridine
  modified_base 300..301: mod_base = OTHER; note = N1-methyl pseudouridine
  modified_base 306: mod_base = OTHER; note = N1-methyl pseudouridine
  modified_base 313: mod_base = OTHER; note = N1-methyl pseudouridine
  modified_base 324: mod_base = OTHER; note = N1-methyl pseudouridine
  modified_base 328: mod_base = OTHER; note = N1-methyl pseudouridine
  modified_base 343: mod_base = OTHER; note = N1-methyl pseudouridine
  modified_base 345..346: mod_base = OTHER; note = N1-methyl pseudouridine
  modified_base 348..349: mod_base = OTHER; note = N1-methyl pseudouridine
  modified_base 366: mod_base = OTHER; note = N1-methyl pseudouridine
  modified_base 385: mod_base = OTHER; note = N1-methyl pseudouridine
  modified_base 390..391: mod_base = OTHER; note = N1-methyl pseudouridine
  modified_base 406: mod_base = OTHER; note = N1-methyl pseudouridine
  modified_base 409: mod_base = OTHER; note = N1-methyl pseudouridine
  modified_base 418: mod_base = OTHER; note = N1-methyl pseudouridine
  modified_base 424: mod_base = OTHER; note = N1-methyl pseudouridine
  modified_base 433: mod_base = OTHER; note = N1-methyl pseudouridine
  modified_base 438..439: mod_base = OTHER; note = N1-methyl pseudouridine
  modified_base 457: mod_base = OTHER; note = N1-methyl pseudouridine
  modified_base 460: mod_base = OTHER; note = N1-methyl pseudouridine
  modified_base 472: mod_base = OTHER; note = N1-methyl pseudouridine
  modified_base 477: mod_base = OTHER; note = N1-methyl pseudouridine
  modified_base 483: mod_base = OTHER; note = N1-methyl pseudouridine
  modified_base 499: mod_base = OTHER; note = N1-methyl pseudouridine
  modified_base 501: mod_base = OTHER; note = N1-methyl pseudouridine
  modified_base 503: mod_base = OTHER; note = N1-methyl pseudouridine
  modified_base 505: mod_base = OTHER; note = N1-methyl pseudouridine
  modified_base 508: mod_base = OTHER; note = N1-methyl pseudouridine
  modified_base 532: mod_base = OTHER; note = N1-methyl pseudouridine
  modified_base 538: mod_base = OTHER; note = N1-methyl pseudouridine
  modified_base 543..544: mod_base = OTHER; note = N1-methyl pseudouridine
  modified_base 550: mod_base = OTHER; note = N1-methyl pseudouridine
  modified_base 562: mod_base = OTHER; note = N1-methyl pseudouridine
  modified_base 577: mod_base = OTHER; note = N1-methyl pseudouridine
  modified_base 583: mod_base = OTHER; note = N1-methyl pseudouridine
  modified_base 594: mod_base = OTHER; note = N1-methyl pseudouridine
  modified_base 613: mod_base = OTHER; note = N1-methyl pseudouridine
  modified_base 628: mod_base = OTHER; note = N1-methyl pseudouridine
  modified_base 631: mod_base = OTHER; note = N1-methyl pseudouridine
  modified_base 634: mod_base = OTHER; note = N1-methyl pseudouridine
  modified_base 648: mod_base = OTHER; note = N1-methyl pseudouridine
  modified_base 652: mod_base = OTHER; note = N1-methyl pseudouridine
  modified_base 663: mod_base = OTHER; note = N1-methyl pseudouridine
  modified_base 670: mod_base = OTHER; note = N1-methyl pseudouridine
  modified_base 698: mod_base = OTHER; note = N1-methyl pseudouridine
  modified_base 703: mod_base = OTHER; note = N1-methyl pseudouridine
  modified_base 706: mod_base = OTHER; note = N1-methyl pseudouridine
  modified_base 709: mod_base = OTHER; note = N1-methyl pseudouridine
  modified_base 712: mod_base = OTHER; note = N1-methyl pseudouridine
  modified_base 717..718: mod_base = OTHER; note = N1-methyl pseudouridine
  modified_base 721: mod_base = OTHER; note = N1-methyl pseudouridine
  modified_base 736: mod_base = OTHER; note = N1-methyl pseudouridine
  modified_base 740: mod_base = OTHER; note = N1-methyl pseudouridine
  modified_base 742: mod_base = OTHER; note = N1-methyl pseudouridine
  modified_base 748: mod_base = OTHER; note = N1-methyl pseudouridine
  modified_base 757: mod_base = OTHER; note = N1-methyl pseudouridine
  modified_base 759: mod_base = OTHER; note = N1-methyl pseudouridine
  modified_base 765: mod_base = OTHER; note = N1-methyl pseudouridine
  modified_base 776..777: mod_base = OTHER; note = N1-methyl pseudouridine
  modified_base 780..781: mod_base = OTHER; note = N1-methyl pseudouridine
  modified_base 786: mod_base = OTHER; note = N1-methyl pseudouridine
  modified_base 790..791: mod_base = OTHER; note = N1-methyl pseudouridine
  modified_base 793: mod_base = OTHER; note = N1-methyl pseudouridine
  modified_base 801..802: mod_base = OTHER; note = N1-methyl pseudouridine
  modified_base 806..807: mod_base = OTHER; note = N1-methyl pseudouridine
  modified_base 809: mod_base = OTHER; note = N1-methyl pseudouridine
  modified_base 815: mod_base = OTHER; note = N1-methyl pseudouridine
  modified_base 820..821: mod_base = OTHER; note = N1-methyl pseudouridine
  modified_base 823..824: mod_base = OTHER; note = N1-methyl pseudouridine
  modified_base 826: mod_base = OTHER; note = N1-methyl pseudouridine
  modified_base 828: mod_base = OTHER; note = N1-methyl pseudouridine
  modified_base 832..833: mod_base = OTHER; note = N1-methyl pseudouridine
  modified_base 839: mod_base = OTHER; note = N1-methyl pseudouridine
  modified_base 841: mod_base = OTHER; note = N1-methyl pseudouridine
  modified_base 845: mod_base = OTHER; note = N1-methyl pseudouridine
  modified_base 847..848: mod_base = OTHER; note = N1-methyl pseudouridine
  modified_base 851: mod_base = OTHER; note = N1-methyl pseudouridine
  modified_base 853..855: mod_base = OTHER; note = N1-methyl pseudouridine
  modified_base 859: mod_base = OTHER; note = N1-methyl pseudouridine
  modified_base 866: mod_base = OTHER; note = N1-methyl pseudouridine
  modified_base 870: mod_base = OTHER; note = N1-methyl pseudouridine
  source 1..996: mol_type = other RNA
aggaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0109977 A1
(43) Pub. Date: Apr. 23, 2026

(54) MODIFIED RNA FOR INCREASING PROTEIN EXPRESSION

(71) Applicant: TriLink BioTechnologies, LLC, San Diego, CA (US)

(72) Inventors: Chunping Xu, San Diego, CA (US); Chanfeng Zhao, Rancho Santa Fe, CA (US); Paul Theodore Ludford, III, San Diego, CA (US); Dhamodharan Venugopal, Monrovia, CA (US); Alexandre V. Lebedev, San Diego, CA (US); Hengyuan Lang, San Diego, CA (US)

(73) Assignee: TriLink BioTechnologies, LLC, San Diego, CA (US)

(21) Appl. No.: 19/386,605

(22) Filed: Nov. 12, 2025

Related U.S. Application Data
(63) Continuation of application No. PCT/US25/10301, filed on Jan. 3, 2025.
(60) Provisional application No. 63/617,664, filed on Jan. 4, 2024.

(51) Int. Cl. C12N 15/11; A61K 31/7115; A61K 31/7125; C12N 15/10

(52) U.S. Cl. CPC C12N 15/11; A61K 31/7115; A61K 31/7125; C12N 15/10; C12N 2310/3515; C12N 2310/531

(57) ABSTRACT

Described herein are modified RNA molecules where a 3′-stabilizing region is covalently attached to the RNA, and where the 3′-stabilizing region comprises one or more modified nucleosides. Methods of synthesizing said RNAs are also provided herein.

### CROSS-REFERENCE TO PRIORITY APPLICATION

This application is a continuation of International Application No. PCT/US2025/010301 filed Jan. 3, 2025, which claims priority to U.S. Provisional Application No. 63/617,664, filed Jan. 4, 2024, each of which are incorporated herein by reference in its entirety for any purpose.

### SEQUENCE LISTING

This application contains a Sequence Listing, which has been submitted electronically in XML format and is hereby incorporated by reference in its entirety. Said XML copy, created on Feb. 27, 2025, is named “01355-0004-00US_seq listing” and is 206,036 bytes in size.

### TECHNICAL FIELD

The field of this invention relates to modified RNA and methods of producing the same. Additionally, the invention relates to using said modified RNAs to efficiently produce protein.

### BACKGROUND

Eukaryotic mRNA has five important parts, which include the cap at the 5′-end, the 5′-untranslated region (5′-UTR), the open reading frame (ORF), the 3′-untranslated region (3′-UTR) and the 3′-tail consisting of 100-250 adenyl residues (poly-A-tail), the length of which varies in different cell types (Youn, H. and Chung, J. K., (2015) Expert Opin. Biol. Ther. 15:1337-1348).

While mRNA therapeutics are promising, they face concerns regarding their instability and high immunogenicity (Kormann et al., (2011) Nature Biotech. 29:154-157). As mRNAs naturally degrade in biological systems, high dose or repeated administration is commonly required. The main pathway of mRNA degradation in eukaryotic cells occurs in the cytoplasm within the ribonucleic complexes called P-bodies, which contain 5′-3′-exonucleases, decapping and deadenylating enzymes. Once the poly-A-tail is shortened to 12 residues or less, mRNA degradation occurs through cap cleavage and 5′→3′ or 3′→5′ cleavage (Melo et al., (2019) Mol. Ther. 27:2080-2090). Endonucleases may also be involved in mRNA degradation.

The use of chemical modifications in mRNA allowed for increasing its stability and improving translational properties and immunogenicity of mRNA (Anderson et al., (2010) Nucleic Acids Res. 38:5884-5892; Jemielity et al., (2010) New J Chem. 34:829-844; Kariko et al., (2012) Mol. Ther. 20:948-953; Sahin et al., (2014) Nat. Rev. Drug Discov. 13:759-780).

One of the structural elements that affect mRNA half-life and translation is the 5′ terminal 7-methylguanosine cap (Topisirovic et al., (2011) Interdiscip. Rev. RNA 2:277-298). Modifications of the 5′ cap may lead to augmented mRNA stability and expression in living cells (Ziemniak et al., (2013) Future Med. Chem. 5:1141-1172; Kowalska et al., (2014) Nucleic Acids Res. 42:10245-10264; WO2017/053297).

The poly-A-tail is another key element responsible for efficient translation and increased mRNA stability (Chang et al., (2014)Mol. Cell 53:1044-1052). The role of the poly-A-tail in translation consists of binding with numerous polyadenosyl-binding proteins (PABP), which in their turn bind with the eukaryotic translation initiation factor 4G (eIF4G). The ring structure with the cap-eIF4E-eIF4G-PABP— poly-A closed loop is formed, which facilitates ribosome binding and protects mRNA from nuclease degradation (Newbury, S. F., (2006) Biochem. Soc. Trans. 34:30-34).

Woolf et al. described modifications of the poly-A tail that increase stability against nucleases (WO 1999014346). They recognized that phosphorothioate linkages, or other stabilizing modifications of RNA, may be incorporated into a poly-A tail to add further stabilization to an mRNA molecule and that other modifications may be made downstream of the poly-A tail to retain the poly-A binding sites and further block 3′ exonucleases.

Despite recent clinical successes, mRNA therapeutics still face challenges of instability, toxicity, short-term efficacy, and potential immunological responses. Thus, increasing the stability of mRNAs to enhance their efficacy and reduce their immunogenicity in vivo remains an important problem that must be solved to increase the feasibility of mRNA therapeutics for clinical applications.

### BRIEF SUMMARY OF THE INVENTION

Described herein are RNA molecules covalently linked to a 3′-stabilizing region, where the 3′-stabilizing region comprises one or more purification handles which are covalently attached, optionally via a linker (L). While not wishing to be bound by theory, after purification of precursor RNA comprising a 5′-cap, an ORF encoding a protein, and a poly-A region 3′ to the ORF (the A in Formula I or II) on oligo dT column, only precursor RNA comprising at least a partial poly-A tail is linked with the 3′-stabilizing region comprising one or more purification handles. Thus, only full-length RNA molecules are stabilized with the 3′-stabilizing region and easily separated from the full length RNA molecules which were not linked to the 3′-stabilizing region. Surprisingly, the RNA molecules described herein, show improved stability and improved translation efficiency.

In an aspect, provided herein is an RNA molecule comprising the structure of Formula I: A-B (Formula I), wherein A comprises: a) a 5′-cap; b) an open reading frame (ORF) encoding a protein; and c) a poly-A region, wherein the poly-A region is 3′ to the open reading frame; and B comprises a 3′-stabilizing region comprising 1 to 50 nucleosides, wherein one or more nucleosides within the 3′-stabilizing region comprise one or more purification handles.

In an aspect, provided herein is an RNA molecule comprising the structure of Formula II: A-B-L (Formula II), wherein A comprises: a) a 5′-cap; b) an open reading frame (ORF) encoding a protein; and c) a poly-A region, wherein the poly-A region is 3′ to the open reading frame; and B comprises a 3′-stabilizing region comprising 1 to 50 nucleosides, wherein one or more nucleosides within the 3′-stabilizing region comprise one or more linkers (L), wherein the linker (L) is capable of binding a purification handle.

In embodiments, the 3′-stabilizing region is covalently linked to a precursor RNA by a linker that can be formed by ligation. In embodiments, the linker is a linker that can be formed by an enzymatic ligation or a chemical ligation. In embodiments, the 3′-stabilizing region is covalently linked to a precursor RNA by a linker that can be formed using a polymerase. In embodiments, the purification handle is linked to the 3′-stabilizing region via a linker (L).

In embodiments, one or more purification handles are covalently linked to one or more nucleosides or one or more linkers within the 3′-stabilizing region. In embodiments, the purification handle comprises a lipid. In embodiments, the 3′-stabilizing region forms a secondary structure. In embodiments, the secondary structure is a hairpin loop.

In embodiments, the 3′-stabilizing region comprises one or more unmodified nucleosides and one or more unmodified internucleotide linkages. In embodiments, the 3′-stabilizing region comprises one or more modified nucleosides and/or one or more modified internucleotide linkages. In embodiments, the modified nucleoside comprises a modified nucleobase and/or a modified sugar. In embodiments, the modified nucleoside comprises a modified nucleobase. In embodiments, the modified nucleobase is a modified uracil, a modified cytosine, a modified guanine, or a modified adenine. In embodiments, the modified nucleobase is pseudouracil (y), 2-thio-uracil, 4-thio-uracil, 4-thio-pseudouridine, 2-thio-pseudouridine, 5-hydroxy-uracil, 5-halo-uracil, 3-methyl-uracil, 5-aza-uracil, or 2-thio-5-aza-uracil. In embodiments, the modified nucleobase is 5-aza-cytosine, 6-aza-cytosine, pseudoisocytidine, 3-methyl-cytosine, 5-methyl-cytosine, 5-halo-cytosine, 2-thio-cytosine, or 2-thio-5-methyl-cytosine. In embodiments, the modified nucleobase is 2-amino-purine, 2,6-diaminopurine, 2-amino-6-halo-purine, 6-halo-purine, 2-amino-6-methyl-purine, 8-azido-adenine, 7-deaza-adenine, N6-methyl-adenine, or 2-methylthio-N6-methyl-adenine. In embodiments, the modified nucleobase is inosine, 1-methyl-inosine, 7-cyano-7-deaza-guanine, 7-aminomethyl-7-deaza-guanine, 6-thio-guanine, 6-thio-7-deaza-guanine, or 6-methoxy-guanine. In embodiments, the modified nucleoside comprises a modified sugar. In embodiments, the modified sugar has a 5-membered ring or a 6-membered ring, or is a modified ribose. In embodiments, the modified ribose is 2′-thioribose, 2′, 3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, or 3′-amino-2′,3′-dideoxyribose. In embodiments, the modified nucleoside comprises a morpholino ring. In embodiments, the internucleotide linkage comprises a modified phosphate. A modified phosphate has one or more modifications relative to an unmodified phosphate, such as a substitution replacing an oxygen with a different atom or group. In embodiments, the modified phosphate is phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate, methylphosphonate, or guanidinopropyl phosphoramidate. In embodiments, the last nucleoside of the 3′-stabilizing region does not comprise a 3′-hydroxyl. In embodiments, the last nucleoside of the 3′-stabilizing region is ddC, inverted dT, 3′-phosphate nucleoside, 3′-oxime nucleoside, 3′-azidomethyl nucleoside, or 3′-methyl nucleoside.

In embodiments, the poly-A region is 10 or greater nucleosides in length. In embodiments, the poly-A region is 30 or greater nucleosides in length. In embodiments, the poly-A region is 70 or greater nucleosides in length. In embodiments, the poly-A region is 100 or greater nucleosides in length. In embodiments, the poly-A region is from 2 to 500 nucleosides in length. In embodiments, the poly-A region is from 5 to 250 nucleosides in length. In embodiments, the poly-A region is from 10 to 200 nucleosides in length. In embodiments, the poly-A region is from 15 to 150 nucleosides in length. In embodiments, the RNA molecule is a messenger RNA (mRNA).

In an aspect, provided herein is a cell comprising any one of the RNA molecules described herein. In embodiments, the cell is an isolated cell. In an aspect, provided herein is a cell comprising a protein or a peptide translated from any of the RNA molecules described herein.

In an aspect, provided herein is a pharmaceutical composition comprising any of the RNA molecules described herein and a pharmaceutically acceptable carrier. In embodiments, the pharmaceutically acceptable carrier is a solvent, dispersion media, diluent, surface active agent, isotonic agent, thickening or emulsifying agent, lipid, liposome, nanoparticle, lipid nanoparticle (LNP), polymer, lipoplex, protein, or a mixture thereof. In embodiments, the pharmaceutically acceptable carrier is an LNP. In an aspect, provided herein is a pharmaceutical composition comprising a cell comprising any one of the RNA molecules described herein.

In an aspect, provided herein is a method of increasing the expression of a protein or a peptide of interest in a cell, comprising contacting the cell with any one of the RNA molecules described herein, wherein the RNA molecule encodes the protein or peptide of interest, wherein the expression is increased when compared to that of an RNA molecule without the 3′-stabilizing region. In embodiments, the cell is isolated, in vitro, or ex vivo. In an aspect, provided herein is a method of expressing a protein or a peptide of interest in a cell, comprising contacting the cell with any one of the RNA molecules described herein, wherein the RNA molecule encodes the protein or peptide of interest and the cell translates the protein or peptide of interest from the RNA molecule. In embodiments, the cell is isolated, in vitro, or ex vivo. In an aspect, provided herein is a method of increasing the half-life of an RNA molecule in a cell comprising contacting the cell with any one of the RNA molecules described herein, wherein the RNA molecule encodes the protein or peptide of interest and the cell translates the protein or peptide of interest from the RNA molecule. In embodiments, the cell is isolated, in vitro, or ex vivo.

In an aspect, provided herein is a method of preparing any one of the RNA molecules described herein, comprising covalently joining a stabilizing region to a precursor RNA comprising a 5′-cap, an ORF encoding a protein, and a poly-A region 3′ of the ORF, wherein the stabilizing region is added 3′ to the poly-A region and the stabilizing region comprises one or more purification handles and/or one or more linkers capable of binding a purification handle. In embodiments, the 3′-stabilizing region is joined by ligation. In embodiments, the 3′-stabilizing region is joined by chemical or enzymatic ligation. In embodiments, the 3′-stabilizing region is joined using a polymerase.

In an aspect, provided herein is a method of treating a disease in a subject in need thereof comprising introducing an effective amount of any one of the RNA molecules, cells, or pharmaceutical compositions described herein. In an aspect, provided herein is a method of preventing a disease in a subject in need thereof comprising introducing an effective amount of any one of the RNA molecules, cells, or pharmaceutical compositions described herein.

In an aspect, provided herein is a compound of Formula (III) or (IV):

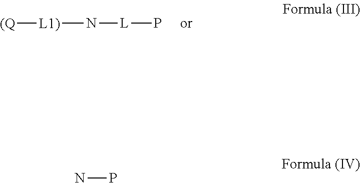

wherein N is a nucleoside;
L is a linker capable of binding a purification handle;
P is a purification handle;
Q-L1 is optionally present, wherein L1 is a linker covalently bound to N and to Q;
and
Q is a hydrogen or a chain terminating nucleoside.

In embodiments, the linker capable of binding the purification handle is linked to the nucleoside via a 3′-carbon or a 2′-carbon of a sugar of the nucleoside. In embodiments, the linker capable of binding the purification handle is linked to the nucleoside via a nucleobase of the nucleoside and L1 linker is linked to the nucleoside via a 3′-carbon or a 2′-carbon of a sugar of the nucleoside. In embodiments, the purification handle is linked to the nucleoside via a 3′-carbon or a 2′-carbon of a sugar of the nucleoside. In embodiments, the purification handle is linked to the nucleoside via a nucleobase of the nucleoside.

In an aspect, provided herein is a method of increasing the expression of a protein or a peptide of interest in a cell, comprising contacting the cell with an RNA molecule comprising any one of the compounds of Formula (III) or Formula (IV) described herein, wherein the RNA molecule encodes the protein or peptide of interest, wherein the expression is increased when compared to that of an RNA molecule not comprising the compound of Formula (III) or Formula (IV). In embodiments, the cell is isolated, in vitro, or ex vivo. In an aspect, provided herein is a method of increasing the half-life of an RNA molecule in a cell comprising contacting the cell with the RNA molecule comprising any one of the compounds of Formula (III) or Formula (IV) described herein, wherein the half-life is increased when compared to that of an RNA molecule not comprising the compound of Formula (III) or Formula (IV). In embodiments, the cell is isolated, in vitro, or ex vivo.

### BRIEF DESCRIPTION OF THE DRAWINGS

FIGS. 1A-B show HPLC traces of (A) 15-mer oligo (SEQ ID NO:1) and (B) Sequence 1a which forms after reaction of SEQ ID NO:1 with 2,5-dioxopyrrolidin-1-yl hexanoate.

FIGS. 2A-C show HPLC traces of (A) 39-mer oligo (SEQ ID NO:2); (B) Sequence 2a which forms after ligation of SEQ ID NO:2 with compound 1; and (C) Sequence 2b which forms after ligation of SEQ ID NO:2 with compound 2.

FIGS. 3A-D show HPLC traces of (A) 15-mer oligo (SEQ ID NO:1); (B) 39-mer oligo (SEQ ID NO:2); (C) Sequence 2c which forms after ligation of SEQ ID NO:1 with SEQ ID NO:2, also unreacted SEQ ID NO:1; (D) Sequence 2d which forms after ligation of Sequence 1a with SEQ ID NO:2, also unreacted SEQ ID NO:2 and Sequence 1a.

FIGS. 4A-C show HPLC traces of (A) Firefly luciferase (FLuc) mRNA; (B) Sequence 3 which forms after ligation of FLuc mRNA with compound 1, and unreacted FLuc mRNA is also detected in the same peak; (C) Sequence 5 which forms after ligation of FLuc mRNA with compound 2, and unreacted FLuc mRNA.

FIGS. 5A-C show HPLC traces of (A) Firefly luciferase (FLuc) mRNA; (B) Sequence 4 which forms after ligation of FLuc mRNA with SEQ ID NO:1, and unreacted FLuc mRNA is also detected in the same peak; (C) Sequence 6 which forms after ligation of FLuc mRNA with Sequence 1a, and unreacted FLuc mRNA.

FIGS. 6A-C show HPLC traces of (A) Firefly luciferase (FLuc) mRNA; (B) Sequence 3 which forms after ligation of FLuc mRNA with compound 1, and unreacted FLuc mRNA is also detected in the same peak; (C) Sequence 7 which forms after ligation of FLuc mRNA with compound 4, and unreacted FLuc mRNA.

FIGS. 7A-C show HPLC traces of (A) Firefly luciferase (FLuc) mRNA; (B) Sequence 4 which forms after ligation of FLuc mRNA with SEQ ID NO:1, and unreacted FLuc mRNA is also detected in the same peak; (C) Sequence 8 which forms after ligation of FLuc mRNA with Sequence 1b, and unreacted FLuc mRNA.

FIGS. 8A-C show HPLC traces of (A) Firefly luciferase (FLuc) mRNA; (B) Sequence 3 which forms after ligation of FLuc mRNA with compound 1, and unreacted FLuc mRNA is also detected in the same peak; (C) Sequence 9 which forms after ligation of FLuc mRNA with compound 6, and unreacted FLuc mRNA.

FIGS. 9A-C show HPLC traces of (A) Firefly luciferase (FLuc) mRNA; (B) Sequence 4 which forms after ligation of FLuc mRNA with SEQ ID NO:1, and unreacted FLuc mRNA is also detected in the same peak; (C) Sequence 10 which forms after ligation of FLuc mRNA with Sequence 1c, and unreacted FLuc mRNA.

FIGS. 10A-B shows HPLC traces of (A) enhanced green fluorescent protein (eGFP) mRNA (SEQ ID NO:21) and (B) eGFP mRNA with a tail modification (Sequence 23).

FIG. 11 shows HPLC trace of a co-injection of eGFP mRNA (SEQ ID NO:21) and eGFP mRNA with a tail modification (Sequence 12).

FIG. 12 shows translation of eGFP encoding mRNA, made with various tail modifications, in 293T cells as a function of time (10ng/well of mRNA).

FIG. 13 shows total protein expression 96 hours post transfection of eGFP encoding mRNA, made with various tail modifications, in T-293 cells (10ng/well of mRNA).

FIG. 14 shows translation of eGFP encoding mRNA, made with various tail modifications, in A549 cells as a function of time (10 ng/well of mRNA).

FIG. 15 shows total protein expression 96 hours post transfection of eGFP encoding mRNA, made with various tail modifications, in A549 cells (10 ng/well of mRNA).

### DETAILED DESCRIPTION OF THE INVENTION

### Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”. For example, reference to “a transcript” may include a plurality of transcripts.

As used herein “or” is used in the inclusive sense, i.e., equivalent to “and/or”, unless the context clearly indicates otherwise.

As used herein “or a mixture thereof” means any combination of the recited components including any amounts of each and in any combination. The components can be present individually or in combination with each other (at any ratio). For example, when stated that a material is composed of substances A, B, C, or a mixture thereof, it means that the material can consist of either A alone, B alone, C alone, or a combination (mixture) of A and B, A and C, B and C, or all A, B, and C.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present as well as instances where it does not occur or is not present.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The abbreviations used herein have their conventional meaning within the chemical and biological arts. The chemical structures and formulae set forth herein are constructed according to the standard rules of chemical valency known in the chemical arts.

Ranges include the endpoints of the range. For example, “between 0 and 2” includes 0, 1, 2, and (unless the context requires otherwise) fractional values greater than 0 and less than 2.

Where substituent groups are specified by their conventional chemical formulae, written from left to right, they equally encompass the chemically identical substituents that would result from writing the structure from right to left, e.g., —CH₂O— is equivalent to —OCH₂-.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂-. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂- and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, P, and S where the ring is saturated or unsaturated, but not aromatic. The 3 or 4 membered ring contains 1 heteroatom selected from the group consisting of O, N, P, and S. The 5 membered ring can contain zero or one double bond and one, two or three heteroatoms selected from the group consisting of O, N, P, and S. The 6 or 7 membered ring contains zero, one or two double bonds and one, two or three heteroatoms selected from the group consisting of O, N, P, and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent ma optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

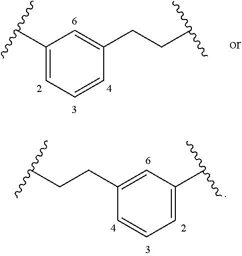 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, =0, =NR′, =N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, −halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T—C(O)-(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRRψ)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo,
   halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

The term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganiccounter ions well known in the art and when the molecule contains a basic functionality, salts of organic or inorganic acids. The pharmaceutically acceptable salts of the compounds described herein are suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. Salts may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1; and Remington: The Science and Practice of Pharmacy, 23d Edition, Adejare et al. eds., Academic Press (2020); which are incorporated herein by reference in their entireties.)

As used herein, the term “cap analog” means a structural derivative of the natural RNA cap. “natural 5′-cap” refers to a cap structure found on the 5′-end of an mRNA molecule and generally consists of a guanosine 5′-triphosphate (Gppp) which is connected via its triphosphate moiety to the 5′-end of the next nucleotide of the mRNA (i.e., the guanosine is connected via a 5′ to 5′ triphosphate linkage to the rest of the mRNA). The guanosine may be methylated at position N⁷ (resulting in the cap structure m⁷Gppp). Cap analogs include those described in International Patent Publications Nos. WO2017/053297, WO2023/147352, WO2021/162566, WO2021/162567, WO2022/006368, WO2022/086140, WO2023/033551, WO2018/075827, WO2023/07019, and in U.S. Provisional Applications Nos. 63/528,990, and 63/536,844, the cap structures of each of which are incorporated herein by reference. In embodiments, the term 5′-cap as used herein refers to a cap analog as described herein or to any moiety with the biological function of a cap.

As used herein, the term “complement,” “complementary,” or “complementarity” refers to specific base pairing between nucleotides or nucleic acids. Complementary nucleotides are, generally, A and T (or A and U), and G and C. Complementarity, for example, between a capped oligonucleotide primer and a DNA template, may be “complete” or “total” where all of the nucleotide bases of two nucleic acid strands are matched according to recognized base pairing rules, it may be “partial” in which only some of the nucleotide bases of an initiating capped oligonucleotide primer and a DNA template are matched according to recognized base pairing rules, or it may be “absent” where none of the nucleotide bases of two nucleic acid strands are matched according to recognized base pairing rules. Complementarity can also be “substantial complementarity” where the nucleotide bases of two nucleic acids are matched according to recognized base pairing rules, but include one or more mismatches (e.g., 1, 2, 3, 4) from total complementarity.

As used herein, a “deoxyribonuclease” (abbreviated as “DNase”) is an enzyme that catalyzes the hydrolytic cleavage of phosphodiester linkages in the DNA backbone, thus degrading DNA.

As used herein, the term “impurities” refers to substances which differ from the chemical composition of the target material (e.g., mRNA transcripts). Impurities are also referred to as contaminants.

“Inorganic pyrophosphatase” refers to an enzyme that catalyzes the conversion of one ion of pyrophosphate to two phosphate ions, thus inhibiting aggregation and in some instances preventing interaction of pyrophosphate with magnesium ions during T7 transcription reactions.

As used herein, the term “in vitro” refers to a process that takes place outside a living organism (e.g., a multi-cellular organism, such as a human or a non-human animal), for example, in a test tube, culture dish, or elsewhere outside a living organism.

As used herein, the term “in vivo” refers to events that occur within a living organism.

As used herein the term “in vivo assays” refer to methods used to detect and/or measure capacity of one or more of the compounds or molecules including the compounds (e.g., mRNA molecules in, for example, a therapeutic dose) to increase or decrease a property relative to a control (e.g., biomarker levels). Optionally, in vivo assays as described herein can be used to determine a subject's tolerability levels to a given compound or molecule. Exemplary measurements for assessing tolerability include one or more of body weight, organ weight, aspartate aminotransferase (AST) levels, alanine transaminase (ALT) levels, C-reactive protein (CRP) levels, procalcitonin (PCT) levels, interleukin-6 (IL-6) levels, erythrocyte sedimentation rate (ESR), serum amyloid A levels, and serum ferritin levels.

As used herein, “locked nucleic acid” (LNA) ring means a ribonucleotide having a bridge between the 2′O and 4′C methylene bicyclonucleotide monomers. An LNA moiety can have the following structure:

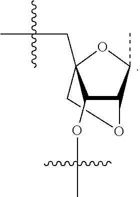

As used herein, “unlocked nucleic acid” (UNA) ring means a ribonucleotide comprising an acyclic ring, where the bond between 2′C and 3′C is absent. An UNA moiety can have the following structure:

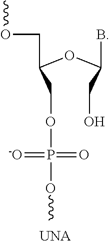

As used herein, “messenger RNA transcript,” or “mRNA transcript,” is a transcript transcribed from a DNA template encoding a desired polypeptide. The mRNA transcript may contain coding and non-coding regions. For example, the DNA template can comprise an RNA polymerase promoter sequence, a 5′ UTR sequence, an open reading frame, and a 3′ UTR sequence. In some examples, the DNA template also comprises a nucleic acid sequence encoding a poly-A tail. In embodiments, the DNA template can comprise a 5′ UTR sequence, an open reading frame, and a 3′ UTR sequence.

As used herein, the term “nucleoside” refers to a nitrogenous base linked to a 5-carbon sugar (e.g., ribose or deoxyribose). The term includes all nucleosides, including all forms of nucleoside bases and furanoses. There are five natural unmodified nucleosides: adenosine (A), Guanosine (G), Cytidine (C), Thymidine (T), and Uridine (U). According to Aduri et al (Aduri, R. et al., AMBER force field parameters for the naturally occurring modified nucleotides in RNA. Journal of Chemical Theory and Computation. 2006. 3(4):1464-75) there are 107 naturally occurring modified nucleosides, including 1-methyladenosine, 2-methylthio-N6-hydroxynorvalyl carbamoyladenosine, 2-methyladenosine, 2-O-ribosylphosphate adenosine, N6-methyl-N6-threonylcarbamoyladenosine, N6-acetyladenosine, N6-glycinylcarbamoyladenosine, N6-isopentenyladenosine, N6-methyladenosine, N6-threonylcarbamoyladenosine, N6,N6-dimethyladenosine, N6-(cis-hydroxyisopentenyl)adenosine, N6-hydroxynorvalylcarbamoyladenosine, 1,2-O-dimethyladenosine, N6,2-O-dimethyladenosine, 2-O-methyladenosine, N6,N6,O-2-trimethyladenosine, 2-methylthio-N6-(cis-hydroxyisopentenyl) adenosine, 2-methylthio-N6-methyladenosine, 2-methylthio-N6-isopentenyladenosine, 2-methylthio-N6-threonyl carbamoyladenosine, 2-thiocytidine, 3-methylcytidine, N4-acetylcytidine, 5-formylcytidine, N4-methylcytidine, 5-methylcytidine, 5-hydroxymethylcytidine, lysidine, N4-acetyl-2-O-methylcytidine, 5-formyl-2-O-methylcytidine, 5,2-O-dimethylcytidine, 2-O-methylcytidine, N4,2-O-dimethylcytidine, N4,N4,2-O-trimethylcytidine, 1-methylguanosine, N2,7-dimethylguanosine, N2-methylguanosine, 2-O-ribosylphosphate guanosine, 7-methylguanosine, under modified hydroxywybutosine, 7-aminomethyl-7-deazaguanosine, 7-cyano-7-deazaguanosine, N2,N2-dimethylguanosine, 4-demethylwyosine, epoxyqueuosine, hydroxywybutosine, isowyosine, N2,7,2-O-trimethylguanosine, N2,2-O-dimethylguanosine, 1,2-O-dimethylguanosine, 2-O-methylguanosine, N2,N2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine, 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine, 5-(isopentenylaminomethyl)uridine, 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl)pseudouridine, 1-methylpseudouridine, 3-methylpseudouridine, 2-O-methylpseudouridine, inosine, 1-methylinosine, 1,2-O-dimethylinosine and 2-O-methylinosine. Each of these or the modified nucleobase thereof may be components of nucleic acids of the present invention.

All other nucleosides (not including the ones described above as natural nucleosides or modified natural nucleosides) are unnatural nucleosides.

As used herein, the term “nucleoside base” refers to a nitrogenous base. A “natural nucleoside base” includes purine and pyrimidine rings. Purine rings include, for example, adenine and guanine. Pyrimidine rings include, for example, cytosine, thymine, and uracil.

As used herein, the term “modified nucleoside base” describes natural modified nucleoside bases, including but is not limited to, for example, pseudouracil, 5-methylcytosine, N6-methyladenine, inosine, 5-hydroxymethylcytosine, 5-carboxylcytosine, N4-acetylcytosine, N4-methylcytosine, n1-methyladenine, N2, N2-dimethylguanine and the like. Also, see naturally occurring modified nucleosides above.

As used herein, the term “unnatural nucleoside base” refers to all nucleoside bases that are not natural (whether modified or not; see naturally occurring modified nucleosides above) including but is not limited to, for example, 7-deazaadenine, 2-aminoadenine, 5-methylisocytosine, 5-fluorouracil, 5-bromouracil, 5-iodouracil, 2-thiouracil, 2-methylthioadenine, 2-thio-5-methyluracil, 2-amino-6-methylthiopurine and the like. Natural nucleosides are described above,

As used herein, the terms “nucleoside analogs,” “modified nucleosides,” or “nucleoside derivatives” include synthetic nucleosides as described herein. Nucleoside derivatives also include nucleosides having modified base or/and sugar moieties, with or without protecting groups and include, for example, 2′-deoxy-2′-fluorouridine, 5-fluorouridine and the like. The compounds and methods provided herein include such base rings and synthetic analogs thereof, as well as unnatural heterocycle-substituted base sugars, and acyclic substituted base sugars. Other nucleoside derivatives that may be utilized with the present disclosure include, for example, LNA nucleosides, halogen-substituted purines (e.g., 6-fluoropurine), halogen-substituted pyrimidines, N⁶-ethyladenine, N⁴-(alkyl)-cytosines, 5-ethylcytosine, and the like (U.S. Pat. No. 6,762,298).

As used herein, the term “nucleoside triphosphate,” “nucleoside 5′ triphosphate” or “NTP” refers to a nucleoside linked to three phosphate groups. The term encompasses natural NTPs (for example, adenosine triphosphate (ATP), uridine triphosphate (UTP), guanine triphosphate (GTP), and cytosine triphosphate (CTP)) as well as modified NTPs.

As used herein, the term “modified NTP” refers to a nucleoside 5′-triphosphate having a chemical moiety group bound at any position or substituted at any position, including the sugar, base, triphosphate chain, or any combination of these three locations. Optionally, the chemical moiety group may be a group of any nature compatible with the process of transcription. Examples of such NTPs include inosine triphosphate, dihydrouridine triphosphate, 2′-fluoro-2′-deoxycytidine triphosphate, pseudouridine triphosphate, N1-methylpseudouridine triphosphate, and 5-methyluridine triphosphate, and can be found, for example in “Nucleoside Triphosphates and Their Analogs: Chemistry, Biotechnology and Biological Applications,” Vaghefi, M., ed., Taylor and Francis, Boca Raton (2005).

As used herein, the term “modified RNA” or “modified mRNA” includes, for example, an RNA containing a modified nucleoside, a modified internucleotide linkage, or having any combination of modified nucleosides and internucleotide linkages. Non-limiting examples of internucleotide linkage modifications include, but are not limited to, phosphorothioate, phosphotriester and methylphosphonate derivatives (Stec, W. J., et al., Chem. Int. Ed. Engl., 33:709-722 (1994); Lebedev, A. V., et al., E., Perspect. Drug Discov. Des., 4:17-40 (1996); and Zon, et al., U.S. Patent Application No. 20070281308). Other examples of internucleotide linkage modifications may be found in Waldner, et al., Bioorg. Med. Chem. Letters 6:2363-2366 (1996).

As used herein, the term “internucleotide linkage” refers to the bond or bonds that connect two nucleosides of an oligonucleotide or nucleic acid and may be a natural phosphodiester linkage or modified linkage. Some non-limiting examples of modified internucleotide linkage include, for example, phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, and methylphosphonate. As used herein, “phosphorothioate linkage” refers to a linkage between nucleosides in which the phosphorodiester linkage is modified by replacing one of the oxygen atoms, connected to a phosphorus atom, with a sulfur atom.

As used herein, “oligo dT purification” is an affinity chromatography method for purification of mRNA comprising or including a poly-A tail. The process specifically targets and isolates RNA molecules based on their poly-A tails (RNA molecules with poly-A tails bind to the solid support comprising oligo dT, enabling their separation from the RNA molecules without poly-A tails).

As used herein, the term “prematurely aborted RNA transcript” refers to incomplete products of an in vitro transcription reaction. Prematurely aborted RNA sequences may be any length that is less than the intended length of the desired transcriptional product.

The term “promoter” as used herein refers to a nucleotide sequence in a DNA template that directs and controls the initiation of transcription of a particular DNA sequence. Promoters are typically immediately adjacent to (or partially overlap with) the DNA sequence to be transcribed. Promoter sequences are typically located directly upstream or at the 5′ end of the transcription initiation site. Nucleotide positions in the promoter are designated relative to the transcriptional start site, where transcription of DNA begins (position+1).

As used herein, the term “purified” or “purify” refers to separating a substance from at least some of the components (e.g., impurities or contaminants) with which it was associated when initially produced. For example, RNA transcripts are purified by removal of contaminating proteins or other undesired nucleic acid species (e.g., double-stranded RNA, DNA, and/or incomplete or aborted RNA transcripts). Purified substances (e.g., capped mRNA transcripts) can be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated.

As used herein, the term “RNase inhibitor” or “ribonuclease inhibitor” refers to a protein that inhibits RNAse activity for example, during an in vitro transcription reaction.

As used herein, the term “RNA polymerase” refers to an enzyme that synthesizes RNA using a DNA template. For in vitro transcription methods, single subunit phage RNA polymerases derived from T7, T3, SP6, K1-5, KlE, KlF or K11 bacteriophages, or variants thereof, are typically used. This family of polymerases has simple, minimal promoter sequences of about 17 nucleotides which require no accessory proteins and have minimal constraints of the initiating nucleotide sequence.

As used herein, “self-amplifying RNA,” or “saRNA,” is a linear, single-stranded RNA molecule that encodes the gene of interest. saRNA is a type of mRNA, but also includes non-structural proteins that encode a viral replicase. The viral replicase enables the RNA to self-replicate once delivered into the cell.

As used herein, the term “substantially free” refers to a state in which relatively little or no amount of an undesired substance (e.g., prematurely aborted RNA sequences, DNA, and/or double-stranded RNA) is present in a sample. “Substantially free of impurities” means impurities are present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) in a sample. For example, “substantially free of double-stranded RNA” means double-stranded RNA is present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) in a sample.

As used herein, “tangential flow filtration (TFF)” is a type of filtration wherein the material to be filtered is passed tangentially across a filter rather than through it. In TFF, undesired permeate passes through the filter, while the desired retentate passes along the filter and is collected downstream. In TFF, the desired material is typically contained in the retentate, which is the opposite of what is encountered when performing traditional membrane or dead-end filtration.

As used herein, the term “transcription” refers to enzymatically making or synthesizing RNA that is complementary to a DNA template, thereby producing a number of RNA copies of a DNA sequence. The RNA molecule synthesized in a transcription reaction is an “RNA transcript,” “primary transcript,” or “transcript.” Transcription reactions involving the compositions and methods provided herein employ initiating capped oligonucleotide primers described herein. Transcription of a DNA template may be exponential, nonlinear or linear. A DNA template may be a double-stranded linear DNA, a partially double-stranded linear DNA, circular double-stranded DNA, DNA plasmid, PCR amplified product, or a modified nucleic acid template that is compatible with RNA polymerase.

A “ligase,” as used herein, refers to an enzyme that is capable of forming a covalent bond between two nucleotides, and the process of “ligation” refers to the formation of the covalent bond between the two nucleotides. When two nucleotides are ligated a linker may be formed as a result of the ligation process. Said linker can be formed by an enzymatic ligation or a chemical ligation.

As used herein, the terms “universal base,” “degenerate base,” “universal base analog” and “degenerate base analog” include, for example, a nucleoside analog with an artificial base which is, in certain embodiments, recognizable by RNA polymerase as a substitute for one of the natural NTPs (e.g., ATP, UTP, CTP and GTP) or other specific NTP. Universal bases or degenerate bases are disclosed in Loakes, D., Nucleic Acids Res., 29:2437-2447 (2001); Crey-Desbiolles, C., et. al., Nucleic Acids Res., 33:1532-1543 (2005); Kincaid, K., et. al., Nucleic Acids Res., 33:2620-2628 (2005); Preparata, FP, Oliver, J S, J. Comput. Biol. 753-765 (2004); and Hill, F., et. al., Proc Natl Acad. Sci. USA, 95:4258-4263 (1998)).

As used herein, the term “subject” or “patient” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

The terms “effective amount”, “therapeutically effective amount” or “effective dose” or related terms may be used interchangeably and refer to an amount of the therapeutic agent that when administered to a subject, is sufficient to achieve the desired therapeutic result or to have an effect on undesired symptoms, but is generally insufficient to cause adverse side effects. Therapeutically effective amounts of the therapeutic agents provided herein, will vary depending upon the relative activity of the therapeutic agent, and depending upon the subject and disease condition being treated, the weight and age and sex of the subject, the severity of the disease condition in the subject, the manner of administration, drugs used in combination or coincidental with the specific compound employed and the like, which can readily be determined by one of ordinary skill in the art. In one embodiment, a therapeutically effective amount will depend on certain aspects of the subject to be treated and the disorder to be treated and may be ascertained by one skilled in the art using known techniques. In addition, as is known in the art, adjustments for age as well as the body weight, general health, sex, diet, time of administration, drug interaction, and the severity of the disease may be necessary. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium deoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

The term “administering”, “administered” and grammatical variants refers to the physical introduction of a therapeutic agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

“Treating” is to be understood broadly and encompasses any beneficial effect, including, e.g., delaying, slowing, or arresting the worsening of symptoms associated with a viral disease or remedying such symptoms, at least in part. The term is intended to include the cure or elimination of the disease, disorder or condition. Those in need of treatment include those who already have the disease or disorder, as well as those who should prevent the disease or disorder. The patient to be treated is preferably a mammal, in particular a human being.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. In addition to the RNA molecules described herein, provided below are some non-limiting examples of other therapeutic agents. The following therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the term “bioconjugate” refers to the association between atoms or molecules of “bioconjugate reactive groups” or “bioconjugate reactive moieties”. “Bioconjugate linker” refers to the linkage in a bioconjugate formed by bioconjugate reactive groups or bioconjugate reactive moieties. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g.,-sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

The term “linker” as used herein refers to a chemical group linking two molecules or moieties together (the term “linker” includes the term “bioconjugate linker, but is broader). In embodiments, the linker can be formed by ligation (for example, where a phosphate reacts with a hydroxyl creating a link between oxygen and phosphorous). In other embodiments, the linker may be linker (L) capable of binding a purification handle. A linker (L) may comprise a combination of one or more groups such as —S(O)₂—, —N(R)—, —O—, —S—, —C(O)—, —C(O)N(R)-, —N(R)C(O)-, —N(R)C(O)NH—, —NHC(O)N(R)-, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; where R is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or any combination thereof.

The term “precursor RNA” as used herein refers to “A” in Formula I: A-B or in Formula II: A-B-L. The precursor RNA comprises a 5′-cap, an open reading frame (ORF), and a poly-A region. In embodiments, the precursor RNA comprises a 5′-cap, a 5′-UTR, an open reading frame (ORF), a 3′-UTR, and a poly-A region. In embodiments, the precursor RNA comprises a 5′-cap, an open reading frame (ORF), a 3′-UTR, and a poly-A region. In embodiments, the precursor RNA comprises a 5′-cap, a 5′-UTR, an open reading frame (ORF), and a poly-A region. The terms poly-A region and poly-A tail are used interchangeably herein. In embodiments, the term “poly-A region” refers to string of ATPs. In embodiments, the term “poly-A region” refers to string of ATPs followed by several GTPs, or several UTPs, or several CTPs. In embodiments, the term “poly-A region” refers to string of ATPs interrupted by a several GTPs, or several UTPs, or several CTPs, and then followed by more ATPs.

The term “3′-stabilizing region” or “stabilizing region” as used herein refers to “B” in Formula I: A-B or in Formula II: A-B-L. These stabilizing regions result in increased stability of the RNA molecule as compared to a molecule without such stabilizing regions. In embodiments, the stabilizing region inhibits the degradation of the RNA molecule. In embodiments, a 3′-stabilizing region is covalently linked to the 3′-end of precursor RNA (an RNA molecule comprising a 5′-cap, an open reading frame (ORF), and a poly-A region or an RNA molecule comprising a 5′-cap, a 5′-UTR, an open reading frame (ORF), a 3′-UTR, and a poly-A region) by a linker that can be formed by ligation. In embodiments, the linker is a bioconjugate linker. In embodiments, the linker can be formed by an enzymatic ligation, a splint ligation, or a chemical ligation. In embodiments, the 3′-stabilizing region is covalently linked to the 3′-end of the precursor RNA by a linker that can be formed using a polymerase. In embodiments, the 3′-stabilizing region includes one or more unmodified nucleosides and one or more unmodified internucleotide linkages. The unmodified nucleoside is a natural unmodified nucleoside. In embodiments, the 3′-stabilizing region includes one or more modified nucleosides and/or one or more modified internucleotide linkages. In embodiments, a modified nucleoside comprises a modified nucleobase and/or a modified sugar. In embodiments, the 3′-stabilizing region forms a secondary structure. In embodiments, any combination of one or more modified nucleosides and/or one or more modified internucleotide linkages and/or one or more secondary structures is contemplated for stabilization of the RNA molecules. In embodiments, one or more nucleosides within the 3′-stabilizing region comprise one or more purification handles. In embodiments, one or more nucleosides within the 3′-stabilizing region comprise one or more linkers (L), wherein the linker (L) is capable of binding a purification handle. In embodiments, the last nucleoside of the 3′-stabilizing region does not comprise a 3′-hydroxyl. In embodiments, the last nucleoside of the 3′-stabilizing region is blocked and is not able to react with any further NTPs.

As used herein, the term “secondary structure” refers to a 3′-stabilizing region that is capable of forming a secondary structure, such as for example, a G-quadruplex (a secondary structure formed by guanine-rich nucleic acid sequences), triplex, bulge, kissing hairpin, loop e-loop, branched multiloop, stem loop (also known as “hairpin loop”), tetraloop, helix, or pseudoknot, which prevents exonucleases from accessing the 3′ terminal nucleotides of the RNA molecules, and protects the RNA from degradation. Thus, the secondary structure improves the stability and half-life of said RNA molecules.

As used herein, the term “purification handle” refers to a hydrophobic group which is covalently linked to the 3′-stabilizing region of the RNA molecules described herein or to a nucleoside, e.g., via a linker; such group may be removable or non-removable. The purification handle allows for HPLC separation of RNA molecules comprising a 3′-stabilizing region, which comprise covalently linked purification handles, from RNA molecules lacking the 3′-stabilizing region. In embodiments, the purification handle may be a protecting group. In embodiments, the purification handle includes, for example, but is not limited to C₆-C₂₄ alkyl, C₄-C₂₄ alkenyl, C₄-C₂₄ alkynyl, C₃-C₈cycloalkyls, C₆-C₁₀aryls, silyl compounds, trityl compounds, lipids, dyes, steroids, vinyl ether compounds, modified and unmodified Fmoc compounds, and the like, and any combinations thereof. Fluoro substituents or fluoro substituted groups can be used to increase the hydrophobicity of a hydrophobic group. As used herein, the terms “hydrophobic moiety” or “hydrophobic group” may be used interchangeably and refer to hydrophobic substituent or a combination of hydrophobic substituents that are carbon rich. The hydrophobicity of a substituent can be determined, measured or calculated through the value of its partition coefficient (log P). The partition coefficient (log P) of a substance defines the ratio of its solubility in two immiscible solvents, normally octanol:water. When this value is calculated rather than measured, it is called cLog P. In embodiments, a hydrophobic group has a cLog P of at least 2 or a combination of two, three or four “partial hydrophobic groups” has a collective value of cLog P of at least 2. Nucleoside bases cytosine, thymine, uracil, adenine, and guanine, whose cLog P is less than 2, are not considered “hydrophobic groups” as defined herein.

A purification handle may be, for example, S (ethyl)carbonyl(azadibenzocyclooctyne) (DBCO)

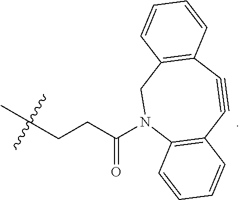 [SMILES: *CCC(=O)N1Cc2ccccc2C#Cc2ccccc21]

A purification handle may be, for example, 4-ethylphenol

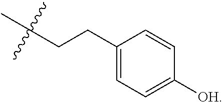 [SMILES: *CCc1ccc(O)cc1]

A purification handle may be, for example, a mixture of the two isomers of dibenzohexyltriazoloazocine

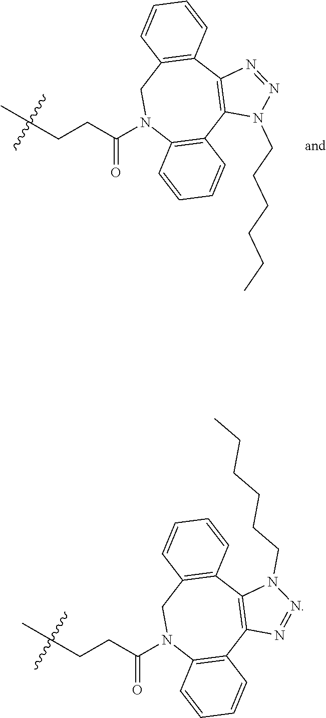 [SMILES: *CCC(=O)N1Cc2ccccc2-c2nnn(CCCCCC)c2-c2ccccc21 ; *CCC(=O)N1Cc2ccccc2-c2c(nnn2CCCCCC)-c2ccccc21]

A purification handle may be, for example, 1′-O-butyl 3′, 4′, 6′-triacetyl GalNAc

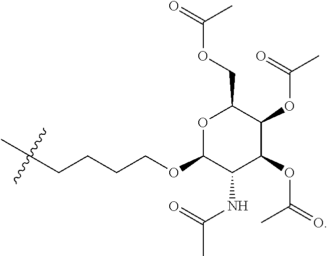 [SMILES: *CCCCO[C@H]1O[C@@H](COC(C)=O)[C@@H](OC(C)=O)[C@@H](OC(C)=O)[C@@H]1NC(C)=O]

Alternatively, the “purification handle” can refer to an affinity tag such as, for example, biotin, polyhistidine, Myc-Tag, MBP-Tag, or GST-Tag.

As used herein, the terms “cleavable purification handle” or “removable purification handle” may be used interchangeably and refer to hydrophobic groups such as for example saturated alkyl groups C₃-C₂₀ or longer, cycloalkyl rings, aryl rings, silyls, and the like, which can be chemically or thermally removed from the RNA molecules described herein using mild conditions. In embodiments, such group(s) can be removed under mild acidic conditions, with pH no lower than about 5 (at room temperature for 1 hour). In embodiments, such group(s) can be removed under mild basic conditions, with a pH no higher than about 9 (at room temperature for 1 hour). In embodiments, such group(s) can be removed by mild heating, at no higher than about 65° C. for 1 hour. In embodiments, such group(s) can be removed by reductive amination. In embodiments, such group(s) can be removed by desilylation. In embodiments, such group(s) can be removed by oxidation. In embodiments, such group(s) can be removed by photolysis. In embodiments, such group(s) are photocleavable (photolabile) groups. In this application, a purification handle is considered “cleavable” or “removable” if it can be removed under conditions wherein RNA molecule is not denatured.

As used herein, the terms “non-cleavable purification handle” or “non-removable purification handle” may be used interchangeably and refer to hydrophobic groups such as for example saturated alkyl groups C₃-C₂₀ or longer, C₃-C₈cycloalkyls, C₆-C₁₀aryls, and the like, which cannot be easily removed from the RNA molecules described herein. In embodiments, said hydrophobic groups cannot be removed using mild conditions described above for the cleavable purification handle. In embodiments, the non-cleavable purification handle cannot be removed at pH between about 5 to about 9 (at room temperature for 1 hour). In embodiments, the non-cleavable purification handle cannot be removed by heating below about 65° C. for 1 hour. In embodiments, at most 5% of the non-cleavable hydrophobic group(s) are cleaved from the RNA molecule. In embodiments, at most 10% of the non-cleavable hydrophobic group(s) are cleaved from the RNA molecule. In embodiments, at most 15% of the non-cleavable hydrophobic group(s) are cleaved from the RNA molecule. In embodiments, at most 20% of the non-cleavable hydrophobic group(s) are cleaved from the RNA molecule.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically, a protecting group is bound to a heteroatom (e.g., O or N) during a part of a multipart synthesis wherein it is not desired to have the heteroatom react (e.g., a chemical reduction) with the reagent. Following protection, the protecting group may be removed (e.g., by modulating the pH or temperature). In embodiments the protecting group is an alcohol protecting group. Non-limiting examples of alcohol protecting groups include acyls, acetyl, benzoyl, benzyl, methoxymethyl ether (MOM), tetrahydropyranyl (THP), tert-butyldimethyl silyl (TBDMS), and silyl ether (e.g., trimethylsilyl (TMS)). In embodiments the protecting group is an amine protecting group. Non-limiting examples of amine protecting groups include trityl, monomethoxytrityl (MMT), dimethoxytrityl (DMT), or other modified trityls, dimethylcarbobenzyloxy (Cbz), tert-butyloxycarbonyl (Boc), 9-Fluorenylmethyloxycarbonyl (Fmoc), acyls, acetyl, benzoyl, benzyl, carbamate, p-methoxybenzyl ether (PMB), tert-butyldiphenyl silyl (TBDPS), and tosyl (Ts).

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

Combination therapy or “in combination with” refer to the use of more than one therapeutic agent to treat a particular disorder or condition. By “in combination with,” it is not intended to imply that the therapeutic agents must be administered at the same time and/or formulated for delivery together, although these methods of delivery are within the scope of this disclosure. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “nucleophile” as used herein refers to a chemical species that donates an electron pair to form a chemical bond with an electrophile in relation to a reaction. All molecules or ions with a free pair of electrons or at least one pi bond can act as nucleophiles.

### RNA Molecules

Described herein are RNA molecules covalently linked to a 3′-stabilizing region, where the 3′-stabilizing region comprises one or more purification handles which are covalently attached, optionally via a linker (L). Additionally, described herein are pharmaceutical compositions comprising said RNA molecules, and methods of preparing said RNA molecules.

In an aspect, provided herein is an RNA molecule comprising the structure of Formula I:

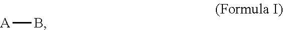

wherein A comprises:
a) a 5′-cap;
b) an open reading frame (ORF) encoding a protein; and
c) a poly-A region, wherein the poly-A region is 3′ to the open reading frame; and
   B comprises a 3′-stabilizing region comprising 1 to 50 nucleosides, wherein one or more nucleosides within the 3′-stabilizing region comprise one or more purification handles.

In embodiments, B comprises a 3′-stabilizing region comprising 1 to 50 nucleosides, wherein one or more nucleosides within the 3′-stabilizing region comprise one or more purification handles and one or more linker(s) (L) capable of binding a purification handle.

In embodiments, B comprises a 3′-stabilizing region comprising one nucleoside, wherein the nucleoside within the 3′-stabilizing region comprises one or more purification handles. In embodiments, B comprises a 3′-stabilizing region comprising one nucleoside, wherein the nucleoside within the 3′-stabilizing region comprises one purification handle. In embodiments, B comprises a 3′-stabilizing region comprising one nucleoside, wherein the nucleoside within the 3′-stabilizing region comprises one or more purification handles and one or more linker(s) (L) capable of binding a purification handle.

In embodiments, B comprises a 3′-stabilizing region comprising two nucleosides, wherein one or both nucleosides within the 3′-stabilizing region comprise one or more purification handles. In embodiments, B comprises a 3′-stabilizing region comprising two nucleosides, wherein one or both nucleosides within the 3′-stabilizing region comprise one purification handle. In embodiments, B comprises a 3′-stabilizing region comprising two nucleosides, wherein one or both nucleosides within the 3′-stabilizing region comprise one or more purification handles and one or more linker(s) (L) capable of binding a purification handle.

In embodiments, a purification handle is on the last nucleoside of the RNA molecule comprising the structure of Formula I. In embodiments, a purification handle is on the penultimate nucleoside of the RNA molecule comprising the structure of Formula I.

In an aspect, provided herein is an RNA molecule comprising the structure of Formula II:

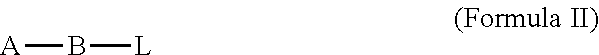

wherein A comprises:
a) a 5′-cap;
b) an open reading frame (ORF) encoding a protein; and
c) a poly-A region, wherein the poly-A region is 3′ to the open reading frame; and
   B comprises a 3′-stabilizing region comprising 1 to 50 nucleosides, wherein one or more nucleosides within the 3′-stabilizing region comprise one or more linkers (L), wherein the linker (L) is capable of binding a purification handle.

In embodiments, B comprises a 3′-stabilizing region comprising one nucleoside, wherein the nucleoside within the 3′-stabilizing region comprises one or more linkers (L), wherein the linker (L) is capable of binding a purification handle. In embodiments, B comprises a 3′-stabilizing region comprising one nucleoside, wherein the nucleoside within the 3′-stabilizing region comprises one linker (L), wherein the linker (L) is capable of binding a purification handle.

In embodiments, B comprises a 3′-stabilizing region comprising two nucleosides, wherein one or both nucleosides within the 3′-stabilizing region comprise one or more linkers (L), wherein the linker (L) is capable of binding a purification handle. In embodiments, B comprises a 3′-stabilizing region comprising two nucleosides, wherein one or both nucleosides within the 3′-stabilizing region comprise one linker (L), wherein the linker (L) is capable of binding a purification handle.

In embodiments, a linker (L), capable of binding a purification handle, is on the last nucleoside of the RNA molecule comprising the structure of Formula II. In embodiments, a linker (L), capable of binding a purification handle, is on the penultimate nucleoside of the RNA molecule comprising the structure of Formula II.

In embodiments, the RNA molecule is an mRNA molecule. In embodiments, the RNA molecule is a linear RNA molecule. In embodiments, the RNA molecule is a circular RNA molecule. In embodiments, the RNA molecule is a linear mRNA molecule. In embodiments, the RNA molecule is a circular mRNA molecule.

In embodiments, precursor RNA comprises a 5′cap, an open reading frame (ORF) encoding a protein, and a poly-A region, wherein the poly-A region is 3′ to the open reading frame. In embodiments, precursor RNA comprises a 5′cap, a 5′-UTR region, an open reading frame (ORF) encoding a protein, a 3′-UTR region, and a poly-A region, wherein the poly-A region is 3′ to the 3′-UTR region. In embodiments, precursor RNA comprises a 5′cap, a 5′-UTR region, an open reading frame (ORF) encoding a protein, and a poly-A region, wherein the poly-A region is 3′ to the open reading frame. In embodiments, precursor RNA comprises a 5′cap, an open reading frame (ORF) encoding a protein, a 3′-UTR region, and a poly-A region, wherein the poly-A region is 3′ to the 3′-UTR region.

In embodiments, precursor RNA comprises a 5′cap, a 5′-UTR region, an open reading frame (ORF) encoding a protein, a 3′-UTR region, and a poly-A region, wherein at least one of the 5′-cap structure, 5′-UTR, coding region, 3′-UTR, and/or poly-A region include at least one modified nucleotide. In embodiments, precursor RNA comprises a 5′cap, a 5′-UTR region, an open reading frame (ORF) encoding a protein, a 3′-UTR region, and a poly-A region, wherein at least one of the 5′-cap structure, 5′-UTR, coding region, 3′-UTR, and/or poly-A region include at least two modified nucleotides. In embodiments, precursor RNA comprises a 5′cap, a 5′-UTR region, an open reading frame (ORF) encoding a protein, a 3′-UTR region, and a poly-A region, wherein at least one of the 5′-cap structure, 5′-UTR, coding region, 3′-UTR, and/or poly-A region include at least three modified nucleotides. In embodiments, precursor RNA comprises a 5′cap, a 5′-UTR region, an open reading frame (ORF) encoding a protein, a 3′-UTR region, and a poly-A region, wherein at least one of the 5′-cap structure, 5′-UTR, coding region, 3′-UTR, and/or poly-A region include at least four modified nucleotides. In embodiments, precursor RNA comprises a 5′cap, a 5′-UTR region, an open reading frame (ORF) encoding a protein, a 3′-UTR region, and a poly-A region, wherein at least one of the 5′-cap structure, 5′-UTR, coding region, 3′-UTR, and/or poly-A region include at least five modified nucleotides.

In embodiments, modified nucleotides may include a modified nucleoside and/or a modified internucleotide linkage. In embodiments, modified nucleotides may include a modified nucleoside and a modified internucleotide linkage. In embodiments, modified nucleotides may include a modified nucleoside or a modified internucleotide linkage. In embodiments, modified nucleotides may include a modified nucleobase and/or a modified sugar and/or a modified internucleotide linkage. In embodiments, modified nucleotides may include a modified nucleobase, a modified sugar, and a modified internucleotide linkage. In embodiments, modified nucleotides may include a modified nucleobase. In embodiments, modified nucleotides may include a modified sugar. In embodiments, modified nucleotides may include a modified internucleotide linkage. In embodiments, modified nucleotides may include a modified nucleobase and a modified sugar. In embodiments, modified nucleotides may include a modified nucleobase and a modified internucleotide linkage. In embodiments, modified nucleotides may include a modified internucleotide linkage and a modified sugar. Some non-limiting examples of modified nucleosides, 5-methyl cytidine, 5′-methoxy uridine, pseudouridine, N1-methylpseudouridine, and the like.

In embodiments, the modified nucleobase is a modified uracil, a modified cytosine, a modified guanine, or a modified adenine. In embodiments, the modified nucleobase is a modified uracil. In embodiments, the modified nucleobase is a modified cytosine. In embodiments, the modified nucleobase is a modified guanine. In embodiments, the modified nucleobase is a modified adenine. In embodiments, the modified nucleobase is pseudouracil (y), 2-thio-uracil, 4-thio-uracil, 4-thio-pseudouridine, 2-thio-pseudouridine, 5-hydroxy-uracil, 5-halo-uracil, 3-methyl-uracil, 5-aza-uracil, or 2-thio-5-aza-uracil. In embodiments, the modified nucleobase is 5-aza-cytosine, 6-aza-cytosine, pseudoisocytidine, 3-methyl-cytosine, 5-methyl-cytosine, 5-halo-cytosine, 2-thio-cytosine, or 2-thio-5-methyl-cytosine. In embodiments, the modified nucleobase is 2-amino-purine, 2,6-diaminopurine, 2-amino-6-halo-purine, 6-halo-purine, 2-amino-6-methyl-purine, 8-azido-adenine, 7-deaza-adenine, N6-methyl-adenine, or 2-methylthio-N6-methyl-adenine. In embodiments, the modified nucleobase is inosine, 1-methyl-inosine, 7-cyano-7-deaza-guanine, 7-aminomethyl-7-deaza-guanine, 6-thio-guanine, 6-thio-7-deaza-guanine, or 6-methoxy-guanine.

Some non-limiting examples of modified nucleosides and nucleobases include pseudouridine (ψ), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (rm⁵U), 1-taurinomethyl-pseudouridine, 5-taurinomethyl-2-thio-uracil(m⁵s²U), 1-taurinomethyl-4-thio-pseudouridine, 5-methyl-uracil (m⁵U, i.e., having the nucleobase deoxythymine), 1-methyl-pseudouridine (m¹ψ), 5-methyl-2-thio-uracil (m⁵s²U), 1-methyl-4-thio-pseudouridine (mis⁴ψ), 4-thio-1-methyl-pseudouridine, 3-methyl-pseudouridine (m¹ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ψ), 5-(isopentenylaminomethyl)uracil (inm⁵U), 5-(isopentenylaminomethyl)-2-thio-uracil (inm⁵s²U), 5,2′-O-dimethyl-uridine (m⁵Um), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s²C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, 5-(2-azidoethyl)-cytosine 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (mlAm), 2-amino-N6-methyl-purine, 1-thio-adenine, 8-azido-adenine, N6-(19-amino-pentaoxanonadecyl)-adenine, 2,8-dimethyl-adenine, N6-formyl-adenine, N6-hydroxymethyl-adenine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine. In embodiments, the modified nucleobase may be any of the foregoing nucleobases.

In embodiments, the modified sugar has a 5-membered ring or a 6-membered ring, or is a modified ribose. In embodiments, the ribose is replaced with a morpholino ring. In embodiments, the modified nucleoside comprises a morpholino ring. In embodiments, the modified ribose is 2′-thioribose, 2′, 3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, or 3′-amino-2′,3′-dideoxyribose.

Some non-limiting examples of modifications to sugars include modifications of the 2′-hydroxy group of the ribose ring, replacement of the oxygen in the ribose ring, expansion or contraction of the ribose ring. In embodiments, a modified sugar may comprise any of the foregoing modifications. In embodiments, the 2′-hydroxy group of the ribose ring can be replaced with a hydrogen, halo, methoxy, azido, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In embodiments, the 2′-hydroxy group of the ribose ring can be replaced with a hydrogen. In embodiments, the 2′-hydroxy group of the ribose ring can be replaced with a halogen. In embodiments, the 2′-hydroxy group of the ribose ring can be replaced with an azido group. In embodiments, the 2′-hydroxy group of the ribose ring can be replaced with a methoxy.

In embodiments, 2′-hydroxy group of the ribose ring is replaced with a substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, 2′-hydroxy group of the ribose ring is replaced with a substituted (e.g., with a substituent group, a size-limited substituent group or a lower substituent group) alkyl (e.g., C₁-C₈ alkyl, C₁-C₆ alkyl, or C₁-C₄ alkyl). In embodiments, 2′-hydroxy group of the ribose ring is replaced with unsubstituted alkyl (e.g., C₁-C₈ alkyl, C₁-C₆ alkyl, or C₁-C₄ alkyl). In embodiments, 2′-hydroxy group of the ribose ring is replaced with a substituted (e.g., with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl). In embodiments, 2′-hydroxy group of the ribose ring is replaced with a substituted (e.g., with a substituent group, a size-limited substituent group or a lower substituent group) heteroalkyl (e.g., 2 to 8 membered heteroalkyl, 2 to 6 membered heteroalkyl, or 2 to 4 membered heteroalkyl). In embodiments, 2′-hydroxy group of the ribose ring is replaced with an unsubstituted heteroalkyl (e.g., 2 to 8 membered heteroalkyl, 2 to 6 membered heteroalkyl, or 2 to 4 membered heteroalkyl). In embodiments, 2′-hydroxy group of the ribose ring is replaced with a substituted (e.g., with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl). In embodiments, 2′-hydroxy group of the ribose ring is replaced with a substituted (e.g., with a substituent group, a size-limited substituent group or a lower substituent group) cycloalkyl (e.g., C₃-C₈ cycloalkyl, C₃-C₆ cycloalkyl, or C₅-C₆ cycloalkyl). In embodiments, 2′-hydroxy group of the ribose ring is replaced with an unsubstituted cycloalkyl (e.g., C₃-C₈ cycloalkyl, C₃-C₆ cycloalkyl, or C₅-C₆ cycloalkyl). In embodiments, 2′-hydroxy group of the ribose ring is replaced with a substituted (e.g., with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, 2′-hydroxy group of the ribose ring is replaced with a substituted (e.g., with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, 2′-hydroxy group of the ribose ring is replaced with an unsubstituted heterocycloalkyl (e.g., 3 to 8 membered heterocycloalkyl, 3 to 6 membered heterocycloalkyl, or 5 to 6 membered heterocycloalkyl). In embodiments, 2′-hydroxy group of the ribose ring is replaced with a substituted (e.g., with a substituent group, a size-limited substituent group or a lower substituent group) or unsubstituted aryl (e.g., C₆-C₁₀ aryl, C₁₀ aryl, or phenyl). In embodiments, 2′-hydroxy group of the ribose ring is replaced with a substituted (e.g., with a substituent group, a size-limited substituent group or a lower substituent group) aryl (e.g., C₆-C₁₀ aryl, C₁₀ aryl, or phenyl). In embodiments, 2′-hydroxy group of the ribose ring is replaced with an unsubstituted aryl (e.g., C₆-C₁₀ aryl, C₁₀ aryl, or phenyl). In embodiments, 2′-hydroxy group of the ribose ring is replaced with a substituted (e.g., with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, 2′-hydroxy group of the ribose ring is replaced with a substituted (e.g., with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, 2′-hydroxy group of the ribose ring is replaced with an unsubstituted heteroaryl (e.g., 5 to 10 membered heteroaryl, 5 to 9 membered heteroaryl, or 5 to 6 membered heteroaryl).

In embodiments, the oxygen in the ribose ring can be replaced with —S—, —Se—, —NH—, or —CH₂—. In embodiments, the oxygen in the ribose ring can be replaced with —S—. In embodiments, the oxygen in the ribose ring can be replaced with —Se—. In embodiments, the oxygen in the ribose ring can be replaced with-NH-. In embodiments, the oxygen in the ribose ring can be replaced with —CH₂—. In embodiments, the ribose ring can be replaced with another ring, for example, the ring can be a cyclobutene, mannitol, cyclohexanyl, or a morpholino ring. In embodiments, the ribose ring can be replaced with a locked nucleic acid ring (LNA). In embodiments, the ribose ring can be replaced with an unlocked nucleic acid ring (UNA).

In embodiments, the internucleotide linkage comprises a modified phosphate. In embodiments, the modified phosphate is phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, methylphosphonate, or guanidinopropyl phosphoramidate.

In embodiments, the modified internucleotide linkages include, for example, phosphorothioates, phosphoroselenates, boranophosphates, boranophosphate esters, hydrogen phosphonates, phosphoramidates, phosphorodiamidates, alkyl or aryl phosphonates, and phosphotriesters. In embodiments, the modified internucleotide linkages may be phosphorodithioates where both non-linking oxygens are replaced by sulfur. In embodiments, the modified internucleotide linkages may include replacing the linking oxygen with-HN-, —S—, or —CH₂—. In embodiments, the modified internucleotide linkages may include replacing the non-linking oxygen (single bond to phosphor) with methyl, ethyl, methoxy, —SH, or —BH₃, or any combination thereof.

Various 5′-cap structures may be used in the synthesis of the RNA molecules described herein (RNA molecules of Formula I: A-B or Formula II: A-B-L). The 5′-cap structure increases the stability of RNA and its resistance to exonuclease degradation. The 5′-cap is also essential for the initiation of translation, where it serves as a recognition site for the translation initiation complex.

5′-cap structures include those described in International Patent Publications Nos. WO2017/053297, WO2023/147352, WO2021/162566, WO2021/162567, WO2022/006368, WO2022/086140, WO2023/033551, WO2018/075827, WO2023/07019, and in U.S. Provisional Applications Nos. 63/528,990, and 63/536,844, the cap structures of each of which are incorporated herein by reference.

In embodiments, 5′-cap structures (cap analogs) may cap the RNA molecules during the in vitro transcription (IVT) reaction. In embodiments, RNA molecules may be capped using enzymes following transcription reaction. In embodiments, the RNA molecules described herein may contain a cap analog. In embodiments, the cap analogs may increase the stability of the RNA molecules. In embodiments, the cap analogs may increase the half-life of the RNA molecules. In embodiments, the cap analogs may increase the translational efficiency of the RNA molecules. In embodiments, the cap analogs may include a purification handle. In embodiments, the cap analogs may include a linker capable of binding a purification handle. In embodiments, the purification handle is a cleavable purification handle. In embodiments, the purification handle is a non-cleavable purification handle.

Some non-limiting examples of cap analogs include, but are not limited to, ^{m7}G_{3′OMe}pppA_{2′OMe}pG, ^{m7}G_{3′OMe}ppp(N-6methyladenine)_{2′OMe}pG, ^{m7}G_{3′OMe}pppApG, ^{m7}G_{3′OMe}ppp(N-6methyladenine)pG, ^{m7}G_{3′OMe}pppG_{2′OMe}pG, ^{m7}G_{3′OMe}pppGpG, ^{m7}GpppA_{2′OMe}pG, ^{m7}Gppp(N-6methyladenine)_{2′OMe}pG, ^{m7}GpppApG, ^{m7}Gppp(N-6methyladenine)pG, ^{m7}GpppG_{2′OMe}pG, ^{m7}GpppGpG, ^{m7}GpppA_{2′OMe}pU, ^{m7}GpppApU, ^{m7}G_{3′OMe}pppA_{2′OMe}pU, and ^{m7}G_{3′OMe}pppApU.

In embodiments, a 5′-UTR is upstream of the translation initiation site. In embodiments, 5′-UTR is adjacent to the 5′-end of the open reading frame (ORF) encoding a protein and downstream of the 5′-cap.

In embodiments, a 3′-UTR is downstream of the translation initiation site. In embodiments, 3′-UTR is adjacent to the 3′-end of the open reading frame (ORF) encoding a protein. In embodiments, a 3′-UTR is upstream of the poly-A region.

In embodiments, the poly-A region includes from about 2 to about 500 nucleosides in length. In embodiments, the poly-A region is 10 or greater nucleosides in length. In embodiments, the poly-A region is 20 or greater nucleosides in length. In embodiments, the poly-A region is 30 or greater nucleosides in length. In embodiments, the poly-A region is 40 or greater nucleosides in length. In embodiments, the poly-A region is 50 or greater nucleosides in length. In embodiments, the poly-A region is 60 or greater nucleosides in length. In embodiments, the poly-A region is 70 or greater nucleosides in length. In embodiments, the poly-A region is 80 or greater nucleosides in length. In embodiments, the poly-A region is 90 or greater nucleosides in length. In embodiments, the poly-A region is 100 or greater nucleosides in length. In embodiments, the poly-A region is 200 or greater nucleosides in length. In embodiments, the poly-A region is 300 or greater nucleosides in length. In embodiments, the poly-A region is 400 or greater nucleosides in length. In embodiments, the poly-A region is 500 or greater nucleosides in length.

In embodiments, the poly-A region is from 2 to 500 nucleosides in length. In embodiments, the poly-A region is from 5 to 500 nucleosides in length. In embodiments, the poly-A region is from 5 to 400 nucleosides in length. In embodiments, the poly-A region is from 5 to 300 nucleosides in length. In embodiments, the poly-A region is from 5 to 350 nucleosides in length. In embodiments, the poly-A region is from 10 to 300 nucleosides in length. In embodiments, the poly-A region is from 10 to 250 nucleosides in length. In embodiments, the poly-A region is from 10 to 200 nucleosides in length. In embodiments, the poly-A region is from 10 to 150 nucleosides in length. In embodiments, the poly-A region is from 15 to 150 nucleosides in length. In embodiments, the poly-A region is from 15 to 100 nucleosides in length. In embodiments, the poly-A region is from 15 to 90 nucleosides in length. In embodiments, the poly-A region is from 15 to 80 nucleosides in length. In embodiments, the poly-A region is from 15 to 70 nucleosides in length. In embodiments, the poly-A region is from 15 to 60 nucleosides in length. In embodiments, the poly-A region is from 15 to 50 nucleosides in length. In embodiments, the poly-A region is from 15 to 40 nucleosides in length.

As used herein “B” in Formula I: A-B or in Formula II: A-B-L comprises a 3′-stabilizing region.

In embodiments, B includes a 3′-stabilizing region comprising 1 to 1000 nucleosides, 1 to 900 nucleosides, 1 to 800 nucleosides, 1 to 700 nucleosides, 1 to 600 nucleosides, 1 to 500 nucleosides, 1 to 450 nucleosides, 1 to 400 nucleosides, 1 to 350 nucleosides, 1 to 300 nucleosides, 1 to 250 nucleosides, 1 to 240 nucleosides, 1 to 230 nucleosides, 1 to 220 nucleosides, 1 to 210 nucleosides, 1 to 200 nucleosides, 1 to 190 nucleosides, 1 to 180 nucleosides, 1 to 170 nucleosides, 1 to 160 nucleosides, 1 to 150 nucleosides, 1 to 140 nucleosides, 1 to 130 nucleosides, 1 to 120 nucleosides, 1 to 110 nucleosides, 1 to 100 nucleosides, 1 to 90 nucleosides, 1 to 80 nucleosides, 1 to 70 nucleosides, 1 to 60 nucleosides, 1 to 50 nucleosides, 1 to 45 nucleosides, 1 to 40 nucleosides, 1 to 35 nucleosides, 1 to 30 nucleosides, 1 to 25 nucleosides, 1 to 20 nucleosides, 1 to 15 nucleosides, 1 to 10 nucleosides, 1 to 5 nucleosides, 1 to 4 nucleosides, 1 to 3 nucleosides, or 1 to 2 nucleosides.

In embodiments, B includes a 3′-stabilizing region comprising 1 to 100 nucleosides, 1 to 90 nucleosides, 1 to 80 nucleosides, 1 to 75 nucleosides, 1 to 70 nucleosides, 1 to 65 nucleosides, 1 to 60 nucleosides, 1 to 55 nucleosides, 1 to 50 nucleosides, 1 to 45 nucleosides, 1 to 40 nucleosides, 1 to 35 nucleosides, 1 to 30 nucleosides, 1 to 25 nucleosides, 1 to 20 nucleosides, 1 to 15 nucleosides, 1 to 10 nucleosides, 1 to 5 nucleosides, or 1 to 2 nucleosides.

In embodiments, B includes a 3′-stabilizing region comprising 1 nucleoside. In embodiments, B includes a 3′-stabilizing region comprising 2 nucleosides.

In embodiments, the 3′-stabilizing region includes one or more unmodified nucleosides and one or more unmodified internucleotide linkages. In embodiments, the 3′-stabilizing region includes one or more modified nucleosides and/or one or more modified internucleotide linkages. In embodiments, the 3′-stabilizing region includes one or more modified nucleosides and one or more modified internucleotide linkages. In embodiments, the 3′-stabilizing region includes one or more modified nucleosides or one or more modified internucleotide linkages.

In embodiments, the 3′-stabilizing region includes one unmodified nucleoside and one unmodified internucleotide linkage. In embodiments, the 3′-stabilizing region includes one unmodified nucleoside and more than one unmodified internucleotide linkages. In embodiments, the 3′-stabilizing region includes more than one unmodified nucleoside and one unmodified internucleotide linkage. In embodiments, the 3′-stabilizing region includes two unmodified nucleosides and two unmodified internucleotide linkages. In embodiments, the 3′-stabilizing region includes three unmodified nucleosides and three unmodified internucleotide linkages. In embodiments, the 3′-stabilizing region includes four unmodified nucleosides and four unmodified internucleotide linkages. In embodiments, the 3′-stabilizing region includes five unmodified nucleosides and five unmodified internucleotide linkages. In embodiments, the 3′-stabilizing region includes six unmodified nucleosides and six unmodified internucleotide linkages. In embodiments, the 3′-stabilizing region includes seven unmodified nucleosides and seven unmodified internucleotide linkages. In embodiments, the 3′-stabilizing region includes eight unmodified nucleosides and eight unmodified internucleotide linkages. In embodiments, the 3′-stabilizing region includes nine unmodified nucleosides and nine unmodified internucleotide linkages. In embodiments, the 3′-stabilizing region includes ten unmodified nucleosides and ten unmodified internucleotide linkages.

In embodiments, the 3′-stabilizing region includes one modified nucleoside and one modified internucleotide linkage. In embodiments, the 3′-stabilizing region includes one modified nucleoside and more than one modified internucleotide linkage. In embodiments, the 3′-stabilizing region includes more than one modified nucleoside and one modified internucleotide linkage. In embodiments, the 3′-stabilizing region includes two modified nucleosides and two modified internucleotide linkages. In embodiments, the 3′-stabilizing region includes more than one modified nucleoside and more than one modified internucleotide linkage.

In embodiments, the 3′-stabilizing region includes one modified nucleoside or one modified internucleotide linkage. In embodiments, the 3′-stabilizing region includes two modified nucleosides or two modified internucleotide linkages. In embodiments, the 3′-stabilizing region includes three modified nucleosides or three modified internucleotide linkages. In embodiments, the 3′-stabilizing region includes four modified nucleosides or four modified internucleotide linkages. In embodiments, the 3′-stabilizing region includes five modified nucleosides or five modified internucleotide linkages. In embodiments, the 3′-stabilizing region includes six modified nucleosides or six modified internucleotide linkages. In embodiments, the 3′-stabilizing region includes seven modified nucleosides or seven modified internucleotide linkages. In embodiments, the 3′-stabilizing region includes eight modified nucleosides or eight modified internucleotide linkages. In embodiments, the 3′-stabilizing region includes nine modified nucleosides or nine modified internucleotide linkages. In embodiments, the 3′-stabilizing region includes ten modified nucleosides or ten modified internucleotide linkages.

In embodiments, the 3′-stabilizing region includes one or more unmodified nucleosides and/or one or more unmodified internucleotide linkages and/or forms a secondary structure. In embodiments, the 3′-stabilizing region includes one or more unmodified nucleosides or one or more unmodified internucleotide linkages or forms a secondary structure. In embodiments, the 3′-stabilizing region includes one or more unmodified nucleosides and one or more unmodified internucleotide linkages and forms a secondary structure. In embodiments, the 3′-stabilizing region includes one or more unmodified nucleosides and one or more unmodified internucleotide linkages or forms a secondary structure. In embodiments, the 3′-stabilizing region includes one or more unmodified nucleosides or one or more unmodified internucleotide linkages and forms a secondary structure. In embodiments, the 3′-stabilizing region forms a secondary structure, which prevents exonucleases from accessing the 3′ terminal nucleotides and protects the 3′end from degradation. In embodiments, the 3′-stabilizing region comprises an aptamer that protects the RNA from degradation.

In embodiments, the 3′-stabilizing region includes one or more modified nucleosides and/or one or more modified internucleotide linkages and/or forms a secondary structure. In embodiments, the 3′-stabilizing region includes one or more modified nucleosides or one or more modified internucleotide linkages or forms a secondary structure. In embodiments, the 3′-stabilizing region includes one or more modified nucleosides and one or more modified internucleotide linkages and forms a secondary structure. In embodiments, the 3′-stabilizing region includes one or more modified nucleosides and one or more modified internucleotide linkages or forms a secondary structure. In embodiments, the 3′-stabilizing region includes one or more modified nucleosides or one or more modified internucleotide linkages and forms a secondary structure. In embodiments, the 3′-stabilizing region forms a secondary structure, which prevents exonucleases from accessing the 3′ terminal nucleotides and protects the 3′end from degradation. In embodiments, the 3′-stabilizing region comprises an aptamer that protects the RNA from degradation.

In embodiments, the 3′-stabilizing region forms a secondary structure. In embodiments, the secondary structure may be, for example, a G-quadruplex (a secondary structure formed by guanine-rich nucleic acid sequences), a triplex, bulge, kissing hairpin, loop e-loop, branched multiloop, a stem loop (also known as “hairpin loop”), a tetraloop, helix, or a pseudoknot. In embodiments, the secondary structure may be a G-quadruplex. In embodiments, the secondary structure may be a triplex. In embodiments, the secondary structure may be a hairpin loop (stem loop). In embodiments, the secondary structure may be a tetraloop. In embodiments, the secondary structure may be a helix. In embodiments, the secondary structure may be a bulge. In embodiments, the secondary structure may be a kissing hairpin. In embodiments, the secondary structure may be a loop e-loop. In embodiments, the secondary structure may be a branched multiloop. In embodiments, the secondary structure may be a pseudoknot.

In embodiments, a modified nucleoside comprises a modified nucleobase and/or a modified sugar. In embodiments, a modified nucleoside comprises a modified nucleobase and a modified sugar. In embodiments, a modified nucleoside comprises a modified nucleobase. In embodiments, a modified nucleoside comprises a modified sugar.

In embodiments, the modified nucleobase is a modified uracil, a modified cytosine, a modified guanine, or a modified adenine. In embodiments, the modified nucleobase is a modified uracil. In embodiments, the modified nucleobase is a modified cytosine. In embodiments, the modified nucleobase is a modified guanine. In embodiments, the modified nucleobase is a modified adenine. In embodiments, the modified nucleobase is pseudouracil (y), 2-thio-uracil, 4-thio-uracil, 4-thio-pseudouridine, 2-thio-pseudouridine, 5-hydroxy-uracil, 5-halo-uracil, 3-methyl-uracil, 5-aza-uracil, or 2-thio-5-aza-uracil. In embodiments, the modified nucleobase is 5-aza-cytosine, 6-aza-cytosine, pseudoisocytidine, 3-methyl-cytosine, 5-methyl-cytosine, 5-halo-cytosine, 2-thio-cytosine, or 2-thio-5-methyl-cytosine. In embodiments, the modified nucleobase is 2-amino-purine, 2,6-diaminopurine, 2-amino-6-halo-purine, 6-halo-purine, 2-amino-6-methyl-purine, 8-azido-adenine, 7-deaza-adenine, N6-methyl-adenine, or 2-methylthio-N6-methyl-adenine. In embodiments, the modified nucleobase is inosine, 1-methyl-inosine, 7-cyano-7-deaza-guanine, 7-aminomethyl-7-deaza-guanine, 6-thio-guanine, 6-thio-7-deaza-guanine, or 6-methoxy-guanine.

Some non-limiting examples of modified nucleosides and nucleobases include pseudouridine (W), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm^{S}U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (τm⁵U), 1-taurinomethyl-pseudouridine, 5-taurinomethyl-2-thio-uracil(m⁵s²U), 1-taurinomethyl-4-thio-pseudouridine, 5-methyl-uracil (m⁵U, i.e., having the nucleobase deoxythymine), 1-methyl-pseudouridine (m¹ψ), 5-methyl-2-thio-uracil (m⁵s²U), 1-methyl-4-thio-pseudouridine (m¹s⁴ψ), 4-thio-1-methyl-pseudouridine, 3-methyl-pseudouridine (m¹ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ψ), 5-(isopentenylaminomethyl)uracil (inm⁵U), 5-(isopentenylaminomethyl)-2-thio-uracil (inm⁵s²U), 5,2′-O-dimethyl-uridine (m⁵Um), 2-thio-2′-O-methyl-uridine (s²Um), 5-methoxycarbonylmethyl-2′-O-methyl-uridine (mcm^{S}Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s²C), 2-thio-5-methyl-cytosine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-i-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (f5Cm), N4,N4,2′-O-trimethyl-cytidine (m42Cm), 1-thio-cytosine, 5-hydroxy-cytosine, 5-(3-azidopropyl)-cytosine, 5-(2-azidoethyl)-cytosine 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-O-dimethyl-adenosine (m6Am), N6,N6,2′-O-trimethyl-adenosine (m62Am), 1,2′-O-dimethyl-adenosine (mlAm), 2-amino-N6-methyl-purine, 1-thio-adenine, 8-azido-adenine, N6-(19-amino-pentaoxanonadecyl)-adenine, 2,8-dimethyl-adenine, N6-formyl-adenine, N6-hydroxymethyl-adenine, inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine. In embodiments, the modified nucleobase may be any of the foregoing nucleobases.

In embodiments, the modified sugar has a 5-membered ring or a 6-membered ring, or is a modified ribose. In embodiments, the ribose is replaced with a morpholino ring. In embodiments, the modified nucleoside comprises a morpholino ring. In embodiments, the modified ribose is 2′-thioribose, 2′, 3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, or 3′-amino-2′,3′-dideoxyribose.

Some non-limiting examples of modifications of sugars include modifications of the 2′-hydroxy group of the ribose ring, replacement of the oxygen in the ribose ring, expansion or contraction of the ribose ring. In embodiments, a modified sugar may comprise any of the foregoing modifications. In embodiments, the 2′-hydroxy group of the ribose ring can be replaced with a hydrogen, halo, methoxy, azido, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

In embodiments, the 2′-hydroxy group of the ribose ring can be replaced with a hydrogen. In embodiments, the 2′-hydroxy group of the ribose ring can be replaced with a halogen. In embodiments, the 2′-hydroxy group of the ribose ring can be replaced with an azido group. In embodiments, the 2′-hydroxy group of the ribose ring can be replaced with a methoxy.

In embodiments, 2′-hydroxy group of the ribose ring is replaced with a substituted (e.g., with a substituent group, a size-limited substituent group or a lower substituent group) or unsubstituted alkyl (e.g., C₁-C₈ alkyl, C₁-C₆ alkyl, or C₁-C₄ alkyl). In embodiments, 2′-hydroxy group of the ribose ring is replaced with a substituted (e.g., with a substituent group, a size-limited substituent group or a lower substituent group) alkyl (e.g., C₁-C₈ alkyl, C₁-C₆ alkyl, or C₁-C₄ alkyl). In embodiments, 2′-hydroxy group of the ribose ring is replaced with unsubstituted alkyl (e.g., C₁-C₈ alkyl, C₁-C₆ alkyl, or C₁-C₄ alkyl). In embodiments, 2′-hydroxy group of the ribose ring is replaced with a substituted (e.g., with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl). In embodiments, 2′-hydroxy group of the ribose ring is replaced with a substituted (e.g., with a substituent group, a size-limited substituent group or a lower substituent group) heteroalkyl (e.g., 2 to 8 membered heteroalkyl, 2 to 6 membered heteroalkyl, or 2 to 4 membered heteroalkyl). In embodiments, 2′-hydroxy group of the ribose ring is replaced with an unsubstituted heteroalkyl (e.g., 2 to 8 membered heteroalkyl, 2 to 6 membered heteroalkyl, or 2 to 4 membered heteroalkyl). In embodiments, 2′-hydroxy group of the ribose ring is replaced with a substituted (e.g., with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl). In embodiments, 2′-hydroxy group of the ribose ring is replaced with a substituted (e.g., with a substituent group, a size-limited substituent group or a lower substituent group) cycloalkyl (e.g., C₃-C₈ cycloalkyl, C₃-C₆ cycloalkyl, or C₅-C₆ cycloalkyl). In embodiments, 2′-hydroxy group of the ribose ring is replaced with an unsubstituted cycloalkyl (e.g., C₃-C₈ cycloalkyl, C₃-C₆ cycloalkyl, or C₅-C₆ cycloalkyl). In embodiments, 2′-hydroxy group of the ribose ring is replaced with a substituted (e.g., with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, 2′-hydroxy group of the ribose ring is replaced with a substituted (e.g., with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, 2′-hydroxy group of the ribose ring is replaced with an unsubstituted heterocycloalkyl (e.g., 3 to 8 membered heterocycloalkyl, 3 to 6 membered heterocycloalkyl, or 5 to 6 membered heterocycloalkyl). In embodiments, 2′-hydroxy group of the ribose ring is replaced with a substituted (e.g., with a substituent group, a size-limited substituent group or a lower substituent group) or unsubstituted aryl (e.g., C₆-C₁₀ aryl, C₁₀ aryl, or phenyl). In embodiments, 2′-hydroxy group of the ribose ring is replaced with a substituted (e.g., with a substituent group, a size-limited substituent group or a lower substituent group) aryl (e.g., C₆-C₁₀ aryl, C₁₀ aryl, or phenyl). In embodiments, 2′-hydroxy group of the ribose ring is replaced with an unsubstituted aryl (e.g., C₆-C₁₀ aryl, C₁₀ aryl, or phenyl). In embodiments, 2′-hydroxy group of the ribose ring is replaced with a substituted (e.g., with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, 2′-hydroxy group of the ribose ring is replaced with a substituted (e.g., with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, 2′-hydroxy group of the ribose ring is replaced with an unsubstituted heteroaryl (e.g., 5 to 10 membered heteroaryl, 5 to 9 membered heteroaryl, or 5 to 6 membered heteroaryl).

In embodiments, the oxygen in the ribose ring can be replaced with —S—, —Se—, —NH—, or —CH₂—. In embodiments, the oxygen in the ribose ring can be replaced with —S—. In embodiments, the oxygen in the ribose ring can be replaced with —Se—. In embodiments, the oxygen in the ribose ring can be replaced with-NH-. In embodiments, the oxygen in the ribose ring can be replaced with —CH₂—. In embodiments, the ribose ring can be replaced with another ring, for example, the ring can be a cyclobutene, mannitol, cyclohexanyl, or a morpholino ring. In embodiments, the ribose ring can be replaced with a morpholino ring. In embodiments, the ribose ring can be replaced with a mannitol ring. In embodiments, the ribose ring can be replaced with a cyclohexanyl ring. In embodiments, the ribose ring can be replaced with a locked nucleic acid ring (LNA). In embodiments, the ribose ring can be replaced with an unlocked nucleic acid ring (UNA).

In embodiments, the 3′-stabilizing region includes one or more modified internucleotide linkages. In embodiments, the internucleotide linkage comprises a modified phosphate. In embodiments, the modified phosphate is phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, methylphosphonate, or guanidinopropyl phosphoramidate.

In embodiments, the modified internucleotide linkages include, for example, phosphorothioates, phosphoroselenates, boranophosphates, boranophosphate esters, hydrogen phosphonates, phosphoramidates, phosphorodiamidates, alkyl or aryl phosphonates, and phosphotriesters. In embodiments, the modified internucleotide linkages may be phosphorodithioates where both non-linking oxygens are replaced by sulfur. In embodiments, the modified internucleotide linkages may include replacing the linking oxygen with-HN-, —S—, or —CH₂—. In embodiments, the modified internucleotide linkages may include replacing the non-linking oxygen (single bond to phosphor) with methyl, ethyl, methoxy, —SH, or —BH₃, or any combination thereof.

In embodiments, one or more unmodified nucleosides and one or more unmodified internucleotide linkages and one or more secondary structures is contemplated for stabilization of the RNA molecules. In embodiments, one or more unmodified nucleosides and one or more unmodified internucleotide linkages is contemplated for stabilization of the RNA molecules. In embodiments, any combination of one or more modified nucleosides and/or one or more modified internucleotide linkages and/or one or more secondary structures is contemplated for stabilization of the RNA molecules. For example, it is contemplated that the 3′-stabilized region may include at least one PS modification (of the internucleotide linkage) and/or at least one modification of the 2′-hydroxy group of the ribose ring. In embodiments, it is contemplated, for example, that the 3′-stabilized region may include at least one PS modification (of the internucleotide linkage) and/or at least one modification of the 2′-hydroxy group of the ribose ring and/or at least one secondary structure.

In embodiments, the last nucleoside of the 3′-stabilizing region does not comprise a 3′-hydroxyl. In embodiments, the last nucleoside of the 3′-stabilizing region is a chain terminating nucleoside. In embodiments, the last nucleoside of the 3′-stabilizing region is blocked and is not able to react with any further NTPs. In embodiments, the last nucleoside of the 3′-stabilizing region is ddC, inverted dT, 3′-phosphate nucleoside, 3′-oxime nucleoside, 3′-azidomethyl nucleoside, or 3′-methyl nucleoside. In embodiments, the last nucleoside of the 3′-stabilizing region is ddC. In embodiments, the last nucleoside of the 3′-stabilizing region is inverted dT. In embodiments, the last nucleoside of the 3′-stabilizing region is 3′-phosphate nucleoside. In embodiments, the last nucleoside of the 3′-stabilizing region is 3′-oxime nucleoside. In embodiments, the last nucleoside of the 3′-stabilizing region is 3′-methyl nucleoside.

In embodiments, one or more nucleosides within the 3′-stabilizing region comprise one or more purification handles. In embodiments, one nucleoside within the 3′-stabilizing region comprises one or more purification handles. In embodiments, one nucleoside within the 3′-stabilizing region comprises one purification handle. In embodiments, two nucleosides within the 3′-stabilizing region comprise two purification handles. In embodiments, three nucleosides within the 3′-stabilizing region comprise three purification handles. In embodiments, four nucleosides within the 3′-stabilizing region comprise four purification handles. In embodiments, five nucleosides within the 3′-stabilizing region comprise five purification handles. In embodiments, six nucleosides within the 3′-stabilizing region comprise six purification handles. In embodiments, seven nucleosides within the 3′-stabilizing region comprise seven purification handles. In embodiments, eight nucleosides within the 3′-stabilizing region comprise eight purification handles. In embodiments, nine nucleosides within the 3′-stabilizing region comprise nine purification handles. In embodiments, ten nucleosides within the 3′-stabilizing region comprise ten purification handles.

In embodiments, one nucleoside within the 3′-stabilizing region comprises one purification handle. In embodiments, one nucleoside within the 3′-stabilizing region comprises two purification handles. In embodiments, one nucleoside within the 3′-stabilizing region comprises three purification handles. In embodiments, one nucleoside within the 3′-stabilizing region comprises four purification handles. In embodiments, one nucleoside within the 3′-stabilizing region comprises five purification handles. In embodiments, one nucleoside within the 3′-stabilizing region comprises at least five purification handles. In embodiments, one nucleoside within the 3′-stabilizing region comprises ten purification handles.

In embodiments, one or more nucleosides within the 3′-stabilizing region comprise one or more linkers (L), wherein the linker (L) is capable of binding a purification handle. In embodiments, one nucleoside within the 3′-stabilizing region comprises one or more linkers (L). In embodiments, one nucleoside within the 3′-stabilizing region comprises one linker (L). In embodiments, two nucleosides within the 3′-stabilizing region comprise two linkers (L). In embodiments, three nucleosides within the 3′-stabilizing region comprise three linkers (L). In embodiments, four nucleosides within the 3′-stabilizing region comprise four linkers (L). In embodiments, five nucleosides within the 3′-stabilizing region comprise five linkers (L). In embodiments, six nucleosides within the 3′-stabilizing region comprise six linkers (L). In embodiments, seven nucleosides within the 3′-stabilizing region comprise seven linkers (L). In embodiments, eight nucleosides within the 3′-stabilizing region comprise eight linkers (L). In embodiments, nine nucleosides within the 3′-stabilizing region comprise nine linkers (L). In embodiments, ten nucleosides within the 3′-stabilizing region comprise ten linkers (L).

In embodiments, one nucleoside within the 3′-stabilizing region comprises one linker (L). In embodiments, one nucleoside within the 3′-stabilizing region comprises two linkers (L). In embodiments, one nucleoside within the 3′-stabilizing region comprises three linkers (L). In embodiments, one nucleoside within the 3′-stabilizing region comprises four linkers (L). In embodiments, one nucleoside within the 3′-stabilizing region comprises five linkers (L). In embodiments, one nucleoside within the 3′-stabilizing region comprises at least five linkers (L). In embodiments, one nucleoside within the 3′-stabilizing region comprises ten linkers (L).

In embodiments, the purification handle is linked to the 3′-stabilizing region via a linker (L). In embodiments, L is a bond, —S(O)₂—, —N(R)—, —O—, —S—, —C(O)—, —C(O)N(R)-, —N(R)C(O)-, —N(R)C(O)NH—, —NHC(O)N(R)-, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or any combination thereof; and R is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or any combination thereof.

In embodiments, L is a substituted (e.g., with a substituent group, a size-limited substituent group or a lower substituent group) or unsubstituted alkylene (e.g., C₁-C₈ alkylene, C₁-C₆ alkylene, or C₁-C₄ alkylene). In embodiments, L is a substituted (e.g., with a substituent group, a size-limited substituent group or a lower substituent group) alkylene (e.g., C₁-C₈ alkylene, C₁-C₆ alkylene, or C₁-C₄ alkylene). In embodiments, L is unsubstituted alkylene (e.g., C₁-C₈ alkylene, C₁-C₆ alkylene, or C₁-C₄ alkylene). In embodiments, L is a substituted (e.g., with a substituent group, a size-limited substituent group or a lower substituent group) or unsubstituted heteroalkylene (e.g., 2 to 8 membered heteroalkylene, 2 to 6 membered heteroalkylene, or 2 to 4 membered heteroalkylene). In embodiments, L is a substituted (e.g., with a substituent group, a size-limited substituent group or a lower substituent group) heteroalkylene (e.g., 2 to 8 membered heteroalkylene, 2 to 6 membered heteroalkylene, or 2 to 4 membered heteroalkylene). In embodiments, L is an unsubstituted heteroalkylene (e.g., 2 to 8 membered heteroalkylene, 2 to 6 membered heteroalkylene, or 2 to 4 membered heteroalkylene). In embodiments, L is a substituted (e.g., with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene). In embodiments, L is a substituted (e.g., with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, L is an unsubstituted cycloalkylene (e.g., C₃-C₈ cycloalkylene, C₃-C₆ cycloalkylene, or C₅-C₆ cycloalkylene). In embodiments, L is a substituted (e.g., with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene). In embodiments, L is a substituted (e.g., with a substituent group, a size-limited substituent group or a lower substituent group) heterocycloalkylene (e.g., 3 to 8 membered heterocycloalkylene, 3 to 6 membered heterocycloalkylene, or 5 to 6 membered heterocycloalkylene). In embodiments, L is an unsubstituted heterocycloalkylene (e.g., 3 to 8 membered heterocycloalkylene, 3 to 6 membered heterocycloalkylene, or 5 to 6 membered heterocycloalkylene). In embodiments, L is a substituted (e.g., with a substituent group, a size-limited substituent group or a lower substituent group) or unsubstituted arylene (e.g., C₆-C₁₀ arylene, C₁₀ arylene, or phenylene). In embodiments, L is a substituted (e.g., with a substituent group, a size-limited substituent group or a lower substituent group) arylene (e.g., C₆-C₁₀ arylene, C₁₀ arylene, or phenylene). In embodiments, L is an unsubstituted arylene (e.g., C₆-C₁₀ arylene, C₁₀ arylene, or phenylene). In embodiments, L is a substituted (e.g., with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene). In embodiments, L is a substituted (e.g., with a substituent group, a size-limited substituent group or a lower substituent group) heteroarylene (e.g., 5 to 10 membered heteroarylene, 5 to 9 membered heteroarylene, or 5 to 6 membered heteroarylene). In embodiments, L is an unsubstituted heteroarylene (e.g., 5 to 10 membered heteroarylene, 5 to 9 membered heteroarylene, or 5 to 6 membered heteroarylene).

In embodiments, R is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHN-2, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl)., or substituted or unsubstituted heteroaryl (e.g., 5 to 10 membered heteroaryl, 5 to 9 membered heteroaryl, or 5 to 6 membered heteroaryl).

In embodiments, R is a substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R is a substituted (e.g. with a substituent group, a size-limited substituent group or a lower substituent group) alkyl (e.g., C₁-C₈ alkyl, C₁-C₆ alkyl, or C₁-C₄ alkyl). In embodiments, R is an unsubstituted alkyl (e.g., C₁-C₈ alkyl, C₁-C₆ alkyl, or C₁-C₄ alkyl). In embodiments, R is a substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl). In embodiments, R is a substituted (e.g. with a substituent group, a size-limited substituent group or a lower substituent group) heteroalkyl (e.g., 2 to 8 membered heteroalkyl, 2 to 6 membered heteroalkyl, or 2 to 4 membered heteroalkyl). In embodiments, R is an unsubstituted heteroalkyl (e.g., 2 to 8 membered heteroalkyl, 2 to 6 membered heteroalkyl, or 2 to 4 membered heteroalkyl). In embodiments, R is a substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl). In embodiments, R is a substituted (e.g. with a substituent group, a size-limited substituent group or a lower substituent group) cycloalkyl (e.g., C₃-C₈ cycloalkyl, C₃-C₆ cycloalkyl, or C₅-C₆ cycloalkyl). In embodiments, R is an unsubstituted cycloalkyl (e.g., C₃-C₈ cycloalkyl, C₃-C₆ cycloalkyl, or C₅-C₆ cycloalkyl). In embodiments, R is a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R is a substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R is an unsubstituted heterocycloalkyl (e.g., 3 to 8 membered heterocycloalkyl, 3 to 6 membered heterocycloalkyl, or 5 to 6 membered heterocycloalkyl). In embodiments, R is a substituted (e.g. with a substituent group, a size-limited substituent group or a lower substituent group) or unsubstituted aryl (e.g., C₆-C₁₀ aryl, C₁₀ aryl, or phenyl). In embodiments, R is a substituted (e.g. with a substituent group, a size-limited substituent group or a lower substituent group) aryl (e.g., C₆-C₁₀ aryl, C₁₀ aryl, or phenyl). In embodiments, R is an unsubstituted aryl (e.g., C₆-C₁₀ aryl, C₁₀ aryl, or phenyl). In embodiments, R is a substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, R is a substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, R is an unsubstituted heteroaryl (e.g., 5 to 10 membered heteroaryl, 5 to 9 membered heteroaryl, or 5 to 6 membered heteroaryl).

In embodiments, L is a substituted or unsubstituted alkylene or substituted or unsubstituted heteroalkylene. In embodiments, L is a substituted (e.g., with a substituent group, a size-limited substituent group or a lower substituent group) or unsubstituted alkylene (e.g., C₁-C₈ alkylene, C₁-C₆ alkylene, or C₁-C₄ alkylene). In embodiments, L is a substituted (e.g., with a substituent group, a size-limited substituent group or a lower substituent group) alkylene (e.g., C₁-C₈ alkylene, C₁-C₆ alkylene, or C₁-C₄ alkylene). In embodiments, L is unsubstituted alkylene (e.g., C₁-C₈ alkylene, C₁-C₆ alkylene, or C₁-C₄ alkylene). In embodiments, L is a substituted (e.g., with a substituent group, a size-limited substituent group or a lower substituent group) or unsubstituted heteroalkylene (e.g., 2 to 8 membered heteroalkylene, 2 to 6 membered heteroalkylene, or 2 to 4 membered heteroalkylene). In embodiments, L is a substituted (e.g., with a substituent group, a size-limited substituent group or a lower substituent group) heteroalkylene (e.g., 2 to 8 membered heteroalkylene, 2 to 6 membered heteroalkylene, or 2 to 4 membered heteroalkylene). In embodiments, L is an unsubstituted heteroalkylene (e.g., 2 to 8 membered heteroalkylene, 2 to 6 membered heteroalkylene, or 2 to 4 membered heteroalkylene).

In embodiments, L is

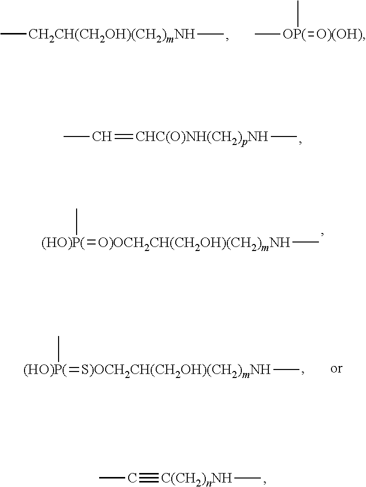

wherein m is an integer from 0 to 8; n is an integer from 0 to 8; and p is an integer from 0 to 10. In embodiments, L is

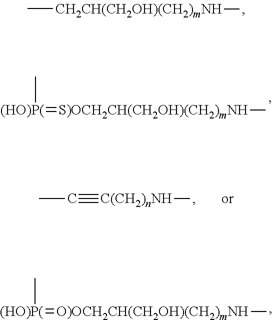

wherein m is an integer from 0 to 8. In embodiments, L is —C≡C(CH₂)ₙNH—, wherein n is an integer from 0 to 8. In embodiments, L is —CH═CHC(O)NH(CH₂)ₚNH-, wherein p is an integer from 0 to 10. In embodiments, L is

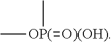

In embodiments, L is —CH₂CH(CH₂OH)(CH₂)₄NH- or —CH═CHC(O)NH(CH₂)₆NH-. In embodiments, L is —CH₂CH(CH₂OH)(CH₂)₄NH-. In embodiments, L is —CH═CHC(O)NH(CH₂)₆NH-.

In embodiments, L is

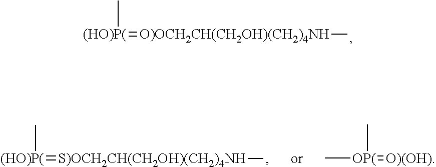

In embodiments, L is

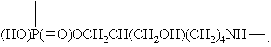

In embodiments, L is

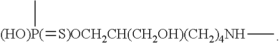

In embodiments, L is

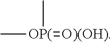

In embodiments, L is —C≡C(CH₂)NH-.

In embodiments, L is a phosphate or a modified phosphate. Some non-limiting examples of modified phosphate include, but are not limited to, phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, methylphosphonate, and guanidinopropyl phosphoramidate.

In embodiments, the modified phosphate may be, for example, phosphorothioate, phosphoroselenate, boranophosphate, boranophosphate ester, hydrogen phosphonate, phosphoramidate, phosphorodiamidate, alkyl or aryl phosphonate, or phosphotriester.

In embodiments, the purification handle is a hydrophobic group which is covalently linked to the 3′-stabilizing region of the RNA molecules described herein, such group may be removable or non-removable. In embodiments, the purification handle is linked, via covalent bond to the 3′-stabilizing region directly. In embodiments, the purification handle is linked, via a covalent bond, to the 3′-stabilizing region via linker (L). In embodiments, the purification handle is a removable group. In embodiments, the purification handle is a non-removable group.

In embodiments, the purification handle includes, for example, but is not limited to C₆-C₂₄ alkyls, C₄-C₂₄ alkenyls, C₄-C₂₄ alkynyls, C₃-C₈cycloalkyls, C₆-C₁₀aryls, silyl compounds, trityl compounds, lipids, dyes, steroids, vinyl ether compounds, modified and unmodified Fmoc compounds, and the like. Fluoro substituents or fluorosubstituted groups can be used to increase the hydrophobicity of a hydrophobic group. In embodiments, the purification handle includes a C₆-C₂₄ alkyl. In embodiments, the purification handle includes a C₄-C₂₄ alkenyl. In embodiments, the purification handle includes a C₄-C₂₄ alkynyl. In embodiments, the purification handle includes a C₃-C₈cycloalkyl. In embodiments, the purification handle includes a C₆-C₁₀aryl. In embodiments, the purification handle includes a silyl compound. In embodiments, the purification handle includes a trityl compound. In embodiments, the purification handle includes a lipid. In embodiments, the purification handle includes a steroid. In embodiments, the purification handle includes a vinyl ether compound. In embodiments, the purification handle includes a modified Fmoc compound. In embodiments, the purification handle includes an unmodified Fmoc compound.

In embodiments, the purification handle includes for example, but is not limited to propyl, butyl, pentyl, hexyl, heptyl, octyl, nonyl, decyl, undecyl, dodecyl, tridecyl, tetradecyl, and pentadecyl. In embodiments, the purification handle includes for example, but is not limited to phenyl, benzyl, (ethyl)carbonyl(azadibenzocyclooctyne) (DBCO), 4-ethylphenol, dibenzohexyltriazoloazocine, and 1′-O-butyl 3′, 4′, 6′-triacetyl GalNAc.

In embodiments, the purification handle includes for example, but is not limited to —C(O)-propyl, —C(O)-butyl, —C(O)-pentyl, —C(O)-hexyl, —C(O)-heptyl, —C(O)-octyl, —C(O)-nonyl, —C(O)-decyl, —C(O)-undecyl, —C(O)-dodecyl, —C(O)-tridecyl, —C(O)-tetradecyl, and —C(O)-pentadecyl. In embodiments, the purification handle includes for example, but is not limited to —C(O)-phenyl, —C(O)-benzyl, —C(O)-4-ethylphenol, —C(O)-(ethyl)carbonyl(azadibenzocyclooctyne), —C(O)-dibenzohexyltriazoloazocine, and —C(O)-1′-O-butyl 3′, 4′, 6′-triacetyl GalNAc.

In embodiments, the purification handle is (ethyl)carbonyl(azadibenzocyclooctyne) (DBCO)

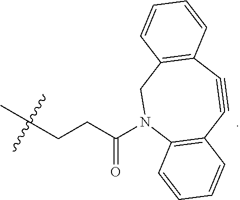 [SMILES: *CCC(=O)N1Cc2ccccc2C#Cc2ccccc21]

In embodiments, the purification handle is 4-ethylphenol

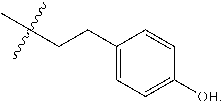 [SMILES: *CCc1ccc(O)cc1]

In embodiments, the purification handle is dibenzohexyltriazoloazocine a mixture of the two isomers

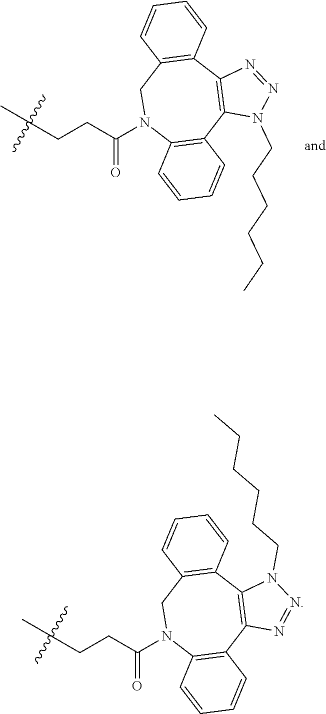 [SMILES: *CCC(=O)N1Cc2ccccc2-c2nnn(CCCCCC)c2-c2ccccc21 ; *CCC(=O)N1Cc2ccccc2-c2c(nnn2CCCCCC)-c2ccccc21]

In embodiments, the purification handle is 1′-O-butyl 3′, 4′, 6′-triacetyl GalNAc

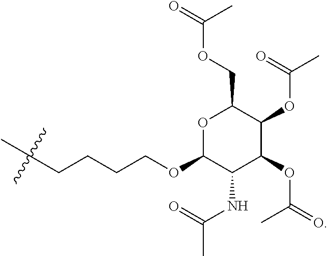 [SMILES: *CCCCO[C@H]1O[C@@H](COC(C)=O)[C@@H](OC(C)=O)[C@@H](OC(C)=O)[C@@H]1NC(C)=O]

In embodiments, the purification handle includes a lipid. In embodiments, without wishing to be bound by any particular theory, the purification handles that include lipids may lead to increased encapsulation of mRNA molecules, bearing lipid purification handles, in pharmaceutical carriers such as, for example, lipid nanoparticles (LNPs).

In embodiments, contemplated herein are RNA molecules comprising a 3′-stabilizing region as described herein and a 5′-cap analog as described herein and in references incorporated herein. In embodiments, contemplated herein are RNA molecules comprising a 5′-cap analog as described herein and in references incorporated herein and/or modified 5′-UTR as described herein and/or modified ORF as described herein and/or modified 3′-UTR as described herein and/or modified poly-A tail as described herein and/or a 3′stabilizing region as described herein.

### Compounds

In an aspect, provided herein is a compound of Formula (III) or (IV):

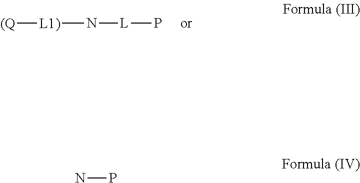

wherein N is a nucleoside;
L is a linker capable of binding a purification handle;
P is a purification handle;
Q-L1 is optionally present, wherein L1 is a linker covalently bound to N and to Q;
and Q is a hydrogen or a chain terminating nucleoside.

In embodiments, the nucleoside is an unmodified nucleoside. In embodiments, the nucleoside is a modified nucleoside. In embodiments the modified nucleoside is as defined herein, including in embodiments.

In embodiments, the linker (L) is as defined herein, including in embodiments.

In embodiments, the purification handle (P) is as defined herein, including in embodiments.

In embodiments, L1 is a linker. In embodiments, L1 linker can be the same or different than linker (L). In embodiments, linker L1 is the same as linker (L) as defined herein, including in embodiments. In embodiments, linker L1 is a phosphate (—(HO)P(═O)—). In embodiments, the linker L1 is a modified phosphate. In embodiments, the modified phosphate is phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, methylphosphonate, or guanidinopropyl phosphoramidate.

In embodiments, Q is a hydrogen or a chain terminating nucleoside. Some non-limiting examples of a chain terminating nucleoside include, but are not limited to, for example, ddC, inverted dT, 3′-phosphate nucleoside, 3′-oxime nucleoside, 3′-azidomethyl nucleoside, or 3′-methyl nucleoside. In embodiments, Q is a hydrogen. In embodiments, Q is ddC. In embodiments, Q is inverted dT. In embodiments, Q is 3′-phosphate nucleoside. In embodiments, Q is 3′-oxime nucleoside. In embodiments, Q is 3′-azidomethyl nucleoside. In embodiments, Q is 3′-methyl nucleoside.

In embodiments, the linker (L) capable of binding the purification handle is linked to the nucleoside via a 3′-carbon or a 2′-carbon of a sugar of the nucleoside. In embodiments, the linker (L) capable of binding the purification handle is linked to the nucleoside via a nucleobase of the nucleoside. In embodiments, the nucleoside is an unmodified nucleoside. In embodiments, the nucleoside is a modified nucleoside. The modified nucleoside is as defined herein including in embodiments. In embodiments, the sugar is a modified sugar. In embodiments, the sugar is an unmodified sugar. The modified sugar is as defined herein including in embodiments. In embodiments, the nucleobase is an unmodified nucleobase. In embodiments, the nucleobase is a modified nucleobase. The modified nucleobase is as defined herein including in embodiments.

In embodiments, the linker (L) capable of binding the purification handle is linked to the nucleoside via a nucleobase of the nucleoside and L1 linker is linked to the nucleoside via a 3′-carbon or a 2′-carbon of a sugar of the nucleoside. In embodiments, the linker (L) capable of binding the purification handle is linked to the nucleoside via a 3′-carbon or a 2′-carbon of a sugar of the nucleoside and L1 linker is linked to the nucleoside via a nucleobase of the nucleoside.

In embodiments, the purification handle is linked to the nucleoside via a 3′-carbon or a 2′-carbon of a sugar of the nucleoside. In embodiments, the purification handle is linked to the nucleoside via a nucleobase of the nucleoside. In embodiments, the nucleoside is an unmodified nucleoside. In embodiments, the nucleoside is a modified nucleoside. The modified nucleoside is as defined herein including in embodiments. In embodiments, the sugar is a modified sugar. In embodiments, the sugar is an unmodified sugar. The modified sugar is as defined herein including in embodiments. In embodiments, the nucleobase is an unmodified nucleobase. In embodiments, the nucleobase is a modified nucleobase. The modified nucleobase is as defined herein including in embodiments.

Some non-limiting examples of compounds of Formula (III) include, but are not limited to, for example:

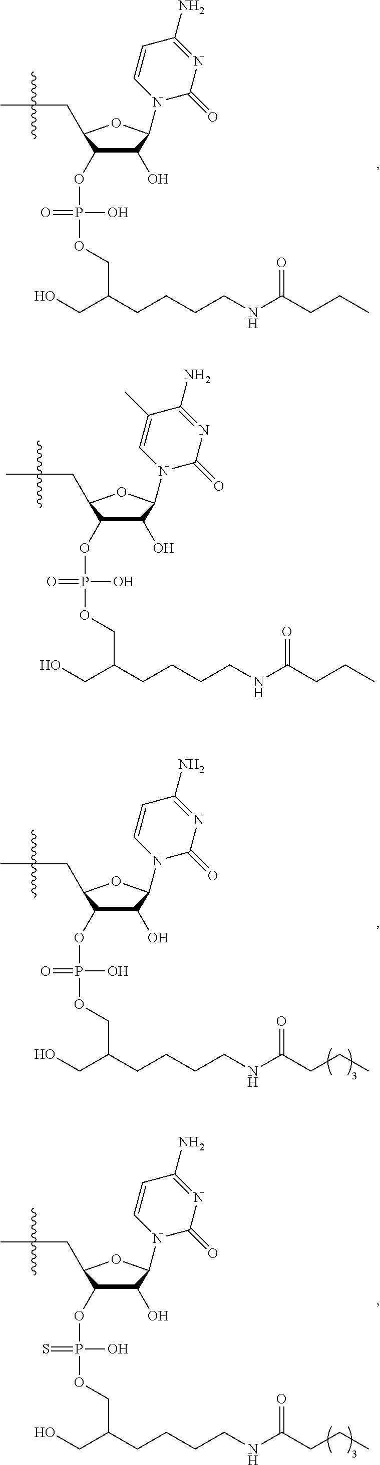

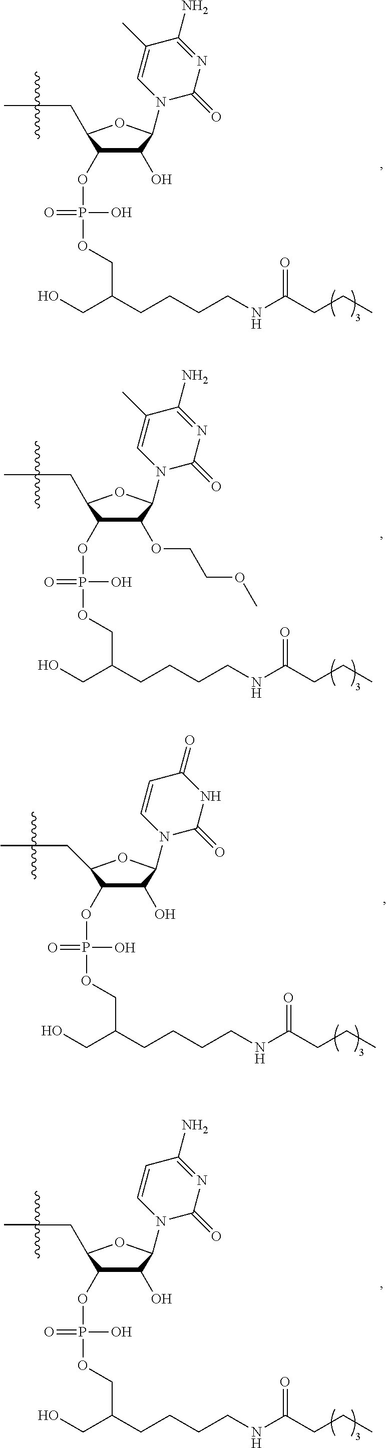

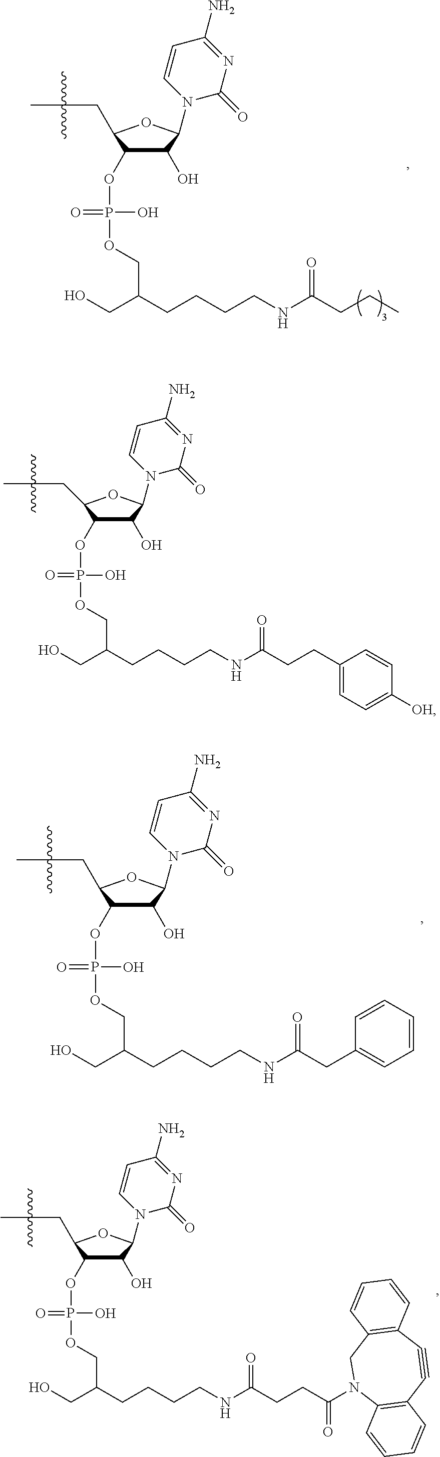

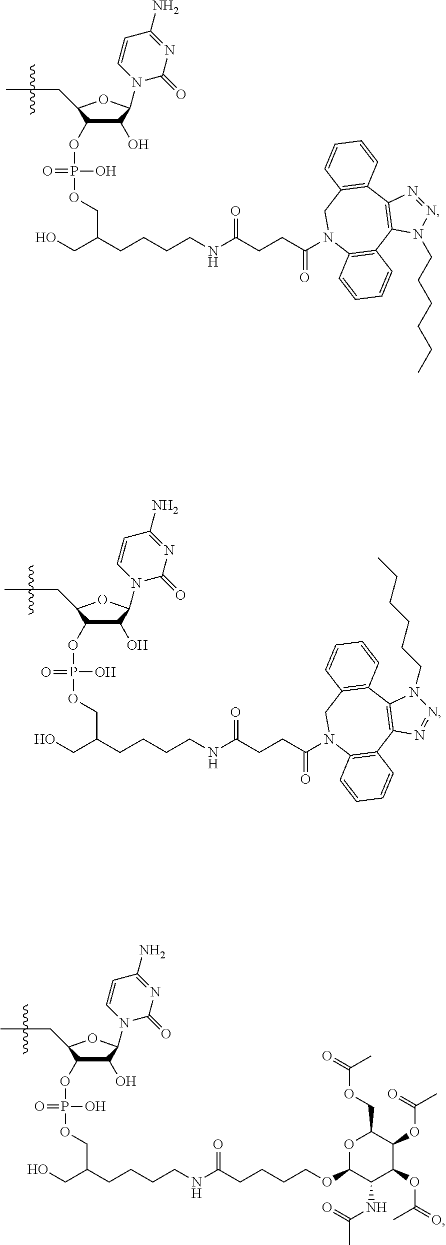

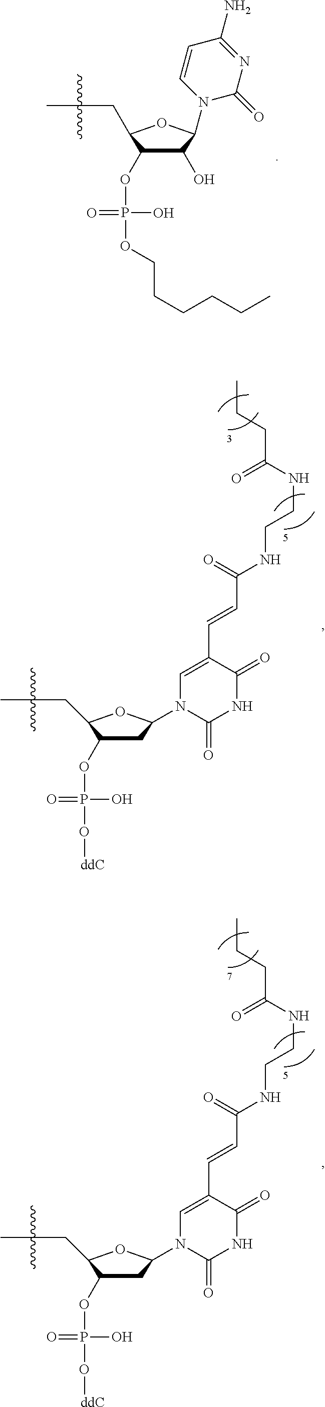

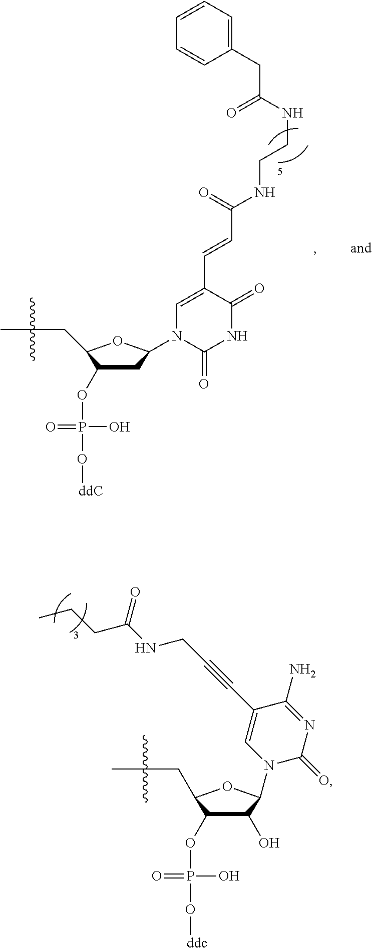

wherein the wavy line ( [symbol] ) indicates connection to a hydroxyl group (OH), phosphate, diphosphate, triphosphate, or modified phosphate. In embodiments, the modified phosphate is phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, methylphosphonate, or guanidinopropyl phosphoramidate.

In embodiments, a compound of Formula (III) comprises, for example,

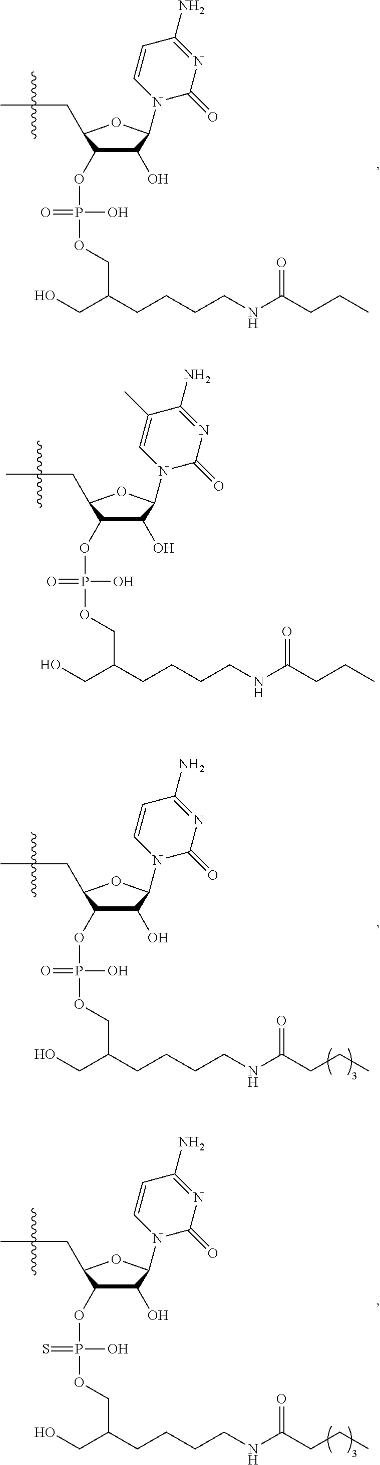

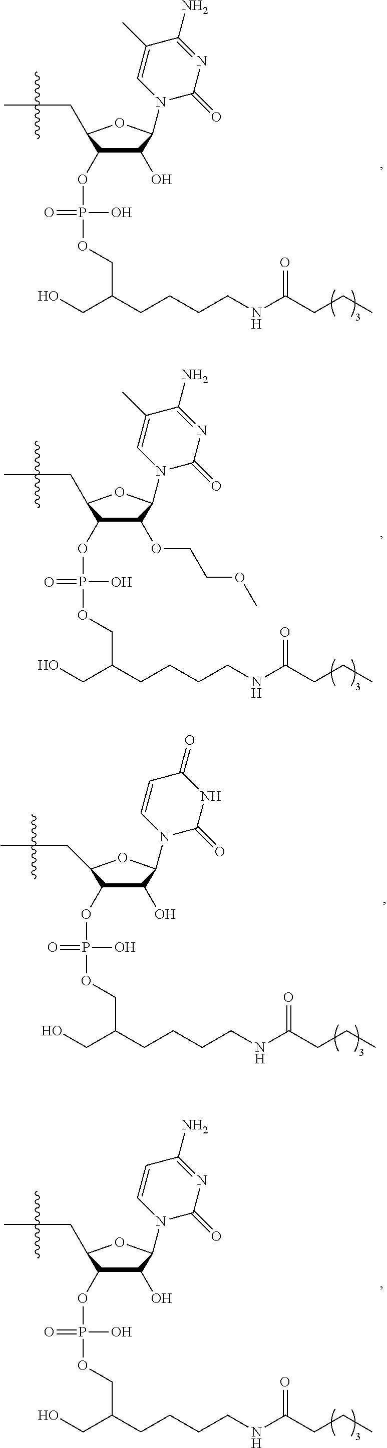

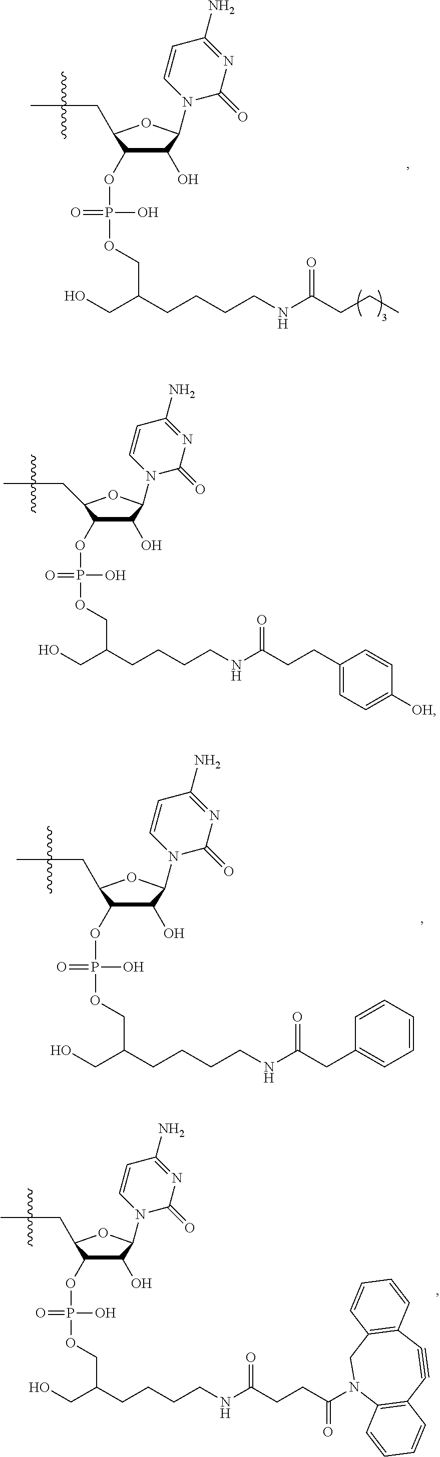

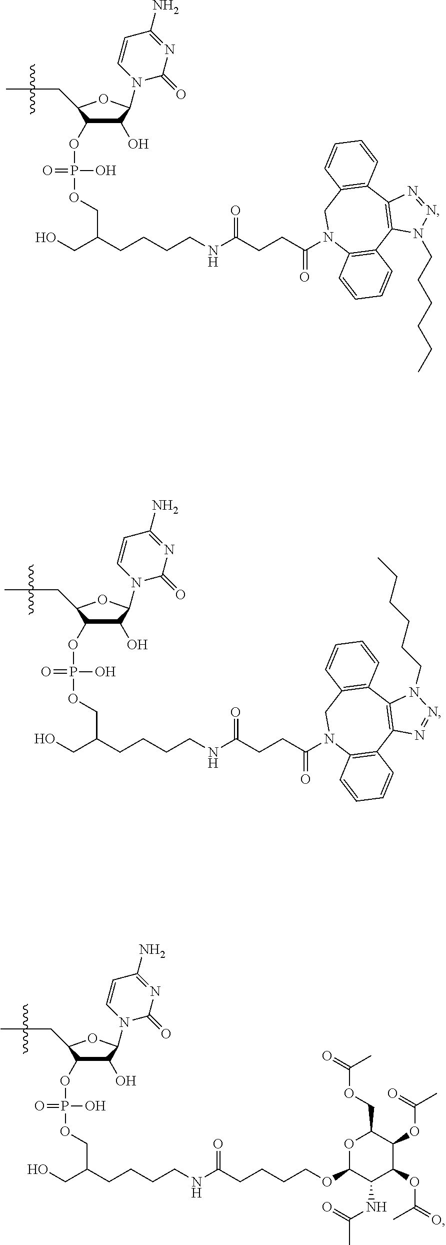

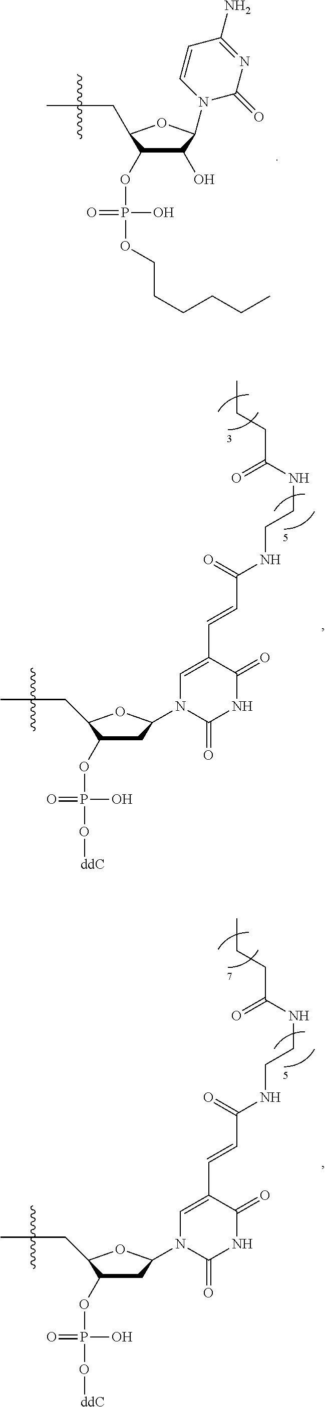

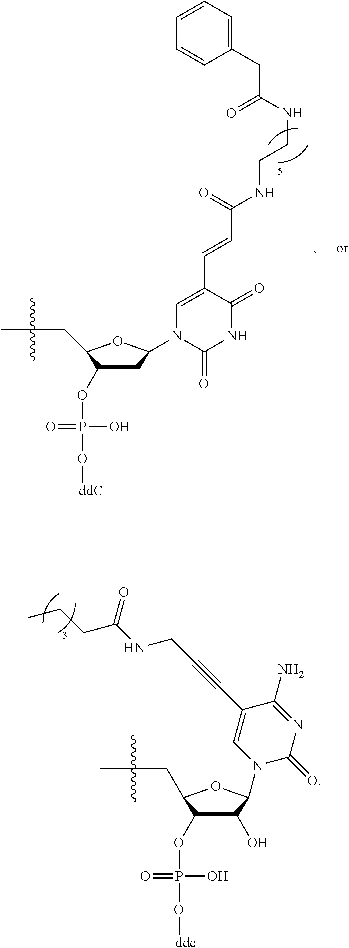

Some non-limiting examples of compounds of Formula (IV) include, but are not limited to, for example:

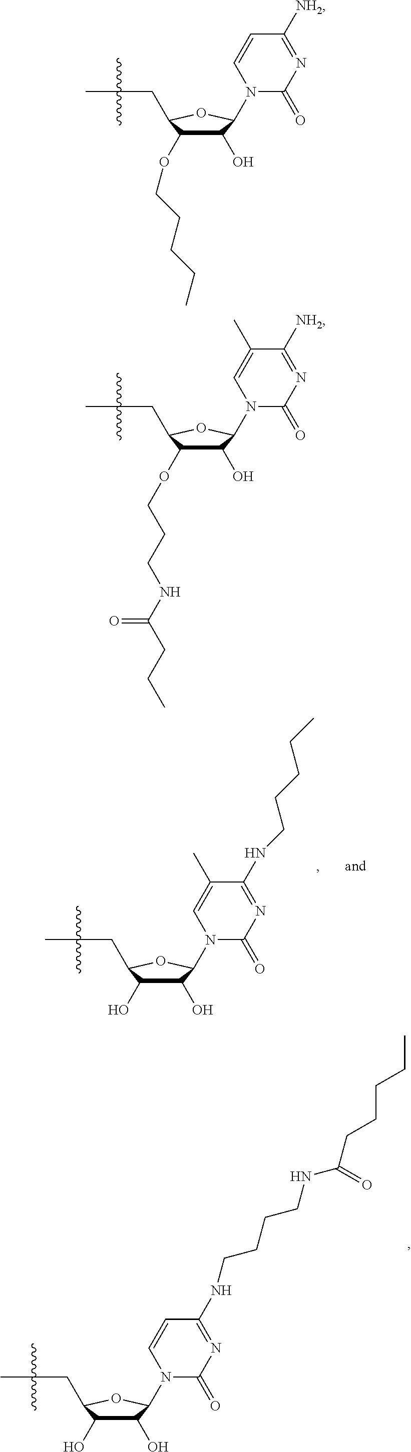

wherein the wavy line ( [symbol] ) indicates connection to a hydroxyl group (OH), phosphate, diphosphate, triphosphate, or modified phosphate. In embodiments, the modified phosphate is phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, methylphosphonate, or guanidinopropyl phosphoramidate.

In embodiments, a compound of Formula (IV) comprises, for example,

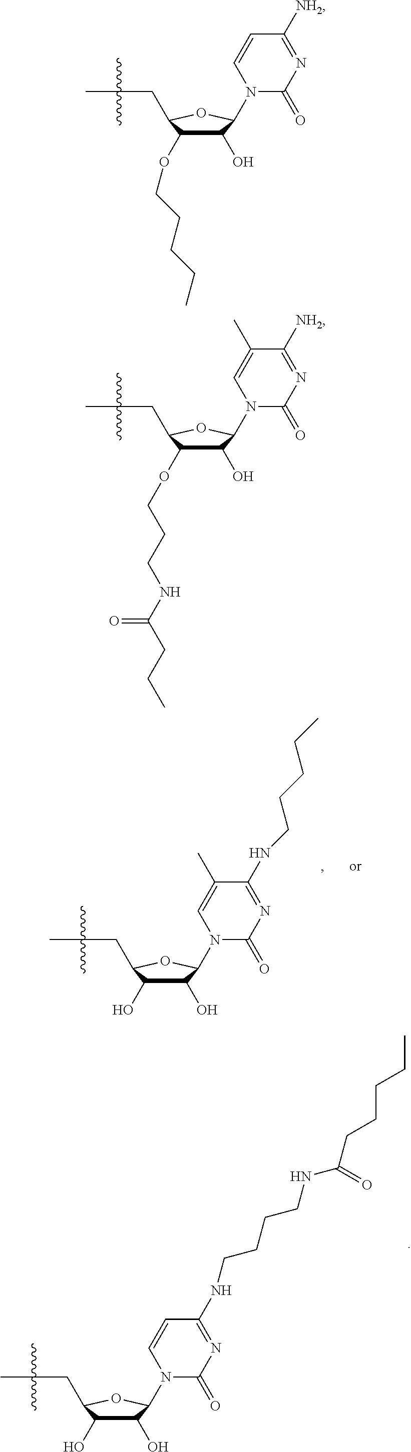

In an aspect, provided herein is a method of increasing the expression of a protein or a peptide of interest in a cell, comprising contacting the cell with an RNA molecule comprising any one of the compounds of Formula (III) or Formula (IV) described herein, wherein the RNA molecule encodes the protein or peptide of interest, wherein the expression is increased when compared to that of an RNA molecule not comprising any one of the compounds of Formula (III) or Formula (IV) described herein, optionally wherein the cell is isolated, in vitro, or ex vivo. In an aspect, provided herein is a method of increasing the expression of a protein or a peptide of interest in a cell, comprising contacting the cell with an RNA molecule comprising any one of the compounds of Formula (III) or Formula (IV) described herein, wherein the RNA molecule encodes the protein or peptide of interest, wherein the expression is increased when compared to that of an RNA molecule not comprising any one of the compounds of Formula (III) or Formula (IV) described herein, optionally wherein the cell is isolated, in vitro. In an aspect, provided herein is a method of increasing the expression of a protein or a peptide of interest in a cell, comprising contacting the cell with an RNA molecule comprising any one of the compounds of Formula (III) or Formula (IV) described herein, wherein the RNA molecule encodes the protein or peptide of interest, wherein the expression is increased when compared to that of an RNA molecule not comprising any one of the compounds of Formula (III) or Formula (IV) described herein, optionally wherein the cell is isolated, ex vivo.

In an aspect, provided herein is a method of increasing the half-life of an RNA molecule in a cell comprising contacting the cell with the RNA molecule comprising any one of the compounds of Formula (III) or Formula (IV) described herein, wherein the half-life is increased when compared to that of an RNA molecule not comprising any one of the compounds of Formula (III) or Formula (IV) described herein, optionally wherein the cell is isolated, in vitro, or ex vivo. In an aspect, provided herein is a method of increasing the half-life of an RNA molecule in a cell comprising contacting the cell with the RNA molecule comprising any one of the compounds of Formula (III) or Formula (IV) described herein, wherein the half-life is increased when compared to that of an RNA molecule not comprising any one of the compounds of Formula (III) or Formula (IV) described herein, optionally wherein the cell is isolated, in vitro. In an aspect, provided herein is a method of increasing the half-life of an RNA molecule in a cell comprising contacting the cell with the RNA molecule comprising any one of the compounds of Formula (III) or Formula (IV) described herein, wherein the half-life is increased when compared to that of an RNA molecule not comprising any one of the compounds of Formula (III) or Formula (IV) described herein, optionally wherein the cell is isolated, ex vivo.

### RNA Synthesis

In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′UTR, and a poly-A region 3′ of the ORF, wherein the stabilizing region is added 3′ to the poly-A region and the stabilizing region comprises one or more purification handles or one or more linkers capable of binding a purification handle. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises one or more purification handles. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises one purification handle. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises two purification handles. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises three purification handles. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises four purification handles. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises five purification handles. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises more than five purification handles.

In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises one or more linkers capable of binding a purification handle. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises one linker capable of binding a purification handle. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises two linkers capable of binding a purification handle. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises three linkers capable of binding a purification handle. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises four linkers capable of binding a purification handle. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises five linkers capable of binding a purification handle. In an aspect, provided herein is a method of preparing any one of the RNA molecules described herein, comprising covalently joining a stabilizing region to a precursor RNA comprising a 5′-cap, optionally a 5′ UTR, an ORF encoding a protein, optionally a 3′ UTR, and a poly-A region 3′ of the ORF, wherein the stabilizing region is added 3′ to the poly-A region and the stabilizing region comprises more than five linkers capable of binding a purification handle.

In embodiments, the 3′-stabilizing region is added to the precursor RNA by ligation. In embodiments, the ligation is an enzymatic or chemical ligation. In embodiments, the 3′-stabilizing region is added to the precursor RNA using a polymerase. In embodiments, the 3′-stabilizing region is added to the precursor RNA by ligation followed by using a polymerase. In embodiments, the 3′-stabilizing region is added to the precursor RNA by using a polymerase followed by ligation.

In an aspect, provided herein are in vitro methods for synthesizing capped RNA transcripts, including capped messenger RNA (mRNA) transcripts. The methods described herein comprise (a) forming a reaction mixture comprising a cap analog, a DNA template, and an RNA polymerase; and (b) incubating the reaction mixture under conditions that allow transcription of the DNA template to produce capped mRNA transcripts. In the methods described herein, the reaction mixture comprises NTPs, including ATP, CTP, GTP and UTP. One or more of the NTPs in the in vitro transcription reaction mixture can be modified NTPs. Exemplary modified nucleosides include, but are not limited to, inosine, 7-deazaguanosine, 7-methylguanosine, dihyrouridine, 2′-O-methylguanosine, 2′-fluoro-2′-deoxycytidine, pseudouridine, N1-methylpseudouridine, and 5-methyluridine. In some methods, one or more uridines in the in vitro transcribed RNA are replaced by a modified nucleoside(s). In some methods, one or more nucleosides in the in vitro transcribed RNA are replaced by modified nucleoside(s). Optionally, some methods further comprise incubating the reaction mixture comprising the capped mRNA transcripts with a DNase I buffer including Ca2+ and DNase I to degrade and remove the DNA template.

Optionally, some methods further comprise subjecting the DNase treated reaction mixture to eliminate proteins from the in vitro transcription reaction. Optionally, some methods further comprise subjecting the DNase treated reaction mixture to phosphatase treatment. Optionally, the method further comprises subjecting the DNase treated reaction mixture to one or more purification steps. The mRNA transcripts produced by the methods described herein can be purified using one or more purification techniques known to those of skill in the art. See, Baronti et al., (2018) Anal. Bioanal. Chem. 410(14): 3239-33252.

For example, the mRNAs can be purified by liquid chromatography (e.g., HPLC, reversed-phase ion pairing HPLC (RP—IP-HPLC), anion-exchange chromatography, cation exchange chromatography, affinity chromatography, size-exclusion chromatography), precipitation, diafiltration, tangential flow filtration, oligo dT chromatography, silica membrane purification, and hydrophobic interaction chromatography, to name a few. The synthesized capped mRNA transcripts can be substantially free of impurities such as DNA, protein, double-stranded RNA and/or incomplete mRNA transcripts.

In embodiments, a 3′-stabilizing region is covalently linked to the 3′-end of a precursor RNA by a linker that can be formed by ligation. In embodiments, the linker is a bioconjugate linker. In embodiments, the linker can be formed by an enzymatic ligation, a splint ligation, or a chemical ligation. In embodiments, the linker can be formed by an enzymatic ligation or a chemical ligation. In embodiments, the linker can be formed by an enzymatic ligation. In embodiments, the linker can be formed by a chemical ligation. In embodiments, the linker can be formed by a splint ligation.

In embodiments, a linker that can be formed by ligation between a 3′-stabilizing region and the 3′-end of a precursor RNA includes, but is not limited to, for example, reaction of sulfhydryl groups, amino groups, phosphate groups and/or hydroxyls or any appropriate reactive group. Multiple cross-linkers are known for the conjugation of the 3′-stabilizing region with the a precursor RNA. For example, NHS/EDC allows for the conjugation of primary amine groups with carboxyl groups; sulfo-EMCS ([N-ε-Maleimidocaproic acid]hydrazide (maleimide and NHS-ester) are reactive towards sulfhydryl and amino groups.

In embodiments, accessible amine groups present on the 3′-stabilizing region or the 3′-end of a precursor RNA may react with NHS esters. An amide bond is formed when the NHS ester reacts with primary amines. In embodiments, accessible thiol groups present on the 3′-stabilizing region or the 3′-end of a precursor RNA may react with maleimido group, creating a thioether linkage. In embodiments, accessible phosphate groups present on the 3′-stabilizing region or the 3′-end of a precursor RNA may react with imidazole, triazole, or tetrazole activated phosphate.

In embodiments, the linker can be formed by click-chemistry reaction. In embodiments, one of the reactive groups is attached to the 3′-stabilizing region, and the other reactive group is attached to the 3′-end of the precursor RNA. Reactive group pairs include the following, non-limiting examples, alkynyl group and azido group; diene group (for example, 1,3-butadiene, cyclopentadiene, cyclohexadiene, or furan) and a dienophile (for example, any alkenyl or any alkynyl); an aldehyde and an amino group; Michael acceptor (for example, conjugated alkenyl group such as α,β-unsaturated ketone, α,β-unsaturated ester, α,β-unsaturated nitrile) and a Michael donor (for example, thiolate, amine, enolate, enamine). These are only non-limiting examples of various pairs available for click-chemistry reactions.

In embodiments, the 3′-stabilizing region comprises one nucleoside. In embodiments, the 3′-stabilizing region comprises two nucleosides. In embodiments, the 3′-stabilizing region comprises three nucleosides. In embodiments, the 3′-stabilizing region comprises four nucleosides. In embodiments, the 3′-stabilizing region comprises five nucleosides. In embodiments, the 3′-stabilizing region comprises six nucleosides. In embodiments, the 3′-stabilizing region comprises seven nucleosides. In embodiments, the 3′-stabilizing region comprises eight nucleosides. In embodiments, the 3′-stabilizing region comprises nine nucleosides. In embodiments, the 3′-stabilizing region comprises ten nucleosides. In embodiments, the 3′-stabilizing region comprises twenty nucleosides. In embodiments, the 3′-stabilizing region comprises thirty nucleosides. In embodiments, the 3′-stabilizing region comprises forty nucleosides. In embodiments, the 3′-stabilizing region comprises fifty nucleosides.

In embodiments, the 3′-stabilizing region is covalently linked to the 3′-end of the precursor RNA by a linker that can be formed using a polymerase. In embodiments, the polymerase is poly A, poly U, or RNA nucleotidyl transferase. In embodiments, the polymerase is poly A. In embodiments, the polymerase is poly U. In embodiments, the polymerase is poly RNA nucleotidyl transferase. In embodiments, a polymerase catalyzes the reaction between a polyphosphate group (such as for example, diphosphate, triphosphate, or tetraphosphate) at the 5′-end of the 3′-stabilizing region and a nucleophile (for example, hydroxyl, amine, or thiol) at the 3′-end of the precursor RNA.

In embodiments, the polymerase catalyzes the reaction of one NTP with the 3′-end of the precursor RNA. In embodiments, the polymerase catalyzes the reaction of two NTPs with the 3′-end of the precursor RNA. In embodiments, the polymerase catalyzes the reaction of three NTPs with the 3′-end of the precursor RNA. In embodiments, the polymerase catalyzes the reaction of four NTPs with the 3′-end of the precursor RNA. In embodiments, the polymerase catalyzes the reaction of five NTPs with the 3′-end of the precursor RNA. In embodiments, the polymerase catalyzes the reaction of six NTPs with the 3′-end of the precursor RNA. In embodiments, the polymerase catalyzes the reaction of seven NTPs with the 3′-end of the precursor RNA. In embodiments, the polymerase catalyzes the reaction of eight NTPs with the 3′-end of the precursor RNA. In embodiments, the polymerase catalyzes the reaction of nine NTPs with the 3′-end of the precursor RNA. In embodiments, the polymerase catalyzes the reaction of ten NTPs with the 3′-end of the precursor RNA.

In embodiments, the polymerase catalyzes the reaction of one protected NTP with the 3′-end of the precursor RNA, followed by deprotection of the NTP and subsequent reaction with a second protected NTP, followed by deprotection of the NTP and subsequent reaction with a third protected NTP. In embodiments, the polymerase can catalyze at least one reaction with protected NTP followed by its deprotection. In embodiments, the polymerase can catalyze at least two sequential reactions with protected NTPs followed by their deprotection. In embodiments, the polymerase can catalyze at least three sequential reactions with protected NTPs followed by their deprotection. In embodiments, the polymerase can catalyze at least four sequential reactions with protected NTPs followed by their deprotection. In embodiments, the polymerase can catalyze at least ten sequential reactions with protected NTPs followed by their deprotection. In embodiments, the polymerase can catalyze at least twenty sequential reactions with protected NTPs followed by their deprotection.

In embodiments, the purification handle is linked to the 3′-stabilizing region via a linker (L). In embodiments, L is a bond, —S(O)₂—, —N(R)—, —O—, —S—, —C(O)—, —C(O)N(R)-, —N(R)C(O)-, —N(R)C(O)NH—, —NHC(O)N(R)-, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or any combination thereof; and
R is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or any combination thereof.

In embodiments, L is a substituted (e.g., with a substituent group, a size-limited substituent group or a lower substituent group) or unsubstituted alkylene (e.g., C₁-C₈ alkylene, C₁-C₆ alkylene, or C₁-C₄ alkylene). In embodiments, L is a substituted (e.g., with a substituent group, a size-limited substituent group or a lower substituent group) alkylene (e.g., C₁-C₈ alkylene, C₁-C₆ alkylene, or C₁-C₄ alkylene). In embodiments, L is unsubstituted alkylene (e.g., C₁-C₈ alkylene, C₁-C₆ alkylene, or C₁-C₄ alkylene). In embodiments, L is a substituted (e.g., with a substituent group, a size-limited substituent group or a lower substituent group) or unsubstituted heteroalkylene (e.g., 2 to 8 membered heteroalkylene, 2 to 6 membered heteroalkylene, or 2 to 4 membered heteroalkylene). In embodiments, L is a substituted (e.g., with a substituent group, a size-limited substituent group or a lower substituent group) heteroalkylene (e.g., 2 to 8 membered heteroalkylene, 2 to 6 membered heteroalkylene, or 2 to 4 membered heteroalkylene). In embodiments, L is an unsubstituted heteroalkylene (e.g., 2 to 8 membered heteroalkylene, 2 to 6 membered heteroalkylene, or 2 to 4 membered heteroalkylene). In embodiments, L is a substituted (e.g., with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene). In embodiments, L is a substituted (e.g., with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, L is an unsubstituted cycloalkylene (e.g., C₃-C₈ cycloalkylene, C₃-C₆ cycloalkylene, or C₅-C₆ cycloalkylene). In embodiments, L is a substituted (e.g., with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene). In embodiments, L is a substituted (e.g., with a substituent group, a size-limited substituent group or a lower substituent group) heterocycloalkylene (e.g., 3 to 8 membered heterocycloalkylene, 3 to 6 membered heterocycloalkylene, or 5 to 6 membered heterocycloalkylene). In embodiments, L is an unsubstituted heterocycloalkylene (e.g., 3 to 8 membered heterocycloalkylene, 3 to 6 membered heterocycloalkylene, or 5 to 6 membered heterocycloalkylene). In embodiments, L is a substituted (e.g., with a substituent group, a size-limited substituent group or a lower substituent group) or unsubstituted arylene (e.g., C₆-C₁₀ arylene, C₁₀ arylene, or phenylene). In embodiments, L is a substituted (e.g., with a substituent group, a size-limited substituent group or a lower substituent group) arylene (e.g., C₆-C₁₀ arylene, C₁₀ arylene, or phenylene). In embodiments, L is an unsubstituted arylene (e.g., C₆-C₁₀ arylene, C₁₀ arylene, or phenylene). In embodiments, L is a substituted (e.g., with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene). In embodiments, L is a substituted (e.g., with a substituent group, a size-limited substituent group or a lower substituent group) heteroarylene (e.g., 5 to 10 membered heteroarylene, 5 to 9 membered heteroarylene, or 5 to 6 membered heteroarylene). In embodiments, L is an unsubstituted heteroarylene (e.g., 5 to 10 membered heteroarylene, 5 to 9 membered heteroarylene, or 5 to 6 membered heteroarylene).

In embodiments, R is independently hydrogen, halogen, —CCl₃, —CBr₃, —CF₃, —CI₃, —CH₂Cl, —CH₂Br, —CH₂F, —CH₂I, —CHC1₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R is a substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R is a substituted (e.g. with a substituent group, a size-limited substituent group or a lower substituent group) alkyl (e.g., C₁-C₈ alkyl, C₁-C₆ alkyl, or C₁-C₄ alkyl). In embodiments, R is an unsubstituted alkyl (e.g., C₁-C₈ alkyl, C₁-C₆ alkyl, or C₁-C₄ alkyl). In embodiments, R is a substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl). In embodiments, R is a substituted (e.g. with a substituent group, a size-limited substituent group or a lower substituent group) heteroalkyl (e.g., 2 to 8 membered heteroalkyl, 2 to 6 membered heteroalkyl, or 2 to 4 membered heteroalkyl). In embodiments, R is an unsubstituted heteroalkyl (e.g., 2 to 8 membered heteroalkyl, 2 to 6 membered heteroalkyl, or 2 to 4 membered heteroalkyl). In embodiments, R is a substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl). In embodiments, R is a substituted (e.g. with a substituent group, a size-limited substituent group or a lower substituent group) cycloalkyl (e.g., C₃-C₈ cycloalkyl, C₃-C₆ cycloalkyl, or C₅-C₆ cycloalkyl). In embodiments, R is an unsubstituted cycloalkyl (e.g., C₃-C₈ cycloalkyl, C₃-C₆ cycloalkyl, or C₅-C₆ cycloalkyl). In embodiments, R is a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R is a substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R is an unsubstituted heterocycloalkyl (e.g., 3 to 8 membered heterocycloalkyl, 3 to 6 membered heterocycloalkyl, or 5 to 6 membered heterocycloalkyl). In embodiments, R is a substituted (e.g. with a substituent group, a size-limited substituent group or a lower substituent group) or unsubstituted aryl (e.g., C₆-C₁₀ aryl, C₁₀ aryl, or phenyl). In embodiments, R is a substituted (e.g. with a substituent group, a size-limited substituent group or a lower substituent group) aryl (e.g., C₆-C₁₀ aryl, C₁₀ aryl, or phenyl). In embodiments, R is an unsubstituted aryl (e.g., C₆-C₁₀ aryl, C₁₀ aryl, or phenyl). In embodiments, R is a substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, R is a substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, R is an unsubstituted heteroaryl (e.g., 5 to 10 membered heteroaryl, 5 to 9 membered heteroaryl, or 5 to 6 membered heteroaryl).

In embodiments, L is a substituted or unsubstituted alkylene or substituted or unsubstituted heteroalkylene. In embodiments, L is a substituted (e.g., with a substituent group, a size-limited substituent group or a lower substituent group) or unsubstituted alkylene (e.g., C₁-C₈ alkylene, C₁-C₆ alkylene, or C₁-C₄ alkylene). In embodiments, L is a substituted (e.g., with a substituent group, a size-limited substituent group or a lower substituent group) alkylene (e.g., C₁-C₈ alkylene, C₁-C₆ alkylene, or C₁-C₄ alkylene). In embodiments, L is unsubstituted alkylene (e.g., C₁-C₈ alkylene, C₁-C₆ alkylene, or C₁-C₄ alkylene). In embodiments, L is a substituted (e.g., with a substituent group, a size-limited substituent group or a lower substituent group) or unsubstituted heteroalkylene (e.g., 2 to 8 membered heteroalkylene, 2 to 6 membered heteroalkylene, or 2 to 4 membered heteroalkylene). In embodiments, L is a substituted (e.g., with a substituent group, a size-limited substituent group or a lower substituent group) heteroalkylene (e.g., 2 to 8 membered heteroalkylene, 2 to 6 membered heteroalkylene, or 2 to 4 membered heteroalkylene). In embodiments, L is an unsubstituted heteroalkylene (e.g., 2 to 8 membered heteroalkylene, 2 to 6 membered heteroalkylene, or 2 to 4 membered heteroalkylene).

In embodiments, L is

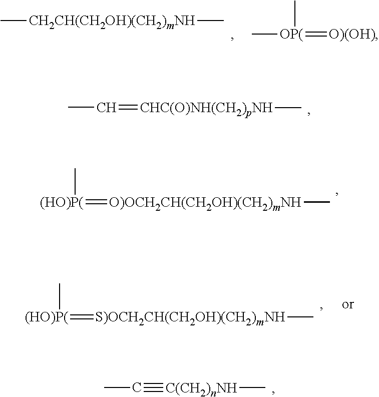

wherein m is an integer from 0 to 8; n is an integer from 0 to 8; and p is an integer from 0 to 10. In embodiments, L is

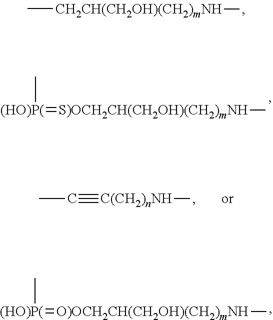

wherein m is an integer from 0 to 8. In embodiments, L is —C≡C(CH₂)ₙNH—, wherein n is an integer from 0 to 8. In embodiments, L is —CH═CHC(O)NH(CH₂)ₚNH-, wherein p is an integer from 0 to 10. In embodiments, L is

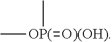

In embodiments, L is —CH₂CH(CH₂OH)(CH₂)₄NH- or —CH═CHC(O)NH(CH₂)₆NH-. In embodiments, L is —CH₂CH(CH₂OH)(CH₂)₄NH-. In embodiments, L is —CH═CHC(O)NH(CH₂)₆NH-.

In embodiments, L is

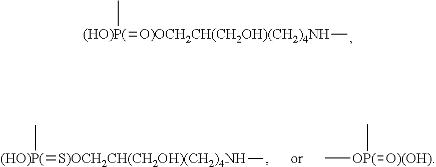

In embodiments, L is

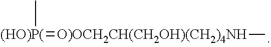

In embodiments, L is

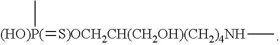

In embodiments, L is

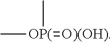

In embodiments, L is —C≡C(CH₂)NH-.

In embodiments, L is a phosphate or a modified phosphate. Some non-limiting examples of modified phosphate include, but are not limited to, phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, methylphosphonate, and guanidinopropyl phosphoramidate.

In embodiments, the modified L may be, for example, phosphorothioate, phosphoroselenate, boranophosphate, boranophosphate ester, hydrogen phosphonate, phosphoramidate, phosphorodiamidate, alkyl or aryl phosphonate, or phosphotriester.

In embodiments, the purification handle is a hydrophobic group which is covalently linked to the 3′-stabilizing region of the RNA molecules described herein, such group may be removable or non-removable. In embodiments, the purification handle is linked, via covalent bond to the 3′-stabilizing region directly. In embodiments, the purification handle is linked, via a covalent bond, to the 3′-stabilizing region via linker (L). In embodiments, the purification handle is a removable group. In embodiments, the purification handle is a non-removable group.

In embodiments, the purification handle includes, for example, but is not limited to C₆-C₂₄ alkyls, C₄-C₂₄ alkenyls, C₄-C₂₄ alkynyls, C₃-C₈cycloalkyls, C₆-C₁₀aryls, silyl compounds, trityl compounds, lipids, dyes, steroids, vinyl ether compounds, modified and unmodified Fmoc compounds, and the like. Fluoro substituents or fluorosubstituted groups can be used to increase the hydrophobicity of a hydrophobic group. In embodiments, the purification handle includes a C₆-C₂₄ alkyl. In embodiments, the purification handle includes a C₄-C₂₄ alkenyl. In embodiments, the purification handle includes a C₄-C₂₄ alkynyl. In embodiments, the purification handle includes a C₃-C₈cycloalkyl. In embodiments, the purification handle includes a C₆-C₁₀aryl. In embodiments, the purification handle includes a silyl compound. In embodiments, the purification handle includes a trityl compound. In embodiments, the purification handle includes a lipid. In embodiments, the purification handle includes a steroid. In embodiments, the purification handle includes a vinyl ether compound. In embodiments, the purification handle includes a modified Fmoc compound. In embodiments, the purification handle includes an unmodified Fmoc compound.

In embodiments, the purification handle includes for example, but is not limited to propyl, butyl, pentyl, hexyl, heptyl, octyl, nonyl, decyl, undecyl, dodecyl, tridecyl, tetradecyl, and pentadecyl. In embodiments, the purification handle includes for example, but is not limited to phenyl, benzyl, (ethyl)carbonyl(azadibenzocyclooctyne) (DBCO), 4-ethylphenol, dibenzohexyltriazoloazocine, and 1′-O-butyl 3′, 4′, 6′-triacetyl GalNAc.

In embodiments, the purification handle includes for example, but is not limited to —C(O)-propyl, —C(O)-butyl, —C(O)-pentyl, —C(O)-hexyl, —C(O)-heptyl, —C(O)-octyl, —C(O)-nonyl, —C(O)-decyl, —C(O)-undecyl, —C(O)-dodecyl, —C(O)-tridecyl, —C(O)-tetradecyl, and —C(O)-pentadecyl. In embodiments, the purification handle includes for example, but is not limited to —C(O)-phenyl, —C(O)-benzyl, —C(O)-4-ethylphenol, —C(O)-(ethyl)carbonyl(azadibenzocyclooctyne), —C(O)-dibenzohexyltriazoloazocine, and —C(O)-1′-O-butyl 3′, 4′, 6′-triacetyl GalNAc.

In embodiments, the purification handle is (ethyl)carbonyl(azadibenzocyclooctyne) (DBCO)

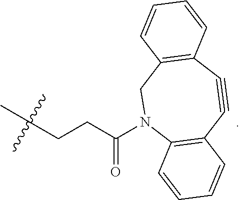 [SMILES: *CCC(=O)N1Cc2ccccc2C#Cc2ccccc21]

In embodiments, the purification handle is 4-ethylphenol

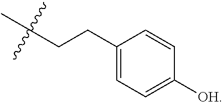 [SMILES: *CCc1ccc(O)cc1]

In embodiments, the purification handle is a mixture of two isomers of dibenzohexyltriazoloazocine

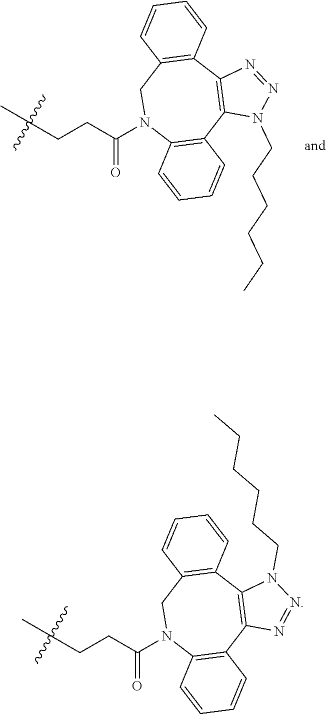 [SMILES: *CCC(=O)N1Cc2ccccc2-c2nnn(CCCCCC)c2-c2ccccc21 ; *CCC(=O)N1Cc2ccccc2-c2c(nnn2CCCCCC)-c2ccccc21]

In embodiments, the purification handle is 1′-O-butyl 3′, 4′, 6′-triacetyl GalNAc

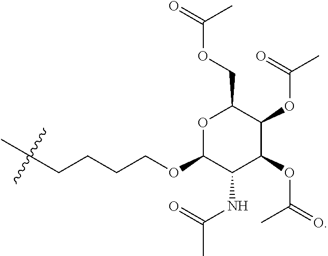 [SMILES: *CCCCO[C@H]1O[C@@H](COC(C)=O)[C@@H](OC(C)=O)[C@@H](OC(C)=O)[C@@H]1NC(C)=O]

In embodiments, the purification handle includes a lipid. In embodiments, without wishing to be bound by any particular theory, the purification handles that include lipids may lead to increased encapsulation of mRNA molecules, bearing lipid purification handles, in pharmaceutical carriers such as, for example, lipid nanoparticles (LNPs).

### Therapeutic Use

In an aspect, provided herein is a cell comprising any one of the RNA molecules described herein. In embodiments, the cell is an isolated cell. In embodiments, the cell is a mammalian cell. In embodiments, the cell is a human cell.

In an aspect, provided herein is a cell comprising a protein or a peptide translated from any one of the RNA molecules described herein. In an aspect, provided herein is a cell comprising a protein translated from any one of the RNA molecules described herein. In an aspect, provided herein is a cell comprising a peptide translated from any one of the RNA molecules described herein. In embodiments, the cell is an isolated cell. In embodiments, the cell is a mammalian cell. In embodiments, the cell is a human cell.

In embodiments, the RNA molecules described herein include pharmaceutically acceptable salts of the RNA molecules described herein. As used herein, “pharmaceutically acceptable salts” refers to derivatives of the disclosed RNA molecules wherein the parent RNA molecule is altered by converting an existing acid or base moiety to its salt form (e.g., by reacting the free base group with a suitable organic acid or inorganic acid). Representative salts include, but are not limited to, the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. Salts may include, for example, cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1; and Remington: The Science and Practice of Pharmacy, 23d Edition, Adejare et al. eds., Academic Press (2020); which are incorporated herein by reference in their entireties.)

In an aspect, provided herein is a pharmaceutical composition comprising any one of the RNA molecules described herein, and a pharmaceutically acceptable carrier. In embodiments, the pharmaceutically acceptable carrier includes, but is not limited to, a solvent, dispersion media, diluent, surface active agent, isotonic agent, thickening or emulsifying agent, lipid, liposome, nanoparticle, lipid nanoparticle (LNP), polymer, lipoplex, protein, or any mixture thereof. In embodiments, the pharmaceutical composition comprises a cell which comprises an RNA molecule described herein.

In embodiments, the pharmaceutically acceptable carrier is a solvent. In embodiments, the pharmaceutically acceptable carrier is a dispersion media. In embodiments, the pharmaceutically acceptable carrier is a diluent. In embodiments, the pharmaceutically acceptable carrier is a surface-active agent. In embodiments, the pharmaceutically acceptable carrier is an isotonic agent. In embodiments, the pharmaceutically acceptable carrier is a thickening agent. In embodiments, the pharmaceutically acceptable carrier is an emulsifying agent. In embodiments, the pharmaceutically acceptable carrier is a lipid. In embodiments, the pharmaceutically acceptable carrier is a liposome. In embodiments, the pharmaceutically acceptable carrier is a nanoparticle. In embodiments, the pharmaceutically acceptable carrier is a lipid nanoparticle (LNP). In embodiments, the pharmaceutically acceptable carrier is a polymer. In embodiments, the pharmaceutically acceptable carrier is a lipoplex. In embodiments, the pharmaceutically acceptable carrier is protein. In embodiments, the pharmaceutically acceptable carrier is a mixture of two or more of the following: a solvent, dispersion media, diluent, surface active agent, isotonic agent, thickening or emulsifying agent, lipid, liposome, nanoparticle, lipid nanoparticle (LNP), polymer, lipoplex, or protein.

In embodiments, the preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012).

Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the RNA molecules described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Administration of the RNA molecules and compositions described herein or pharmaceutically acceptable salts thereof can be carried out using therapeutically effective amounts of the RNA molecules and compositions described herein or pharmaceutically acceptable salts thereof as described herein for periods of time effective to prevent or treat a disease or disorder. Administration of the RNA molecules and compositions described herein or pharmaceutically acceptable salts thereof can be carried out using therapeutically effective amounts of the RNA molecules and compositions described herein or pharmaceutically acceptable salts thereof as described herein for periods of time effective to prevent a disease or disorder. Administration of the RNA molecules and compositions described herein or pharmaceutically acceptable salts thereof can be carried out using therapeutically effective amounts of the RNA molecules and compositions described herein or pharmaceutically acceptable salts thereof as described herein for periods of time effective to treat a disease or disorder. The effective amount of the RNA molecules and compositions described herein or pharmaceutically acceptable salts thereof as described herein may be determined by one of ordinary skill in the art.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

Any suitable formulation of the RNA molecules described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In embodiments, the RNA molecule is formulated for oral administration; in other embodiments, the RNA molecule is formulated for parenteral administration, such as injection or infusion.

Where contemplated RNA molecules are administered in a pharmacological composition, it is contemplated that the RNA molecules can be formulated in admixture with a pharmaceutically acceptable excipient and/or carrier. For example, contemplated RNA molecules can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated RNA molecules may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the RNA molecules described herein or derivatives thereof in combination with a pharmaceutically acceptable carrier and, in addition, may include other medicinal agents, pharmaceutical agents, carriers/excipients or diluents. By pharmaceutically acceptable is meant a material that is not biologically or otherwise undesirable, which can be administered to an individual along with the selected RNA molecule without causing unacceptable biological effects or interacting in a deleterious manner with the other components of the pharmaceutical composition in which it is contained.

To practice the method of the present invention, RNA molecules having formula and pharmaceutical compositions thereof may be administered orally, parenterally, by inhalation, topically (including transdermally, buccally, and sublingually), rectally, nasally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions may be prepared by any method well known in the art of pharmacy.

Such methods include the step of bringing in association RNA molecules of the invention or combinations thereof with any auxiliary agent. The auxiliary agent(s), also named accessory ingredient(s), include those conventional in the art, such as excipients (e.g., starch, lactose), fillers, binders (e.g., gelatin, cellulose, gum tragacanth), diluents, disintegrants (e.g., alginate, Primogel, and corn starch), lubricants (e.g., magnesium stearate, silicon dioxide), colorants, flavouring agents (e.g., glucose, sucrose, saccharin, methyl salicylate, and peppermint), anti-oxidants, wetting agents, or other material well known in the art for use in pharmaceutical formulations.

The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012).

Solid dosage forms for oral administration of the RNA molecules described herein or derivatives thereof include capsules, tablets, pills, powders, and granules. In such solid dosage forms, the RNA molecules described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the RNA molecules described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Routes of topical administration include nasal, bucal, mucosal, rectal, or vaginal applications. Compositions of the RNA molecules described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the RNA molecules described herein or derivatives thereof include ointments, lotions, creams, gels, pastes, suspensions, drops, powders, sprays, inhalants, and transdermal patches. One or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. Methods for preparing transdermal patches are disclosed, e.g., in Brown, et al. (1988) Ann. Rev. Med. 39:221-229 which is incorporated herein by reference. The RNA molecules described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the RNA molecules described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators. A nasal aerosol or inhalation compositions can be prepared according to techniques well-known in the art of pharmaceutical formulation and can be prepared as solutions in, for example saline, employingsuitable preservatives (for example, benzyl alcohol), absorption promoters to enhance bioavailability, and/or other solubilizing or dispersing agents known in the art.

The compositions may be presented in unit-dose or multi-dose containers, for example sealed vials and ampoules, and may be stored in a freeze-dried (lyophilised) condition requiring only the addition of sterile liquid carrier, for example water, prior to use.

In addition, the RNA molecules having Formula I or Formula II or any of the exemplary compounds disclosed herein or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof, may be administered alone or in combination with other therapeutic agents. Combination therapies according to the present invention comprise the administration of at least one exemplary RNA molecule of the present disclosure and at least one other therapeutic agent in a pharmaceutical composition. The at least one exemplary RNA molecule of the present disclosure and at least one other therapeutic agent(s) may be administered as a pharmaceutical composition separately or together. The amounts of the at least one exemplary RNA molecule of the present disclosure and the at least one other therapeutic agent(s) and the relative timings of administration will be selected in order to achieve the desired combined therapeutic effect.

In an aspect, provided herein is a method of treating a disease in a subject in need thereof comprising introducing an effective amount of any one of the RNA molecules described herein. In an aspect, provided herein is a method of treating a disease in a subject in need thereof comprising introducing an effective amount of a cell comprising any one of RNA molecules described herein. In an aspect, provided herein is a method of treating a disease in a subject in need thereof comprising introducing an effective amount of a cell comprising a protein or a peptide translated from any one of RNA molecules described herein. In an aspect, provided herein is a method of treating a disease in a subject in need thereof comprising introducing an effective amount of a cell comprising a peptide translated from any one of RNA molecules described herein. In an aspect, provided herein is a method of treating a disease in a subject in need thereof comprising introducing an effective amount of a cell comprising a protein translated from any one of RNA molecules described herein. In embodiments, the cell is an isolated cell. In embodiments, the cell is a mammalian cell. In embodiments, the cell is a human cell.

In an aspect, provided herein is a method of treating a disease in a subject in need thereof comprising introducing an effective amount of a pharmaceutical composition comprising any one of the RNA molecules described herein and a pharmaceutically acceptable carrier. In embodiments, the pharmaceutically acceptable carrier is a solvent, dispersion media, diluent, surface active agent, isotonic agent, thickening or emulsifying agent, lipid, liposome, nanoparticle, lipid nanoparticle (LNP), polymer, lipoplex, protein, or a mixture thereof. In embodiments, the pharmaceutically acceptable carrier is a lipid nanoparticle (LNP).

In an aspect, provided herein is a method of preventing a disease in a subject in need thereof comprising introducing an effective amount of any one of the RNA molecules described herein. In an aspect, provided herein is a method of preventing a disease in a subject in need thereof comprising introducing an effective amount of a cell comprising any one of RNA molecules described herein. In an aspect, provided herein is a method of preventing a disease in a subject in need thereof comprising introducing an effective amount of a cell comprising a protein or a peptide translated from any one of RNA molecules described herein. In an aspect, provided herein is a method of preventing a disease in a subject in need thereof comprising introducing an effective amount of a cell comprising a peptide translated from any one of RNA molecules described herein. In an aspect, provided herein is a method of preventing a disease in a subject in need thereof comprising introducing an effective amount of a cell comprising a protein translated from any one of RNA molecules described herein. In embodiments, the cell is an isolated cell. In embodiments, the cell is a mammalian cell. In embodiments, the cell is a human cell.

In an aspect, provided herein is a method of preventing a disease in a subject in need thereof comprising introducing an effective amount of a pharmaceutical composition comprising any one of the RNA molecules described herein and a pharmaceutically acceptable carrier. In embodiments, the pharmaceutically acceptable carrier is a solvent, dispersion media, diluent, surface active agent, isotonic agent, thickening or emulsifying agent, lipid, liposome, nanoparticle, lipid nanoparticle (LNP), polymer, lipoplex, protein, or a mixture thereof. In embodiments, the pharmaceutically acceptable carrier is a lipid nanoparticle (LNP).

In an aspect, provided herein is a method of increasing the expression of a protein or a peptide of interest in a cell, comprising contacting the cell with any one of the RNA molecules described herein, wherein the RNA molecule encodes the protein or peptide of interest, wherein the expression is increased when compared to that of an RNA molecule without the 3′-stabilizing region. In an aspect, provided herein is a method of increasing the expression of a peptide of interest in a cell, comprising contacting the cell with any one of the RNA molecules described herein, wherein the RNA molecule encodes the peptide of interest, wherein the expression is increased when compared to that of an RNA molecule without the 3′-stabilizing region. In an aspect, provided herein is a method of increasing the expression of a protein of interest in a cell, comprising contacting the cell with any one of the RNA molecules described herein, wherein the RNA molecule encodes the protein of interest, wherein the expression is increased when compared to that of an RNA molecule without the 3′-stabilizing region. In embodiments, the cell is isolated, in vitro, or ex vivo. In embodiments, the cell is an isolated cell. In embodiments, the cell is an in vitro cell. In embodiments, the cell is an ex vivo cell.

In an aspect, provided herein is a method of expressing a protein or a peptide of interest in a cell, comprising contacting the cell with any one of the RNA molecules described herein, wherein the RNA molecule encodes the protein or peptide of interest and the cell translates the protein or peptide of interest from the RNA molecule. In an aspect, provided herein is a method of expressing a peptide of interest in a cell, comprising contacting the cell with any one of the RNA molecules described herein, wherein the RNA molecule encodes the peptide of interest and the cell translates the peptide of interest from the RNA molecule. In an aspect, provided herein is a method of expressing a protein of interest in a cell, comprising contacting the cell with any one of the RNA molecules described herein, wherein the RNA molecule encodes the protein of interest and the cell translates the protein of interest from the RNA molecule. In embodiments, the cell is isolated, in vitro, or ex vivo. In embodiments, the cell is an isolated cell. In embodiments, the cell is an in vitro cell. In embodiments, the cell is an ex vivo cell.

In an aspect, provided herein is a method of increasing the half-life of an RNA molecule in a cell, comprising contacting the cell with any one of the RNA molecules described herein, wherein the wherein the half-life is increased when compared to that of an RNA molecule without the 3′-stabilizing region, optionally wherein the cell is isolated, in vitro, or ex vivo. In an aspect, provided herein is a method of increasing the half-life of an RNA molecule in a cell, comprising contacting the cell with any one of the RNA molecules described herein, wherein the half-life is increased when compared to that of an RNA molecule without the 3′-stabilizing region, optionally wherein the cell is isolated, in vitro. In an aspect, provided herein is a method of increasing the half-life of an RNA molecule in a cell, comprising contacting the cell with any one of the RNA molecules described herein, wherein the half-life is increased when compared to that of an RNA molecule without the 3′-stabilizing region, optionally wherein the cell is isolated, ex vivo. In embodiments, the cell is isolated, in vitro, or ex vivo. In embodiments, the cell is an isolated cell. In embodiments, the cell is isolated in vitro cell. In embodiments, the cell is isolated ex vivo cell.

In embodiments, the RNA molecules described herein can be used as guide RNAs (gRNAs) in gene editing.

### Kits

In an aspect, provided herein is a kit comprising any of the RNA molecules described herein. In embodiments, the kits may comprise sufficient amounts of the required components to allow for multiple treatments of a subject in need of such treatment. In embodiments, the kits may comprise sufficient amounts of the required components to allow for multiple experiments.

In embodiments, provided herein is a kit for protein production, including an RNA molecule, of Formula I or Formula II, comprising a 5′-cap, optionally a 5′ UTR, a translatable region, optionally a 3′ UTR, a poly-A region, and a 3′-stabilizing region, wherein the RNA molecule exhibits reduced degradation by exonucleases, and is separable from prematurely aborted RNA transcripts which lack poly A tails (using oligo dT column) or a 3′-stabilizing region (using HPLC column), and instructions for using the kit.

In embodiments, provided herein is a kit for protein production, including an RNA molecule, of Formula I or Formula II, comprising a 5′-cap, optionally a 5′ UTR, a translatable region, optionally a 3′ UTR, a poly-A region, a ligase or a polymerase for linking the 3′-stabilizing region to the RNA molecule. In embodiments, the kit comprises a buffer for performing the ligation. In embodiments, the kit further comprises instructions for administering any of the pharmaceutical compositions provided herein to a subject.

LIST OF SEQUENCES:

15 mer oligo SEQ ID NO: 1
5′-monophosphate-AAAAAAAAAAAAA(dTAm)(ddC)
39 mer oligo SEQ ID NO: 2
AAAAAAAAAAAAAAAAAAAAAAAAAAAAAAAAAAAAAAA
FLuc mRNA SEQ ID NO: 20 (all T nucleotides are 5-
methoxy uridines)
AGGAAATAAGAGAGAAAAGAAGAGTAAGAAGAAATATAAGAGCCACCATG
GAGGACGCCAAGAACATCAAGAAGGGCCCCGCCCCCTTCTACCCCCTGGA
GGACGGCACCGCCGGCGAGCAGCTGCACAAGGCCATGAAGCGGTACGCCC
TGGTGCCCGGCACCATCGCCTTCACCGACGCCCACATCGAGGTGGACATC
ACCTACGCCGAGTACTTCGAGATGAGCGTGCGGCTGGCCGAGGCCATGAA
GCGGTACGGCCTGAACACCAACCACCGGATCGTGGTGTGCAGCGAGAACA
GCCTGCAGTTCTTCATGCCCGTGCTGGGCGCCCTGTTCATCGGCGTGGCC
GTGGCCCCCGCCAACGACATCTACAACGAGCGGGAGCTGCTGAACAGCAT
GGGCATCAGCCAGCCCACCGTGGTGTTCGTGAGCAAGAAGGGCCTGCAGA
AGATCCTGAACGTGCAGAAGAAGCTGCCCATCATCCAGAAGATCATCATC
ATGGACAGCAAGACCGACTACCAGGGCTTCCAGAGCATGTACACCTTCGT
GACCAGCCACCTGCCCCCCGGCTTCAACGAGTACGACTTCGTGCCCGAGA
GCTTCGACCGGGACAAGACCATCGCCCTGATCATGAACAGCAGCGGCAGC
ACCGGCCTGCCCAAGGGCGTGGCCCTGCCCCACCGGACCGCCTGCGTGCG
GTTCAGCCACGCCCGGGACCCCATCTTCGGCAACCAGATCATCCCCGACA
CCGCCATCCTGAGCGTGGTGCCCTTCCACCACGGCTTCGGCATGTTCACC
ACCCTGGGCTACCTGATCTGCGGCTTCCGGGTGGTGCTGATGTACCGGTT
CGAGGAGGAGCTGTTCCTGCGGAGCCTGCAGGACTACAAGATCCAGAGCG
CCCTGCTGGTGCCCACCCTGTTCAGCTTCTTCGCCAAGAGCACCCTGATC
GACAAGTACGACCTGAGCAACCTGCACGAGATCGCCAGCGGCGGCGCCCC
CCTGAGCAAGGAGGTGGGCGAGGCCGTGGCCAAGCGGTTCCACCTGCCCG
GCATCCGGCAGGGCTACGGCCTGACCGAGACCACCAGCGCCATCCTGATC
ACCCCCGAGGGCGACGACAAGCCCGGCGCCGTGGGCAAGGTGGTGCCCTT
CTTCGAGGCCAAGGTGGTGGACCTGGACACCGGCAAGACCCTGGGCGTGA
ACCAGCGGGGCGAGCTGTGCGTGCGGGGCCCCATGATCATGAGCGGCTAC
GTGAACAACCCCGAGGCCACCAACGCCCTGATCGACAAGGACGGCTGGCT
GCACAGCGGCGACATCGCCTACTGGGACGAGGACGAGCACTTCTTCATCG
TGGACCGGCTGAAGAGCCTGATCAAGTACAAGGGCTACCAGGTGGCCCCC
GCCGAGCTGGAGAGCATCCTGCTGCAGCACCCCAACATCTTCGACGCCGG
CGTGGCCGGCCTGCCCGACGACGACGCCGGCGAGCTGCCCGCCGCCGTGG
TGGTGCTGGAGCACGGCAAGACCATGACCGAGAAGGAGATCGTGGACTAC
GTGGCCAGCCAGGTGACCACCGCCAAGAAGCTGCGGGGCGGCGTGGTGTT
CGTGGACGAGGTGCCCAAGGGCCTGACCGGCAAGCTGGACGCCCGGAAGA
TCCGGGAGATCCTGATCAAGGCCAAGAAGGGCGGCAAGATCGCCGTGTGA
TTAATTAAGCTGCCTTCTGCGGGGCTTGCCTTCTGGCCATGCCCTTCTTC
TCTCCCTTGCACCTGTACCTCTTGGTCTTTGAATAAAGCCTGAGTAGGAA
GAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAA
eGFP mRNA SEQ ID NO: 21 (all T nucleotides are N1-
methyl pseudouridines)
m6AGGAAATAAGAGAGAAAAGAAGAGTAAGAAGAAATATAAGAGCCACCA
TGGTGAGCAAGGGCGAGGAGCTGTTCACCGGGGTGGTGCCCATCCTGGTC
GAGCTGGACGGCGACGTAAACGGCCACAAGTTCAGCGTGTCCGGCGAGGG
CGAGGGCGATGCCACCTACGGCAAGCTGACCCTGAAGTTCATCTGCACCA
CCGGCAAGCTGCCCGTGCCCTGGCCCACCCTCGTGACCACCCTGACCTAC
GGCGTGCAGTGCTTCAGCCGCTACCCCGACCACATGAAGCAGCACGACTT
CTTCAAGTCCGCCATGCCCGAAGGCTACGTCCAGGAGCGCACCATCTTCT
TCAAGGACGACGGCAACTACAAGACCCGCGCCGAGGTGAAGTTCGAGGGC
GACACCCTGGTGAACCGCATCGAGCTGAAGGGCATCGACTTCAAGGAGGA
CGGCAACATCCTGGGGCACAAGCTGGAGTACAACTACAACAGCCACAACG
TCTATATCATGGCCGACAAGCAGAAGAACGGCATCAAGGTGAACTTCAAG
ATCCGCCACAACATCGAGGACGGCAGCGTGCAGCTCGCCGACCACTACCA
GCAGAACACCCCCATCGGCGACGGCCCCGTGCTGCTGCCCGACAACCACT
ACCTGAGCACCCAGTCCGCCCTGAGCAAAGACCCCAACGAGAAGCGCGAT
CACATGGTCCTGCTGGAGTTCGTGACCGCCGCCGGGATCACTCTCGGCAT
GGACGAGCTGTACAAGTAAGCGGCCGCTTAATTAAGCTGCCTTCTGCGGG
GCTTGCCTTCTGGCCATGCCCTTCTTCTCTCCCTTGCACCTGTACCTCTT
GGTCTTTGAATAAAGCCTGAGTAGGAAGAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAA

As used herein, common organic chemistry abbreviations are defined as follows:
Ac Acetyl
ACN Acetonitrile
AcOH Acetic acid
aq. Aqueous
AX chromatography Anion exchange chromatography
Bz Benzoyl
DBU 1,8-Diazabicyclo[5.4.0]undec-7-ene
DCA Dichloroacetic acid
DCM Dichloromethane
ddC 2′,3′-dideoxy Cytidine
DI water Deionized water
DIAD Diisopropyl azodicarboxylate
DIEA or DIPEA Diisopropylethylamine
DMAP 4-dimethylaminopyridine
DMF N,N-Dimethylformamide
DMS Dimethyl sulfate
DMSO Dimethyl sulfoxide
DMT Dimethoxytrityl
dTAm 2′-deoxy-5-(N-(6-aminohexyl)prop-2-enamide) thymidine
EDC or EDC·HCl 1-Ethyl-3-(3-dimethylaminopropyl)carbodiimide hydrochloride
EtOAc or EA Ethyl acetate
EtOH Ethanol
ETT 5-(ethylthio)-1H-tetrazole
Fmoc Fluorenylmethyloxycarbonyl
g Gram(s)
hrs. Hour (hours)
HCl Hydrochloric acid
HF-TEA Triethylamine trihydrofluoride
HPLC High-performance liquid chromatography
IPA Isopropyl alcohol
LC/MS Liquid chromatography-mass spectrometry
LNP Lipid nanoparticle
mg milligrams
MeOH Methanol
mL Milliliter(s)
μL or μL Microliter(s)
mmol millimoles
μmol or umol micromoles
MS mass spectrometry
NaH Sodium hydride
NaOAc Sodium acetate
NaOH Sodium hydroxide
Pyr Pyridine
RP-HPLC reverse phase HPLC
RT or r.t. room temperature
t-Bu tert-Butyl
TEA Triethylamine
TEAB Tetraethylammonium bromide
TEA salt triethylammonium salt
Tert, t tertiary
TFA Trifluoracetic acid
TFAA Trifluoracetic anhydride
THF Tetrahydrofuran
TMP Trimethylphosphate
TPP Triphenylphosphine

### EXAMPLES

The following examples are meant to be illustrative and can be used to further understand embodiments of the present disclosure and should not be construed as limiting the scope of the present teachings in any way.

The chemical reactions described in the Examples can be readily adapted to prepare a number of other compounds of the present disclosure, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by utilizing other suitable reagents known in the art other than those described, or by making routing modifications of reaction conditions, reagents, and starting materials. Alternatively, other reactions disclosed herein or known in the art will be recognized as having applicability for preparing other compounds of the present disclosure.

### Synthetic Examples

Unless indicated otherwise the amidites were purchased from Chemgene or Glen Research. eGFP mRNA (without a tail modification) and capped with m7G_{3′OMe}ppp^{m6}A_{2′OMe}pG was used as a control for m7G_{3′OMe}ppp^{m6}A_{2′OMe}pG capped eGFP mRNAs with modified 3′-end (all eGFP mRNAs with modified 3′-end were capped with m7G_{3′OMe}ppp^{m6}A_{2′OMe}pG; all FLuc mRNAs with modified 3′-end were capped with m7GpppA_{2′OMe}pG). The retention time for eGFP mRNA without a tail modification was 9.335 minutes.

Where indicated, the following Oligo dT Purification Procedure was used:
Load preparation: 50 μg of mRNA were diluted in High Salt Wash Buffer (250 mM NaCl, 50 mM Sodium Phosphate, 5 mM EDTA, pH 7) at <0.3 mg/mL, and 5 M NaCl was added to make a final concentration of 250 mM in the load.
Column: CIMmultus® Oligo dT18 (C6 Linker)—1 mL (2 μm), Vendor: BIA Separations Inc Catalog no. 311.1218-2
Instrument: AKTA Avant 25 HPLC system
Method Description: Column was equilibrated for >4 Column Volume (CV) in High Salt Wash buffer. Sample loaded onto column. The flow rate was set to 5 mL/minute.

| Step | Description |
|---|---|
| Post Load wash: (250 mM NaCl, 50 mM | High salt wash buffer |
| Sodium Phosphate, 5 mM EDTA, pH 7) | for ≥1 CV |
| High Salt Wash Buffer (250 mM NaCl, 50 | High salt wash buffer |
| mM Sodium Phosphate, 5 mM EDTA, pH 7) | for ≥5 CV |
| Low salt Wash (50 mM Sodium Phosphate, | Low salt wash buffer |
| 5 mM EDTA, pH 7) | for ≥10 CV |
| Elution: RNase/DNase free Water | RNase/DNase free Water |
| | for ≥5 CV |
| Wash: 0.1N NaOH | 0.1N NaOH ≥10 CV |
| Equilibration | ≥10 Column Volume (CV) |

### Example S1: Synthesis of Sequence 1a

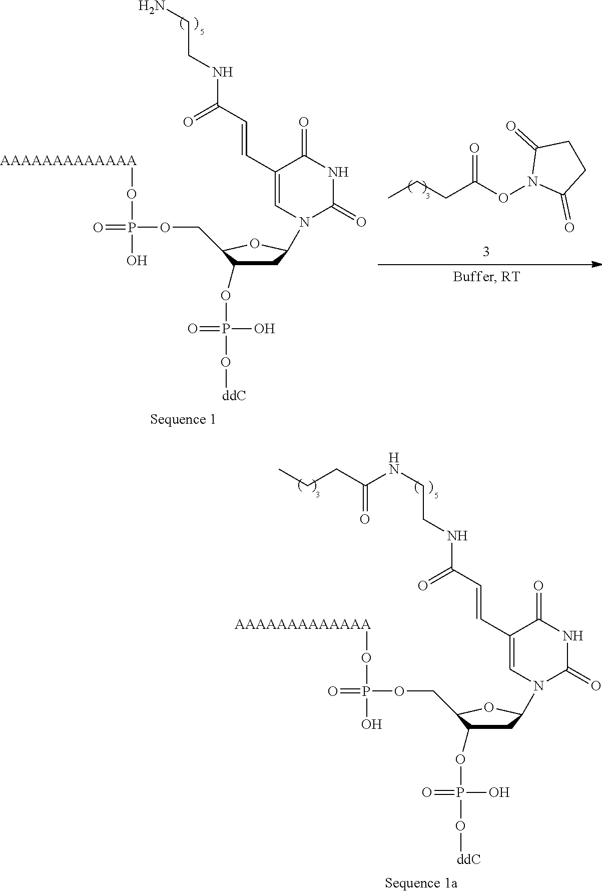

A 10 mM solution of Sequence 1 (includes 13 Adenosine ribonucleotides, dTAm and a ddC) (SEQ ID NO: 1) in water (custom ordered from Trilink Biotechnologies, 2 μL, 20 nmol) was added to a solution of 20 mM sodium phosphate buffer (pH 8.5, 16 μL) in a 1.5 mL Eppendorf tube. The solution was cooled in an ice bath for 3 minutes. A freshly prepared 100 mM solution of compound 3 (2 μL, 200 nmol) was added to the cooled solution in the Eppendorf tube and thoroughly mixed by pipette. The reaction was stirred at room temperature for 21 hours. A small aliquot (1 μL) of the reaction was diluted in water (19 μL) and analyzed by LC-MS. The crude reaction was stored in a −20° C. freezer and used as is. Yield 99% by LC-MS.

MS m/z=5125.8 [M−H].

### Example S2: Synthesis of Sequence 1b

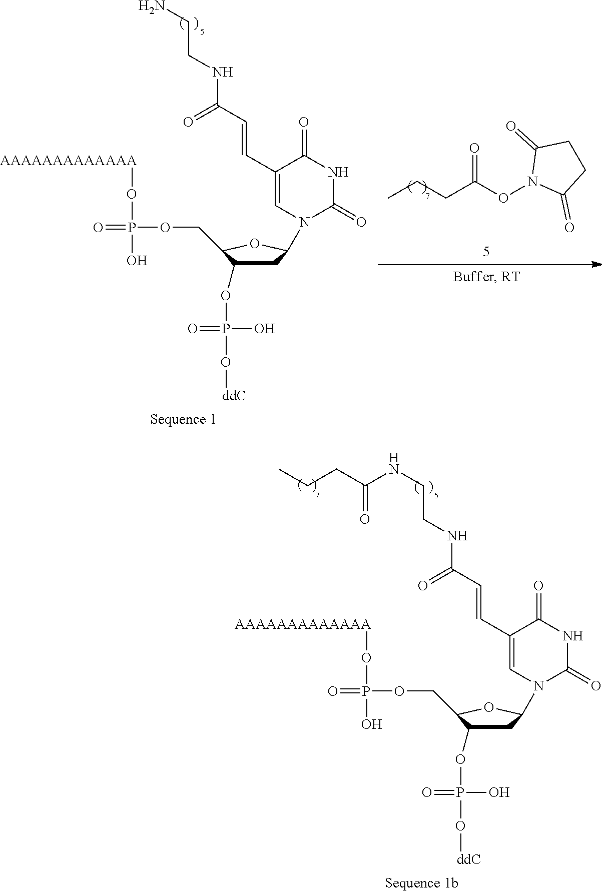

A 10 mM solution of Sequence 1 in water (custom ordered from Trilink Biotechnologies, 2 μL, 20 nmol) was added to a solution of 20 mM sodium phosphate buffer (pH 8.5, 16 μL) in a 1.5 mL Eppendorf tube. The solution was cooled in an ice bath for 3 minutes. A freshly prepared 100 mM solution of compound 5 (2 μL, 200 nmol) was added to the cooled solution in the Eppendorf tube and thoroughly mixed by pipette. The reaction was stirred at room temperature for 21 hours. A small aliquot (1 μL) of the reaction was diluted in water (19 μL) and analyzed by LC-MS. The crude reaction was stored in a −20° C. freezer and used as is. Yield ˜20% by LC-MS.

MS m/z=5182.3 [M−H].

### Example S3: Synthesis of Sequence 1c

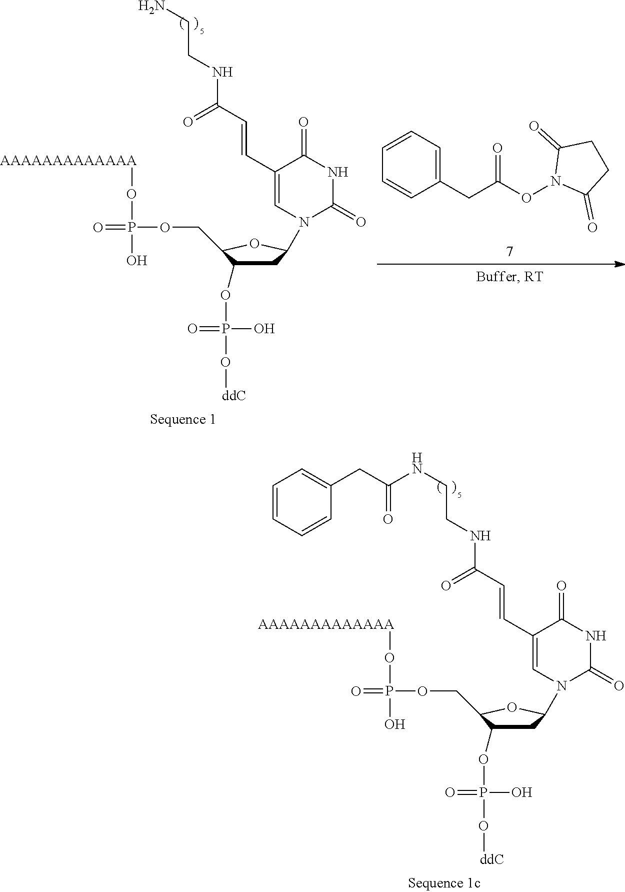

A 10 mM solution of Sequence 1 in water (custom ordered from Trilink Biotechnologies, 2 μL, 20 nmol) was added to a solution of 20 mM sodium phosphate buffer (pH 8.5, 16 μL) in a 1.5 mL Eppendorf tube. The solution was cooled in an ice bath for 3 minutes. A freshly prepared 100 mM solution of compound 7 (2 μL, 200 nmol) was added to the cooled solution in the Eppendorf tube and thoroughly mixed by pipette. The reaction was stirred at room temperature for 21 hours. A small aliquot (1 μL) of the reaction was diluted in water (19 μL) and analyzed by LC-MS. The crude reaction was stored in a −20° C. freezer and used as is. Yield 99% by LC-MS.

MS m/z=5146.6 [M−H].

### Example S4: Synthesis of Sequence 2a

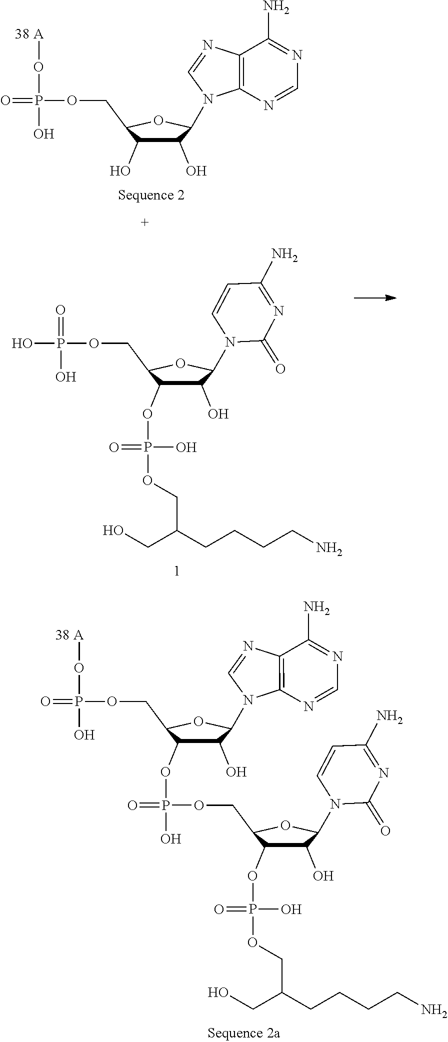

To a 1.5 mL Eppendorf tube were added RNAse free water (13.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 3.0 μL), DMSO (3.0 μL), and Murine RNase Inhibitor (purchased from New England Biolabs Inc. Catalog #M0314B, 40 U/μL, 0.4 μL). To the resulting solution were added adenosine triphosphate (purchased from New England BioLabs Inc. Catalog #: M0204S, 10 mM, 3.0 μL), Sequence 2 (includes 39 Adenosine ribonucleotides; purchased from TriLink Biotechnologies, 1 mM, 3.0 μL), compound 1 (custom ordered from Trilink Biotechnologies; 0.1 mM, 3.0 μL), and T4 RNA Ligase 1 (purchased from New England Biolabs Inc. Catalog #: M0204S, 10 U/μL, 1.0 μL) and thoroughly mixed. The reaction mixture was incubated at room temperature for 21 hours. Analysis by LC-MS confirmed Sequence 2a was obtained (99% yield by HPLC). Retention Time Sequence 2a: 4.771 min.

MS m/z=13287.8 [M−H].

### Example S5: Synthesis of Sequence 2b

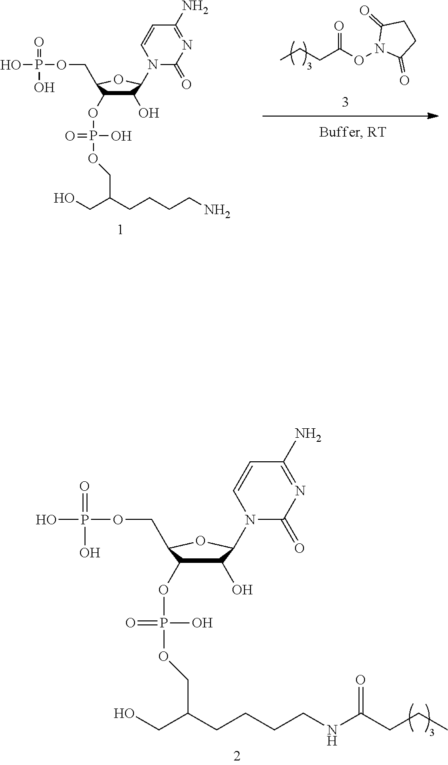

A 10 mM solution of compound 1 in water (custom ordered from Trilink Biotechnologies, 10 μL, 100 nmol) was added to a solution of 20 mM sodium phosphate buffer (pH 8.5, 80 μL) in a 1.5 mL Eppendorf tube. The solution was cooled in an ice bath for 3 minutes. A freshly prepared 100 mM solution of compound 3 (purchased f Aldrich Inc. Catalog #AMBH97F1 164B, 10 μL, 1 μmol) was added to the cooled solution in the Eppendorf tube and thoroughly mixed by pipette. The reaction was stirred at room temperature for 21 hours. A small aliquot (1 μL) of the reaction was diluted in water (19 μL) and analyzed by LC-MS. Upon confirmation of desired product 2, the crude reaction was stored in a −20° C. freezer and used as is.

MS m/z=630.2 [M−H].

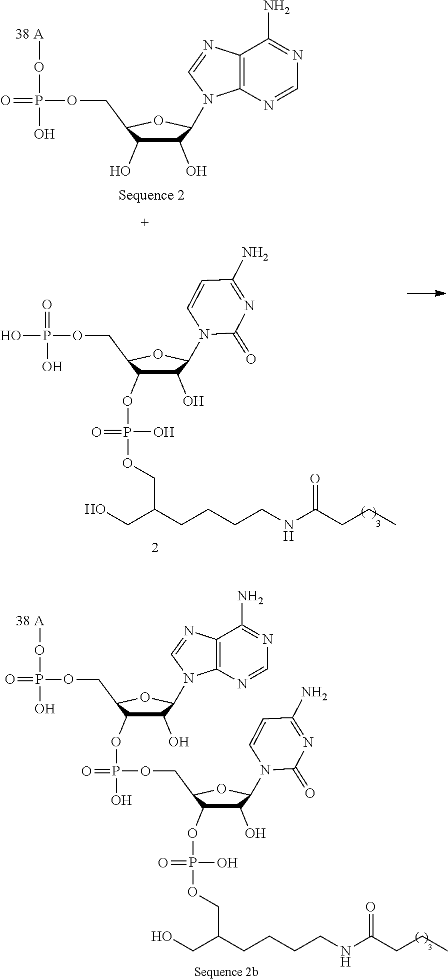

To a 1.5 mL Eppendorf tube were added RNAse free water (13.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 3.0 μL), DMSO (3.0 μL), and Murine RNase Inhibitor (purchased from New England Biolabs Inc. Catalog #M0314B, 40 U/μL, 0.4 μL). To the resulting solution were added adenosine triphosphate (purchased from New England BioLabs Inc. Catalog #: M0204S, 10 mM, 3.0 μL), Sequence 2 (purchased from TriLink Biotechnologies, 1 mM, 3.0 μL), compound 2 (0.1 mM, 3.0 μL), and T4 RNA Ligase 1 (purchased from New England Biolabs Inc. Catalog #: M0204S, 10 U/μL, 1.0 μL) and thoroughly mixed. The reaction was incubated at room temperature for 21 hours. Analysis by LC-MS confirmed Sequence 2b was obtained (99% yield by HPLC). Retention Time Sequence 2b: 5.477 min.

MS m/z=13386.8 [M−H].

### Example S6: Synthesis of Sequence 2c

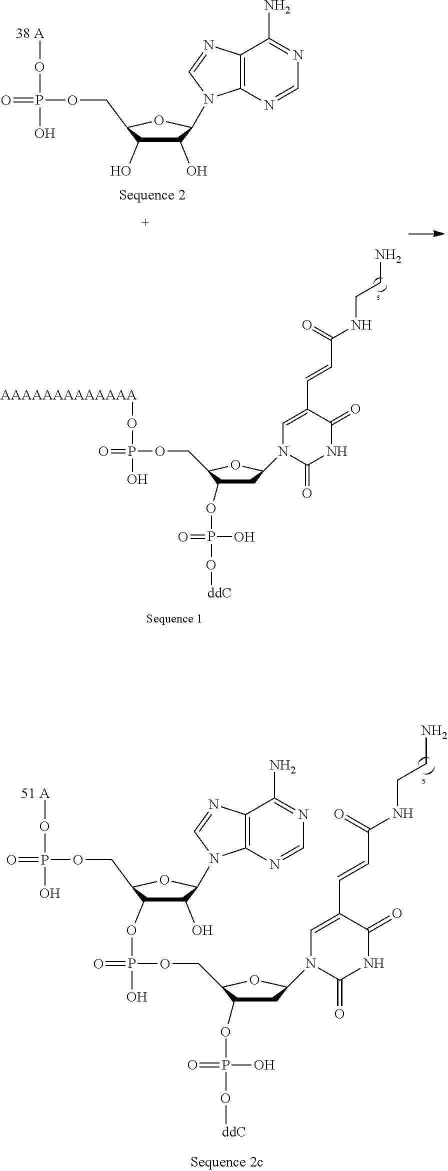

To a 1.5 mL Eppendorf tube were added RNAse free water (6.8 μL), T4 RNA Ligase reaction buffer (purchased from New England Biolabs Inc. Catalog #: M0204S, 500 mM Tris-HCl, 100 mM MgCl₂, 10 mM DTT, pH 7.5, 2.0 μL), 50% PEG 8000 (purchased from New England BioLabs Inc. Catalog #M0204S, 4.0 μL), and Murine RNase Inhibitor (purchased from New England Biolabs Inc. Catalog #M0314B, 40 U/μL, 0.3 μL). To the resulting solution were added adenosine triphosphate (purchased from New England BioLabs Inc. Catalog #: M0204S, 10 mM, 2.0 μL), Sequence 2 (purchased from TriLink Biotechnologies, 10 μM, 2.0 μL), Sequence 1 (0.1 mM, 2.0 μL), and T4 RNA Ligase 1 (purchased from New England Biolabs Inc. Catalog #: M0204S, 10 U/μL, 1.0 μL) and thoroughly mixed. The reaction was incubated at room temperature for 21 hours. Analysis by LC-MS confirmed Sequence 2c was obtained. Retention Time Sequence 2c: 5.135 min.

MS m/z=17783.0 [M−H].

### Example S7: Synthesis of Sequence 2d

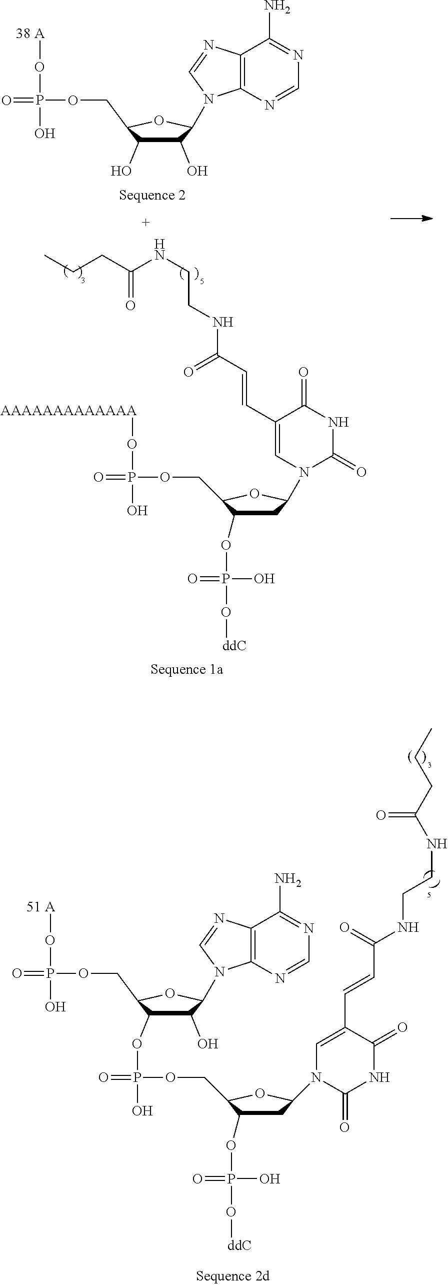

To a 1.5 mL Eppendorf tube were added RNAse free water (6.8 μL), T4 RNA Ligase reaction buffer (purchased from New England Biolabs Inc. Catalog #: M0204S, 500 mM Tris-HCl, 100 mM MgCl₂, 10 mM DTT, pH 7.5, 2.0 μL), 50% PEG 8000 (purchased from New England BioLabs Inc. Catalog #M0204S, 4.0 μL), and Murine RNase Inhibitor (purchased from New England Biolabs Inc. Catalog #M0314B, 40 U/μL, 0.3 μL). To the resulting solution were added adenosine triphosphate (purchased from New England BioLabs Inc. Catalog #: M0204S, 10 mM, 2.0 μL), Sequence 2 (purchased from TriLink Biotechnologies, 10 μM, 2.0 μL), Sequence 1a (0.1 mM, 2.0 μL), and T4 RNA Ligase 1 (purchased from New England Biolabs Inc. Catalog #: M0204S, 10 U/μL, 1.0 μL) and thoroughly mixed. The reaction was incubated at room temperature for 21 hours. Analysis by LC-MS confirmed Sequence 2d was obtained (99% yield by HPLC). Retention Time Sequence 2d: 6.024 min.

MS m/z=17882.8 [M−H].

### Example S8: Synthesis of Sequence 3

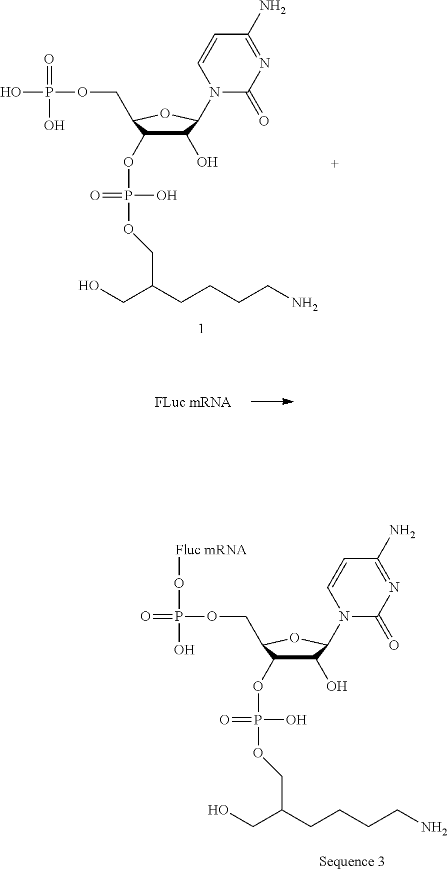

To a 1.5 mL Eppendorf tube were added RNAse free water (1.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), FLuc mRNA (purchased from TriLink Biotechnologies, 1.0 mg/mL, 50.0 μL), and Murine RNase Inhibitor (purchased from New England Biolabs Inc. Catalog #M0314B, 40 U/μL, 1.0 μL). To the resulting solution were added adenosine triphosphate (purchased from New England BioLabs Inc. Catalog #: M0204S, 10 mM, 8.0 μL), compound 1 (1 mM, 0.8 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA isolated using an RNeasy kit (purchased from Qiagen Catalog #: 74004), eluting with RNAse free water (50 μL). Analysis by Nanodrop confirmed mRNA was obtained (49.9 pg). Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes was performed. Retention Time FLuc mRNA+Sequence 3: 11.352 min.

### Example S9: Synthesis of Sequence 5

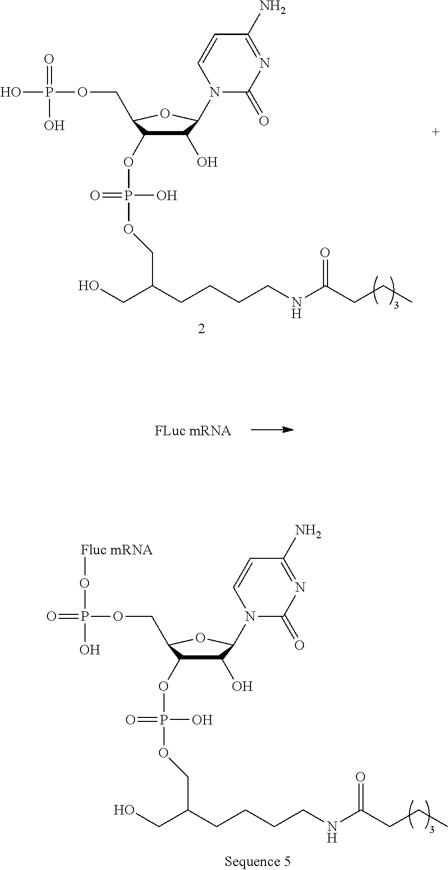

To a 1.5 mL Eppendorf tube were added RNAse free water (1.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), FLuc mRNA (purchased from TriLink Biotechnologies, 1.0 mg/mL, 50.0 μL), and Murine RNase Inhibitor (purchased from New England Biolabs Inc. Catalog #M0314B, 40 U/μL, 1.0 μL). To the resulting solution were added adenosine triphosphate (purchased from New England BioLabs Inc. Catalog #: M0204S, 10 mM, 8.0 μL), compound 2 (1 mM, 0.8 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA isolated using an RNeasy kit (purchased from Qiagen Catalog #: 74004), eluting with RNAse free water (50 μL). Analysis by Nanodrop confirmed mRNA was obtained (50.5 pg). Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes confirmed the presence of a new product (52% yield by HPLC) corresponding to Sequence 5. Retention Time: FLuc mRNA: 11.477 min. Sequence 5: 12.204 min.

### Example S10: Synthesis of Sequence 4

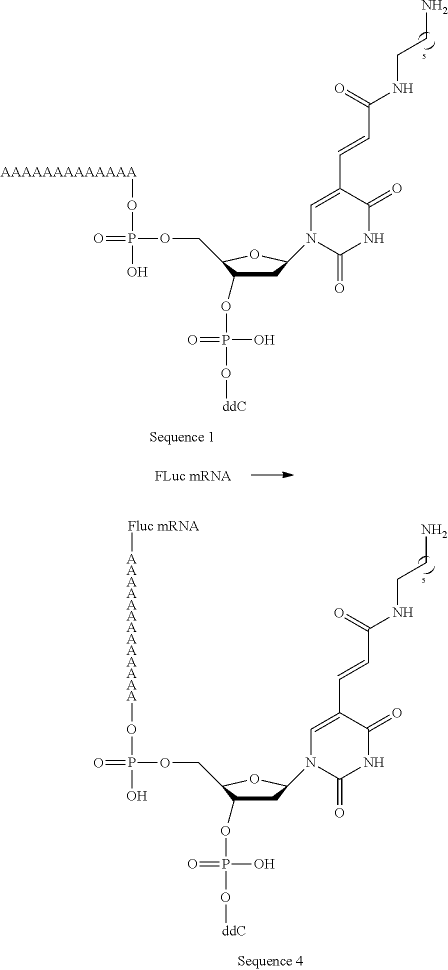

To a 1.5 mL Eppendorf tube were added RNAse free water (1.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), FLuc mRNA (purchased from TriLink Biotechnologies, 1.0 mg/mL, 50.0 μL), and Murine RNase Inhibitor (purchased from New England Biolabs Inc. Catalog #M0314B, 40 U/μL, 1.0 μL). To the resulting solution were added adenosine triphosphate (purchased from New England BioLabs Inc. Catalog #: M0204S, 10 mM, 8.0 μL), Sequence 1 (1 mM, 0.8 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA isolated using an RNeasy kit (purchased from Qiagen Catalog #: 74004), eluting with RNAse free water (50 μL). Analysis by Nanodrop confirmed mRNA was obtained (50.1 pg). Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes was performed. Retention Time FLuc mRNA+Sequence 4: 11.400 min.

### Example S11: Synthesis of Sequence 6

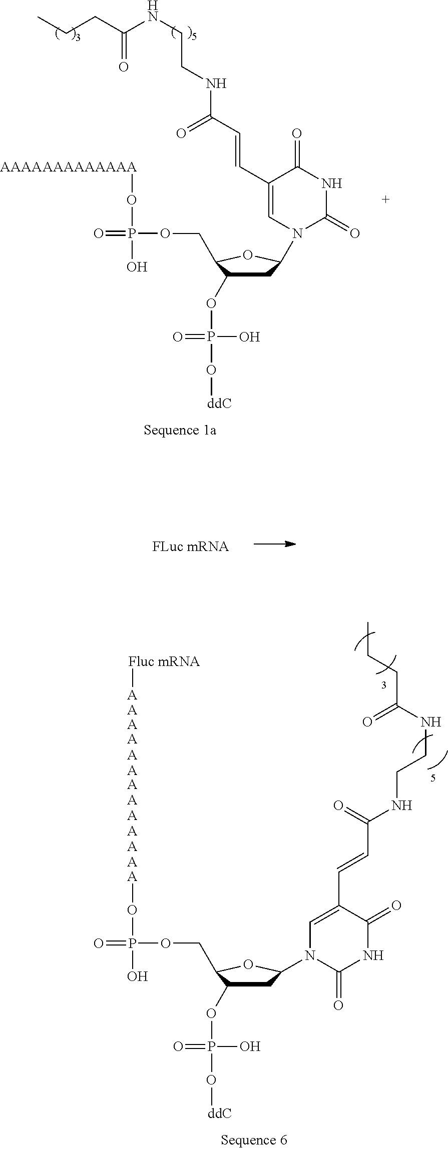

To a 1.5 mL Eppendorf tube were added RNAse free water (1.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), FLuc mRNA (purchased from TriLink Biotechnologies, 1.0 mg/mL, 50.0 μL), and Murine RNase Inhibitor (purchased from New England Biolabs Inc. Catalog #M0314B, 40 U/μL, 1.0 μL). To the resulting solution were added adenosine triphosphate (purchased from New England BioLabs Inc. Catalog #: M0204S, 10 mM, 8.0 μL), Sequence 1a (1 mM, 0.8 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was isolated using an RNeasy kit (purchased from Qiagen Catalog #: 74004), eluting with RNAse free water (50 μL). Analysis by Nanodrop confirmed mRNA was obtained (50.8 pg). Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes confirmed the presence of a new product (30% yield by HPLC) corresponding to Sequence 6. Retention Time FLuc mRNA: 11.433 min.; Retention Time Sequence 6: 13.014 min.

### Example S12: Synthesis of Sequence 7

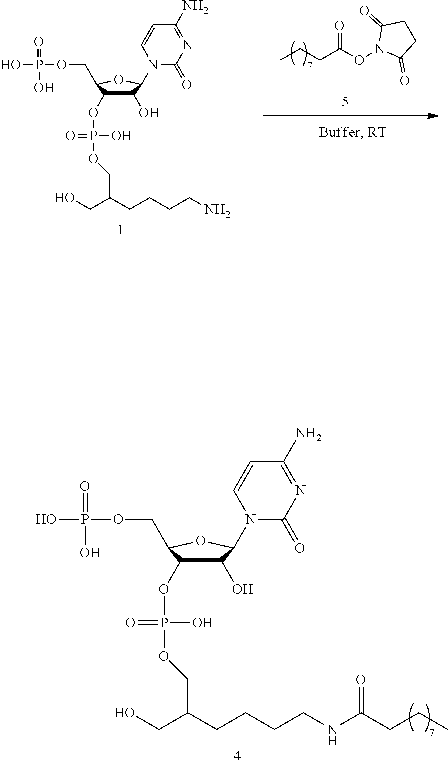

A 10 mM solution of compound 1 in water (custom ordered from Trilink Biotechnologies, 10 μL, 100 nmol) was added to a solution of 20 mM sodium phosphate buffer (pH 8.5, 80 μL) in a 1.5 mL Eppendorf tube. The solution was cooled in an ice bath for 3 minutes. A freshly prepared 100 mM solution of compound 5 (purchased from Sigma-Aldrich Inc. Catalog #AMBH97F116BA, 10 μL, 1 μmol) was added to the cooled solution in the Eppendorf tube and thoroughly mixed by pipette. The reaction was stirred at room temperature for 21 hours. A small aliquot (1 μL) of the reaction was diluted in water (19 μL) and analyzed by LC-MS. Upon confirmation of desired product 4, the crude reaction was stored in a −20° C. freezer and used as is.

MS m/z=686.3 [M−H].

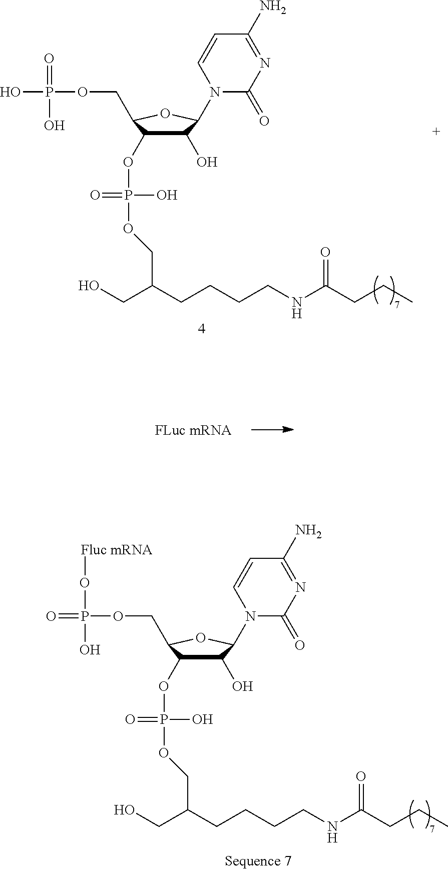

To a 1.5 mL Eppendorf tube were added RNAse free water (1.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), FLuc mRNA (purchased from TriLink Biotechnologies, 1.0 mg/mL, 50.0 μL), and Murine RNase Inhibitor (purchased from New England Biolabs Inc. Catalog #M0314B, 40 U/μL, 1.0 μL). To the resulting solution were added adenosine triphosphate (purchased from New England BioLabs Inc. Catalog #: M0204S, 10 mM, 8.0 μL), compound 4 (1 mM, 0.8 μL), and T4 RNA Ligase 1 (purchased from New England Biolabs Inc. Catalog #: M0204S, 10 U/μL, 2.6 μL) and thoroughly mixed. The reaction incubated at room temperature for 21 hours and the mRNA was isolated using an RNeasy kit (purchased from Qiagen Catalog #: 74004), eluting with RNAse free water (50 μL). Analysis by Nanodrop confirmed mRNA was obtained (51.0 pg). Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes confirmed the presence of a new product (29% yield by HPLC) corresponding to Sequence 7. Retention Time FLuc mRNA: 11.941 min.; Retention Time Sequence 7: 15.422 min.

### Example S13: Synthesis of Sequence 8

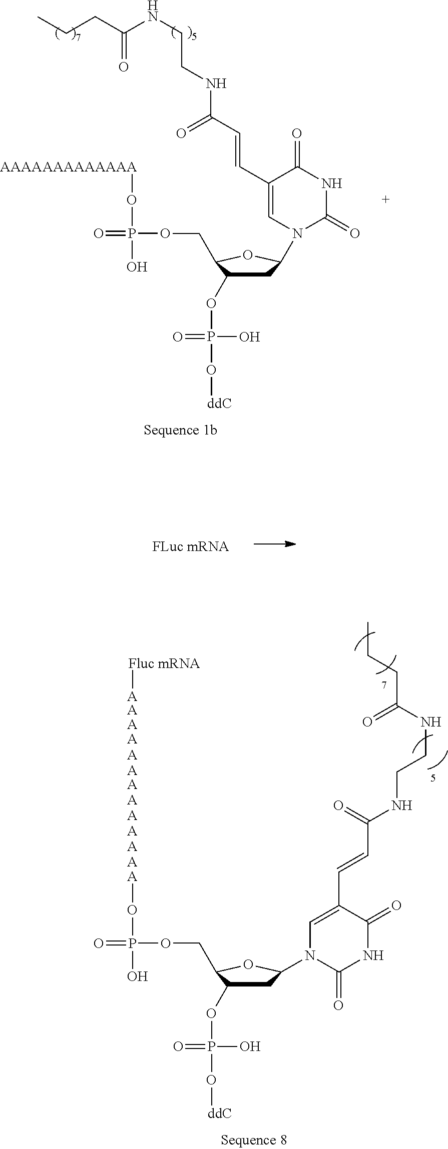

To a 1.5 mL Eppendorf tube were added RNAse free water (1.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), FLuc mRNA (purchased from TriLink Biotechnologies, 1.0 mg/mL, 50.0 μL), and Murine RNase Inhibitor (purchased from New England Biolabs Inc. Catalog #M0314B, 40 U/μL, 1.0 μL). To the resulting solution were added adenosine triphosphate (purchased from New England BioLabs Inc. Catalog #: M0204S, 10 mM, 8.0 μL), Sequence lb (1 mM, 0.8 μL), and T4 RNA Ligase 1 (purchased from New England Biolabs Inc. Catalog #: M0204S, 10 U/μL, 2.6 μL) and thoroughly mixed. The reaction incubated at room temperature for 21 hours and the mRNA was isolated using an RNeasy kit (purchased from Qiagen Catalog #: 74004), eluting with RNAse free water (50 μL). Analysis by Nanodrop confirmed mRNA was obtained (50.2 pg). Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes confirmed the presence of a new product (0.72% yield by HPLC) corresponding to Sequence 8. Retention Time FLuc mRNA: 11.921 min.; Retention Time Sequence 8: 16.794 min.

### Example S14: Synthesis of Sequence 9

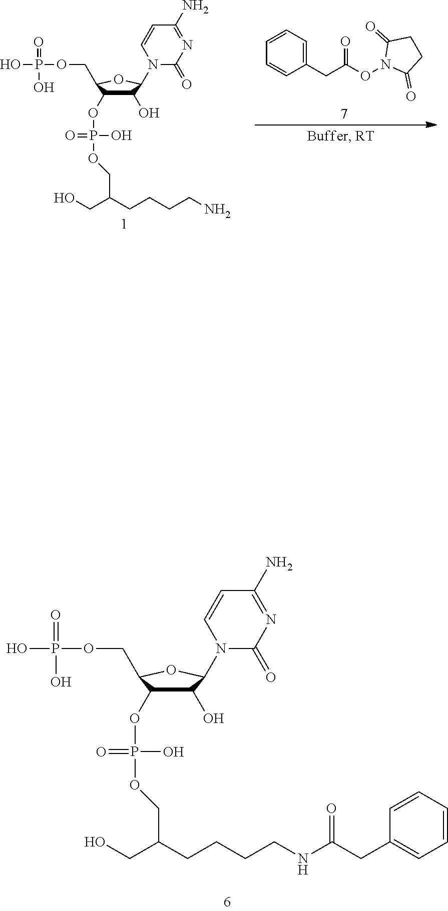

A 10 mM solution of compound 1 in water (custom ordered from Trilink Biotechnologies, 10 μL, 100 nmol) was added to a solution of 20 mM sodium phosphate buffer (pH 8.5, 80 μL) in a 1.5 mL Eppendorf tube. The solution was cooled in an ice bath for 3 minutes. A freshly prepared 100 mM solution of compound 7 (purchased from Sigma-Aldrich Inc. Catalog #ENAH042579A2, 10 μL, 1 μmol) was added to the cooled solution in the Eppendorf tube and thoroughly mixed by pipette. The reaction was stirred at room temperature for 21 hours. A small aliquot (1 μL) of the reaction was diluted in water (19 μL) and analyzed by LC-MS. Upon confirmation of desired product 6, the crude reaction was stored in a −20° C. freezer and used as is.

MS m/z=650.2 [M−H].

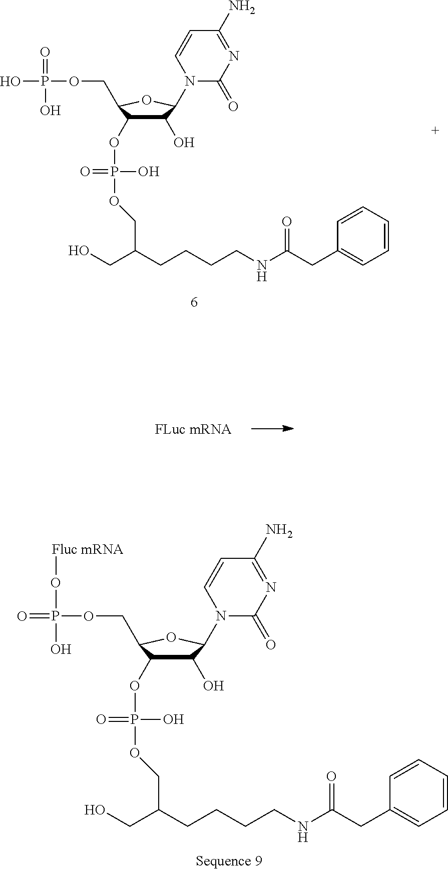

To a 1.5 mL Eppendorf tube were added RNAse free water (1.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), FLuc mRNA (purchased from TriLink Biotechnologies, 1.0 mg/mL, 50.0 μL), and Murine RNase Inhibitor (purchased from New England Biolabs Inc. Catalog #M0314B, 40 U/μL, 1.0 μL). To the resulting solution were added adenosine triphosphate (purchased from New England BioLabs Inc. Catalog #: M0204S, 10 mM, 8.0 μL), compound 6 (1 mM, 0.8 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA isolated using an RNeasy kit (purchased from Qiagen Catalog #: 74004), eluting with RNAse free water (50 μL). Analysis by Nanodrop confirmed mRNA was obtained (49.9 pg). Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes confirmed the presence of a new product (64% yield by HPLC) corresponding to Sequence 9. Retention Time FLuc mRNA: 11.950 min.; Retention Time Sequence 9: 12.493 min.

### Example S15: Synthesis of Sequence 10

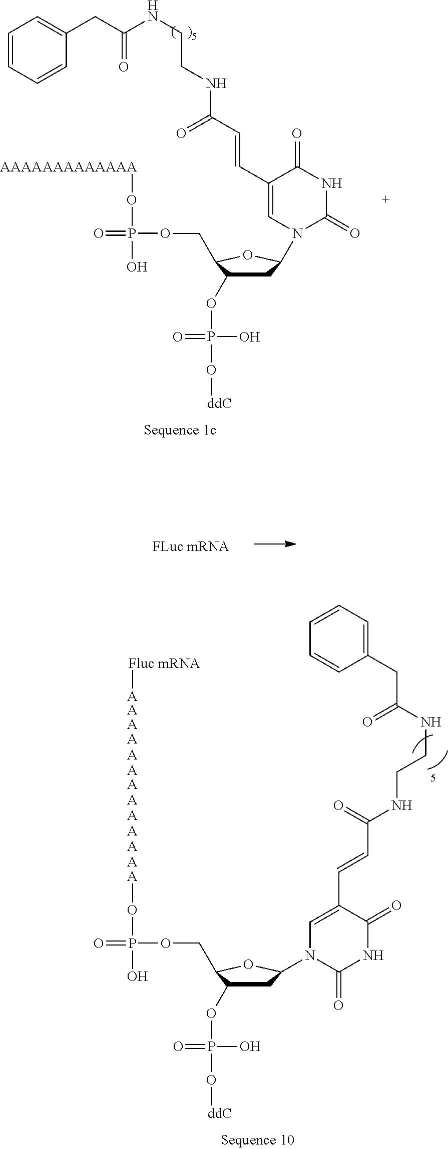

To a 1.5 mL Eppendorf tube were added RNAse free water (1.6 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), FLuc mRNA (purchased from TriLink Biotechnologies, 1.0 mg/mL, 50.0 μL), and Murine RNase Inhibitor (purchased from New England Biolabs Inc. Catalog #M0314B, 40 U/μL, 1.0 μL). To the resulting solution were added adenosine triphosphate (purchased from New England BioLabs Inc. Catalog #: M0204S, 10 mM, 8.0 μL), Sequence 1c (1 mM, 0.8 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA isolated using an RNeasy kit (purchased from Qiagen Catalog #: 74004), eluting with RNAse free water (50 μL). Analysis by Nanodrop confirmed mRNA was obtained (49.7 pg). Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes confirmed the presence of a new product (4.2% yield by HPLC) corresponding to Sequence 10. Retention Time FLuc mRNA: 11.828 min.; Retention Time Sequence 10: 13.240 min.

### Example S16: Synthesis of Sequence 11

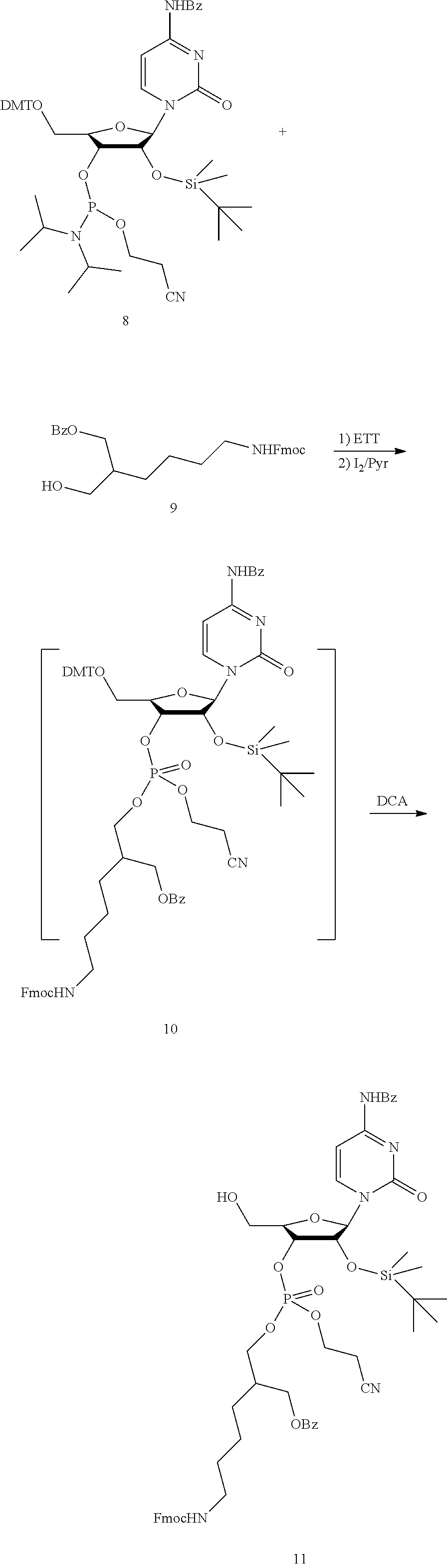

Compound 9 (Shijiazhuang Lidekang Medichem. Ltd., Catalog No. C-200; 4.0 g, 8.5 mmol) was dissolved in acetonitrile (50 ml) and DCM (30 ml), then molecular sieves (15 g) were added to the solution. The mixture was stirred for 1 hr., then compound 8 (8.96 g, 9.3 mmol) and 5-(ethylthio)-1H-tetrazole (ETT) (1.22 g, 9.4 mmol) were added to the mixture and stirred for an additional 3.5 hr. The reaction was followed by LCMS. A solution of iodine (2.14 g, 8.4 mmol), pyridine (2 ml) and water (0.2 ml) in DCM (10 ml) was slowly added to the mixture and stirred for 15 min. Formation of compound 10 was detected by LCMS. Dichloroacetic acid (5.5 ml) was added to compound 10 and the solution was stirred for another 20 min. Sodium bicarbonate solution (80 ml, 10%) was added to neutralize the mixture, followed by addition of sodium bisulfite solution (15 ml, 5%). The mixture was transferred to a separatory funnel and the flask was washed with DCM (350 ml). The solution was washed once with brine and dried over sodium sulfate. The solvent was evaporated and compound 11 was purified using Combi-Flash silica column (DCM/EtOAc). Compound 11 was obtained with 63.5% yield (6.2 g).

MS m/z=1050.5 [M+H].

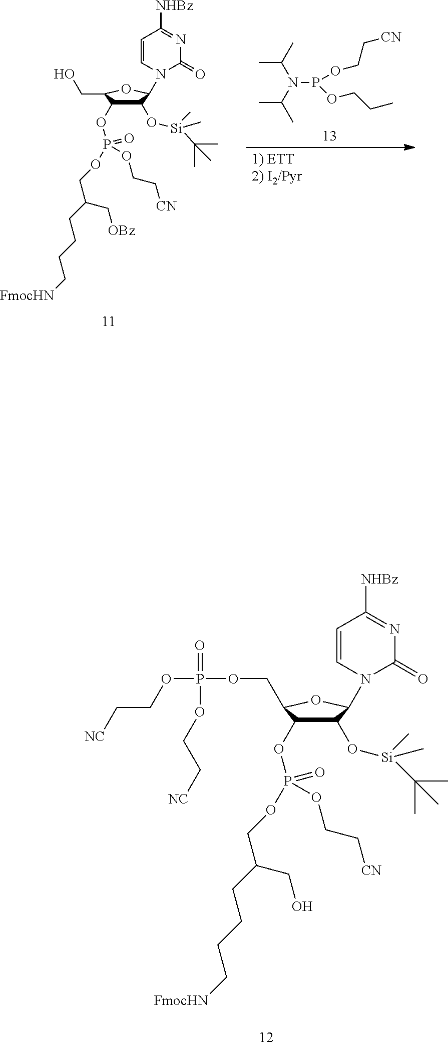

Compound 11 (1.5 g, 1.42 mmol) was dissolved in acetonitrile (10 ml) and DCM (20 ml), then molecular sieves (5 g) were added to the solution. The mixture was stirred for 1 hr., then compound 13 (0.65 g, 2.4 mmol) and 5-(ethylthio)-1H-tetrazole (0.3 g, 2.3 mmol) were added to the mixture and stirred for an additional 0.5 hr. The reaction was followed by LCMS. A solution of iodine (0.58 g, 2.3 mmol), pyridine (0.5 ml) and water (0.1 ml) in DCM (10 ml) was slowly added to the mixture and stirred for 15 min. Sodium bisulfite solution (15 ml, 5%) was added and stirred for 3 min. The solution was transferred to a separatory funnel. The flask was washed with 300 ml of DCM and DCM was transferred to the separatory funnel. The solution was washed once with brine and dried over sodium sulfate. The solvent was evaporated, and the crude compound 12 was obtained and used in the next reaction without purification (2.lg crude).

MS m/z=1236.7 [M+H].

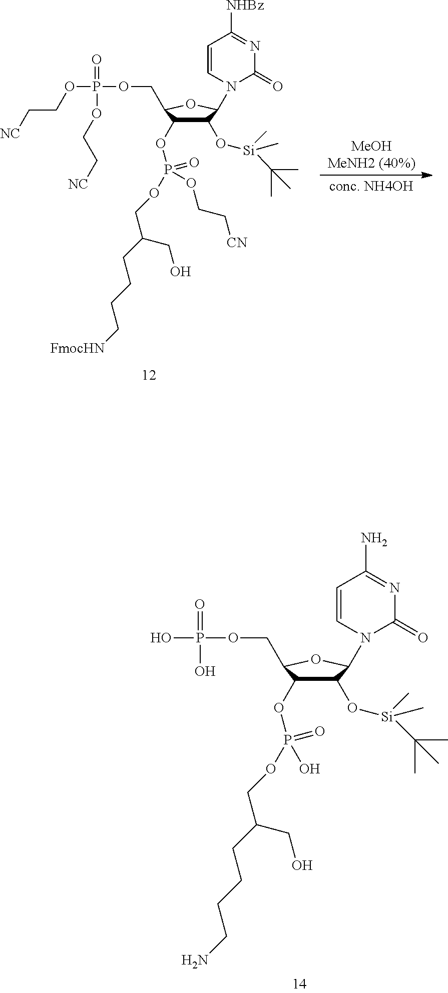

Crude compound 12 was dissolved in MeOH (20 ml). MeNH₂ (30 ml, 40%) and NH40H (15 ml, 36%) were added, and the solution was stirred at r.t. for 18 hrs. Half of the solvent was evaporated, and 50 ml of water was added. The solution was washed with ethyl acetate (200 ml). The aqueous solution was purified on a reverse phase column (YMC Actus Triart C18 250×20.0 mm, I.D. S-5 μm, 12 nm) eluted with a gradient of 0 to 90% acetonitrile in Buffer A (50 mM TEAA, 2% ACN in water) over 10 column volumes (CVs). The desired fractions were collected partially dried to remove the ACN, then diluted with water and purified on a reverse phase column (YMC Actus Triart C18 250×20.0 mm, I.D. S-5 μm, 12 nm) eluted with a gradient of 0 to 35% acetonitrile in Buffer A over 10 CVs. The product was collected as 87% yield over 2 steps (800 mg, 1.24 mmol)

MS m/z=646.6 [M−H].

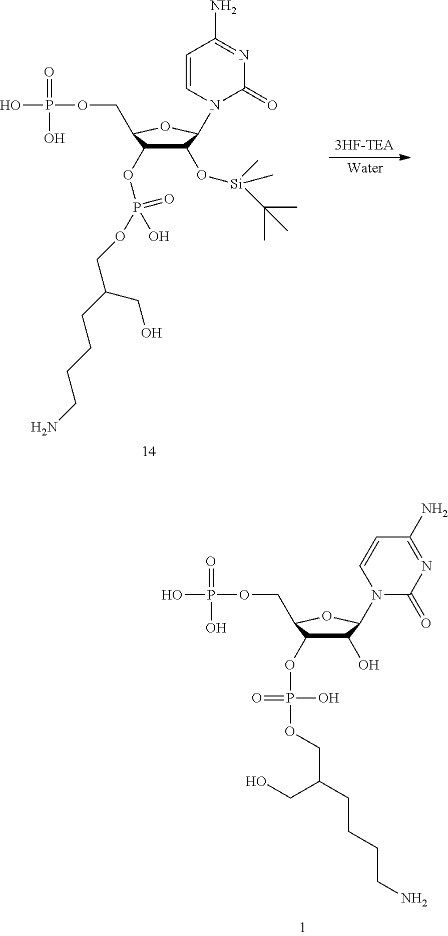

Compound 14 (TEA salt, 800 mg, 1.24 mmol) was suspended in water (20 mL), 3HF-TEA (5 mL) was added, and the solution was stirred at room temperature 24 hours. The solution was neutralized with 1 M TEAB (50 mL), then diluted with water to a conductivity of −5 mS/cm. The solution was filtered, and purified on an anion exchange column (eluted with a gradient of 0 to 100% 0.1 M TEAB in water). The desired fractions were collected, and dried to yield a residue (363 mg, 680 μmol, 55% yield).

MS m/z=531.1 [M−H].

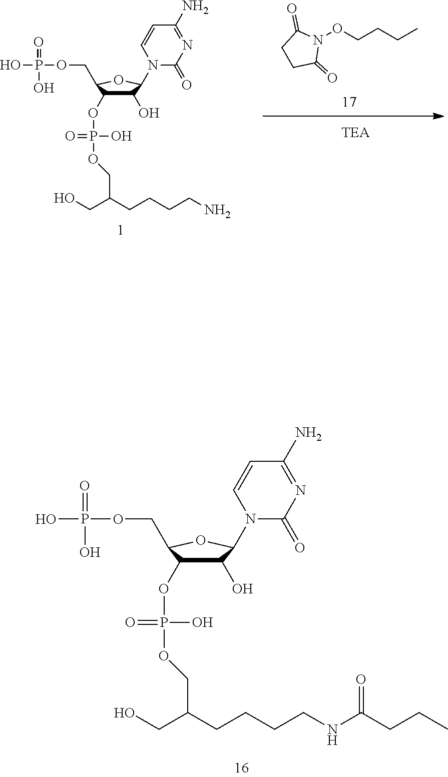

Compound 1 (TEA salt, 80 mg, 56 μmol) was dissolved in de-ionized water (1 mL), and triethylamine (100 μL, 720 mmol) was added. In a separate vial, Compound 17 (21 mg, 110 μmol) was dissolved in DMSO (0.5 mL). The solution of Compound 17 was added to the solution of Compound 1 and stirred for 30 minutes at room temperature. The solution was diluted with 200 mL water and filtered to remove any resulting precipitate. The diluted solution was purified using an anion exchange column and eluted with a gradient of 0 to 35% 1M NaCl in water. The desired fractions were collected, diluted to a conductivity of <5 mS/cm and purified on a reverse phase column (YMC Actus Triart C18 250×20.0 mm, I.D. S-5 μm, 12 nm) with a gradient of 0 to 20% acetonitrile in water. The desired fractions were dried to yield a clear film (28.9 mg, 43 μmol, 76% yield).

MS m/z=601.2 [M−H].

1H NMR (500 MHz, D₂O): δ.8.12 (d, J=7.6 Hz, 1H), 6.16 (d, J=7.7 Hz, 1H), 6.14 (d, J=6.9 Hz, 1H), 4.78-4.65 (m, 1H), 4.50 (t, J=5.5 Hz, 1H), 4.45 (d, J=1.5 Hz, 1H), 4.02-3.85 (m, 4H), 3.61 (d, J=5.8 Hz, 2H), 3.18 (t, J=6.6 Hz, 2H), 2.19 (dt, J=1.3, 7.3 Hz, 2H), 1.85-1.77 (m, 1H), 1.58 (sextet, J=7.1 Hz, 2H), 1.53-1.48 (m, 2H), 1.41-1.32 (m, 4H), 0.88 (t, J=7.5 Hz, 3H).

31P NMR (200 MHz, D₂O): δ.4.10 (s, 1P), 0.78 (d, 1P).

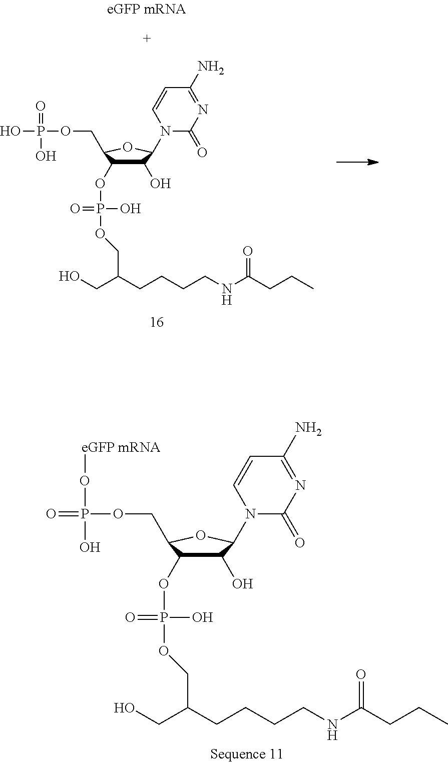

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 16 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.447 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes was performed. The presence of the new product was confirmed by HPLC.

Retention Time eGFP mRNA: 9.335 min.; Retention Time Sequence 11: 10.764 min.

### Example S17: Synthesis of Sequence 12

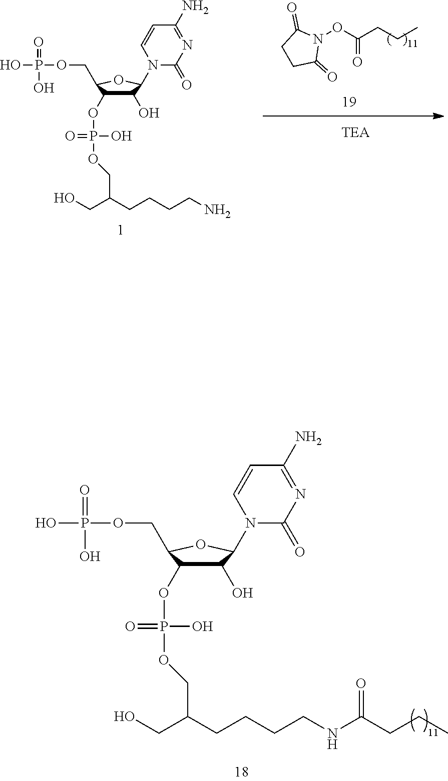

Compound 1 (TEA salt, 80 mg, 56 μmol) was dissolved in dry DMSO (1 mL), and triethylamine (100 μL, 720 mmol) was added. In a separate vial, compound 19 (37 mg, 110 μmol) was dissolved in DMSO (0.5 mL) and DCM (0.5 mL). The solution of compound 19 was added to the solution of compound 1 and stirred for 5 minutes at room temperature. DMSO (1 mL) and DCM (1 mL) were added to dissolve remaining solids. The solution was stirred at room temperature for 20 minutes. The reaction mixture was diluted with 200 mL of water and filtered to remove any resulting precipitate. The diluted solution was purified using an anion exchange column and eluted with a gradient of 0 to 50% 1M NaCl in water. The desired fractions were collected, diluted to a conductivity of <5 mS/cm and purified on a reverse phase column (YMC Actus Triart C18 250×20.0 mm, I.D. S-5 μm, 12 nm) with a gradient of 0 to 50% acetonitrile in water. The desired fractions were dried to yield a clear film (18.5 mg, 23 μmol, 41% yield).

MS m/z=741.3 [M−H].

1H NMR (500 MHz, D₂O): δ.8.05 (d, J=7.7 Hz, 1H), 6.23 (d, J=7.6 Hz, 1H), 6.06 (d, J=5.6 Hz, 1H), 4.75-4.67 (m, 1H), 4.45-4.39 (m, 2H), 4.15-4.09 (m, 2H), 3.98-3.85 (m, 2H), 3.62-3.55 (m, 2H), 3.22-3.14 (m, 3H), 2.22-2.18 (m, 2H), 1.82-1.75 (m, 1H), 1.58-1.52 (m, 2H), 1.52-1.45 (m, 2H), 1.40-1.31 (m, 5H), 1.30-1.16 (m, 22H), 0.83 (t, J=6.5 Hz, 3H).

31P NMR (200 MHz, D₂O): δ.0.82 (s, 1P), 0.65 (s, 1P).

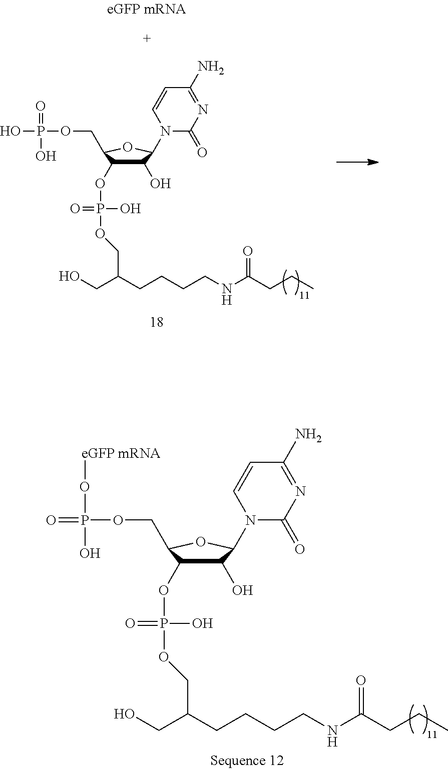

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 18 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.564 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 80% Buffer B in Buffer A over 29.8 minutes was performed. The presence of the new product was confirmed by HPLC.

Retention Time eGFP mRNA: 9.335 min.; Retention Time Sequence 12: 24.515 min.

### Example S18: Synthesis of Sequence 13

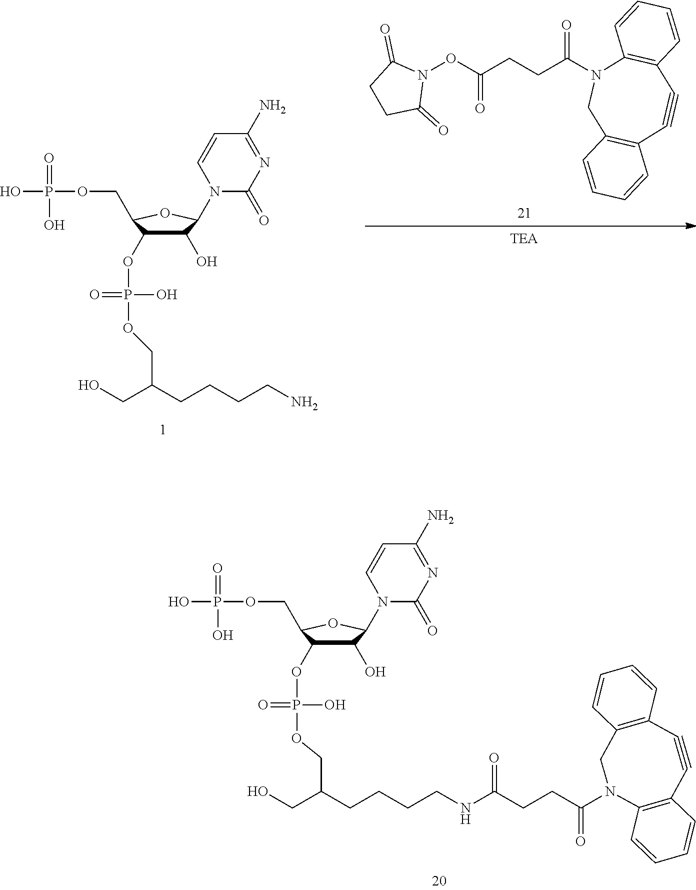

Compound 1 (TEA salt, 80 mg, 56 μmol) was dissolved in dry DMSO (1 mL), and triethylamine (100 μL, 720 mmol) was added. In a separate vial, compound 21 (45 mg, 110 μmol) was dissolved in DMSO (0.5 mL). The solution of compound 21 was added to the solution of compound 1 and stirred for 30 minutes at room temperature. The reaction mixture was diluted with 200 mL of water and filtered to remove any resulting precipitate. The diluted solution was purified using an anion exchange column and eluted with a gradient of 0 to 50% 1M NaCl in water. The desired fractions were collected, diluted to a conductivity of <5 mS/cm and purified on a reverse phase column (YMC Actus Triart C18 250×20.0 mm, I.D. S-5 μm, 12 nm) with a gradient of 0 to 50% acetonitrile in water. The desired fractions were dried to yield a clear film (23.1 mg, 26 μmol, 47% yield).

MS m/z=818.3 [M−H].

1H NMR (500 MHz, D₂O): δ.8.16 (d, J=7.9 Hz, 1H), 7.56 (d, J=7.6 Hz, 1H), 7.46-7.27 (m, 5H), 7.21-7.18 (m, 1H), 6.25 (d, J=7.9 Hz, 1H), 5.98-5.96 (m, 1H), 4.96 (d, J=14.4 Hz, 1H), 4.69-4.64 (m, 1H), 4.47-4.39 (m, 2H), 4.20-4.06 (m, 2H), 3.95-3.83 (m, 2H), 3.64-3.54 (m, 3H), 3.20-3.15 (m, 1H), 2.97-2.84 (m, 2H), 2.53-2.43 (m, 1H), 2.16-2.05 (m, 3H), 1.80-1.68 (m, 1H), 1.33-1.16 (m, 6H).

31P NMR (200 MHz, D₂O): δ.0.62 (s, 1P), 0.36 (s, 1P).

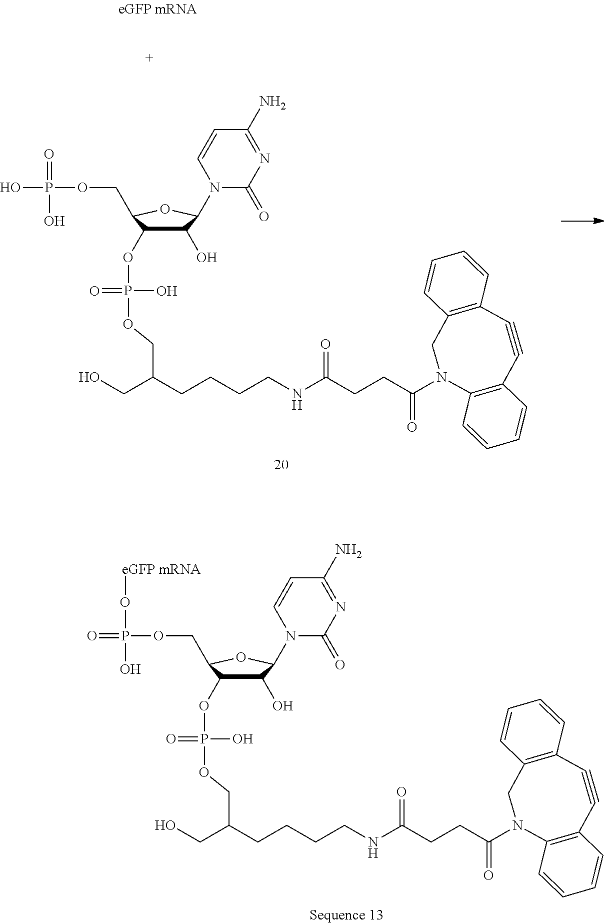

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 20 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.505 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes was performed. The presence of the new product was confirmed by HPLC.

Retention Time eGFP mRNA: 9.335 min.; Retention Time Sequence 13: 14.355 min.

### Example S19: Synthesis of Sequence 14

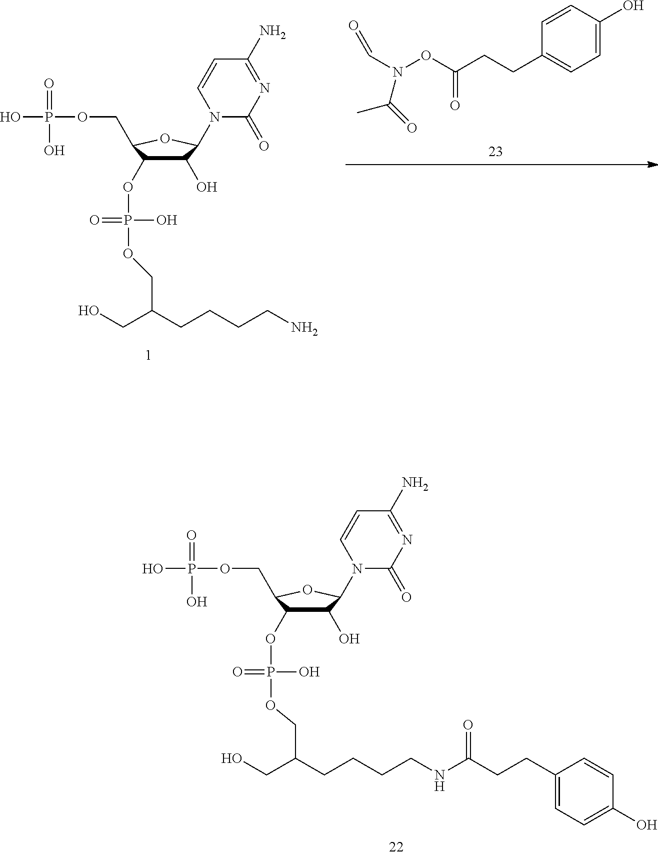

Compound 1 (TEA salt, 80 mg, 56 μmol) was dissolved in de-ionized water (1 mL), and triethylamine (100 μL, 720 mmol) was added. In a separate vial, compound 23 (30 mg, 110 μmol) was dissolved in DMSO (0.5 mL). The solution of compound 23 was added to the solution of compound 1 and stirred for 30 minutes at room temperature. The reaction mixture was diluted with 200 mL of water and filtered to remove any resulting precipitate. The diluted solution was purified using an anion exchange column and eluted with a gradient of 0 to 35% 1M NaCl in water. The desired fractions were collected, diluted to a conductivity of <5 mS/cm and purified on a reverse phase column (YMC Actus Triart C18 250×20.0 mm, I.D. S-5 μm, 12 nm) with a gradient of 0 to 20% acetonitrile in water. The desired fractions were dried to yield a clear film (22.4 mg, 32 μmol, 58% yield).

MS m/z=679.2 [M−H].

1H NMR (500 MHz, D₂O): δ.8.18 (d, J=8.0 Hz, 1H), 7.09 (d, J=8.5 Hz, 2H), 6.80 (d, J=11.4 Hz, 2H), 6.26 (d, J=8.0 Hz, 1H), 5.99 (d, J=4.9 Hz, 1H), 4.67-4.63 (m, 1H), 4.49-4.46 (m, 1H), 4.43 (t, J=5.0 Hz, 1H), 4.24-4.20 (m, 1H), 4.13-4.08 (m, 1H), 3.91-3.81 (m, 2H), 3.57-3.54 (m, 2H), 3.03 (t, J=6.5 Hz, 2H), 2.81 (t, J=6.8 Hz, 2H), 2.47 (t, J=7.0 Hz, 2H), 1.75-1.69 (m, 1H), 1.24-1.18 (m, 4H), 1.05-0.96 (m, 2H).

31P NMR (200 MHz, D₂O): δ.0.58 (s, 1P), 0.34 (s, 1P).

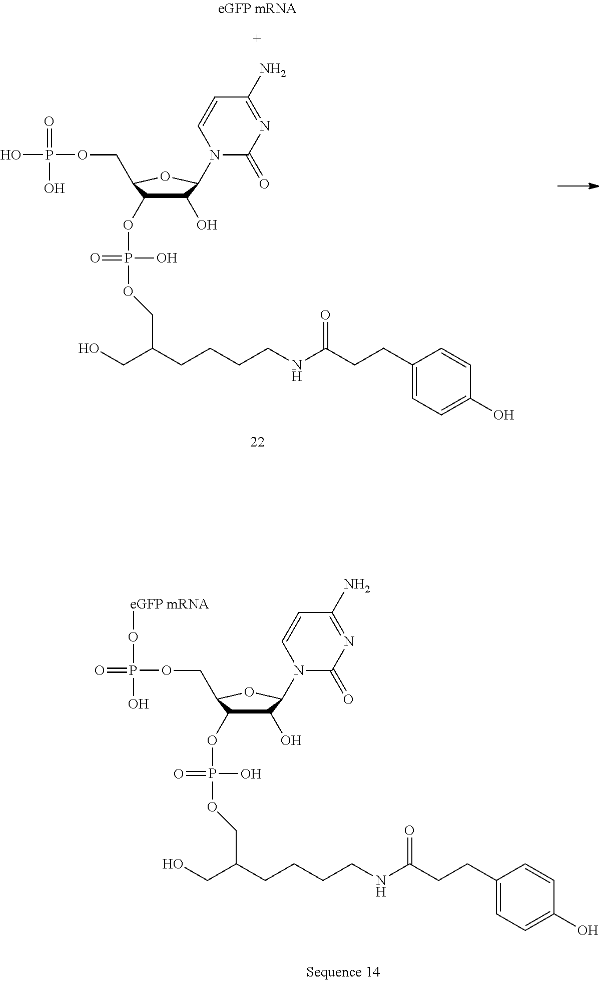

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 22 (Na salt,1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.509 mg/mL. Purification was carried out IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes was performed. The presence of the new product was confirmed by HPLC.

Retention Time eGFP mRNA: 9.335 min.; Retention Time Sequence 14: 10.117 min.

### Example S20: Synthesis of Sequence 15

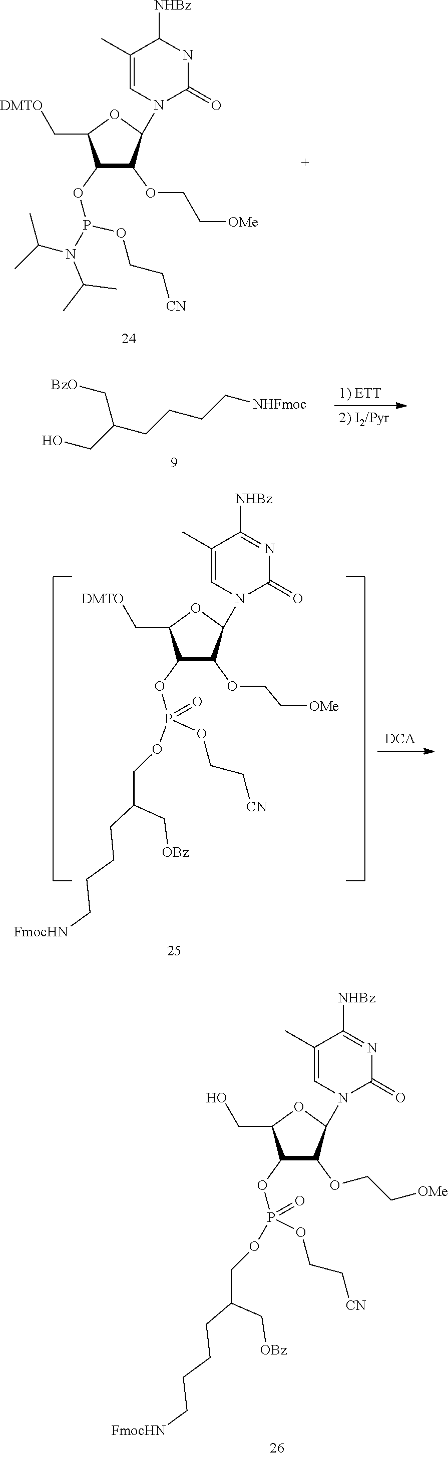

Compound 9 (Shijiazhuang Lidekang Medichem. Ltd., Catalog No. C-200; 1.2 g, 2.5 mmol) was dissolved in acetonitrile (10 ml) and DCM (25 ml), then molecular sieves (5 g) were added to the solution. The mixture was stirred for 1 hr., then compound 24 (2.0 g, 2.2 mmol) and 5-(ethylthio)-1H-tetrazole (ETT) (0.31 g, 2.4 mmol) were added to the mixture and stirred for an additional 3.5 hr. The reaction was followed by LCMS. A solution of iodine (0.65 g, 2.5 mmol), pyridine (1 ml) and water (0.1 ml) in DCM (15 ml) was slowly added to the mixture and stirred for 15 min. Formation of compound 25 was detected by LCMS. Dichloroacetic acid (4 ml) was added to compound 25 and the solution was stirred for another 20 min. Sodium bicarbonate solution (70 ml, 10%) was added to neutralize the mixture, followed by addition of sodium bisulfite solution (15 ml, 5%). The mixture was transferred to a separatory funnel and the flask was washed with DCM (350 ml). The solution was washed once with brine and dried over sodium sulfate. The solvent was evaporated and compound 26 was purified using Combi-Flash silica column (DCM/EtOAc). Compound 26 was obtained with 50.2% yield (1. g).

MS m/z=1008.5 [M+H].

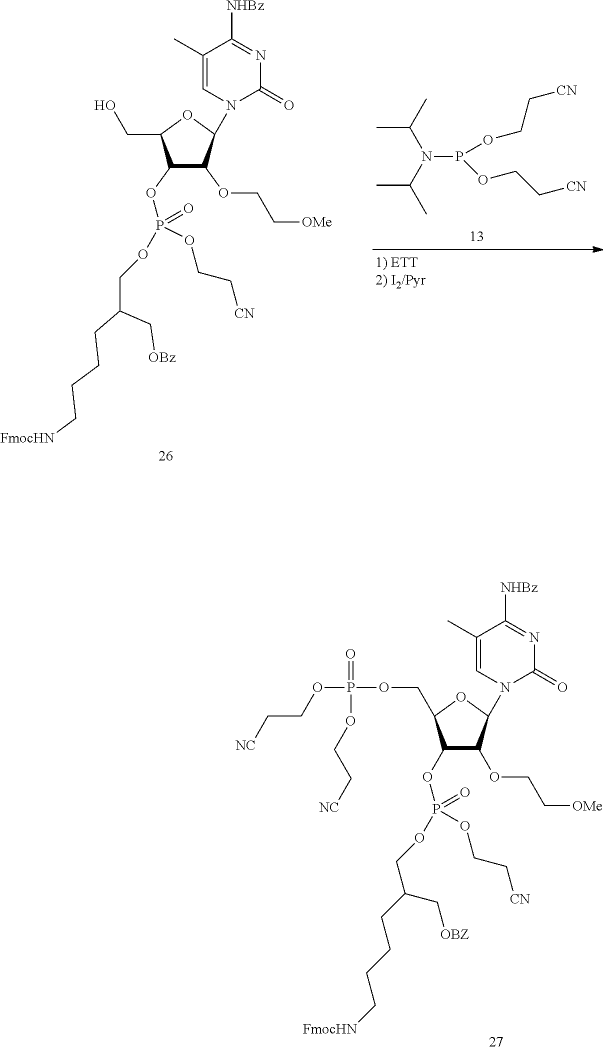

Compound 26 (1.lg, 1.1 mmol) was dissolved in acetonitrile (6 ml) and DCM (15 ml), then molecular sieves (5 g) were added to the solution. The mixture was stirred for 1 hr., then compound 13 (0.60 g, 2.2 mmol) and 5-(ethylthio)-1H-tetrazole (0.28 g, 2.2 mmol) were added to the mixture and stirred for an additional 1 hr. The reaction was followed by LCMS. A solution of iodine (0.38 g, 1.5 mmol), pyridine (0.5 ml) and water (0.1 ml) in DCM (15 ml) was slowly added to the mixture and stirred for 15 min. Sodium bisulfite solution (15 ml, 5%) was added and stirred for 3 min. The solution was transferred to a separatory funnel. The flask was washed with 300 ml of DCM and DCM was transferred to the separatory funnel. The solution was washed once with brine and dried over sodium sulfate. The solvent was evaporated, and the crude compound 27 was obtained and used in the next reaction without purification (1.7 g crude).

MS m/z=1194.6 [M+H].

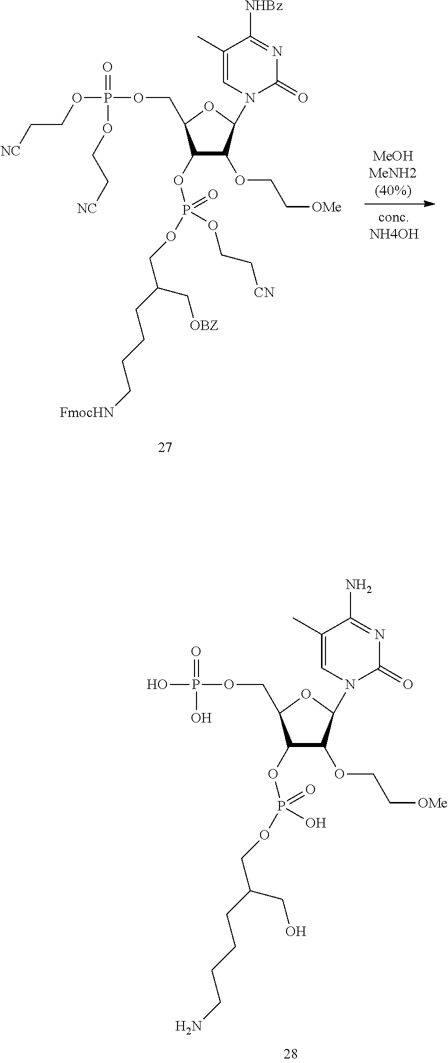

Crude compound 27 (1.7 g) was dissolved in MeOH (20 ml). MeNH₂ (30 ml, 40%) and NH₄OH (15 ml, 36%) were added, and the solution was stirred at r.t. for 18 hrs. Half of the solvent was evaporated, and 50 ml of water was added. The solution was washed with ethyl acetate (200 ml). The aqueous solution was purified on an anion exchange column, eluted with a gradient of 0 to 12.7% 1M NaCl in water over 11.5 column volumes (CVs). The desired fractions were collected diluted with water to a conductivity of <5 mS/cm and purified on a reverse phase column (YMC Actus Triart C18 250×20.0 mm, I.D. S-5 μm, 12 nm) eluted with a gradient of 0 to 20% acetonitrile in water over 10 CVs. The desired fractions were dried to yield compound 28 as a clear film (300 mg, 500 μmol, 45% yield).

MS m/z=603.2 [M−H].

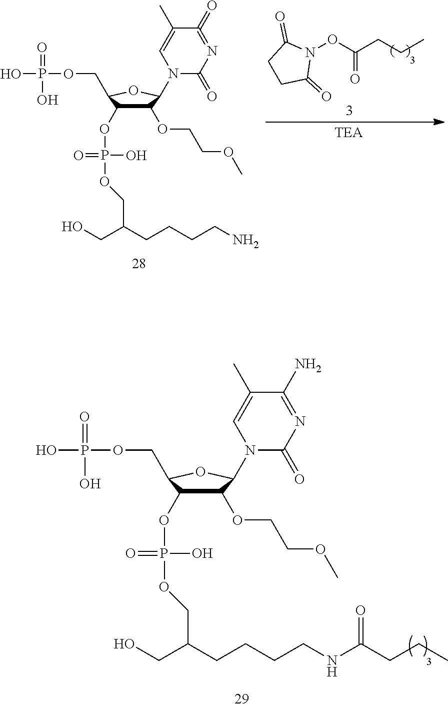

Compound 28 (TEA salt, 30 mg, 50 μmol) was dissolved in de-ionized water (1 mL), and triethylamine (100 μL, 720 mmol) was added. In a separate vial, Compound 3 (21 mg, 110 μmol) was dissolved in DMSO (0.5 mL). The solution of Compound 3 was added to the solution of Compound 28 and stirred for 30 minutes at room temperature. The solution was diluted with 200 mL water and filtered to remove any resulting precipitate. The diluted solution was purified using an anion exchange column and eluted with a gradient of 0 to 35% 1M NaCl in water. The desired fractions were collected, diluted to a conductivity of <5 mS/cm and purified on a reverse phase column (YMC Actus Triart C18 250×20.0 mm, I.D. S-5 μm, 12 nm) with a gradient of 0 to 50% acetonitrile in water. The desired fractions were dried to yield compound 29 as a clear film (32.6 mg, 46 μmol, 92% yield).

MS m/z=701.0 [M−H].

1H NMR (500 MHz, D₂O): δ.7.94 (s, 1H), 6.16 (d, J=6.7 Hz, 1H), 4.48-4.44 (m, 1H), 4.36-4.33 (m, 1H), 4.05-3.99 (m, 1H), 3.98-3.85 (m, 4H), 3.79-3.73 (m, 1H), 3.61-3.55 (m, 4H), 3.28 (s, 3H), 3.17 (t, J=6.7 Hz, 2H), 2.20 (t, J=7.3 Hz, 2H), 2.03 (s, 3H), 1.83-1.76 (m, 1H), 1.60-1.46 (m, 4H), 1.41-1.32 (m, 4H), 1.32-1.20 (m, 4H), 0.85 (t, J=7.0 Hz, 3H).

31P NMR (200 MHz, D₂O): δ.3.98 (s, 1P), 0.74 (d, 1P).

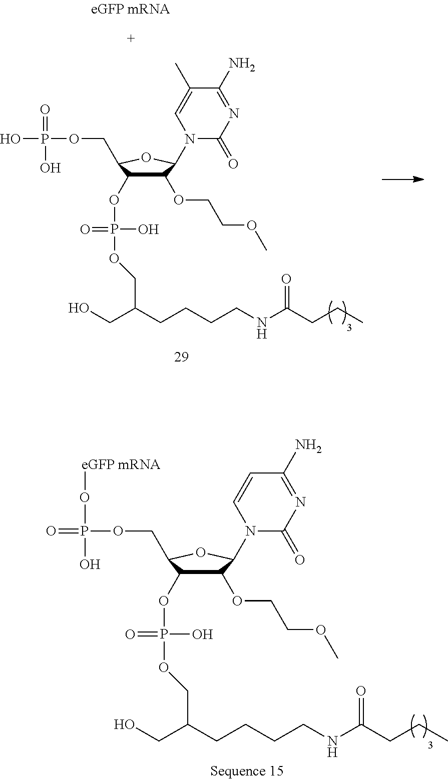

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 29 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.631 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 80% Buffer B in Buffer A over 29.8 minutes was performed. The presence of the new product was confirmed by HPLC.

Retention Time eGFP mRNA: 9.335 min.; Retention Time Sequence 15: 11.598 min.

### Example S21: Synthesis of Sequence 16

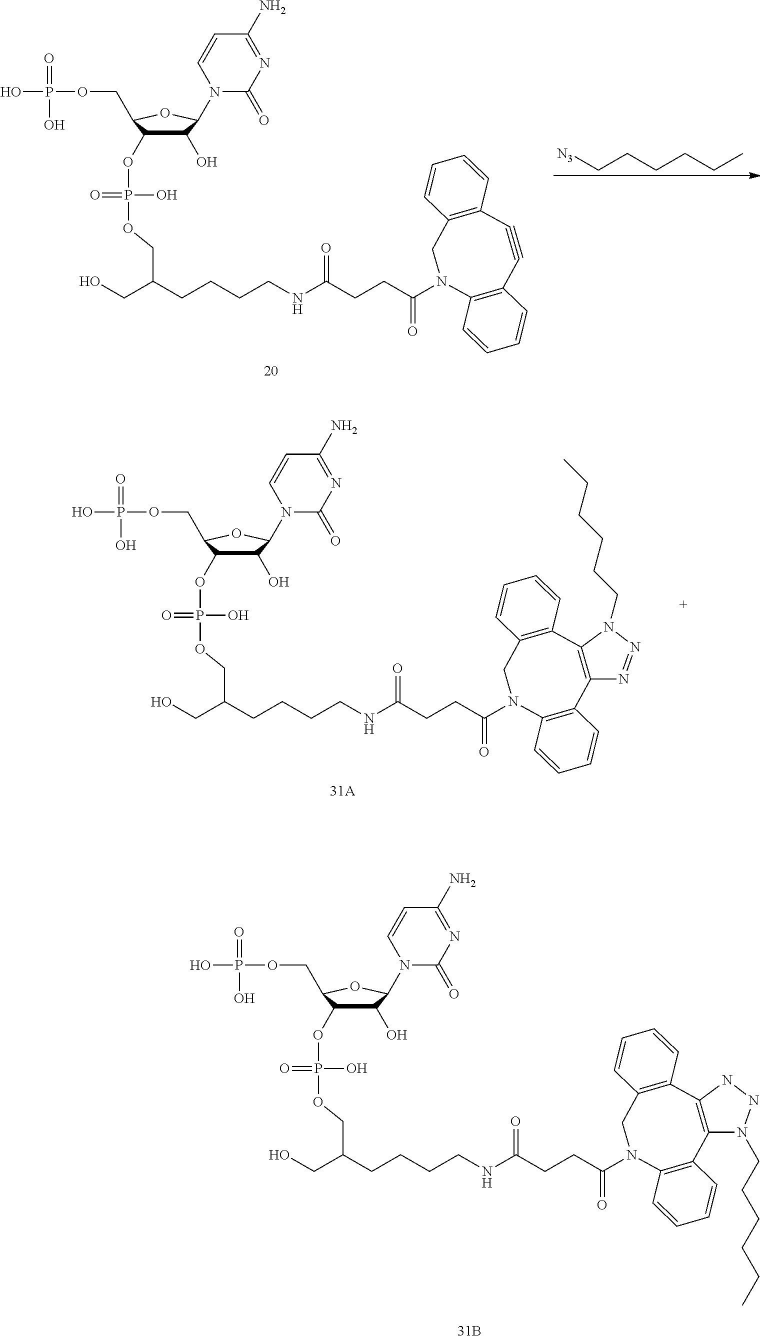

Compound 20 (Na salt, 15 mg, 18 μmol) was suspended in dry DMSO (0.5 mL), 1-azidohexane (4.7 mg, 37 μmol) was added and the mixture was stirred at room temperature for 18 hours. The reaction was diluted with water to conductivity of −5 mS/cm. The solution was filtered and purified on an anion exchange column with a gradient of 0 to 50% 1M NaCl in water. The desired fractions were collected and diluted to a conductivity of <5 mS/cm and then purified on a reverse phase column (YMC Actus Triart C18 250×20.0 mm, I.D. S-5 μm, 12 nm) with a gradient of 0 to 50% acetonitrile in water. The desired fractions were then dried to yield a mix of two isomers of compound 31 (isomer 31A and 31B) as a clear film (14.5 mg, 15 μmol, 85% yield).

MS m/z=945.4 [M−H].

1H NMR (500 MHz, D₂O): δ.8.09 (d, J=7.6 Hz, 1H), 7.69-7.48 (m, 4H), 7.47-7.25 (m, 4H), 7.09 (d, J=7.8 Hz, 1H), 6.13 (t, J=7.5 Hz, 2H), 5.89 (d, J=17 Hz, 1H), 4.68-4.64 (m, 2H), 4.59-4.43 (m, 4H), 4.40-4.36 (m, 1H), 4.05-3.83 (m, 4H), 3.61-3.54 (m, 2H), 3.13-3.05 (m, 2H), 2.40-2.08 (m, 3H), 2.07-1.97 (m, 2H), 1.96-1.56 (m, 4H), 1.47-1.40 (m, 2H), 1.39-1.24 (m, 6H), 1.20-0.92 (m, 4H), 0.84-0.80 (m, 1H), 0.75 (t, J=7.0 Hz, 2H).

31P NMR (200 MHz, D₂O): δ.3.94 (s, 1P), 0.79 (d, 1P).

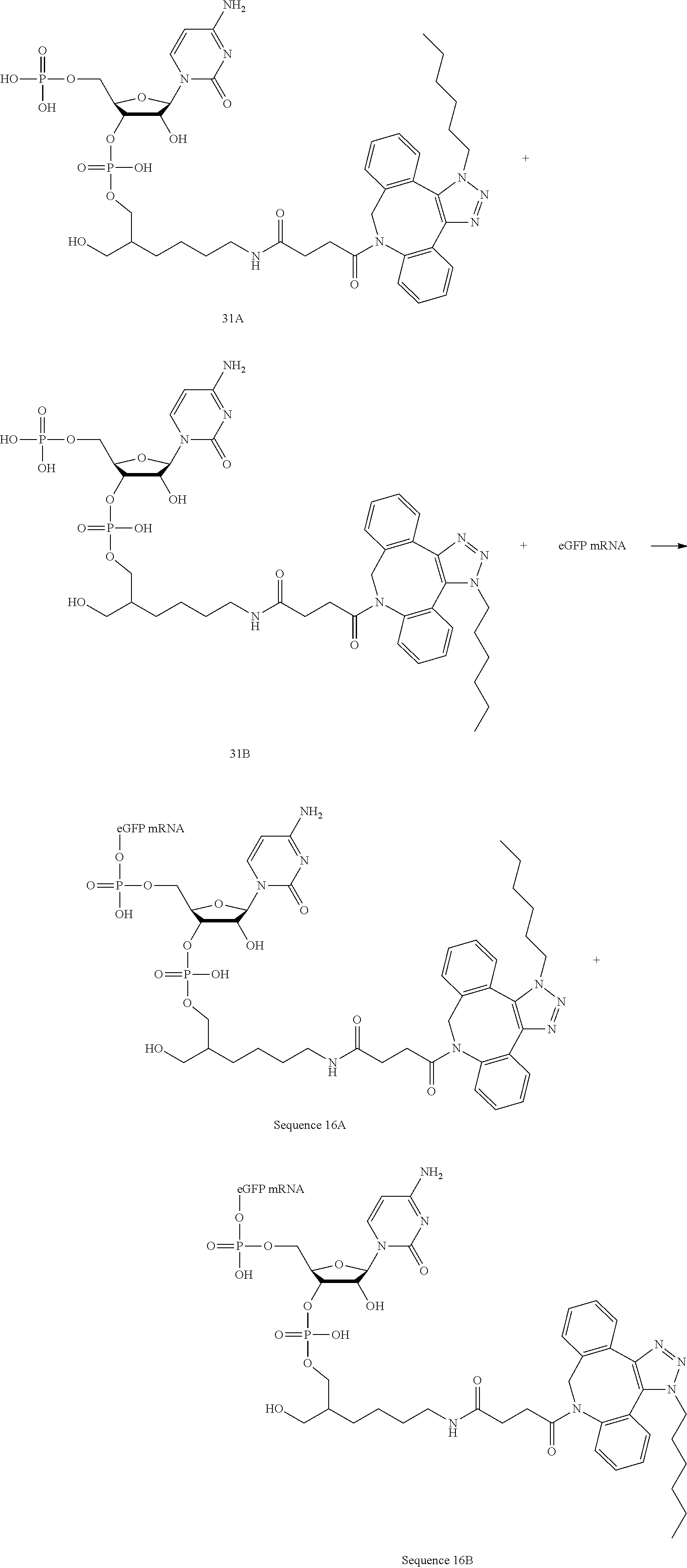

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 31 (mixture of isomers 31A and 31B) (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.721 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 80% Buffer B in Buffer A over 29.8 minutes was performed. The presence of the new product Sequence 16 (which is a mixture of the two isomers Sequences 16A and 16B) was confirmed by HPLC.

Retention Time eGFP mRNA: 9.335 min.; Retention Time Sequence 16: 19.276 min.

### Example S22: Synthesis of Sequence 17

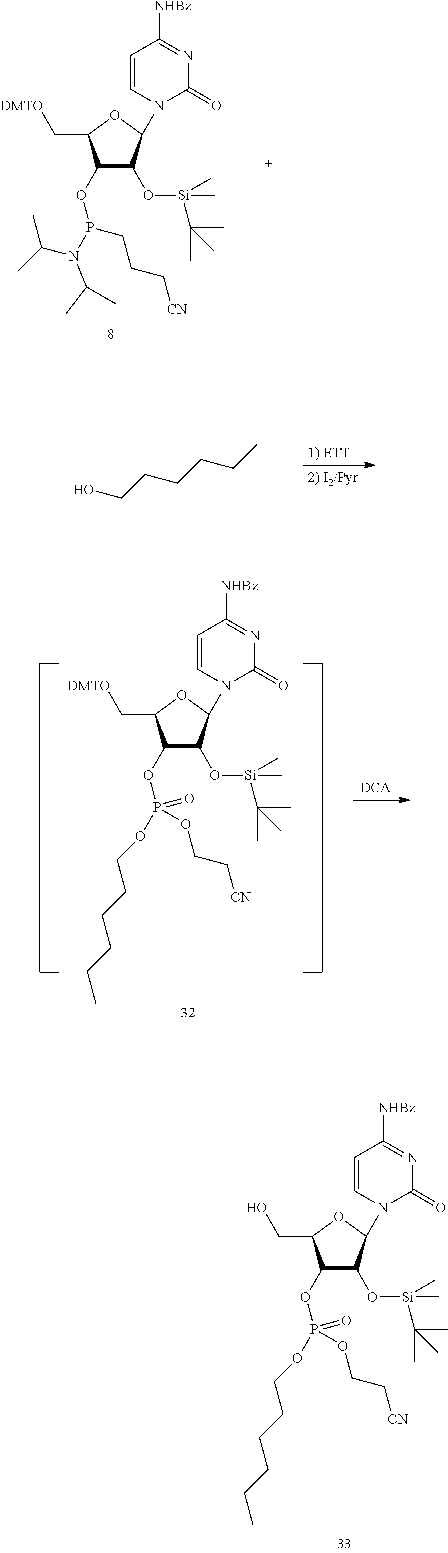

1-hexanol (0.51 g, 5.0 mmol) was dissolved in acetonitrile (35 ml), then molecular sieves (10 g) were added to the solution. The mixture was stirred for 1 hr., then compound 8 (4.82 g, 5.0 mmol) and 5-(ethylthio)-1H-tetrazole (ETT) (0.68 g, 5.2 mmol) were added to the mixture and stirred for an additional 3.5 hr. The reaction was followed by LCMS. A solution of iodine (1.35 g, 8.4 mmol), pyridine (1 ml) and water (0.2 ml) in DCM (10 ml) was slowly added to the mixture and stirred for 15 min. Formation of compound 32 was detected by LCMS. Dichloroacetic acid (5.0 ml) was added to compound 32 and the solution was stirred for another 20 min. Sodium bicarbonate solution (80 ml, 10%) was added to neutralize the mixture, followed by addition of sodium bisulfite solution (15 ml, 5%). The mixture was transferred to a separatory funnel and the flask was washed with DCM (350 ml). The solution was washed once with brine and dried over sodium sulfate. The solvent was evaporated and compound 33 was purified using Combi-Flash silica column (DCM/EtOAc). Compound 33 was obtained with 50.0% yield (1.7 g).

MS m/z=679.3 [M+H].

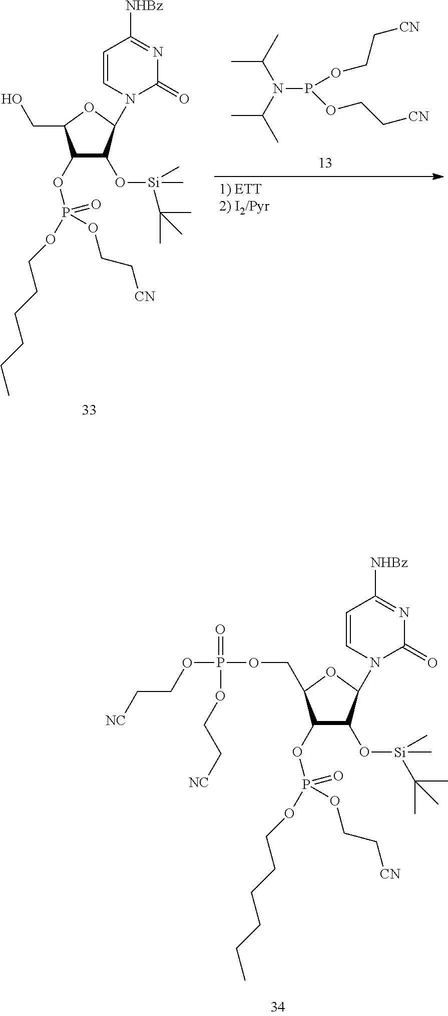

Compound 33 (1.4 g, 2.1 mmol) was dissolved in acetonitrile (10 ml) and DCM (12 ml), then molecular sieves (5 g) were added to the solution. The mixture was stirred for 1 hr., then compound 13 (0.67 g, 2.5 mmol) and 5-(ethylthio)-1H-tetrazole (0.32 g, 2.5 mmol) were added to the mixture and stirred for an additional 0.5 hr. The reaction was followed by LCMS. A solution of iodine (0.53 g, 2.1 mmol), pyridine (0.5 ml) and water (0.1 ml) in DCM (10 ml) was slowly added to the mixture and stirred for 15 min. Sodium bisulfite solution (15 ml, 5%) was added and stirred for 3 min. The solution was transferred to a separatory funnel. The flask was washed with 300 ml of DCM and DCM was transferred to the separatory funnel. The solution was washed once with brine and dried over sodium sulfate. The solvent was evaporated, and the crude compound 34 was obtained and used in the next reaction without purification (2.05 g crude).

MS m/z=865.5 [M+H].

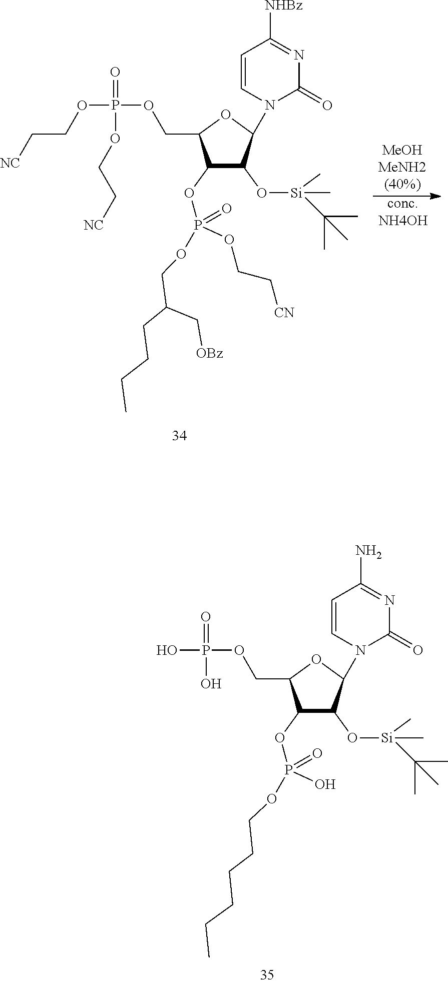

Crude compound 34 was dissolved in MeOH (20 ml). MeNH₂ (30 ml, 40%) and NH40H (15 ml, 36%) were added, and the solution was stirred at r.t. for 18 hrs. Half of the solvent was evaporated, and 50 ml of water was added. The solution was washed with ethyl acetate (200 ml). The aqueous solution was purified on a reverse phase column (YMC Actus Triart C18 250×20.0 mm, I.D. S-5 μm, 12 nm) eluted with a gradient of 0 to 90% acetonitrile in 100 mM TEAB. The desired fractions were combined and dried to yield compound 35 as a clear film (500 mg, 830 μmol, 40% yield over two steps).

MS m/z=600.2 [M−H].

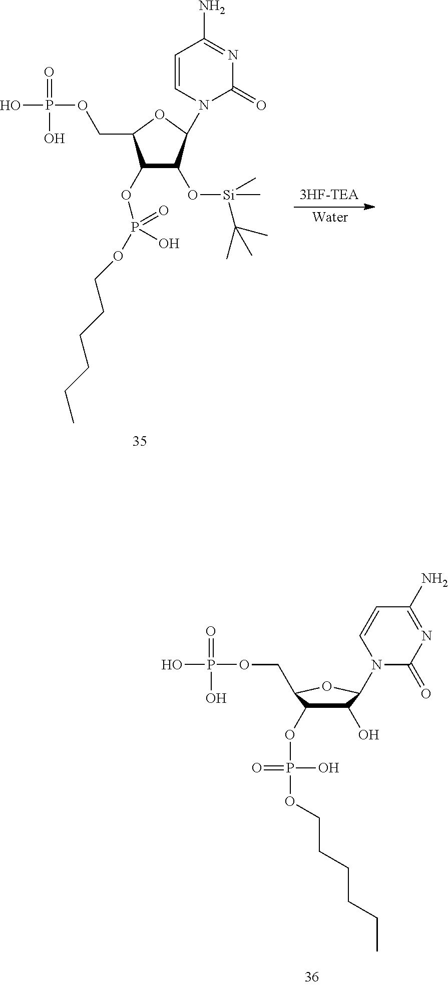

Compound 35 (TEA salt, 500 mg, 830 μmol) was suspended in water (20 mL), 31HF-TEA (5 mL) was added, and the solution was stirred at room temperature 24 hours. Acetonitrile (20 mL) and 31HF-TEA (5 mL) were added to the reaction and stirring continued at room temperature for 4 hours. The solution was neutralized with 1 M TEAB (50 mL), then diluted with water to a conductivity of −5 mS/cm. The solution was filtered, and purified on an anion exchange column (eluted with a gradient of 0 to 50% 1M NaCl in water). The desired fractions were collected and diluted to a conductivity of <5 mS/cm and then loaded onto a reverse phase column (YMC Actus Triart C18 250×20.0 mm, I.D. S-5 μm, 12 nm) and eluted with a gradient of 0 to 50% acetonitrile in water. The desired fractions were collected, and dried to yield compound 36 as a clear film (215 mg, 440 μmol, 53% yield).

MS m/z=486.0 [M−H].

1H NMR (500 MHz, D₂O): δ.8.16 (d, J=8.0 Hz, 1H), 6.27 (d, J=8.0 Hz, 1H), 5.98 (d, J=4.8 Hz, 1H), 4.78-4.61 (m, 1H), 4.48-4.46 (m, 1H), 4.42 (t, J=4.9 Hz, 1H), 4.25-4.11 (m, 2H), 3.91 (q, J=6.7 Hz, 2H), 1.64-1.58 (m, 2H), 1.36-1.23 (m, 6H), 0.84 (t, J=7.0 Hz, 3H).

31P NMR (200 MHz, D₂O): δ.0.36 (s, 1P), 0.20 (s, 1P).

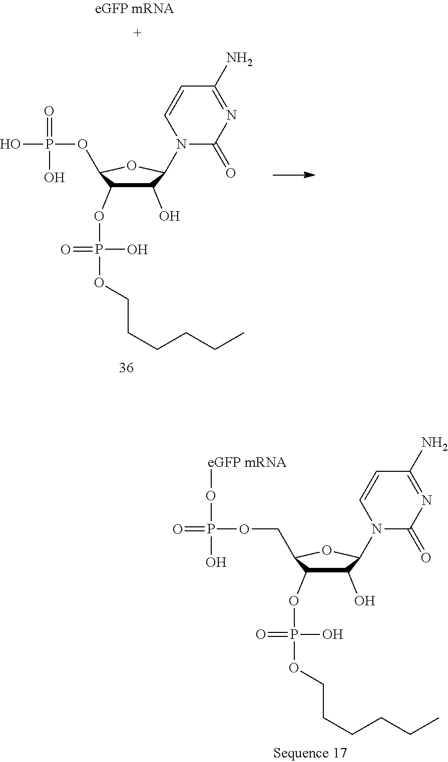

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 36 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.762 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 80% Buffer B in Buffer A over 29.8 minutes was performed. The presence of the new product Sequence 16 (which is a mixture of the two isomers Sequences 16A and 16B) was confirmed by HPLC.

Retention Time eGFP mRNA: 9.335 min.; Retention Time Sequence 17: 11.023 min.

### Example S23: Synthesis of Sequence 18

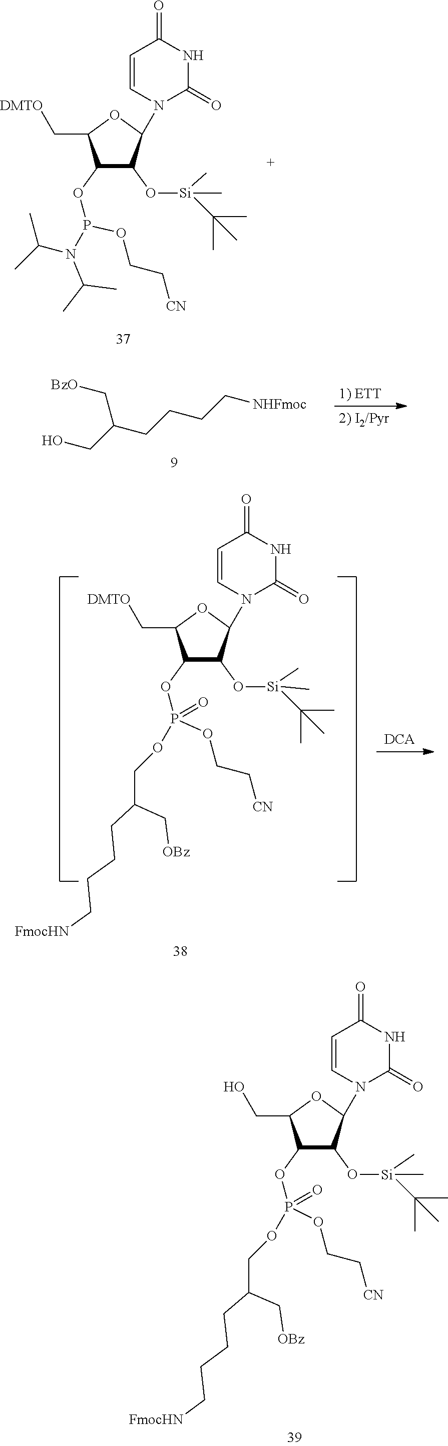

Compound 9 (Shijiazhuang Lidekang Medichem. Ltd., Catalog No. C-200; 2.4 g, 5.0 mmol) was dissolved in acetonitrile (10 ml) and DCM (45 ml), then molecular sieves (8 g) were added to the solution. The mixture was stirred for 1 hr., then compound 37 (4.3 g, 5.0 mmol) and 5-(ethylthio)-1H-tetrazole (ETT) (0.65 g, 5.0 mmol) were added to the mixture and stirred for an additional 3.5 hr. The reaction was followed by LCMS. A solution of iodine (1.35 g, 5.3 mmol), pyridine (1 ml) and water (0.1 ml) in DCM (10 ml) was slowly added to the mixture and stirred for 15 min. Formation of compound 38 was detected by LCMS. Dichloroacetic acid (5.5 ml) was added to compound 38 and the solution was stirred for another 20 min. Sodium bicarbonate solution (80 ml, 10%) was added to neutralize the mixture, followed by addition of sodium bisulfite solution (15 ml, 5%). The mixture was transferred to a separatory funnel and the flask was washed with DCM (350 ml). The solution was washed once with brine and dried over sodium sulfate. The solvent was evaporated and compound 39 was purified using Combi-Flash silica column (DCM/EtOAc). Compound 39 was obtained with 52.7% yield (2.5 g).

MS m/z=946.3 [M−H].

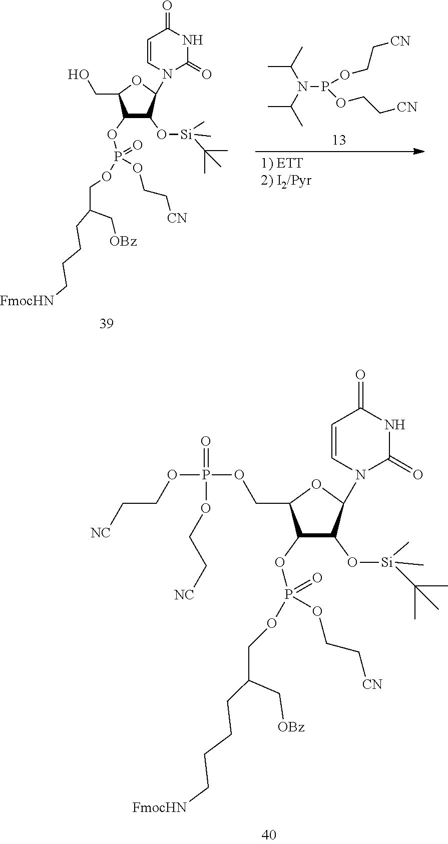

Compound 39 (2.5 g, 2.6 mmol) was dissolved in acetonitrile (10 ml) and DCM (30 ml), then molecular sieves (5 g) were added to the solution. The mixture was stirred for 1 hr., then compound 13 (0.75 g, 2.8 mmol) and 5-(ethylthio)-1H-tetrazole (0.36 g, 2.8 mmol) were added to the mixture and stirred for an additional 0.5 hr. The reaction was followed by LCMS. A solution of iodine (0.67 g, 2.6 mmol), pyridine (0.5 ml) and water (0.1 ml) in DCM (10 ml) was slowly added to the mixture and stirred for 15 min. Sodium bisulfite solution (15 ml, 5%) was added and stirred for 3 min. The solution was transferred to a separatory funnel. The flask was washed with 300 ml of DCM and DCM was transferred to the separatory funnel. The solution was washed once with brine and dried over sodium sulfate. The solvent was evaporated, and the crude compound 40 was obtained and used in the next reaction without purification (3.lg crude).

MS m/z=1133.7 [M+H].

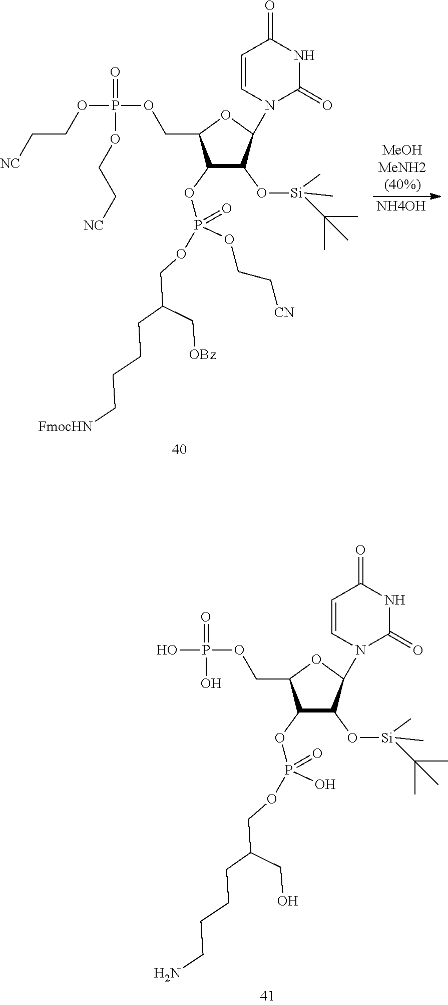

Crude compound 40 was dissolved in MeOH (20 ml). MeNH₂ (30 ml, 40%) and NH40H (15 ml, 36%) were added, and the solution was stirred at r.t. for 18 hrs. Half of the solvent was evaporated, and 50 ml of water was added. The solution was washed with ethyl acetate (200 ml). Crude compound 41 was purified using Reverse Phase chromatography (using a linear gradient from 0% to 80%, of buffer A 100 mM TEAB and buffer B acetonitrile, over 12 column volumes (CV) and holding at 100% for 1.5 CV). The desired fractions were pooled and concentrated and used in the next step without quantification.

MS m/z=646.2 [M−H].

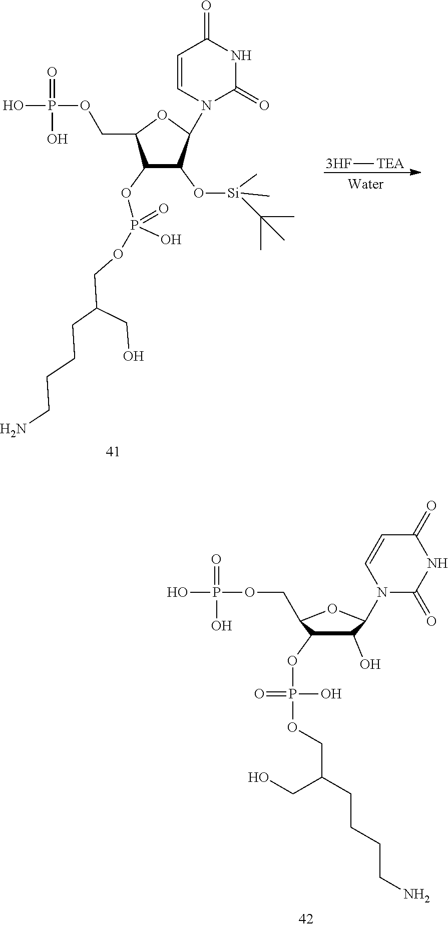

One fifth of the partially purified compound 41 (TEA salt) was suspended in water (20 mL). 31HF-TEA (5 mL) was added, and the solution was stirred at room temperature for 18 hours. The solution was neutralized with 1 M TEAB (50 mL), then diluted with water to a conductivity of −5 mS/cm. The solution was filtered, and purified on an anion exchange column QFF (eluted with a linear gradient from 0% to 100%, of buffer A water and buffer B 100 mM TEAB, over 10 column volumes (CV) and holding at 100% for 1.5 CV). The desired fractions were collected, and dried yielding compound 42 as a TEA salt (546 mg, 0.268 mmol, 26.8% yield over 3 steps; 2040.86 g/mol).

MS m/z=532.1 [M−H].

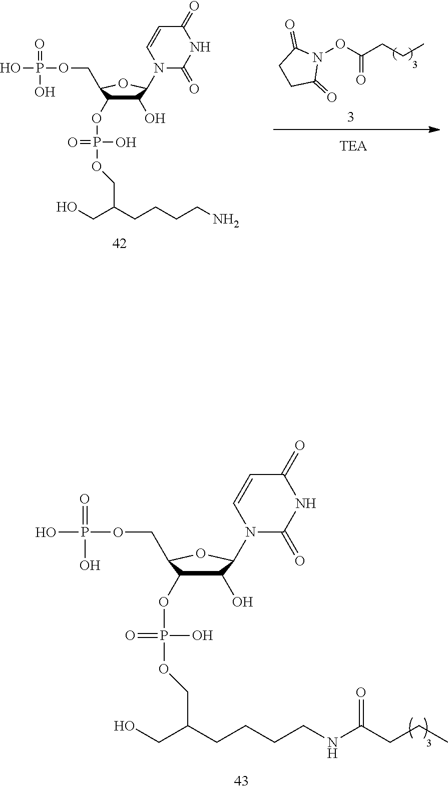

Compound 42 (TEA salt, 110 mg, 56 μmol) was dissolved in de-ionized water (1 mL), and triethylamine (100 μL, 720 mmol) was added. In a separate vial, Compound 3 (24 mg, 110 μmol) was dissolved in DMSO (0.5 mL). The solution of Compound 3 was added to the solution of Compound 42 and stirred for 30 minutes at room temperature. The solution was diluted with 200 mL water and filtered to remove any resulting precipitate. The diluted solution was purified using an anion exchange column and eluted with a gradient of 0 to 35% 1M NaCl in water. The desired fractions were collected, diluted to a conductivity of <5 mS/cm and purified on a reverse phase column (YMC Actus Triart C18 250×20.0 mm, I.D. S-5 μm, 12 nm) with a gradient of 0 to 50% acetonitrile in water. The desired fractions were dried to yield compound 43 as a clear film (12 mg, 19 μmol, 34% yield).

MS m/z=630.2 [M−H].

1H NMR (500 MHz, D₂O): δ.7.94 (d, J=8.2 Hz, 1H), 6.05 (d, J=6.4 Hz, 1H), 5.95 (d, J=8.2 Hz, 1H), 4.67-4.63 (m, 1H), 4.47-4.46 (m, 1H), 4.43 (t, J=5.4 Hz, 1H), 4.17-4.07 (m, 2H), 3.95-3.86 (m, 2H), 3.60 (d, J=5.7 Hz, 2H), 3.17 (t, J=6.6 Hz, 2H), 2.20 (t, J=7.4 Hz, 2H), 1.84-1.76 (m, 1H), 1.60-1.47 (m, 4H), 1.39-1.32 (m, 3H), 1.31-1.21 (m, 4H), 0.85 (t, J=7.0 Hz, 3H).

31P NMR (200 MHz, D₂O): δ.0.67 (d, 1P), 0.41 (s, 1P).

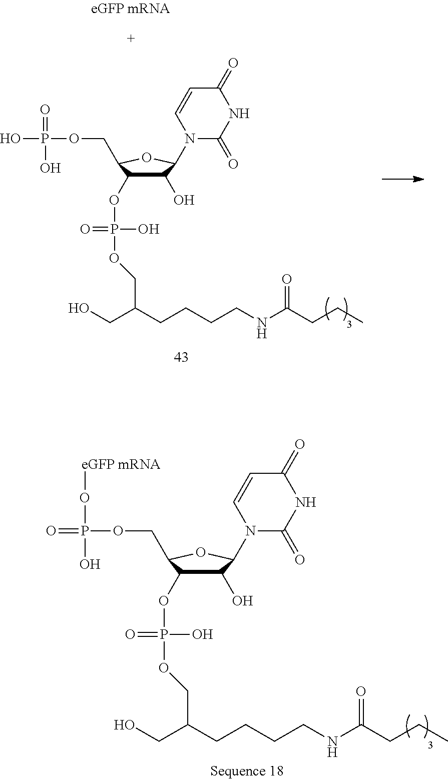

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 43 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.843 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 80% Buffer B in Buffer A over 29.8 minutes was performed. The presence of the new product Sequence 16 was confirmed by HPLC.

Retention Time eGFP mRNA: 9.335 min.; Retention Time Sequence 18: 11.598 min.

### Example S24: Synthesis of Sequence 19

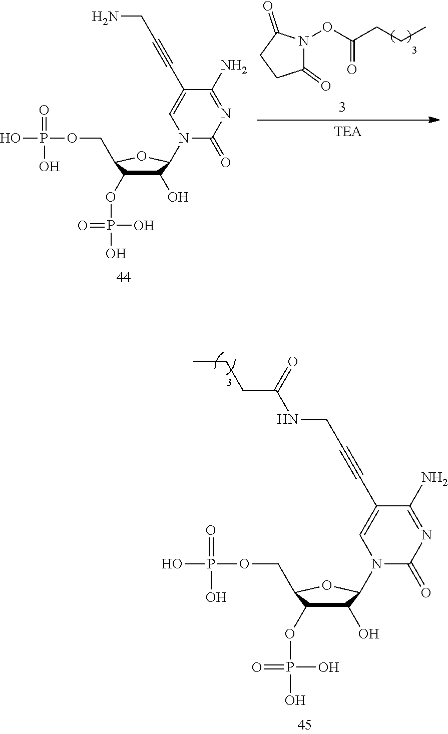

Compound 44 (purchased from NuBlocks, custom synthesis; TEA salt, 30 mg, 46 μmol) was dissolved in 10% water/DMSO (1 mL), and triethylamine (100 μL, 720 mmol) was added. The suspension was stirred at room temperature for 2 hrs., giving a hazy solution. Compound 3 (19 mg, 90 μmol) was added to the solution and stirred for 5 minutes at room temperature. The solution was diluted with 40× water and purified using an anion exchange chromatography, followed by reverse phase chromatography. The desired fractions were dried to yield compound 45 as a white residue (14.9 mg, 43% yield).

MS m/z=553.2 [M−H].

1H NMR (500 MHz, D₂O): 6 8.16 (s, 1H), 8.08 (s, 1H), 6.0-5.98 (m, 1H), 4.62-4.56 (m, 1H), 4.52-4.48 (m, 1H), 4.37-4.32 (m, 2H), 4.22-4.15 (m, 2H), 4.07-3.91 (m, 2H), 2.26 (t, J=7.3 Hz, 2H), 1.59 (quintet, J=7.5 Hz, 2H), 1.32-1.22 (m, 3H), 0.85-0.80 (m, 2H).

31P NMR (200 MHz, D₂O): 6 4.07 (d, 1P), 3.80 (s, 1P)

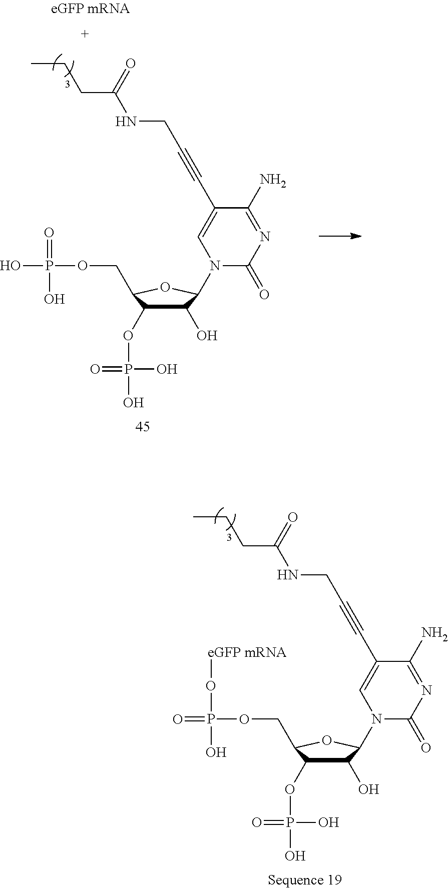

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 45 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.792 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 80% Buffer B in Buffer A over 29.8 minutes was performed. The presence of the new product Sequence 19 was confirmed by HPLC.

Retention Time eGFP mRNA: 9.335 min.; Retention Time Sequence 19: 10.686 min.

### Example S25: Synthesis of Sequence 20

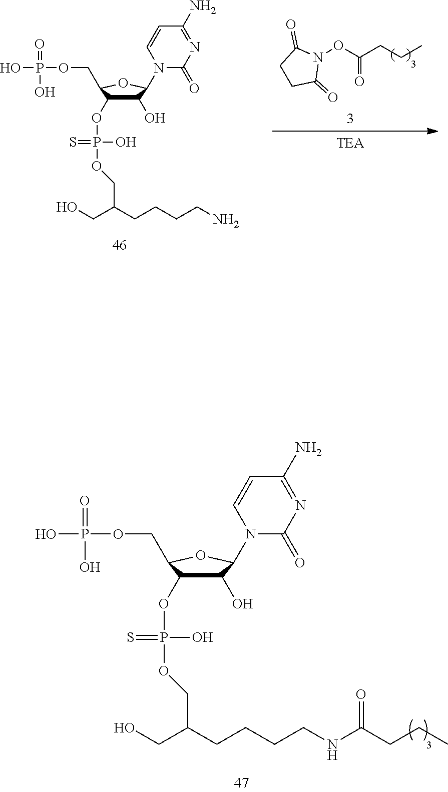

Compound 46 (custom ordered from Trilink; TEA salt, 80 mg, 56 μmole) was dissolved in DMSO (1.5 mL), and triethylamine (100 μL, 720 mmol) was added, followed by addition of compound 3 (10.5 mg, 67.6 μmol), and the solution was stirred for 1 hr. at room temperature. The solution was diluted with 15× water and purified using an anion exchange chromatography (QFF column; using a 0-50% gradient over 10CV, Buffer A Water, Buffer B 1M NaCl), followed by reverse phase chromatography (YMC Actus Triart C18 250×20.0 mm, I.D. S-5 μm, 12 nm) and eluted with a gradient of 0 to 50% acetonitrile in water. The desired fractions were dried to yield compound 47 as a white solid (11.2 mg, 30.4% yield).

MS m/z=645.20 [M−H].

1H NMR (500 MHz, D₂O): 6 8.12 (d, J=8.8 Hz, 1H), 6.13 (m, 2H), 4.52-4.49 (m, 2H), 4.00-3.91 (m, 4H), 3.61-3.58 (m, 4H), 3.18-3.15 (m, 2H), 2.20 (t, J=6.2 Hz, 2H), 1.82 (bs, 1H), 1.59-1.49 (m, 4H), 1.40-1.35 (m, 4H), 1.31-1.22 (m, 4H), 0.85 (t, J=6.9 Hz, 3H).

31P NMR (200 MHz, D₂O): δ.56.38 (t, J=14.7 Hz, 1P), 4.16 (s, 1P).

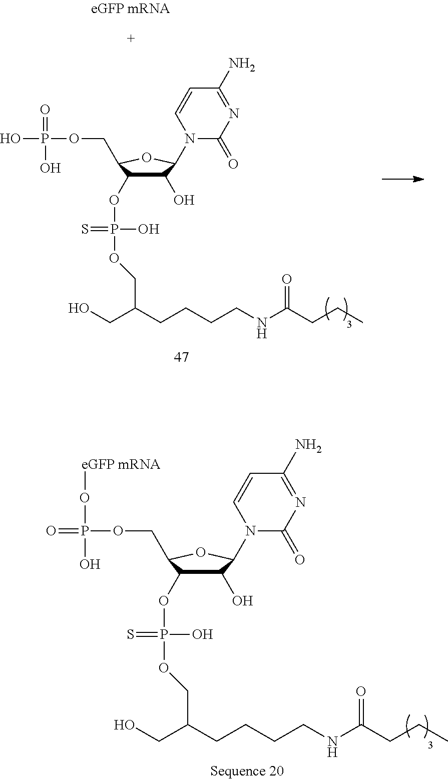

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 47 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.739 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 80% Buffer B in Buffer A over 29.8 minutes was performed. The presence of the new product, Sequence 20 was confirmed by HPLC.

Retention Time eGFP mRNA: 9.335 min.; Retention Time Sequence 20: 11.701 min.

### Example S26: Synthesis of Sequence 21

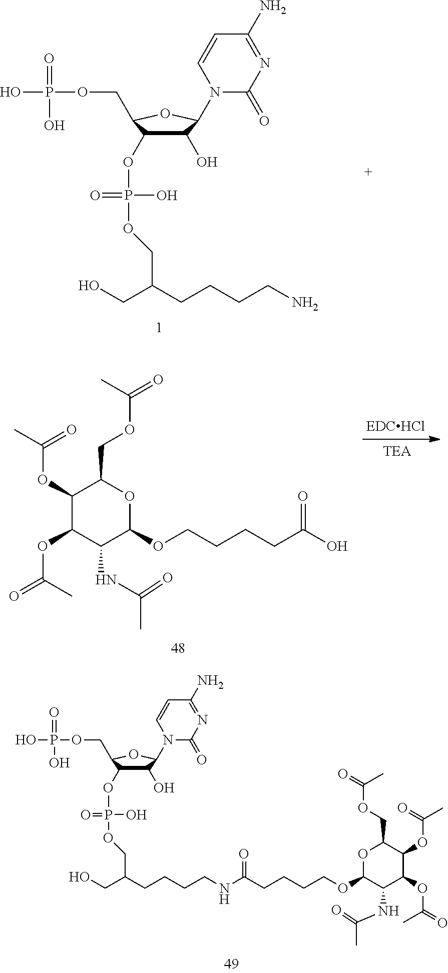

Compound 1 (custom ordered from Trilink; TEA salt, 80 mg, 56 μmole) was dissolved in DMSO (1 mL), and triethylamine (100 μL, 720 mmol) was added. EDC hydrochloride salt (10.5 mg, 67.6 μmol) and compound 48 (purchased from Boc Sciences, Catalog #B2705-000976, 25.2 mg, 56.4 μmol) were added to the solution, and the solution was stirred for 5 hours at r.t. The solution was diluted 15× with water and purified using an anion exchange chromatography (QFF column; using a 0-50% gradient over 10CV, Buffer A Water, Buffer B 1M NaCl), followed by reverse phase chromatography (YMC Actus Triart C18 250×20.0 mm, I.D. S-5 μm, 12 nm) and eluted with a gradient of 0 to 50% acetonitrile in water. The desired fractions were dried to yield compound 49 as a white solid (6.4 mg, 12% yield).

MS m/z=960.40 [M−H].

1H NMR (500 MHz, D₂O): 6 8.28 (d, J=12.1 Hz, 1H), 6.17 (d, J=7.6 Hz 1H), 6.15 (d, J=6.9 Hz 1H), 5.39 (d, J=3.3 Hz 1H), 5.09 (dd, J₁=3.3 Hz, J₂=11.2 Hz, 1H), 4.69-4.65 (m, 2H), 4.51-4.44 (m, 2H), 4.22-4.08 (m, 4H), 3.98-3.87 (m, 5H), 3.63-3.59 (m, 3H), 3.18-3.15 (m, 2H), 2.24-2.21 (m, 5H), 2.08 (s, 3H), 2.00 (s, 3H), 1.90 (s, 3H), 1.61-1.57 (m, 4H), 1.50-1.48 (m, 2H), 1.35 (bs, 4H).

31P NMR (200 MHz, D₂O): δ.4.10 (s, 1P), 0.79 (d, J=11.2 Hz, 1P).

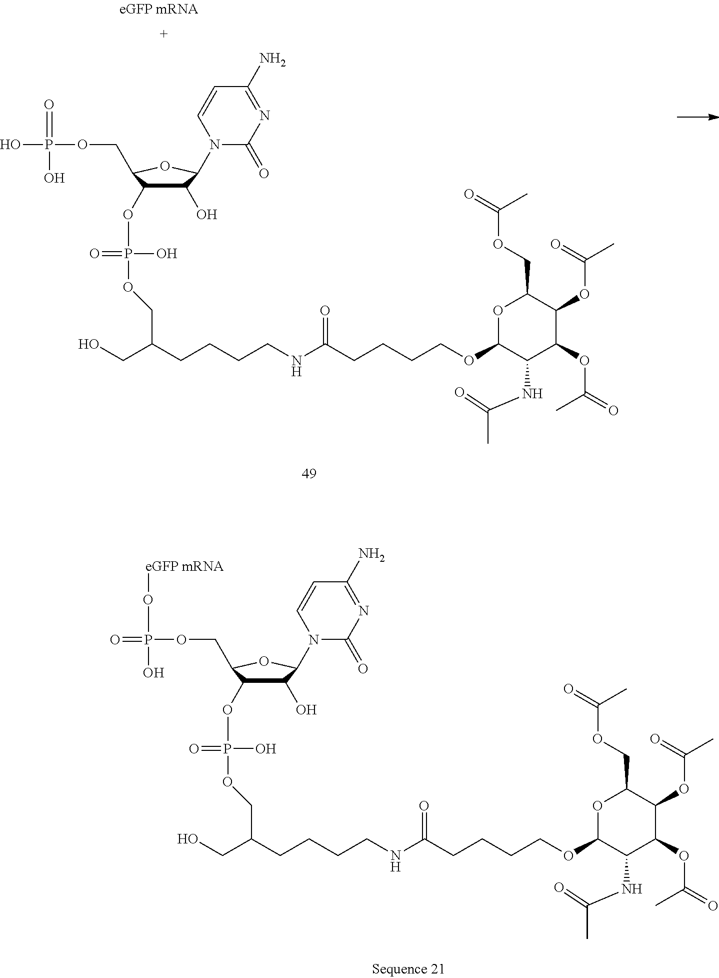

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 49 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.789 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 80% Buffer B in Buffer A over 29.8 minutes was performed. The presence of the new product, Sequence 21 was confirmed by HPLC.

Retention Time eGFP mRNA: 9.335 min.; Retention Time Sequence 21: 11.407 min.

### Example S27: Synthesis of Sequence 22

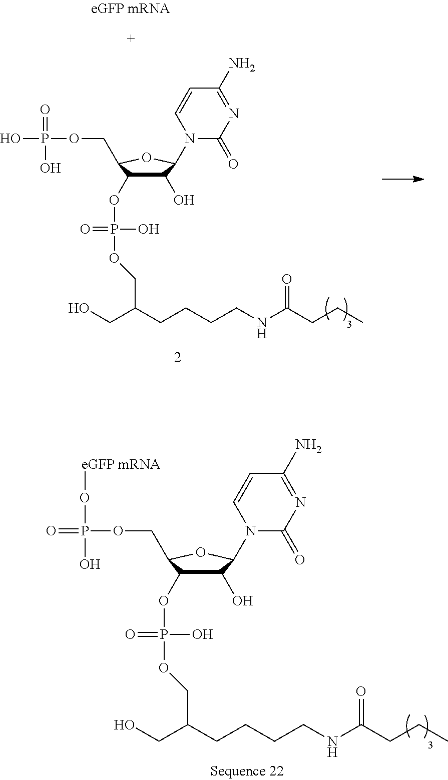

Compound 2 was purified on an anion exchange column eluting with a gradient of 0 to 35% 1M NaCl in water over 10 CVs. The desired fractions were collected, diluted to a conductivity of <5 mS/cm and purified on a reverse phase column (YMC Actus Triart C18 250×20.0 mm, I.D. S-5 μm, 12 nm) with a gradient of 0 to 20% acetonitrile in water. The desired fractions were collected and dried.

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 2 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.447 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes was performed. The presence of the new product, Sequence 22 was confirmed by HPLC.

Retention Time eGFP mRNA: 9.335 min.; Retention Time Sequence 22: 9.938 min.

### Example S28: Synthesis of Sequence 23

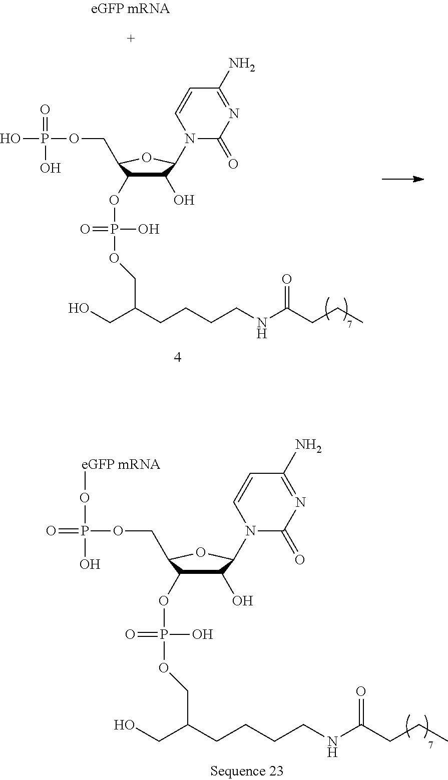

Compound 4 was purified on an anion exchange column eluting with a gradient of 0 to 35% 1M NaCl in water over 10 CVs. The desired fractions were collected, diluted to a conductivity of <5 mS/cm and purified on a reverse phase column (YMC Actus Triart C18 250×20.0 mm, I.D. S-5 μm, 12 nm) with a gradient of 0 to 20% acetonitrile in water. The desired fractions were collected and dried.

To a 1.5 mL Eppendorf tube were added RNAse free water (35.0 μL), T4 RNA Ligase reaction buffer (purchased from New England Biolabs Inc. Catalog #: M0204S, 500 mM Tris-HCl, 100 mM MgCl₂, 10 mM DTT, pH 7.5, 8.0 μL), DMSO (8.0 μL), eGFP mRNA (purchased from TriLink Biotechnologies, 2.433 mg/mL, 20.6 μL), and Murine RNase Inhibitor (purchased from New England Biolabs Inc. Catalog #M0314B, 40 U/μL, 1.0 μL). To the resulting solution were added adenosine triphosphate (100 mM, 0.8 μL), compound 4 (Na salt, 1 mM, 4.0 μL), and T4 RNA Ligase 1 (purchased from New England Biolabs Inc. Catalog #: M0204S, 10 U/μL, 2.6 μL) and thoroughly mixed. The reaction was incubated at room temperature for 21 hours and the mRNA was purified with an Oligo dT column (see above for general procedure). Desired fractions were collected and concentrated to 0.447 mg/mL. Purification was carried out by IPRP HPLC using a DNAPac RP 4 μm 3.0×50 mm column (purchased from Thermofisher Catalog #: 088920) eluting with a gradient of 40% Buffer B (100 mM TEAA, 1 mM EDTA, 25% ACN, pH 7.3) in Buffer A (100 mM TEAA, 1 mM EDTA, pH 7.3) to 60% Buffer B in Buffer A over 14.9 minutes was performed. The presence of the new product, Sequence 23 was confirmed by HPLC.

Retention Time eGFP mRNA: 9.335 min.; Retention Time Sequence 23: 14.637 min.

### Example P1: Purification of 15-Mer Oligonucleotide Models SEQ ID NO:1 and Sequence 1a

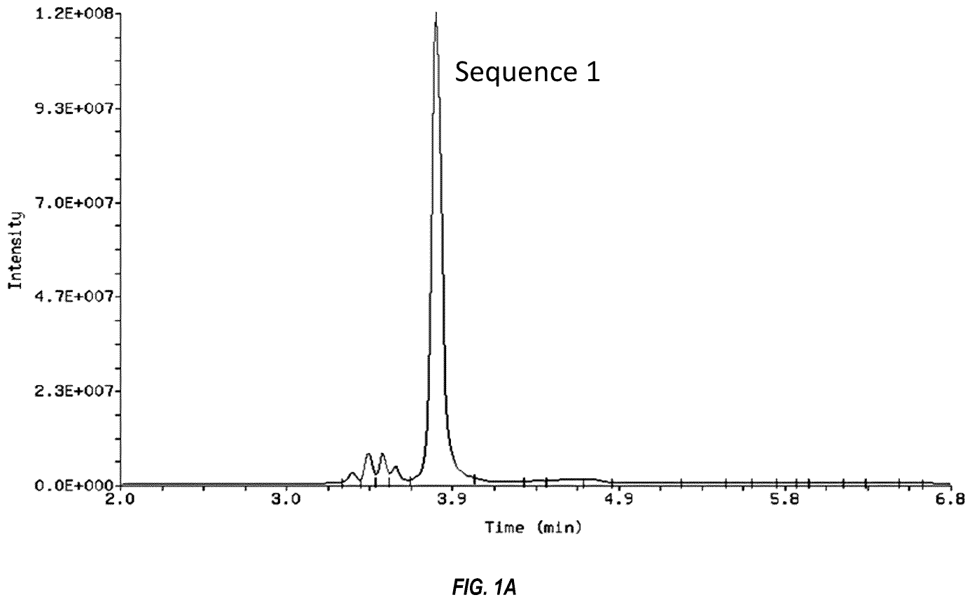

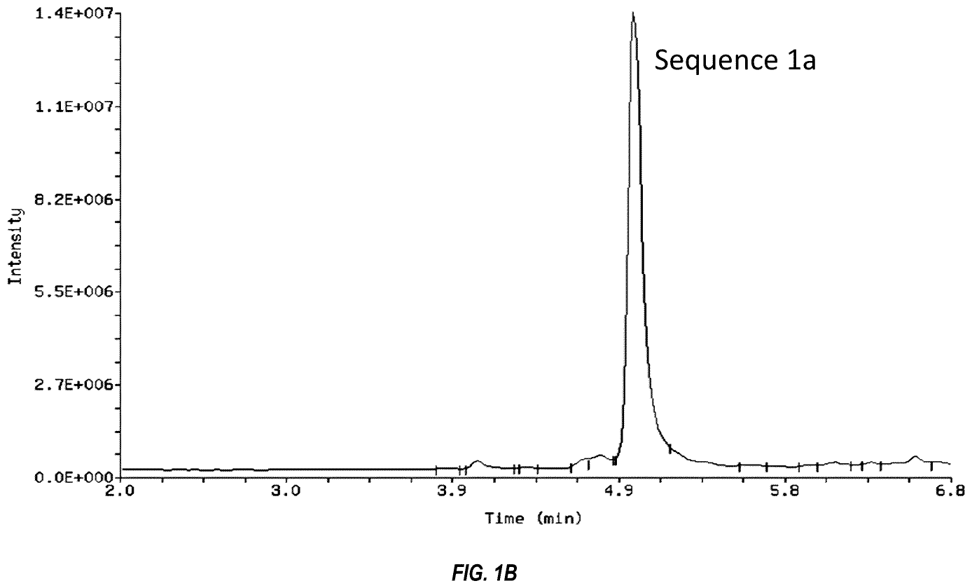

SEQ ID NO:1 is a 15-mer oligonucleotide with a linker capable of binding a purification handle. Sequence 1a is the same 15-mer oligonucleotide after the linker was covalently bound to the purification handle. This purification handle is a pentyl. FIG. 1 shows that the two 15-mer oligos were easily separated by HPLC. Similar results were observed with HPLC separation of the following pairs of 15-mer sequences SEQ ID NO:1 and Sequence 1b; and for 15-mer sequences SEQ ID NO:1 and Sequence 1c. This experiment shows that a hydrophobic group allows to separate 15-mer oligonucleotides using HPLC.

### Example P2: Purification of 40-Mer Oligonucleotide Models Sequence 2a and Sequence 2b

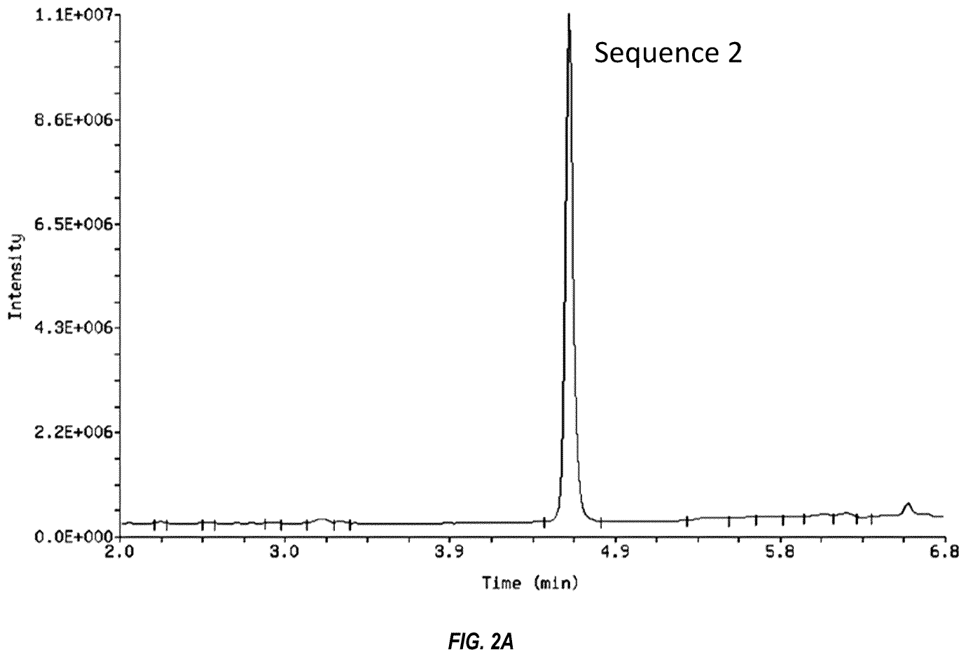

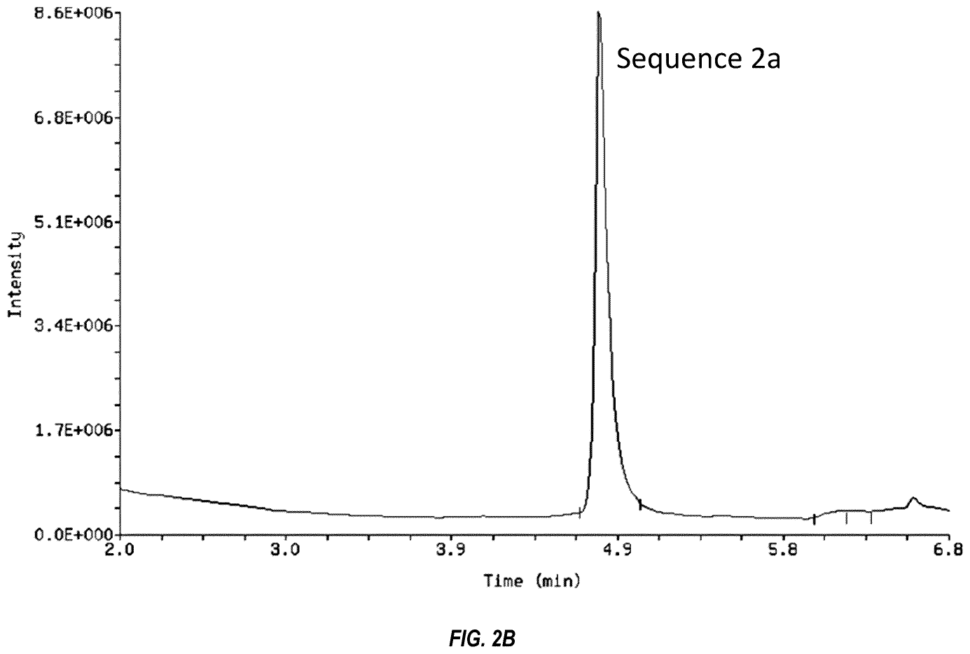

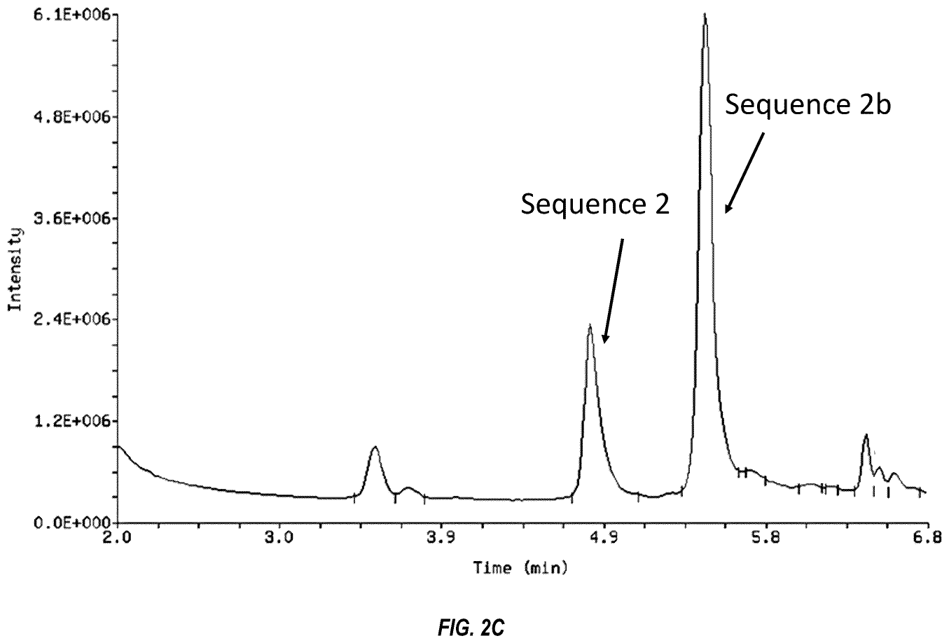

SEQ ID NO:2 is a 39-mer oligonucleotide. Sequence 2a was produced following ligation of SEQ ID NO:2 with compound 1, which is a modified nucleotide with a linker capable of binding a purification handle. Sequence 2b was produced following ligation of SEQ ID NO:2 with compound 2, which is a modified nucleotide with a covalently bound purification handle but otherwise identical to compound 1. This purification handle is a pentyl. FIG. 2 shows that when a modified nucleotide not bearing a purification handle (compound 1) was ligated to the 39-mer oligonucleotide (SEQ ID NO:2) it was impossible to separate the new 40-mer oligonucleotide Sequence 2a from the original 39-mer oligonucleotide (SEQ ID NO:2) using HPLC under the conditions set forth in Example S8. But when a modified nucleotide bearing a purification handle (compound 2) was ligated to the 39-mer oligonucleotide (SEQ ID NO:2) the new 40-mer oligonucleotide Sequence 2b was easily separable from the original 39-mer oligonucleotide (SEQ ID NO:2), as well as from Sequence 2a (which differs from Sequence 2b only by the purification handle) using HPLC. This experiment shows that a hydrophobic group allows to separate 40-mer oligonucleotides using HPLC. These 40-mer sequences are a model for purifying mRNA molecules.

### Example P3: Purification of 54-Mer Oligonucleotide Models Sequence 2c and Sequence 2d

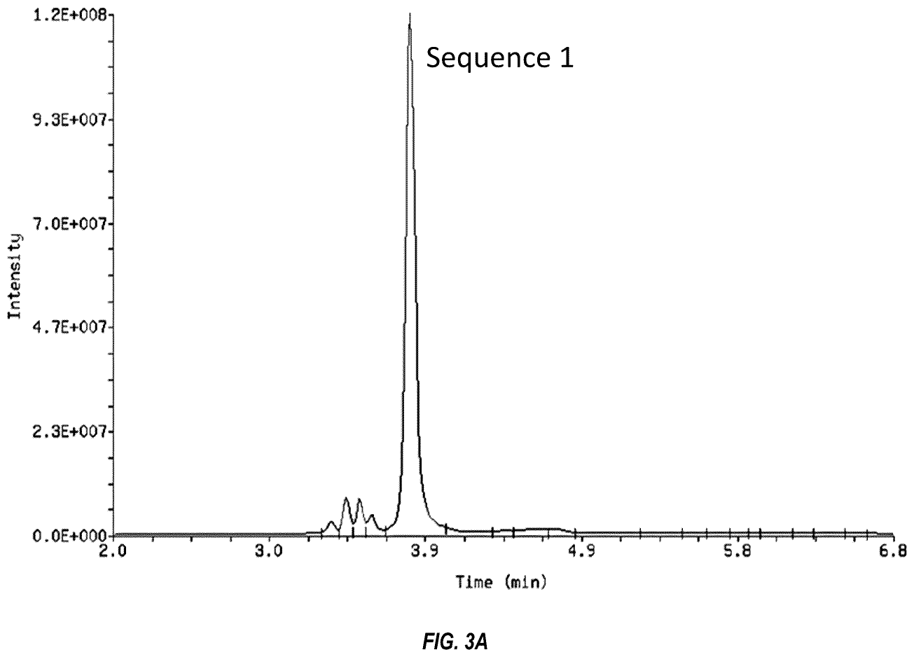

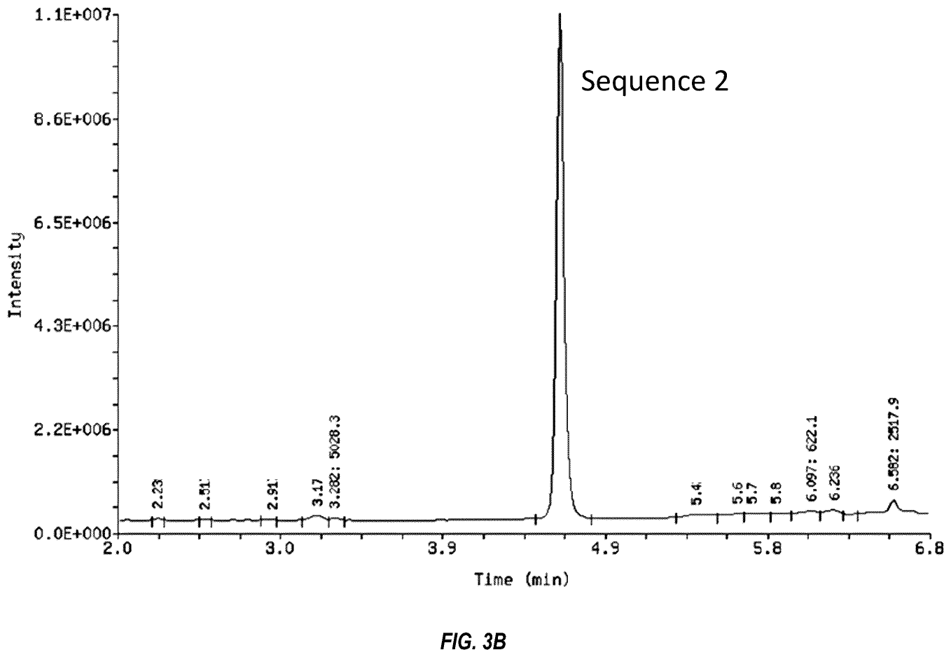

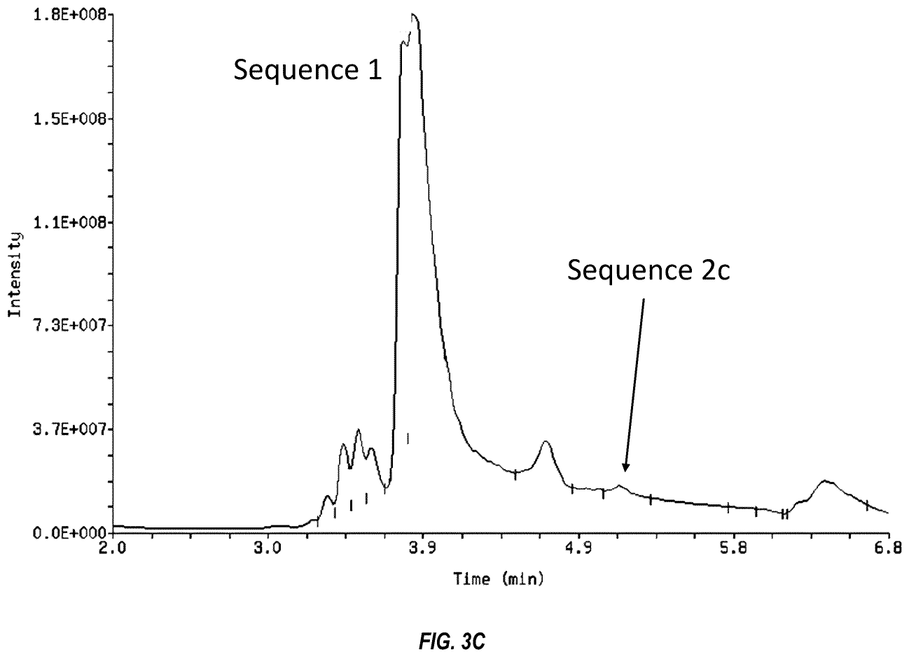

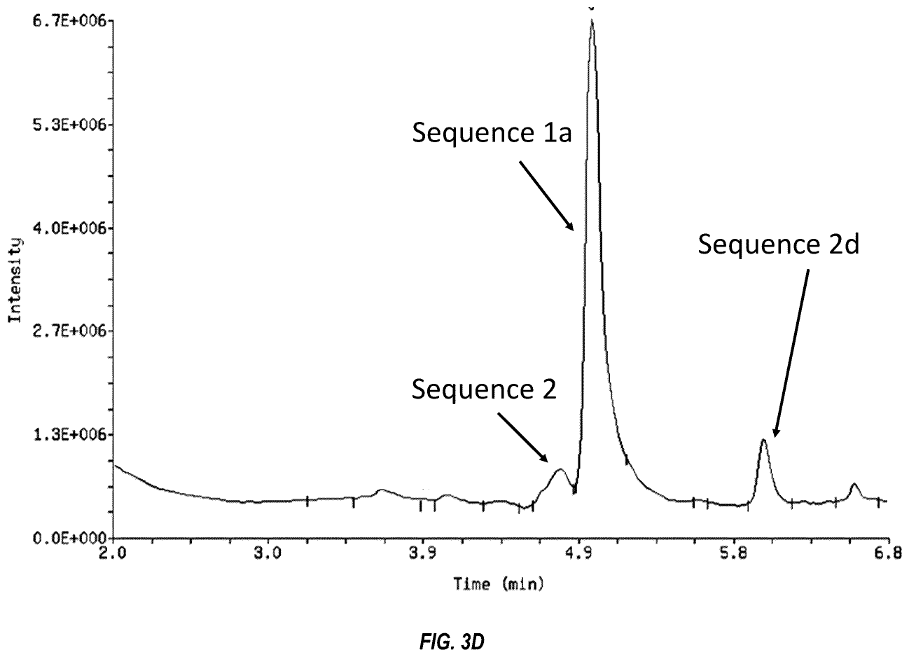

SEQ ID NO:2 is a 39-mer oligonucleotide. SEQ ID NO:1 is a 15-mer oligonucleotide with a linker capable of binding a purification handle. Sequence 2c was produced following ligation of SEQ ID NO:2 with SEQ ID NO:1. Sequence 2d was produced following ligation of SEQ ID NO:2 with Sequence 1a, which is the same oligonucleotide as SEQ ID NO:1 except that the linker is covalently bound to the purification handle. Thus, Sequences 2c and 2d are identical except that Sequence 2c had only a linker capable of binding a purification handle and Sequence 2d had a purification handle covalently bound to said linker. This purification handle is a pentyl. FIG. 3 shows that the two 54-mer oligonucleotides (Sequence 2c and 2d) were easily separable using HPLC. This experiment shows that a hydrophobic group allows to separate 54-mer oligonucleotides using HPLC. These 54-mer sequences are a model for purifying mRNA molecules.

### Example P4: Purification of FLuc mRNA

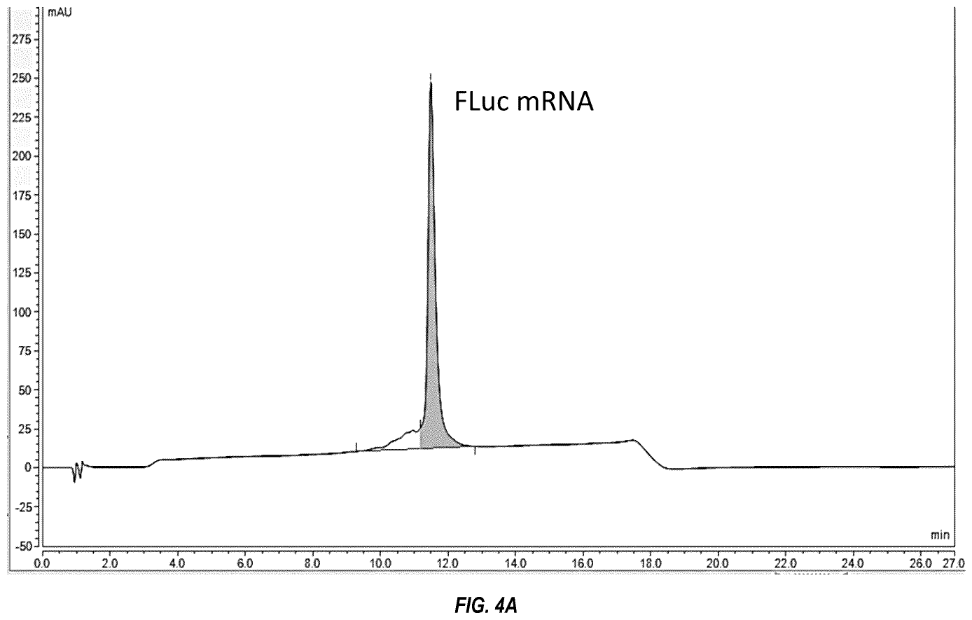

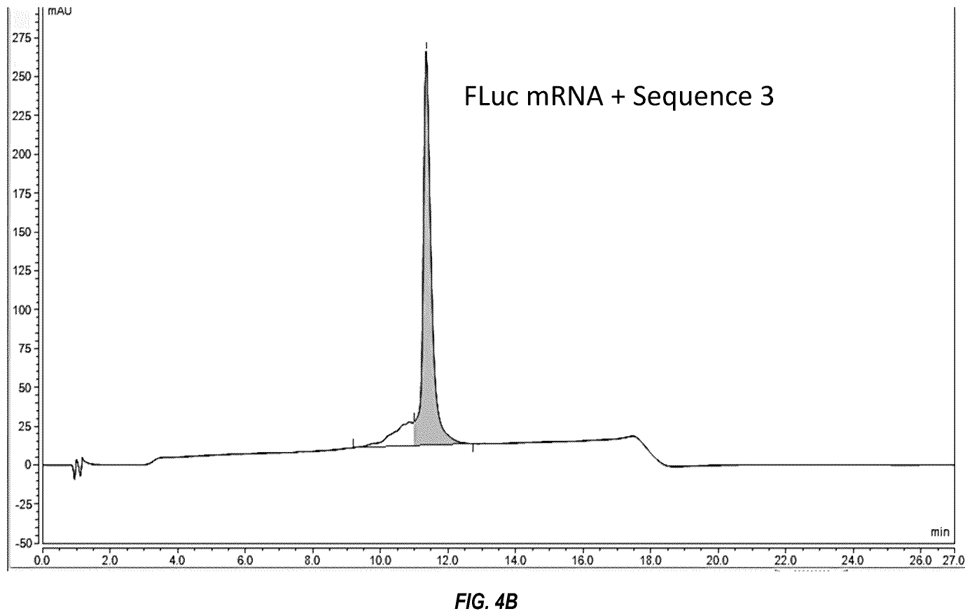

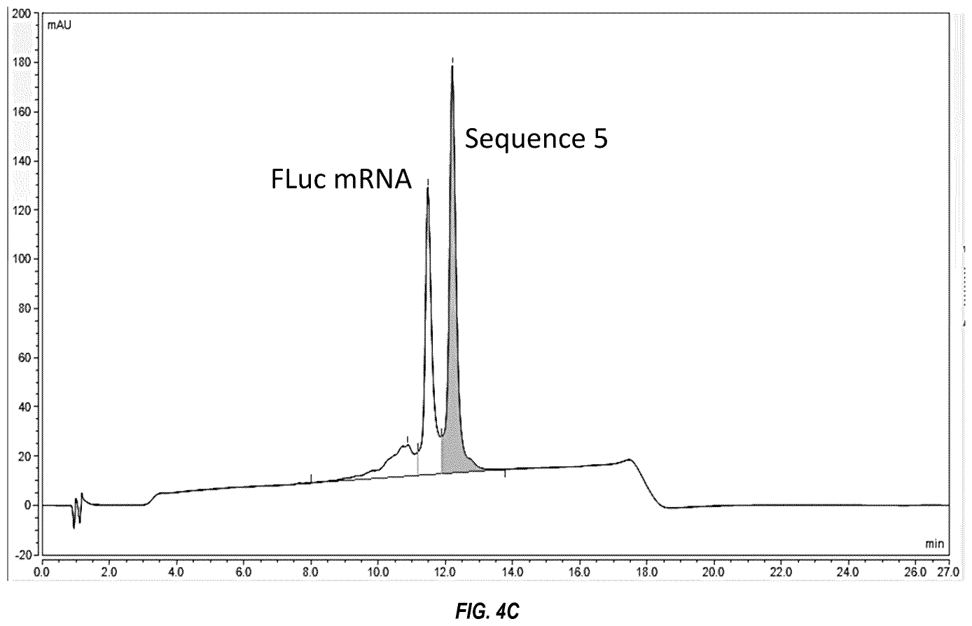

Sequence 3 was produced following ligation of SEQ ID NO:20 (FLuc mRNA) with compound 1, which is a modified nucleotide with a linker capable of binding a purification handle. Sequence 5 was produced following ligation of SEQ ID NO:20 (FLuc mRNA) with compound 2, which is a modified nucleotide identical to compound 1 but with a covalently bound purification handle. This purification handle is a pentyl. FIG. 4 shows that the modified FLuc mRNA (Sequence 3) cannot be separated from SEQ ID NO:20 (FLuc mRNA) using HPLC but modified FLuc mRNA (Sequence 5) was easily separated from SEQ ID NO:20 (FLuc mRNA) using HPLC. This experiment shows that a hydrophobic group allows to purify FLuc mRNA using HPLC.

### Example P5: Purification of FLuc mRNA

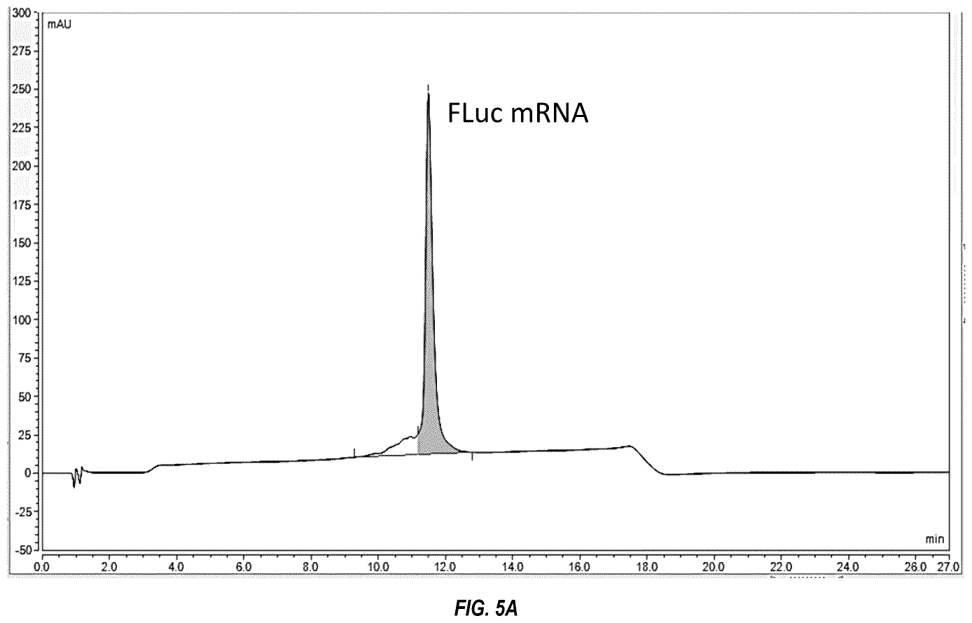

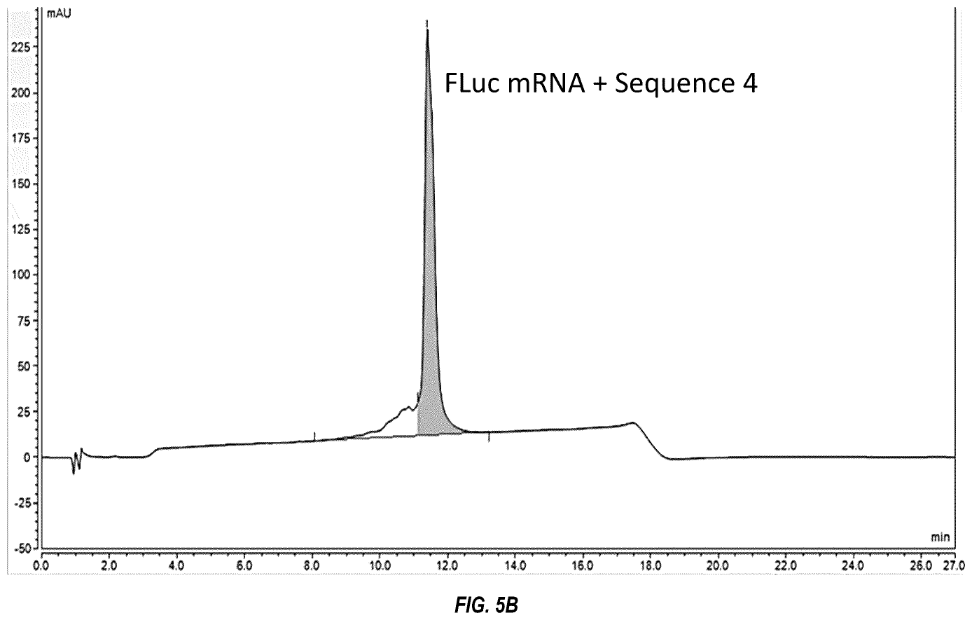

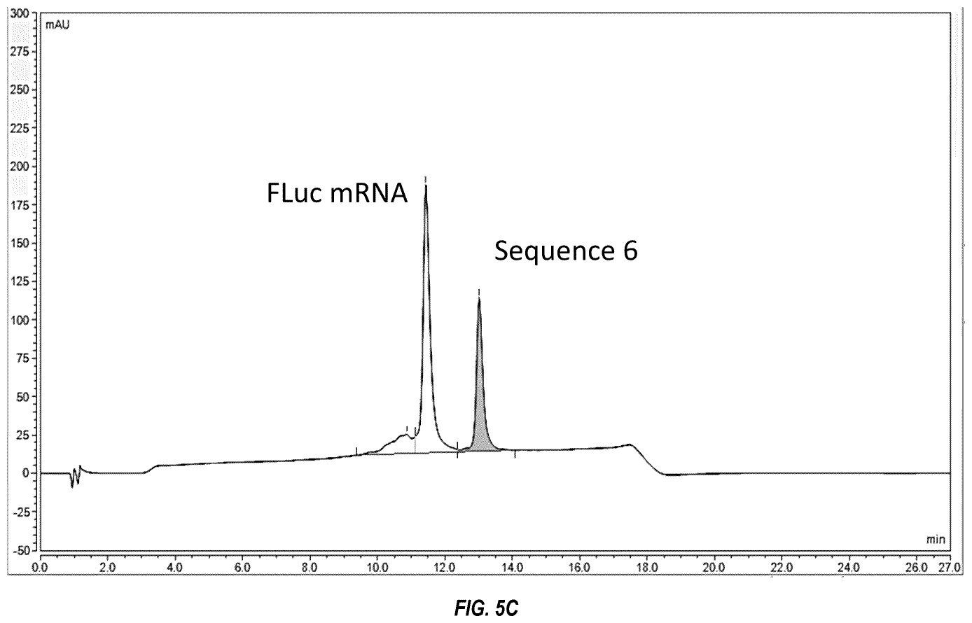

Sequence 4 was produced following ligation of SEQ ID NO:20 (FLuc mRNA) with SEQ ID NO:1, which is a 15-mer oligonucleotide with a linker capable of binding a purification handle. Sequence 6 was produced following ligation of SEQ ID NO:20 (FLuc mRNA) with Sequence 1a, which is the same 15-mer oligonucleotide as SEQ ID NO:1 after the linker was covalently bound to a purification handle. This purification handle is a pentyl. FIG. 5 shows that the modified FLuc mRNA (Sequence 4) cannot be separated from SEQ ID NO:20 (FLuc mRNA) using HPLC but modified FLuc mRNA (Sequence 6) was easily separated from SEQ ID NO:20 (FLuc mRNA) using HPLC. This experiment shows that a hydrophobic group allows to purify FLuc mRNA using HPLC.

### Example P6: Purification of FLuc mRNA

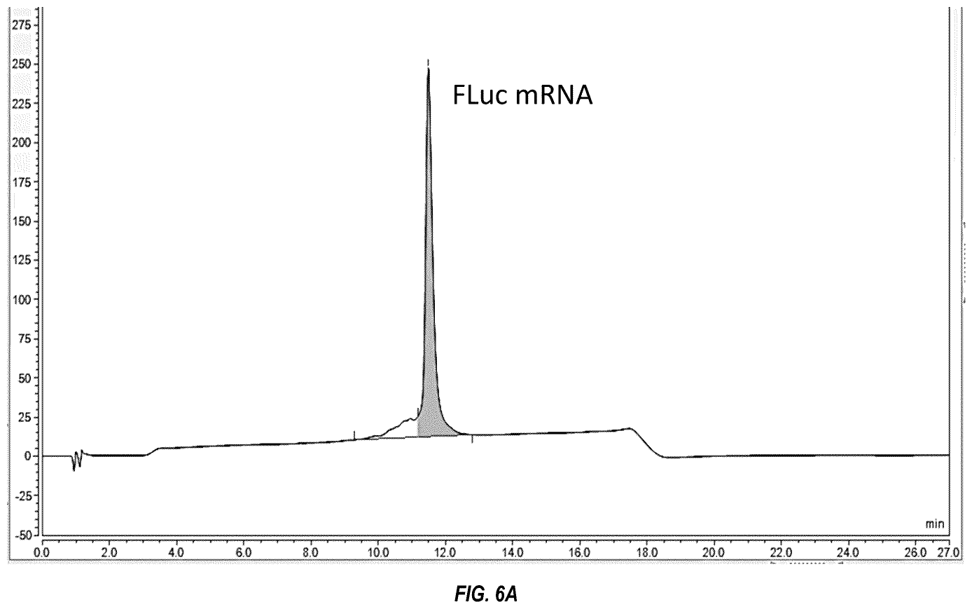

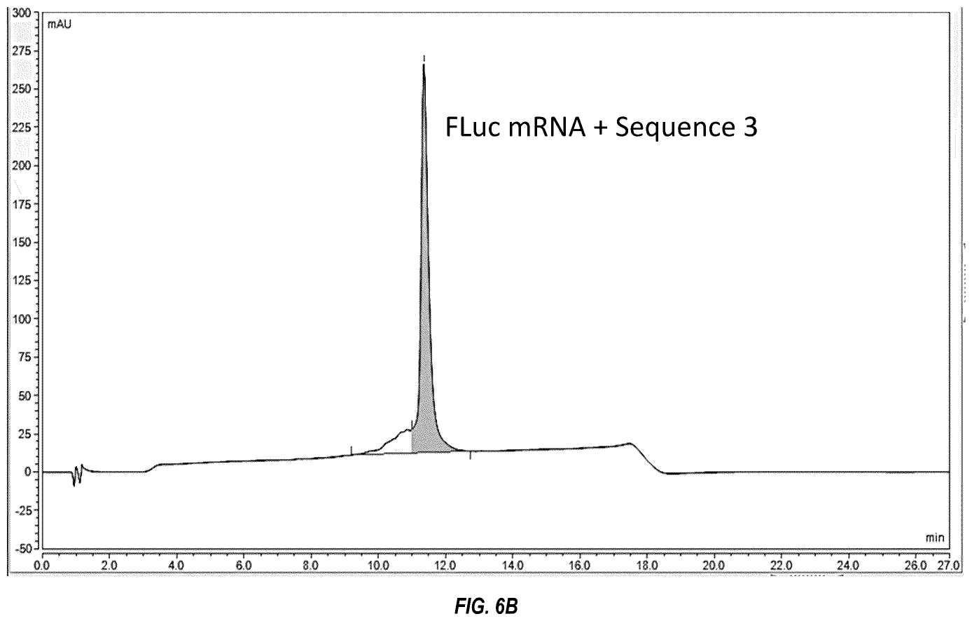

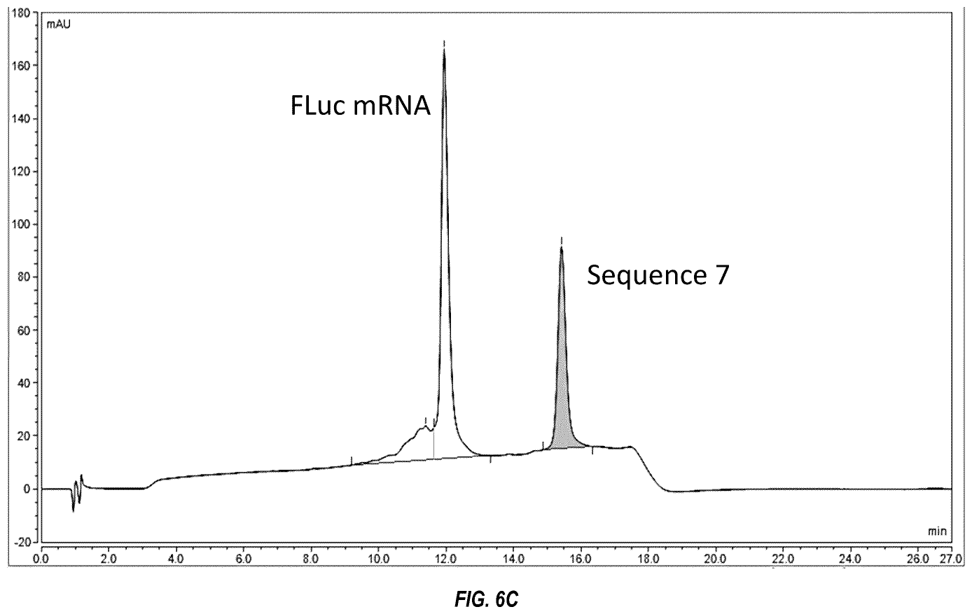

Sequence 3 was produced following ligation of SEQ ID NO:20 (FLuc mRNA) with compound 1, which is a modified nucleotide with a linker capable of binding a purification handle. Sequence 7 was produced following ligation of SEQ ID NO:20 (FLuc mRNA) with compound 4, which is a modified nucleotide identical to compound 1 but with a covalently bound purification handle. This purification handle, an octyl, is longer than that of Sequence 5. FIG. 6 shows that the modified FLuc mRNA (Sequence 3) cannot be separated from SEQ ID NO:20 (FLuc mRNA) using HPLC but modified FLuc mRNA (Sequence 7) was easily separated from SEQ ID NO:20 (FLuc mRNA) using HPLC. This experiment shows that a hydrophobic group allows to purify FLuc mRNA using HPLC. The experiment also shows that as the purification handle becomes more hydrophobic (longer carbon chain compared to the one used in example P4) the separation on HPLC becomes greater.

### Example P7: Purification of FLuc mRNA

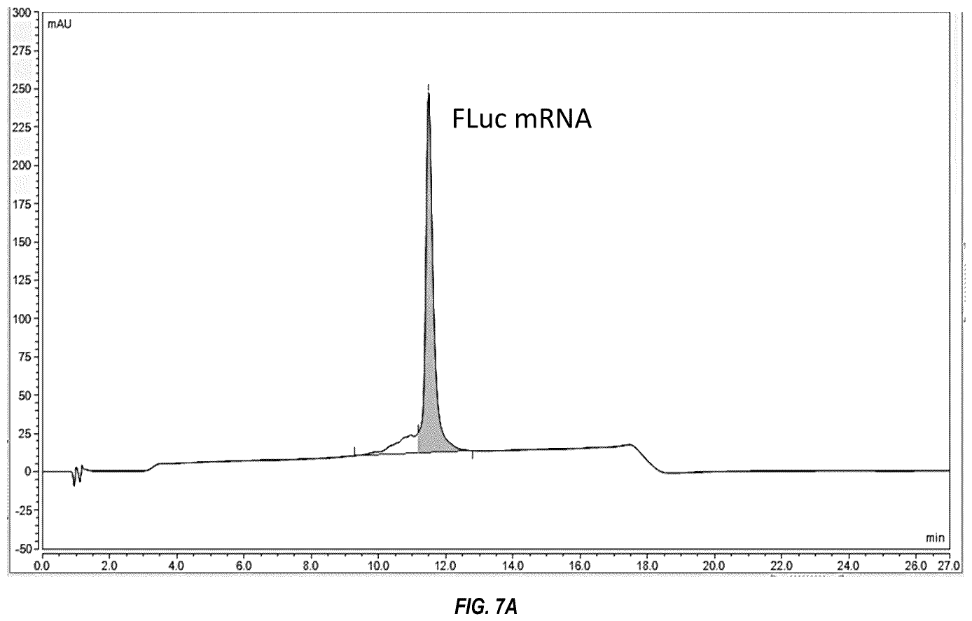

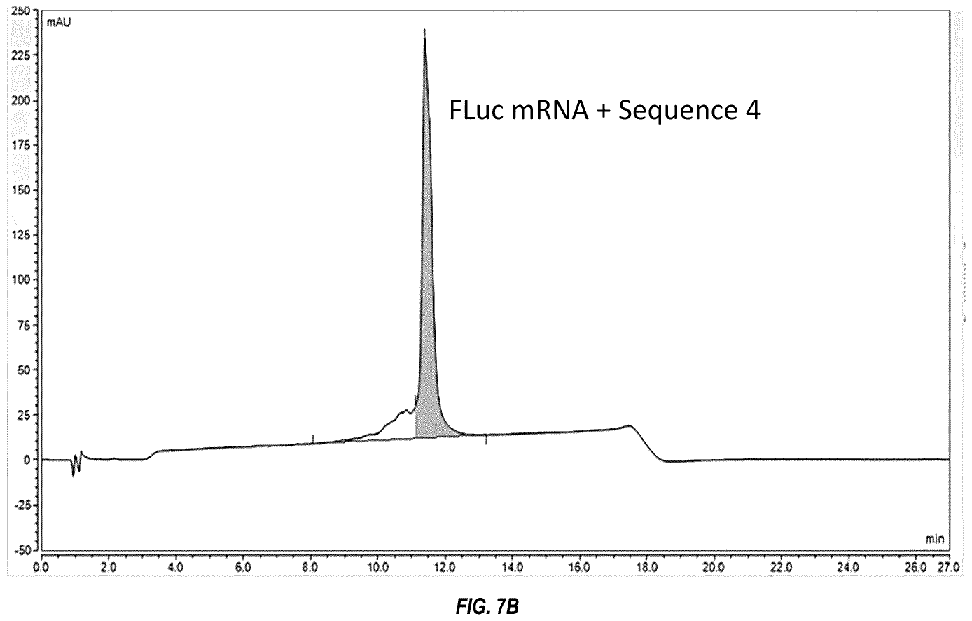

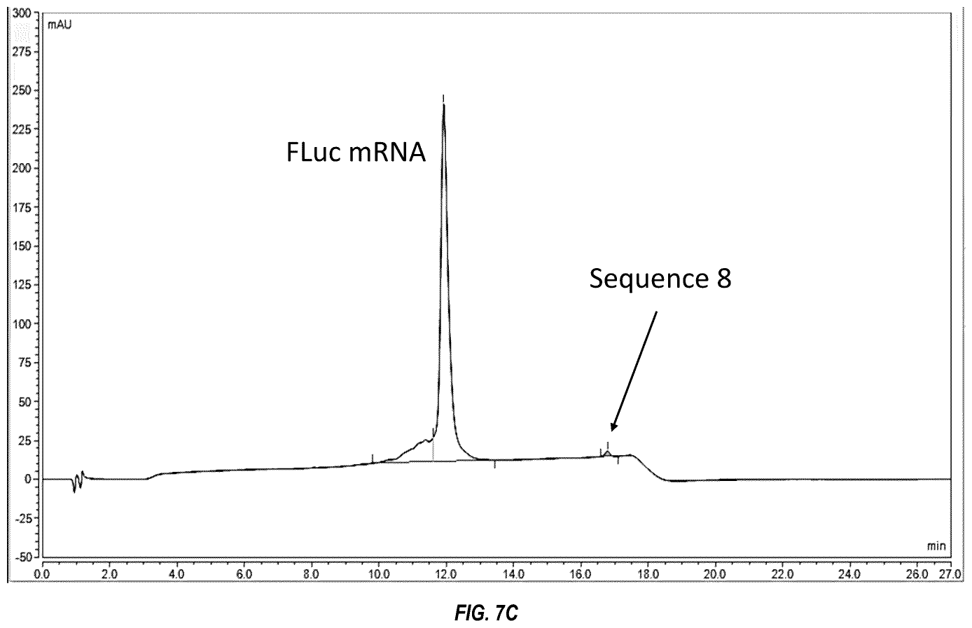

Sequence 4 was produced following ligation of SEQ ID NO:20 (FLuc mRNA) with SEQ ID NO:1, which is a 15-mer oligonucleotide with a linker capable of binding a purification handle. Sequence 8 was produced following ligation of SEQ ID NO:20 (FLuc mRNA) with Sequence 1b, which is the same 15-mer oligonucleotide as SEQ ID NO:1 except that the linker is covalently bound to a purification handle. This purification handle is an octyl. FIG. 7 shows that the modified FLuc mRNA (Sequence 4) cannot be separated from SEQ ID NO:20 (FLuc mRNA) using HPLC but modified FLuc mRNA (Sequence 8) was easily separated from SEQ ID NO:20 (FLuc mRNA) using HPLC. This experiment shows that a hydrophobic group allows to purify FLuc mRNA using HPLC. The experiment also shows that as the purification handle becomes more hydrophobic (longer carbon chain compared to the one used in example P5) the separation on HPLC becomes greater.

### Example P8: Purification of FLuc mRNA

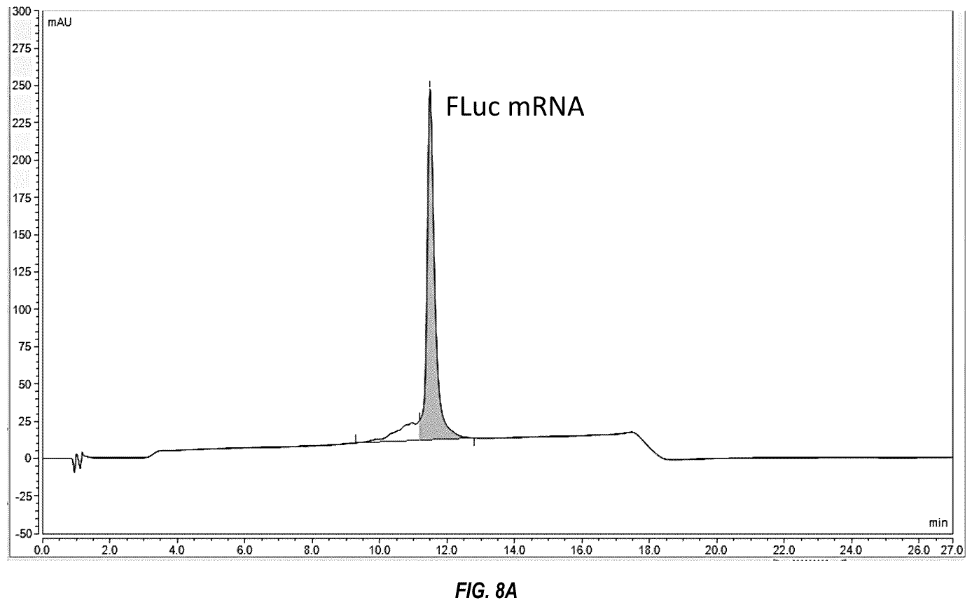

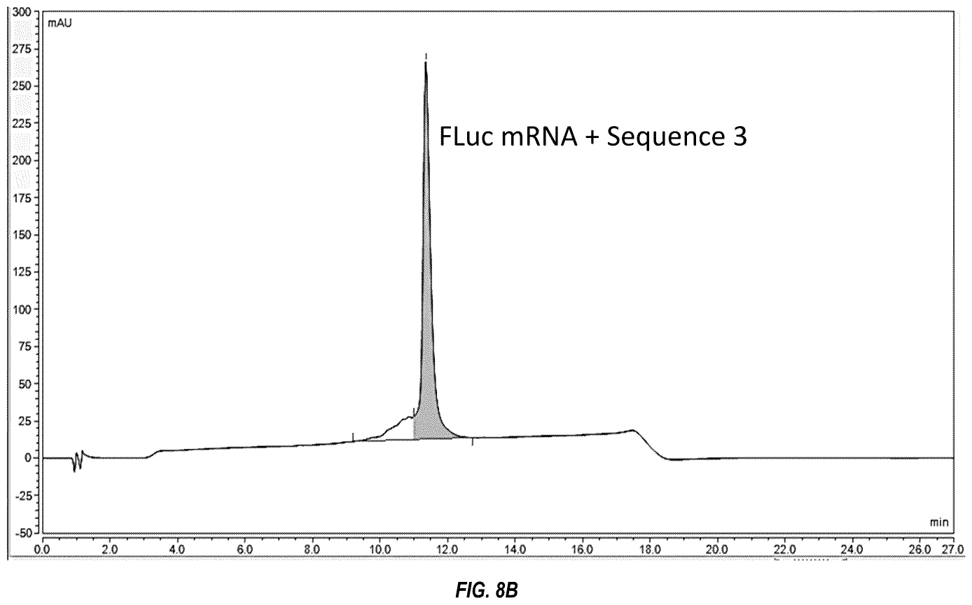

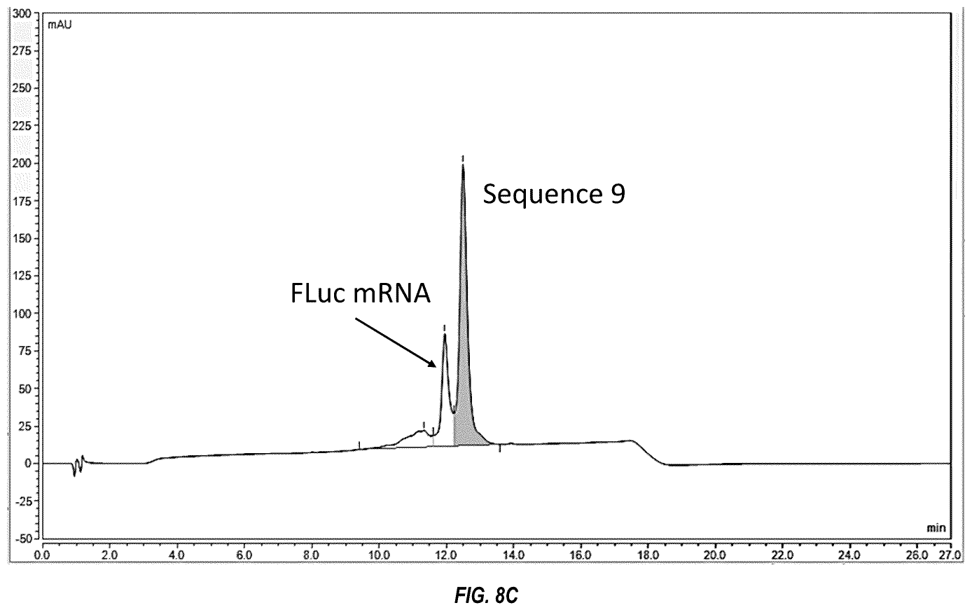

Sequence 3 was produced following ligation of SEQ ID NO:20 (FLuc mRNA) with compound 1, which is a modified nucleotide with a linker capable of binding a purification handle. Sequence 9 was produced following ligation of SEQ ID NO:20 (FLuc mRNA) with compound 6, which is a modified nucleotide identical to compound 1 except for having a covalently bound purification handle. This purification handle is benzyl. FIG. 8 shows that the modified FLuc mRNA (Sequence 3) cannot be separated from SEQ ID NO:20 (FLuc mRNA) using HPLC but modified FLuc mRNA (Sequence 9) was separated from SEQ ID NO:20 (FLuc mRNA) using HPLC. This experiment shows that a hydrophobic group allows to purify FLuc mRNA using HPLC. The experiment also compared an aliphatic purification handle (see Examples P4 and P6) vs aromatic purification handle (benzyl). It appears that aliphatic purification handles achieve a better separation using HPLC.

### Example P9: Purification of FLuc mRNA

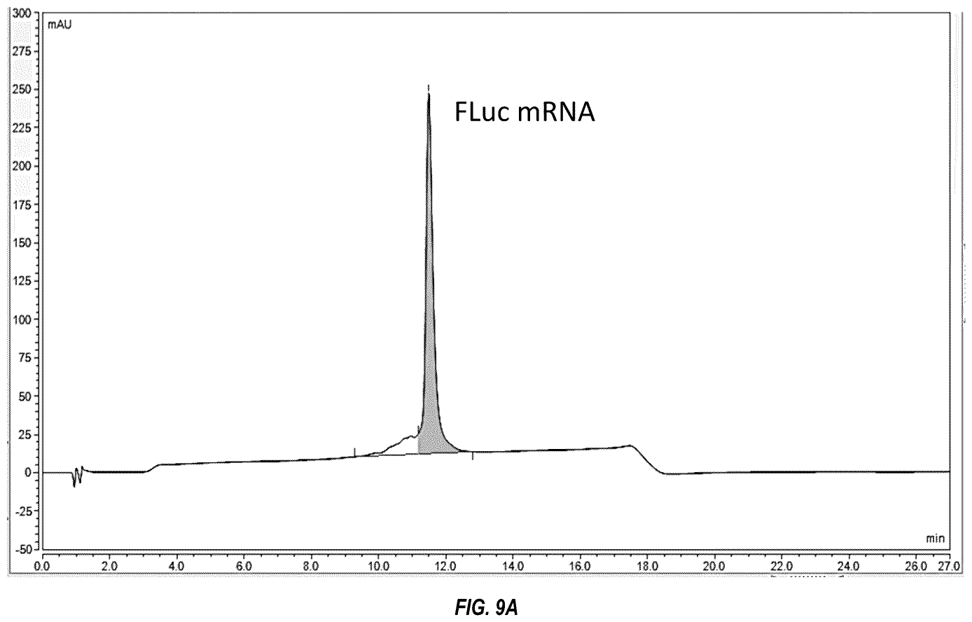

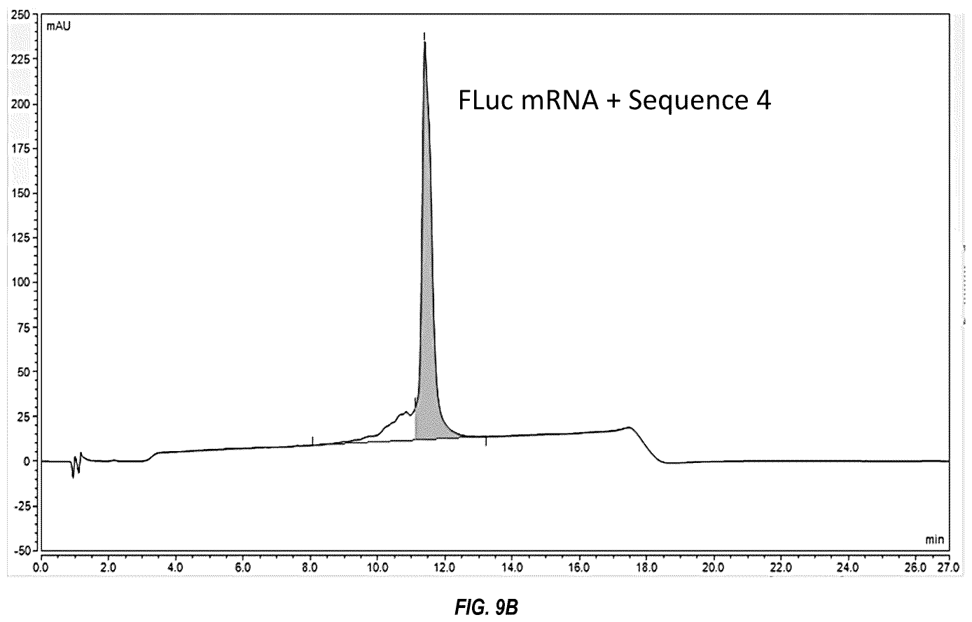

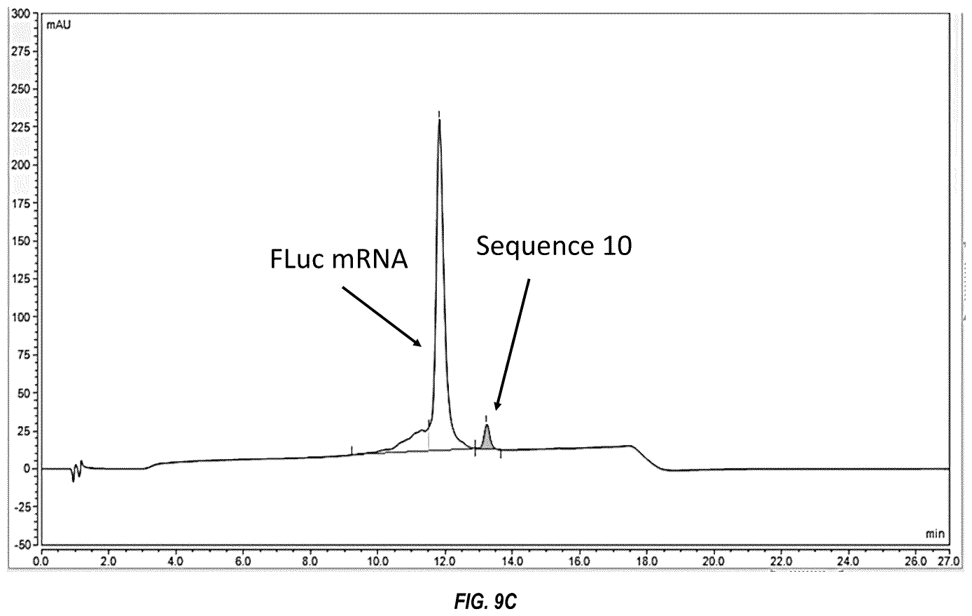

Sequence 4 was produced following ligation of SEQ ID NO:20 (FLuc mRNA) with SEQ ID NO:1, which is a 15-mer oligonucleotide with a linker capable of binding a purification handle. Sequence 10 was produced following ligation of SEQ ID NO:20 (FLuc mRNA) with Sequence 1c, which is the same 15-mer oligonucleotide as SEQ ID NO:1 except that the linker was covalently bound to a purification handle. This purification handle is benzyl. FIG. 9 shows that the modified FLuc mRNA (Sequence 4) cannot be separated from SEQ ID NO:20 (FLuc mRNA) using HPLC but modified FLuc mRNA (Sequence 10) was easily separated from SEQ ID NO:20 (FLuc mRNA) using HPLC. This experiment shows that a hydrophobic group allows to purify FLuc mRNA using HPLC. It appears that aliphatic purification handles allow achievement of a better separation using HPLC.

### Example P10: Purification of eGFP mRNA

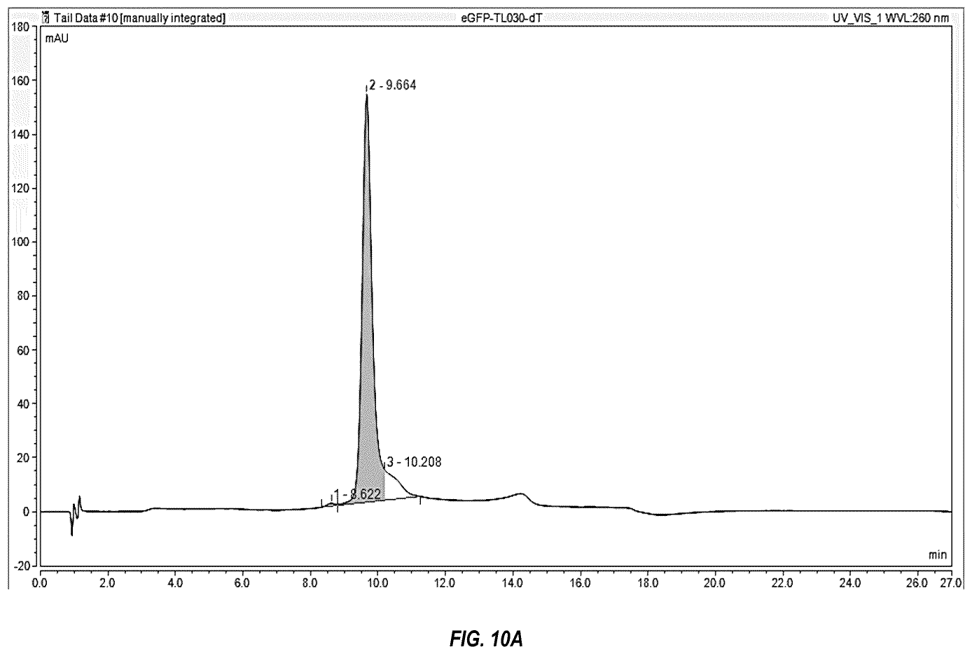

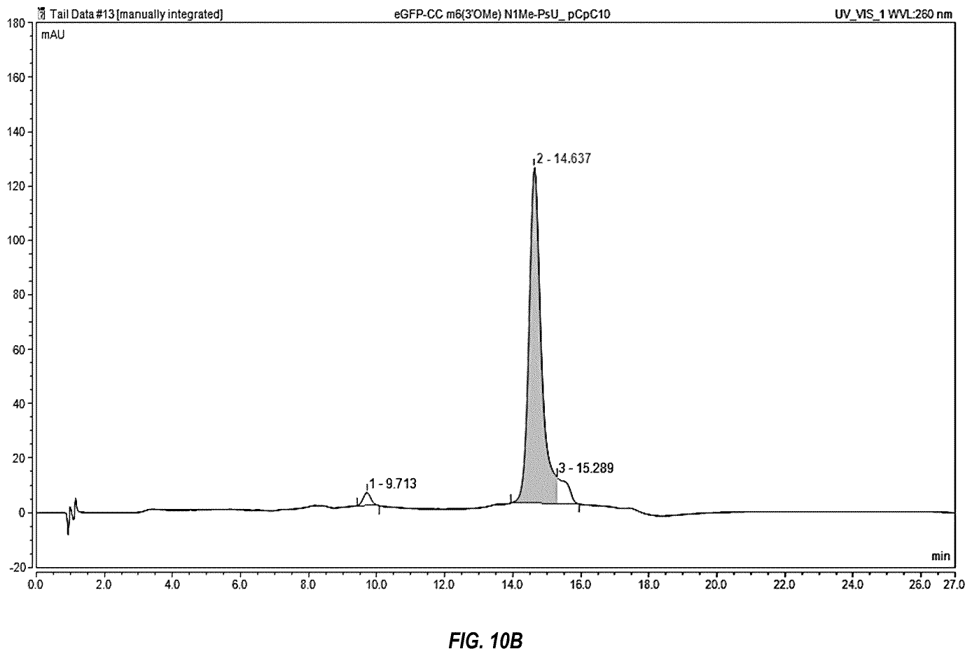

Sequence 23 was produced following ligation of SEQ ID NO:21 (eGFP mRNA) with compound 4. Modified eGFP mRNA (Sequence 23) was easily separated from SEQ ID NO:21 (eGFP mRNA) using HPLC, see FIGS. 10A and 10B. This experiment shows that a hydrophobic group allows for easy purification of eGFP mRNA using HPLC (as was shown with FLuc mRNA).

### Example P11: Purification of eGFP mRNA

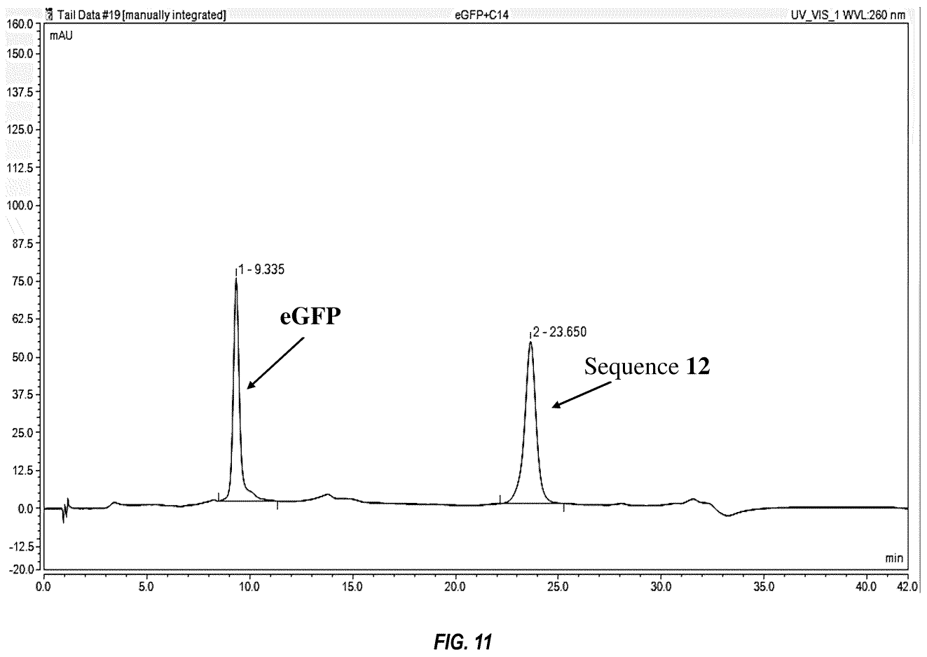

Sequence 12 was produced following ligation of SEQ ID NO:21 (eGFP mRNA) with compound 18. FIG. 11 shows the co-injection of Sequence 12 and SEQ ID NO:21 (eGFP mRNA). This figure shows that unmodified eGFP mRNA can be easily separated from Sequence 12, which has only one additional nucleoside with a linker and purification handle compared to eGFP mRNA.

### Example B1: Protein Epression in Cell-Based Assays

IVT template was produced by methods known in the art. The IVT template was used to prepare FLuc encoding mRNA (SEQ ID NO:20; where all T nucleotides are 5-methoxy uridines and the cap is CleanCap AG) with different tail modifications, and eGFP encoding mRNA (SEQ ID NO:21; where all T nucleotides are N1-methyl pseudouridines and the cap is CleanCap M6) with different tail modifications by in-vitro transcription. The eGFP mRNAs were purified (via oligo dT procedure described above) prior to use in assays described below.

eGFP fluorescence assay:

The day before transfection: cells were dissociated with Trypsin, centrifuged and washed off Trypsin with dPBS, then seeded in 96 well plate as follows:

$$293 T cells : 8 \times {10}^{3} / well$$

$$A 549 cells : 10 \times {10}^{3} / well$$

The cells were selected in 150 μL of complete growth media

### Complete Growth Media:

$$293 T cells : DMEM + 10 \% FBS + 4 mM L - Glutamine + 1 mM Sodium Pyruvate .$$

$$A 549 cells : DMEM Glutamax + 10 \% FBS$$

The day of transfection: Opti-MEM and MessengerMax Transfection Reagent were allowed to reach room temperature. Two tubes were set up for each mRNA (i.e., for each tail modification).

Preparing tube 1: 0.2 μL of MessengerMax was added to 5 μL Opti-MEM (these amounts were multiplied by the number of wells for each tail modification, in this case 6 repeats per tail modification were done), and the mixture was incubated at room temperature for 10 minutes.

Preparing tube 2: During the 10-minute incubation of mixture in tube 1, 10 ng of each mRNA (with different tail modification) was added to tubes containing 5 μL of Opti-MEM each (these amounts were multiplied by the number of wells for each tail modification, in this case 6 repeats per tail modification were done).

After the 10 min incubation of mixture in tube 1, mixture of tube 2 was added to mixture of tube 1, and the new mixture was incubated for 10 minutes.

After the 10 min incubation of the two mixtures, the volume adjusted to 50 μL by adding 40 μL complete media to each tube (these amounts were multiplied by the number of wells for tail modification, in this case 6 repeats per tail mod were done).

To 96-well plates containing the cells, 50 μL of the mixture (mix of tube 1 and tube 2+40 μL complete media) was added to the cells bringing the total volume of media to 200ul.

eGFP fluorescent expression was quantified using Agilent plate reader SH1MF after 24 hours, 48 hours, 72 hours, and 96 hours.

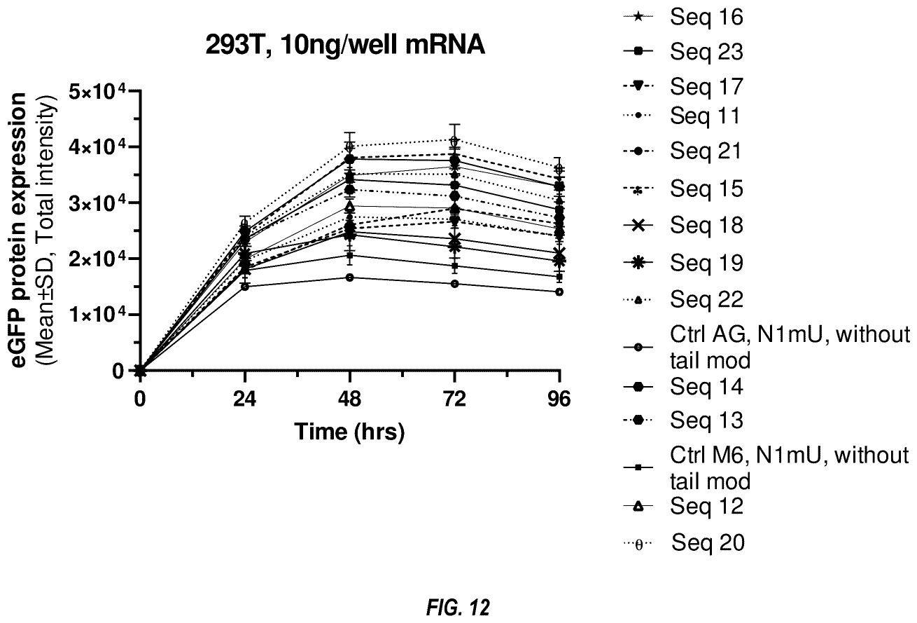

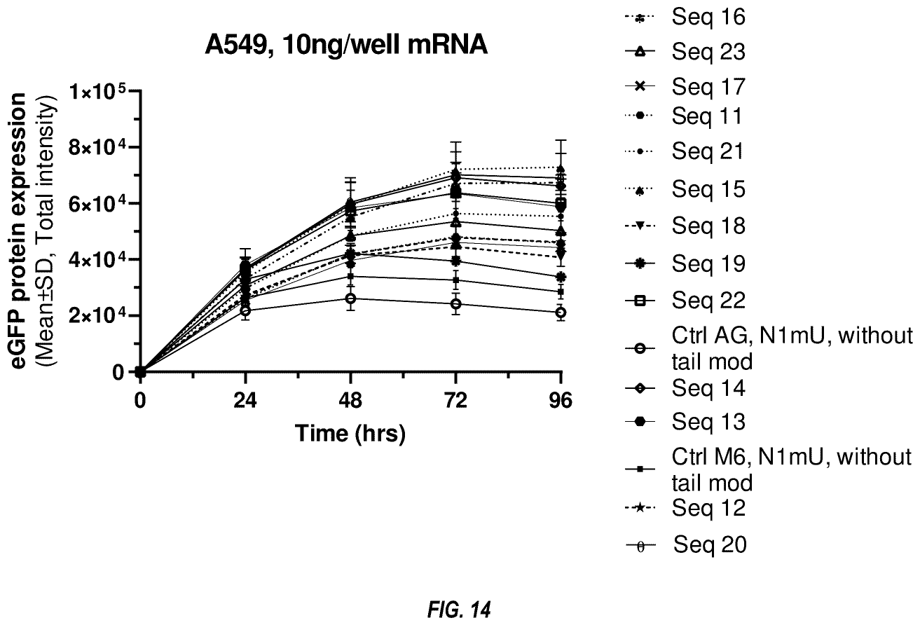

FIG. 12 (293T cells) and FIG. 14 (A549 cells) show that eGFP mRNA molecules comprising one additional nucleoside with a purification handle on the 3′end have longer half-life than eGFP mRNA molecules lacking such nucleoside with purification handle. Thus, surprisingly a purification handle not only allows for separation of mRNAs, but it stabilizes the mRNAs in addition to stabilization conferred by capping the mRNAs with m7G_{3′OMe}^{m6}A_{2′OMe}pG caps.

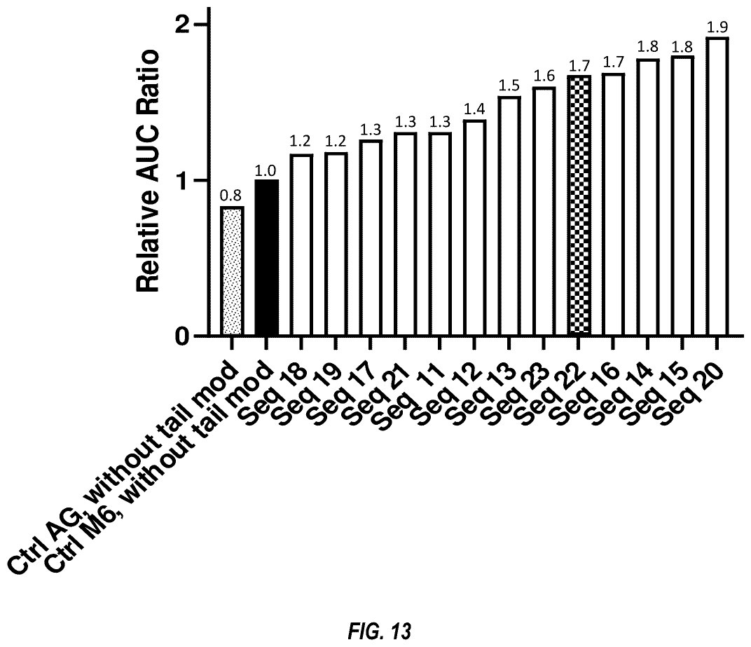

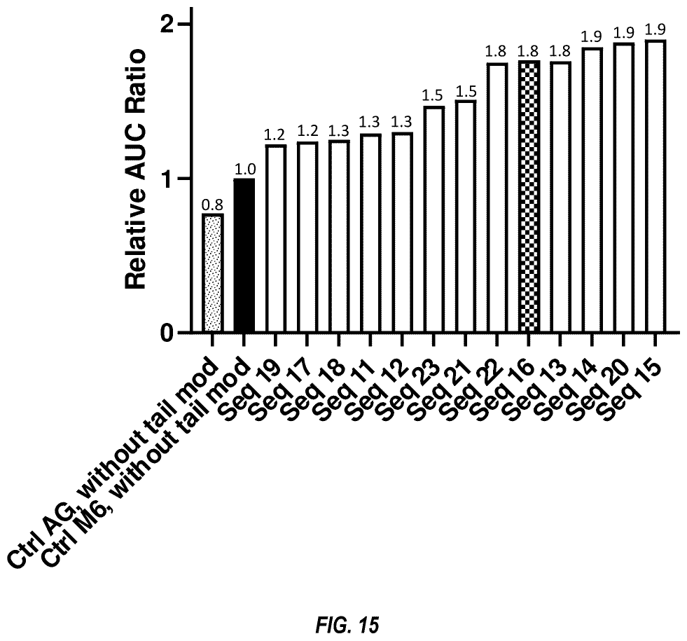

FIG. 13 (293T cells) and FIG. 15 (A549 cells) show that eGFP mRNA molecules comprising one additional nucleoside with a purification handle on the 3′end have higher overall translation yield (as detected after 96 hours) than eGFP mRNA molecules lacking such nucleoside with purification handle. Thus, surprisingly a purification handle not only allows for separation of mRNAs and results in stabilization of mRNAs in addition to stabilization conferred by capping the mRNAs with m7G_{3′OMe}^{m6}A_{2′OMe}pG caps, but the purification handle also results in mRNAs with better translation efficiency.

Not only the purification handles described herein allow for separation of eGFP mRNA molecules (SEQ ID NO:21) from corresponding eGFP mRNA molecules comprising one additional nucleoside with a purification handle, but these mRNA molecules comprising the 3′-stabilizing region with the purification handles have longer half-life and have higher translation yield overall during 96 hours.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims. The description is to be read from the perspective of one of ordinary skill in the art; therefore, information well known to the skilled artisan is not necessarily included.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1-69. (canceled)

70. An RNA molecule comprising the structure of Formula I:
A−B (Formula I)
wherein A comprises:
a) a 5′-cap;
b) an open reading frame (ORF) encoding a protein; and
c) a poly-A region, wherein the poly-A region is 3′ to the open reading frame; and
B comprises a 3′-stabilizing region comprising 1 to 50 nucleosides, wherein one or more nucleosides within the 3′-stabilizing region comprise one or more purification handles.

71. The RNA molecule of claim 70, wherein the 3′-stabilizing region is covalently linked to a precursor RNA by a linker that can be formed by ligation.

72. The RNA molecule of claim 70, wherein the 3′-stabilizing region is covalently linked to a precursor RNA by a linker that can be formed using a polymerase.

73. The RNA molecule of claim 70, wherein the purification handle is linked to the 3′-stabilizing region via a linker (L).

74. The RNA molecule of claim 70, wherein the purification handle comprises a lipid.

75. An RNA molecule comprising the structure of Formula II:
A-B-L (Formula II)
wherein A comprises:
a) a 5′-cap;
b) an open reading frame (ORF) encoding a protein; and
c) a poly-A region, wherein the poly-A region is 3′ to the open reading frame; and
B comprises a 3′-stabilizing region comprising 1 to 50 nucleosides, wherein one or more nucleosides within the 3′-stabilizing region comprise one or more linkers (L), wherein the linker (L) is capable of binding a purification handle.

76. The RNA molecule of claim 75, wherein the 3′-stabilizing region is covalently linked to a precursor RNA by a linker that can be formed by ligation.

77. The RNA molecule of claim 76, wherein the 3′-stabilizing region is covalently linked to a precursor RNA using a polymerase.

78. The RNA molecule of claim 75, wherein one or more purification handles are covalently linked to one or more nucleosides or one or more linkers within the 3′-stabilizing region.

79. The RNA molecule of claim 70, wherein the purification handle comprises a lipid.

80. The RNA molecule of claim 70, wherein the 3′-stabilizing region forms a secondary structure, optionally wherein the secondary structure is a hairpin loop.

81. The RNA molecule of claim 70, wherein the 3′-stabilizing region comprises one or more unmodified nucleosides and one or more unmodified internucleotide linkages.

82. The RNA molecule of claim 70, wherein the 3′-stabilizing region comprises one or more modified nucleosides and/or one or more modified internucleotide linkages.

83. The RNA molecule of claim 82, wherein the modified nucleoside comprises a modified nucleobase and/or a modified sugar.

84. The RNA molecule of claim 82, wherein the modified nucleoside comprises a modified nucleobase and the modified nucleobase is
(a) a modified uracil, a modified cytosine, a modified guanine, or a modified adenine;
(b) pseudouracil (y), 2-thio-uracil, 4-thio-uracil, 4-thio-pseudouridine, 2-thio-pseudouridine, 5-hydroxy-uracil, 5-halo-uracil, 3-methyl-uracil, 5-aza-uracil, or 2-thio-5-aza-uracil;
(c) 5-aza-cytosine, 6-aza-cytosine, pseudoisocytidine, 3-methyl-cytosine, 5-methyl-cytosine, 5-halo-cytosine, 2-thio-cytosine, or 2-thio-5-methyl-cytosine;
(d) 2-amino-purine, 2,6-diaminopurine, 2-amino-6-halo-purine, 6-halo-purine, 2-amino-6-methyl-1-purine, 8-azido-adenine, 7-deaza-adenine, N6-methyl-adenine, or 2-methylthio-N6-methyl-adenine; or
(e) inosine, 1-methyl-inosine, 7-cyano-7-deaza-guanine, 7-aminomethyl-7-deaza-guanine, 6-thio-1-guanine, 6-thio-7-deaza-guanine, or 6-methoxy-guanine.

85. The RNA molecule of claim 82, wherein the modified nucleoside comprises a modified sugar and the modified sugar has a 5-membered ring or a 6-membered ring, or is a modified ribose, wherein the modified ribose is 2′-thioribose, 2′, 3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, or 3′-amino-2′,3′-dideoxyribose.

86. The RNA molecule of claim 82, wherein the modified nucleoside comprises a morpholino ring, or wherein the internucleotide linkage comprises a modified phosphate.

87. The RNA molecule of claim 86, wherein (i) the internucleotide linkage comprises a modified phosphate and the modified phosphate is phosphorothioate, phosphorodithioate, thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, phenylphosphonate, ethylphosphonate, H-phosphonate, guanidinium ring, triazole ring, boranophosphate, methylphosphonate, or guanidinopropyl phosphoramidate; and/or (ii) the last nucleoside of the 3′-stabilizing region is ddC, inverted dT, 3′-phosphate nucleoside, 3′-oxime nucleoside, 3′-azidomethyl nucleoside, or 3′-methyl nucleoside.

88. The RNA molecule of claim 70, wherein the poly-A region is 10 or greater nucleosides in length.

89. The RNA molecule of claim 70, wherein the poly-A region is from 15 to 150 nucleosides in length.

90. The RNA molecule claim 70, wherein the RNA molecule is a messenger RNA (mRNA).

91. A cell comprising the RNA molecule of claim 70, optionally wherein the cell is isolated.

92. A pharmaceutical composition comprising the RNA molecule, or comprising a cell comprising the RNA molecule, of claim 70 and a pharmaceutically acceptable carrier.

93. A method of increasing the expression of a protein or a peptide of interest in a cell, comprising contacting the cell with the RNA molecule of claim 70, wherein the RNA molecule encodes the protein or peptide of interest
(a) wherein the expression is increased when compared to that of an RNA molecule without the 3′-stabilizing region;
(b) wherein the cell translates the protein or peptide of interest from the RNA molecule; or
(c) wherein the half-life is increased when compared to that of an RNA molecule without the 3′-stabilizing region,
optionally wherein the cell is isolated, in vitro, or ex vivo.

94. A method of preparing the RNA molecule of claim 70, comprising covalently joining a stabilizing region to a precursor RNA comprising a 5′-cap, an ORF encoding a protein, and a poly-A region 3′ of the ORF, wherein the stabilizing region is added 3′ to the poly-A region and the stabilizing region comprises one or more purification handles and/or one or more linkers capable of binding a purification handle.

95. A method of preventing or treating a disease in a subject in need thereof comprising introducing an effective amount of the RNA molecule of claim 70.

96. A compound of Formula (III) or (IV): 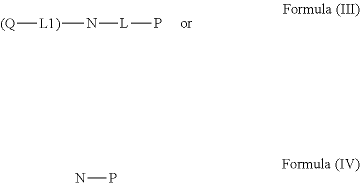
wherein N is a nucleoside;
L is a linker capable of binding a purification handle;
P is a purification handle;
Q-L1 is optionally present, wherein L1 is a linker covalently bound to N and to Q; and
Q is a hydrogen or a chain terminating nucleoside.

97. The compound of claim 96, wherein the nucleoside is a modified nucleoside.

98. The compound of claim 96, wherein the purification handle comprises a lipid.

99. A method of increasing the expression of a protein or a peptide of interest in a cell, or of increasing the half-life of an RNA molecule in a cell, comprising contacting the cell with an RNA molecule comprising the compound of Formula (III) or Formula (IV) of claim 97, wherein the RNA molecule encodes the protein or peptide of interest, wherein the expression is increased when compared to that of an RNA molecule without the compound of Formula (III) or Formula (IV), optionally wherein the cell is isolated, in vitro, or ex vivo, or the half-life is increased when compared to that of an RNA molecule without the compound of Formula (III) or Formula (IV), optionally wherein the cell is isolated, in vitro, or ex vivo.
